SEQUENCE LISTING (119 sequences)

SEQ ID NO: 1 - protein, 15 aa, Homo sapiens
ILTIHFTGHSFIYGK

SEQ ID NO: 2 - protein, 15 aa, Homo sapiens
IHSIHFSGHVFTVRK

SEQ ID NO: 3 - protein, 15 aa, Homo sapiens
KIVFKNMASRPYSIY

SEQ ID NO: 4 - protein, 15 aa, Homo sapiens
FAVFDENKSWYLEDN

SEQ ID NO: 5 - protein, 15 aa, Homo sapiens
ESNIMSTINGYVPES

SEQ ID NO: 6 - protein, 14 aa, Homo sapiens
EKDIHSGLIGPLLI

SEQ ID NO: 7 - protein, 15 aa, Homo sapiens
SHEFHAINGMIYSLP

SEQ ID NO: 8 - protein, 9 aa, Homo sapiens
IHFTGHSFI

SEQ ID NO: 9 - protein, 9 aa, Homo sapiens
IHFSGHVFT

SEQ ID NO: 10 - protein, 9 aa, Homo sapiens
FKNMASRPY

SEQ ID NO: 11 - protein, 9 aa, Homo sapiens
FDENLSWYL

SEQ ID NO: 12 - protein, 9 aa, Homo sapiens
IMSTINGYV

SEQ ID NO: 13 - protein, 9 aa, Homo sapiens
IHSGLIGPL

SEQ ID NO: 14 - protein, 9 aa, Homo sapiens
FHAINGMIY

SEQ ID NO: 15 - protein, 26 aa, Homo sapiens
PAVLQSSGLYSLSSVVTVPSSSLGTQ

SEQ ID NO: 16 - protein, 9 aa, Homo sapiens
IEDFNSGLI

SEQ ID NO: 17 - protein, 15 aa, Homo sapiens
ENLIEDFNSGLIGPL

SEQ ID NO: 18 - protein, 9 aa, Homo sapiens
VKDLNSGLI

SEQ ID NO: 19 - protein, 15 aa, Homo sapiens
VDLVKDLNSGLIGAL

SEQ ID NO: 20 - protein, 9 aa, Homo sapiens
KIVFKNMAS

SEQ ID NO: 21 - protein, 15 aa, Homo sapiens
DTLKIVFKNMASRPY

SEQ ID NO: 22 - protein, 9 aa, Homo sapiens
VITLKNMAS

SEQ ID NO: 23 - protein, 15 aa, Homo sapiens
DTVVITLKNMASHPV

SEQ ID NO: 24 - protein, 9 aa, Homo sapiens
FKNQASRPY

SEQ ID NO: 25 - protein, 15 aa, Homo sapiens
LTIFKNQASRPYNIY

SEQ ID NO: 26 - protein, 9 aa, Homo sapiens
LKNMASHPV

SEQ ID NO: 27 - protein, 15 aa, Homo sapiens
VITLKNMASHPVSLH

SEQ ID NO: 28 - protein, 9 aa, Homo sapiens
FRNQASRPY

SEQ ID NO: 29 - protein, 15 aa, Homo sapiens
MVTFRNQASRPYSFY

SEQ ID NO: 30 - protein, 9 aa, Homo sapiens
IMHSINGYV

SEQ ID NO: 31 - protein, 15 aa, Homo sapiens
ASNIMSSINGYVFDS

SEQ ID NO: 32 - protein, 9 aa, Homo sapiens
LLLKQSNSS

SEQ ID NO: 33 - protein, 15 aa, Homo sapiens
SDLLLLKQSNSSKIL

SEQ ID NO: 34 - protein, 9 aa, Homo sapiens
RVLFQDNSS

SEQ ID NO: 35 - protein, 15 aa, Homo sapiens
YLTRVLFQDNSSHLP

SEQ ID NO: 36 - protein, 9 aa, Homo sapiens
FKNLASRPY

SEQ ID NO: 37 - protein, 15 aa, Homo sapiens
QVRFKNLASRPYSLH

SEQ ID NO: 38 - protein, 9 aa, Homo sapiens
LKNMASHPV

SEQ ID NO: 39 - protein, 15 aa, Homo sapiens
VITLKNMASHPVSLH

SEQ ID NO: 40 - protein, 9 aa, Homo sapiens
FKNQASRPY

SEQ ID NO: 41 - protein, 15 aa, Homo sapiens
LIIFKNQASRPYNIY

SEQ ID NO: 42 - protein, 9 aa, Homo sapiens
FRNQASRPY

SEQ ID NO: 43 - protein, 15 aa, Homo sapiens
MVTFRNQASRPYSFY

SEQ ID NO: 44 - protein, 9 aa, Homo sapiens
FHAINGYIM

SEQ ID NO: 45 - protein, 15 aa, Homo sapiens
NYRFHAINGYIMDTL

SEQ ID NO: 46 - protein, 9 aa, Homo sapiens
IKKITAIIT

SEQ ID NO: 47 - protein, 15 aa, Homo sapiens
LLKIKKITAIITQGC

SEQ ID NO: 48 - protein, 9 aa, Homo sapiens
FKKVTPLIH

SEQ ID NO: 49 - protein, 15 aa, Homo sapiens
DTEFKKVTPLIHDRM

SEQ ID NO: 50 - protein, 9 aa, Homo sapiens
VKNFFNPPI

SEQ ID NO: 51 - protein, 15 aa, Homo sapiens
KGHVKNFFNPPIISR

SEQ ID NO: 52 - protein, 9 aa, Homo sapiens
KHNIFNPPI

SEQ ID NO: 53 - protein, 15 aa, Homo sapiens
SGIKHNIFNPPIIAR

SEQ ID NO: 54 - protein, 9 aa, Homo sapiens
FDETKSWYF

SEQ ID NO: 55 - protein, 15 aa, Homo sapiens
FTIFDETKSWYFTEN

SEQ ID NO: 56 - protein, 9 aa, Homo sapiens
FDENRSWYL

SEQ ID NO: 57 - protein, 15 aa, Homo sapiens
FSVFDENRSWYLTEN

SEQ ID NO: 58 - protein, 9 aa, Homo sapiens
VHSGLIGPL

SEQ ID NO: 59 - protein, 14 aa, Homo sapiens
EKDVHSGLIGPLIV

SEQ ID NO: 60 - protein, 9 aa, Human Influenza A virus
PRYVKQNTL

SEQ ID NO: 61 - protein, 9 aa, Human Influenza A virus
RYVKQNTLK

SEQ ID NO: 62 - protein, 9 aa, Human Influenza A virus
YVKQNTLKL

SEQ ID NO: 63 - protein, 9 aa, Human Influenza A virus
VKQNTLKLA

SEQ ID NO: 64 - protein, 9 aa, Human Influenza A virus
KQNTLKLAT

SEQ ID NO: 65 - DNA, 32 bp, Artificial Sequence
  Sythetic primer
gttaactagttcagctggaccacagccgcagc

SEQ ID NO: 66 - DNA, 40 bp, Artificial Sequence
  Synthetic primer
cgggttaactagttcagaaatcctttctcttgaccatggc

SEQ ID NO: 67 - DNA, 25 bp, Artificial Sequence
  Synthetic primer
ctagcctctggaatcctttctcttg

SEQ ID NO: 68 - protein, 21 aa, Homo sapiens
RIHMVYSKRSGKPRGYAFIEY

SEQ ID NO: 69 - protein, 2366 aa, Artificial Sequence
  Synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGTVNRSLPGLIGCHRKSVYWHVIGMGTTPEVHSIFLEGHTFLVRNHRQASLEISPITFLTAQTLLMDLGQFLLFCHISSHQHDGMEAYVKCDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSESIPQANRSPLPIAKVSSFPSIRPIYLTRVLFQDNSSNLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPSNIQMEDPTFKENYRFHAINGYIMDTLFGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYILTIHFTGHSFIYGK

SEQ ID NO: 70 - protein, 2366 aa, Artificial Sequence
  Sythetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGTVNRSLPGLIGCHRKSVYWHVIGMGTTPEVHSIFLEGHTFLVRNHRQASLEISPITFLTAQTLLMDLGQFLLFCHISSHQHDGMEAYVKCDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSESIPQANRSPLPIAKVSSFPSIRPIYLTRVLFQDNSSNLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTILTIHFTGHSFIYGKIFDETKSWYFTENMERNCRAPSNIQMEDPTFKENYRFHAINGYIMDTLFGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 71 - protein, 2366 aa, Artificial Sequence
  Synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGTVNRSLPGLIGCHRKSVYWHVIGMGTTPEVHSIFLEGHTFLVRNHRQASLEISPITFLTAQTLLMDLGQFLLFCHISSHQHDGMEAYVKCDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSESIPQANRSPLPIAKVSSFPSIRPIYLTRVLFQDNSSNLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPSNIQMEDPTFKENYRFHAINGYIMDTLFGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYIHSIHFSGHVFTVRK

SEQ ID NO: 72 - protein, 2366 aa, Artificial Sequence
  Synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGTVNRSLPGLIGCHRKSVYWHVIGMGTTPEVHSIFLEGHTFLVRNHRQASLEISPITFLTAQTLLMDLGQFLLFCHISSHQHDGMEAYVKCDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSESIPQANRSPLPIAKVSSFPSIRPIYLTRVLFQDNSSNLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIHSIHFSGHVFTVRKIFDETKSWYFTENMERNCRAPSNIQMEDPTFKENYRFHAINGYIMDTLFGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 73 - protein, 2381 aa, Artificial Sequence
  Synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGTVNRSLPGLIGCHRKSVYWHVIGMGTTPEVHSIFLEGHTFLVRNHRQASLEISPITFLTAQTLLMDLGQFLLFCHISSHQHDGMEAYVKCDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSESIPQANRSPLPIAKVSSFPSIRPIYLTRVLFQDNSSNLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPSNIQMEDPTFKENYRFHAINGYIMDTLFGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYILTIHFTGHSFIYGKIHSIHFSGHVFTVRK

SEQ ID NO: 74 - protein, 9 aa, Homo sapiens
ILTIHFTGH

SEQ ID NO: 75 - protein, 9 aa, Homo sapiens
LTIHFTGHS

SEQ ID NO: 76 - protein, 9 aa, Homo sapiens
TIHFTGHSF

SEQ ID NO: 77 - protein, 9 aa, Homo sapiens
FTGHSFIYG

SEQ ID NO: 78 - protein, 9 aa, Homo sapiens
TGHSFIYGK

SEQ ID NO: 79 - protein, 9 aa, Homo sapiens
IHSIHFSGH

SEQ ID NO: 80 - protein, 9 aa, Homo sapiens
HSIHFSGHV

SEQ ID NO: 81 - protein, 9 aa, Homo sapiens
SIHFSGHVF

SEQ ID NO: 82 - protein, 9 aa, Homo sapiens
HFSGHVFTV

SEQ ID NO: 83 - protein, 9 aa, Homo sapiens
FSGHVFTVR

SEQ ID NO: 84 - protein, 9 aa, Homo sapiens
SGHVFTVRK

SEQ ID NO: 85 - protein, 9 aa, Homo sapiens
KIVFKNMAS

SEQ ID NO: 86 - protein, 9 aa, Homo sapiens
IVFKNMASR

SEQ ID NO: 87 - protein, 9 aa, Homo sapiens
VFKNMASRP

SEQ ID NO: 88 - protein, 9 aa, Homo sapiens
KNMASRPYS

SEQ ID NO: 89 - protein, 9 aa, Homo sapiens
NMASRPYSI

SEQ ID NO: 90 - protein, 9 aa, Homo sapiens
MASRPYSIY

SEQ ID NO: 91 - protein, 9 aa, Homo sapiens
FAVFDENKS

SEQ ID NO: 92 - protein, 9 aa, Homo sapiens
AVFDENKSW

SEQ ID NO: 93 - protein, 9 aa, Homo sapiens
VFDENKSWY

SEQ ID NO: 94 - protein, 9 aa, Homo sapiens
FDENKSWYL

SEQ ID NO: 95 - protein, 9 aa, Homo sapiens
DENKSWYLE

SEQ ID NO: 96 - protein, 9 aa, Homo sapiens
ENKSWYLED

SEQ ID NO: 97 - protein, 9 aa, Homo sapiens
NKSWYLEDN

SEQ ID NO: 98 - protein, 9 aa, Homo sapiens
ESNIMSTIN

SEQ ID NO: 99 - protein, 9 aa, Homo sapiens
SNIMSTING

SEQ ID NO: 100 - protein, 9 aa, Homo sapiens
NIMSTINGY

SEQ ID NO: 101 - protein, 9 aa, Homo sapiens
MSTINGYVP

SEQ ID NO: 102 - protein, 9 aa, Homo sapiens
STINGYVPE

SEQ ID NO: 103 - protein, 9 aa, Homo sapiens
TINGYVPES

SEQ ID NO: 104 - protein, 9 aa, Homo sapiens
EKDIHSGLI

SEQ ID NO: 105 - protein, 9 aa, Homo sapiens
KDIHSGLIG

SEQ ID NO: 106 - protein, 9 aa, Homo sapiens
DIHSGLIGP

SEQ ID NO: 107 - protein, 9 aa, Homo sapiens
HSGLIGPLL

SEQ ID NO: 108 - protein, 9 aa, Homo sapiens
SGLIGPLLI

SEQ ID NO: 109 - protein, 9 aa, Homo sapiens
SHEFHAING

SEQ ID NO: 110 - protein, 9 aa, Homo sapiens
HEFHAINGM

SEQ ID NO: 111 - protein, 9 aa, Homo sapiens
EFHAINGMI

SEQ ID NO: 112 - protein, 9 aa, Homo sapiens
HAINGMIYS

SEQ ID NO: 113 - protein, 9 aa, Homo sapiens
AINGMIYSL

SEQ ID NO: 114 - protein, 9 aa, Homo sapiens
INGMIYSLP

SEQ ID NO: 115 - protein, 10 aa, Homo sapiens
HFTGHSFIYG

SEQ ID NO: 116 - protein, 10 aa, Homo sapiens
IHFSGHVFTV

SEQ ID NO: 117 - protein, 15 aa, Homo sapiens
GTDFTLTISSLQPED

SEQ ID NO: 118 - protein, 26 aa, Homo sapiens
PAVLQSSGLYSLSSVVTVPSSSLGTQ

SEQ ID NO: 119 - protein, 21 aa, Homo sapiens
EEQYNSTYRVVSVLTVLHQDW

(19) United States
(12) Patent Application Publication
(10) Pub. No.: US 2021/0300985 A1
(43) Pub. Date: Sep. 30, 2021

(54) REGULATORY T CELL EPITOPES

(71) Applicant: EpiVax Inc., Providence, RI (US)

(72) Inventors: Anne De Groot, Providence, RI (US); William Martin, Providence, RI (US)

(21) Appl. No.: 17/214,189

(22) Filed: Mar. 26, 2021

Related U.S. Application Data
(60) Provisional application No. 63/031,006, filed on May 28, 2020.
(60) Provisional application No. 63/000,633, filed on Mar. 27, 2020.

(51) Int. Cl. C07K 14/705; C07K 14/755; C07K 14/745; A61P 37/06

(52) U.S. Cl. CPC C07K 14/705; C07K 14/755; A61K 38/00; A61P 37/06; C07K 2319/00; C07K 14/745

(57) ABSTRACT

The present is directed to compositions comprising regulatory T cell epitopes, wherein said epitopes comprise a polypeptide comprising at least a portion of SEQ NOS: 1-14 and 74-116, fragments and/or variants thereof, as well as methods of producing and using the same.

### CROSS-REFERENCE TO RELATED APPLICATION

This application depends from and claims priority to U.S. Provisional Application No. 63/000,633 filed Mar. 27, 2020, and U.S. Provisional Application No. 63/031,006 filed May 28, 2020, the entire contents of each of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on May 25, 2021, is named EPV0008PA1_ ST25.txt and is 121 KB in size.

### FIELD

The present disclosure generally relates to a novel class of regulatory T cell epitopes (termed “Tregitopes”). The present disclosure provides Tregitope compounds and compositions (including one or more of e.g., polypeptides (which may be termed herein as “T_{reg} activating regulatory T-cell epitope”, “Tregitope”, or “T-cell epitope polypeptide”) having a sequence comprising, consisting of, or consisting essentially of one or more of SEQ ID NOS: 1-14 and 74-116 (and/or fragments and variants thereof), and optionally 1 to 12 additional amino acids distributed in any ratio on the N-terminus and/or C-terminus of the polypeptide of SEQ ID NOS: 1-14 and 74-116 as disclosed herein; concatemeric peptides as disclosed herein; nucleic acids, chimeric or fusion polypeptide compositions as disclosed herein; nucleic acids as disclosed herein, expression cassettes, plasmids, expression vectors, recombinant viruses, or cells as disclosed herein; and/or pharmaceutical compositions or formulations as disclosed herein), as well as methods for their preparation and use of the same.

### BACKGROUND

Artificial induction of tolerance to self or to foreign antigens is the goal of therapy for autoimmunity, transplantation allergy and other diseases. Immune response targeting autologous and non-autologous therapeutic proteins often limits clinical efficacy. Immune modulating treatments, inducing tolerance to therapeutic proteins compositions, may reduce the formation of anti-drug antibodies (ADA) improving clinical outcomes. Until recently, therapeutic tolerance induction relied on broad-based immune cell depleting therapies. These broad-based approaches weaken the immune system in general and leave many subjects vulnerable to opportunistic infections, autoimmune attack and cancer. There is a need in the art for less aggressive and more targeted approaches to the induction of immune tolerance.

Immune tolerance is regulated by a complex interplay between antigen presenting cells (APC), T cells, B cells, cytokines, chemokines, and surface receptors. Initial self/non-self discrimination occurs in the thymus during neonatal development where medullary epithelial cells express specific self protein epitopes to immature T cells. T cells recognizing self antigens with high affinity are deleted, but autoreactive T cells with moderate affinity sometimes avoid deletion and can be converted to so called ‘natural’ regulatory T cells (T_{Reg}) cells. These natural T_{Reg} cells are exported to the periphery and help to control latent autoimmune response.

A second form of tolerance develops in the periphery. In this case activated T cells are converted to an ‘adaptive’ T_{Reg} phenotype through the action of certain immune suppressive cytokines and chemokines such as IL-10, TGF-β and CCL19. The possible roles for these ‘adaptive’ T_{Reg} cells include dampening immune response following the successful clearance of an invading pathogen, controlling excessive inflammation caused by an allergic reaction, controlling excessive inflammation caused by low level or chronic infection, or possibly controlling inflammatory response targeting beneficial symbiotic bacteria.

Naturally occurring T_{Regs} (including both natural T_{Regs} and adaptive T_{Regs}) are a critical component of immune regulation in the periphery. For example, upon activation of natural T_{Regs} through their TCR, natural T_{Regs} express immune modulating cytokines and chemokines. Activated natural T_{Regs} may suppress nearby effector T cells through contact dependent and independent mechanisms. In addition, the cytokines released by these cells including, but not limited to, IL-10 and TGF-β, are capable of inducing antigen-specific adaptive T_{Regs}. Despite extensive efforts, with few exceptions, the antigen specificity of natural T_{Regs}, and more importantly natural T_{Regs} circulating in clinically significant volumes, is still unknown.

There is need in the art for the identification of regulatory T cell epitopes contained in common autologous proteins (“Tregitopes”), compositions containing such Tregitopes, and for methods related to their preparation and use.

### SUMMARY

Accordingly, the present disclosure provides novel, therapeutic regulatory T cell epitope compositions. Such compositions include one or more of e.g., polypeptides (which may be termed herein as “Treg activating regulatory T-cell epitope”, “Tregitope”, “Tregitope peptide”, or “T-cell epitope polypeptide”) having a sequence comprising, consisting of, or consisting essentially of one or more of SEQ ID NOS: 1-14 and 74-116 (and/or fragments and variants thereof), and optionally 1 to 12 additional amino acids distributed in any ratio on the N terminus and/or C-terminus of the polypeptide of SEQ ID NOS: 1-14 and 74-116 as disclosed herein; concatemeric peptides as disclosed herein; nucleic acids, chimeric or fusion polypeptide compositions as disclosed herein; nucleic acids as disclosed herein, expression cassettes, plasmids, expression vectors, recombinant viruses, or cells as disclosed herein; expression cassettes, plasmids, expression vectors, recombinant viruses, or cells as disclosed herein; chimeric or fusion polypeptide compositions as disclosed herein; and/or pharmaceutical compositions or formulations as disclosed herein, and use of the same, e.g., to suppress an immune response in the body or more specifically to suppress an immune response in the body caused by the administration of a therapeutic agent to treat a medical condition.

The selective engagement and activation of naturally occurring T_{Regs} (in aspects, including natural T_{Regs} and/or adaptive T_{Regs}) through the use of Tregitope compounds and compositions and Tregitope-antigen compositions as disclosed herein, is therapeutically valuable as a means of treatment for any disease or condition marked by the presence of an unwanted immune response. Examples of such an unwanted immune response include the following: Autoimmune disease such as type 1 diabetes, MS, Lupus, and RA; Transplant related disorders such as Graft vs. Host disease (GVHD) and Host vs. Graft disease (HVGD); Allergic reactions; Immune rejection of biologic medicines such as monoclonal antibodies; the management of immune response targeting replacement proteins, e.g., but not limited to, Insulin, coagulation Factor VIII (FVIII), and/or coagulation Factor VIII supplements; the management of immune response targeting therapeutic toxins such as Botulinum toxin; and the management of immune response to infectious disease whether acute or chronic. In aspects, the present disclosure harnesses the functions of naturally occurring TRegs (in aspects, including natural TRegs and/or adaptive TRegs), and in particular aspects, those cells that already regulate immune responses to foreign and self-proteins in the periphery (pre-existing or natural TReg). In aspects, a Tregitope compound or composition of the present disclosure may be either covalently bound, non-covalently bound, or in admixture with a specific target antigen.

In aspects, a Tregitope compound or composition of the present disclosure includes one or more peptides or polypeptides a disclosed herein. In aspects, the present disclosure is directed to a peptide or polypeptide having a sequence comprising, consisting of, or consisting essentially of one or more of SEQ ID NOS: 1-14 and 74-116, or fragments and variants thereof. The phrase “consisting essentially of” is intended to mean that a polypeptide according to the present disclosure, in addition to the sequence according to any of SEQ ID NOS: 1-14 and 74-116 or a fragment or variant thereof, contains additional amino acids or residues that may be present at either terminus of the peptide and/or on a side chain that are not necessarily forming part of the peptide that functions as an MHC ligand and provided they do not substantially impair the activity of the peptide to function as a Tregitope. In aspects, an isolated, synthetic, or recombinant polypeptide (Treg activating regulatory T-cell epitope, Tregitope, Tregitope peptide, or T-cell epitope polypeptide) comprises, consists of, or consists essentially of one or more of SEQ ID NOS: 1-2, or fragments and variants thereof. In aspects, an isolated, synthetic, or recombinant polypeptide (Treg activating regulatory T-cell epitope, Tregitope, Tregitope peptide, or T-cell epitope polypeptide) comprises, consists of, or consists essentially of the amino acid sequence of SEQ ID NO: 1, or a fragment or variant thereof. The polypeptides of the present disclosure may be isolated, synthetic, and/or recombinant, and may comprise post-transcriptional modifications such as glycosylation, added chemical groups, etc. In aspects, the peptides or polypeptides can be either in neutral (uncharged) or salt forms, and may be either free of or include modifications such as glycosylation, side chain oxidation, or phosphorylation. In certain aspects, the Tregitopes can be capped with an N-terminal acetyl and/or C-terminal amino group.

In aspects, the instant disclosure is directed to a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116. In aspects, the instant disclosure is directed to a peptide or polypeptide have a core amino acid sequence comprising, consisting of, or consisting essentially of one or more peptides or polypeptides having an amino acid sequence of SEQ ID NOS: 1-14 and 74-116, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide having a core sequence comprising, consisting of, or consisting essentially of one or more peptides or polypeptides having an amino acid sequence of SEQ ID NOS: 1-14 and 74-116 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to a same HLA molecule (i.e., retain MHC binding propensity) as said polypeptide core sequence without said flanking amino acids. In aspects, said polypeptide with the flanking amino acids is still able to bind to a same HLA molecule (i.e., retain MHC binding propensity) and/or retain the same TCR specificity, and/or retain regulatory T cell stimulating or suppressive activity. as said polypeptide core sequence without said flanking amino acids. In aspects, said flanking amino acid sequences are those that also flank the peptides or polypeptides included therein in the naturally occurring protein, e.g., as found in Factor V or Factor VIII. In aspects, said flanking amino acid sequences as described herein may serve as a MHC stabilizing region. In aspects, the use of a longer peptide may allow endogenous processing by patient cells and may lead to more effective antigen presentation and induction of T cell responses. In aspects, the extension(s) may serve to improve the biochemical properties of the peptides or polypeptides (e.g., but not limited to, solubility or stability) or to improve the likelihood for efficient proteasomal processing of the peptide. In aspects, the polypeptides of the present disclosure may be isolated, synthetic, and/or recombinant, and may comprise post-transcriptional modifications such as glycosylation, added chemical groups, etc. In aspects, the peptides or polypeptides can be either in neutral (uncharged) or salt forms, and may be either free of or include modifications such as glycosylation, side chain oxidation, or phosphorylation. In certain aspects, the peptides or polypeptides of the instant disclosure can be capped with an n-terminal acetyl and/or c-terminal amino group.

In aspects, the instant disclosure is directed to a polypeptide comprising an amino acid sequence having at least 75%, 80%, 85%, 90%, or 95% homology to any one of SEQ ID NOS: 1-14 and 74-116 (and/or fragments thereof), wherein said polypeptide is still able to bind to a same HLA molecule (i.e., retain MHC binding propensity) and/or retain the same TCR specificity, and/or retain regulatory T cell stimulating or suppressive activity.

In aspects, the present disclosure is directed to a concatemeric polypeptide or peptide that comprises at one or more of the instantly-disclosed polypeptides or peptides (e.g., but not limited to, a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects a concatemeric peptide is composed of 1 or more, 2 or more, 3 or more, 4 or more, 5 or more 6 or more 7 or more, 8 or more, 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage sensitive sites, adjacent to their N and/or C terminal end. In such a concatemeric peptide, two or more of the peptide epitopes may have a cleavage sensitive site between them. Alternatively two or more of the peptide epitopes may be connected directly to one another or through a linker that is not a cleavage sensitive site. In aspects, the instantly-disclosed concatemeric polypeptide or peptide sequences do not correspond to a naturally occurring sequence, i.e., each of the one or more of the instantly-disclosed polypeptides or peptides (e.g., but not limited to, a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) are linked, fused, or joined together (e.g., fused in-frame, chemically-linked, or otherwise bound) to an additional peptide or polypeptide (which may be one or more of the instantly-disclosed peptides) in such a fashion such that the overall concatemeric polypeptide does not correspond to a naturally occurring Factor V or Factor VIII sequence. In aspects, the concatemeric polypeptides of the present disclosure may be isolated, synthetic, and/or recombinant, and may comprise post-transcriptional modifications such as glycosylation, added chemical groups, etc. In aspects, the concatemeric polypeptides can be either in neutral (uncharged) or salt forms, and may be either free of or include modifications such as glycosylation, side chain oxidation, or phosphorylation. In certain aspects, the concatemeric polypeptides of the instant disclosure can be capped with an n-terminal acetyl and/or c-terminal amino group.

In aspects, one or more peptides or polypeptides or concatemeric polypeptides of the instant disclosure (e.g., but not limited to, a peptide or polypeptide comprising, consisting, or consisting essentially of an amino acid sequence of SEQ ID NOS. 1-14 and 74-116 (and/or fragments or variants thereof), and optionally 1 to 12 additional amino acids distributed in any ratio on the N terminus and/or C-terminus of the polypeptide of SEQ ID NOS. 1-14 and 74-116) may be joined to, linked to (e.g., fused in-frame, chemically-linked, or otherwise bound), and/or inserted into a heterologous polypeptide. In aspects, the one or more peptides or polypeptides of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide.

In aspects, the present disclosure is directed to polypeptide (which, in aspects, may be an isolated, synthetic, or recombinant) having a sequence comprising one or more of SEQ ID NOS: 1-14 and 74-116 (and fragments or variants thereof), wherein said one or more of SEQ ID NOS: 1-14 and 74-116 is not naturally included in the polypeptide and/or said one or more of SEQ ID NOS: 1-14 and 74-116 is not located at its natural position in the polypeptide. In aspects, one or more Tregitopes of the instant disclosure (which, in aspects, may be an isolated, synthetic, or recombinant) having a sequence comprising one or more of SEQ ID NOS: 1-14 and 74-116 (and fragments or variants thereof), and optionally 1 to 12 additional amino acids distributed in any ratio on the N terminus and/or C-terminus of the polypeptide of SEQ ID NOS: 1-14 and 74-116, may also be fused to or inserted internally within (e.g., but not limited to, site directed mutagenesis or other recombinant techniques) a Factor V or Factor VIII molecule or replacement protein/supplement (or fragments thereof), such as in instances where the Tregitope is not located in its natural position within the Factor V or Factor VIII molecule or replacement protein/supplement (or fragments thereof) or wherein the Factor V or Factor VIII molecule or replacement protein/supplement (or fragments thereof) is missing such a Tregitope (e.g., if a particular Factor V or Factor VIII molecule or replacement protein/supplement (or fragments thereof) has a mutated or missing corresponding section). In aspects, such polypeptides of the present disclosure, which are further described below, may be isolated, synthetic, and/or recombinant, and may comprise post-transcriptional modifications such as glycosylation, added chemical groups, etc. In aspects, such polypeptides can be either in neutral (uncharged) or salt forms, and may be either free of or include modifications such as glycosylation, side chain oxidation, or phosphorylation. In certain aspects, such polypeptides can be capped with an n-terminal acetyl and/or c-terminal amino group.

In aspects, a polypeptide (which, in aspects, may be an isolated, synthetic, or recombinant) comprises one or more of SEQ ID NOS: 1, 3-8, 10-14, 74-78, and 85-114 (and fragments or variants thereof), and optionally 1 to 12 additional amino acids distributed in any ratio on the N terminus and/or C-terminus of the polypeptide of SEQ ID NOS: 1, 3-8, 10-14, 74-78, and 85-114, wherein said polypeptide does not comprise human coagulation Factor V molecule or replacement protein/supplement (or a fragments thereof). In aspects, if a polypeptide does comprise human coagulation Factor V molecule or replacement protein/supplement (or a fragments thereof), then said one or more of SEQ ID NOS: 1, 3-8, 10-14, 74-78, and 85-114 (and fragments or variants thereof), and optionally 1 to 12 additional amino acids distributed in any ratio on the N terminus and/or C-terminus of the polypeptide of SEQ ID NOS: 1, 3-8, 10-14, 74-78, and 85-114, is not located in its natural position in the human coagulation Factor V molecule or replacement protein/supplement (or a fragments thereof). In aspects, one or more Tregitopes having a sequence comprising SEQ ID NOS: 1, 3-8, 10-14, 74-78, and 85-114 (and fragments or variants thereof), and optionally 1 to 12 additional amino acids distributed in any ratio on the N terminus and/or C-terminus of the polypeptide of SEQ ID NOS: 1, 3-8, 10-14, 74-78, and 85-114, may also be fused to or inserted internally within (e.g., but not limited to, using immune engineering techniques such as but not limited to, site directed mutagenesis or other recombinant techniques) a Factor V molecule or replacement protein/supplement (or a fragments thereof), such as in instances where the Tregitope is not located in its natural position within the Factor V molecule or replacement protein/supplement (or a fragments thereof) or where the Factor V molecule or replacement protein/supplement (or a fragments thereof is missing such a Tregitope (e.g., if a particular human coagulation Factor V molecule or replacement protein/supplement (or a fragments thereof) has a mutated or missing corresponding section).

In aspects, a polypeptide (which, in aspects, may be an isolated, synthetic, or recombinant) comprises one or more of SEQ ID NOS: 2, 9, and 79-84 (and fragments or variants thereof), and optionally 1 to 12 additional amino acids distributed in any ratio on the N terminus and/or C-terminus of the polypeptide of SEQ ID NOS: 2, 9, and 79-84, wherein said polypeptide does not comprise human coagulation Factor VIII molecule or replacement protein/supplement (or a fragments thereof). In aspects, if a polypeptide does comprise human coagulation Factor VIII molecule or a fragment thereof, then said one or more of SEQ ID NOS: 2, 9, and 79-84 (and fragments or variants thereof), and optionally 1 to 12 additional amino acids distributed in any ratio on the N terminus and/or C-terminus of the polypeptide of SEQ ID NOS: 2, 9, and 79-84, is not located in its natural position in the human coagulation Factor VIII molecule or replacement protein/supplement (or a fragments thereof). In aspects, one or more Tregitopes having a sequence comprising SEQ ID NOS: 2, 9, and 79-84 (and fragments or variants thereof), and optionally 1 to 12 additional amino acids distributed in any ratio on the N terminus and/or C-terminus of the polypeptide of SEQ ID NOS: 2, 9, and 79-84, may also be fused to or inserted internally within (e.g., but not limited to, using immune engineering techniques such as but not limited to, site directed mutagenesis or other recombinant techniques) an Factor VIII molecule or replacement protein/supplement (or a fragments thereof), such as in instances where the Tregitope is not located in its natural position within the human coagulation Factor VIII molecule or replacement protein/supplement (or a fragments thereof) or where the human coagulation Factor VIII molecule or replacement protein/supplement (or a fragments thereof is missing such a Tregitope (e.g., if a particular human coagulation Factor VIII molecule or replacement protein/supplement (or a fragments thereof) has a mutated or missing corresponding section).

In aspects, the present disclosure is directed to a chimeric or fusion polypeptide composition (which in aspects may be isolated, synthetic, or recombinant) comprising one or more peptides, polypeptides, or concatemeric peptides of the present disclosure. In aspects, a chimeric or fusion polypeptide composition of the present disclosure comprises one or more peptides, polypeptides, or concatemeric peptides of the present disclosure (e.g., a peptide or polypeptide comprising, consisting of, or consisting essentially of one or more peptides or polypeptides having an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) joined to, linked to (e.g., fused in-frame, chemically-linked, or otherwise bound), and/or inserted into a heterologous polypeptide. In aspects, the one or more isolated, synthetic, or recombinant polypeptides of the present disclosure comprises, consists of, or consists essentially of a sequence one or more of SEQ ID NOS: 1-2 (and/or fragments or variants thereof). In aspects, the one or more isolated, synthetic, or recombinant polypeptides of the present disclosure comprises, consists of, or consists essentially the amino acid sequence of SEQ ID NO: 1 (and/or fragments or variants thereof). In aspects, the one or more peptides, polypeptides, and/or concatemeric peptides of the present disclosure may be inserted into the heterologous polypeptide, may be added to the C-terminus (with or without the use of linkers, as is known in the art), and/or added to the N-terminus (with or without the use of linkers, as is known in the art) of the heterologous polypeptide. In aspects of the instantly disclosed chimeric or fusion polypeptide compositions, the one or more peptides, polypeptides, and/or concatemeric peptides of the present disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition of the present disclosure comprises a peptide, polypeptide, and/or concatemeric peptide of the present disclosure, wherein said peptide, polypeptide, and/or concatemeric peptide having a sequence that is not naturally included in the heterologous polypeptide and/or is not located at its natural position in the heterologous polypeptide. In aspects of above-described chimeric or fusion polypeptide compositions, the chimeric or fusion polypeptides may be isolated, synthetic, or recombinant. In aspects of the instantly-disclosed chimeric or fusion polypeptide compositions, the heterologous polypeptide or polypeptide comprises a biologically active molecule. In aspects, the biologically active molecule is selected from the group consisting of an immunogenic molecule, a T cell epitope, a viral protein, and a bacterial protein. In aspects, the one or more of Tregitopes of the present disclosure can be joined or linked to (e.g., fused in-frame, chemically linked, or otherwise bound) to a small molecule, drug, or drug fragment. In aspects, the chimeric or fusion polypeptides can be in either neutral (uncharged) or salt forms, and may be either free of or include modifications such as glycosylation, side chain oxidation, or phosphorylation.

In aspects, the present disclosure is directed to a nucleic acid (e.g., DNAs, such as cDNA, or RNAs, such as mRNA), which in aspects may be isolated, synthetic, or recombinant, encoding one or more peptides, polypeptides, concatemeric peptides, and/or chimeric or fusion polypeptides as described herein. For example, in aspects, the instant disclosure is directed to a nucleic acid encoding a peptide or polypeptide comprising, consisting of, or consisting essentially of one or more peptides or polypeptides having an amino acid sequence of SEQ ID NOS: 1-14 and 74-116 (and/or fragments or variants thereof), and optionally 1 to 12 additional amino acids distributed in any ratio on the N-terminus and/or C-terminus of the polypeptide of SEQ ID NOS: 1-14 and 74-116. In aspects, the present disclosure is directed to a vector, such as an expression vector, comprising such a nucleic acid as described. In aspects, the present disclosure is directed to expression cassettes, plasmids, expression vectors, recombinant viruses, or cells comprising a nucleic acid as described herein. In aspects, the present disclosure is directed to a cell or vaccine comprising such a vector as described. In aspects, the present disclosure is directed to a cell comprising a vector of the present disclosure.

In aspects, the instant disclosure is directed to a pharmaceutical composition, the pharmaceutical composition comprising a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein) and a pharmaceutically acceptable carrier, excipient, and/or adjuvant. In aspects, the one or more nucleic acids encoding said peptides or polypeptides are DNA, RNA, or mRNA. In aspects of the above-described pharmaceutical compositions, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 100, at least 200, at least 300, at least 400, at least 500, at least 600, at least 700, at least 800, at least 900, or at least 1000, peptides, polypeptides, and/or concatemeric peptides, as disclosed herein, including every value or range therebetween.

In aspects, the present disclosure is directed to a method of stimulating, inducing, and/or expanding regulatory T-cells (in aspects, naturally occurring T_{Regs}, including natural T_{Regs} and/or adaptive T_{Regs}) in a subject in need thereof and/or suppressing an immune response in a subject in need thereof by administering to the subject a therapeutically effect amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein). In aspects, the subject is a human.

In aspects, the present disclosure is directed to a method of treating or preventing a medical condition in a subject in need thereof comprising administering a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein). In aspects, the medical condition is selected from the group consisting of: an allergy, an autoimmune disease, a transplant related disorder, graft versus host disease, a blood clotting disorder, an enzyme or protein deficiency disorder, a hemostatic disorder, cancer, infertility; and a viral, bacterial or parasitic infection. In another embodiment, the medical condition is hemophilia A, B, or C. In aspects, the subject is a human.

In aspects, the present disclosure is directed to a method of stimulating, inducing, and/or expanding regulatory T-cells (e.g., naturally occurring T_{Reg}s (in aspects, including natural T_{Regs} and/or adaptive T_{Regs})) to suppress an immune response in a subject in need thereof by administering to the subject a therapeutically effect amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein). In aspects, the immune response is the result of one or more therapeutic treatments with at least one therapeutic protein, treatment with a vaccine or treatment with at least one antigen. In a particular embodiment, the immune response is the result of one or more therapeutic treatments with a Coagulation Factor VIII supplement. Thus, the administration of one or more Tregitope compound and composition of the present disclosure can be used to prevent the development of, or terminate, an already-established immune response to establish tolerance induction to Factor VIII (and Coagulation Factor VIII supplements) in patients suffering from Hemophilia A. In another aspect, the administration of a Tregitope compound or composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method for expanding a population of regulatory T cells, comprising: (a) providing a biological sample from a subject; (b) isolating regulatory T-cells from the biological sample; (c) contacting the isolated regulatory T-cells with an effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), under conditions wherein the T-regulatory cells increase in number to yield an expanded regulatory T-cell composition, thereby expanding the regulatory T-cells in the biological sample; and, additionally, (d) returning the sample to the subject in need of treatment.

In aspects, the present disclosure is directed to a method for stimulating regulatory T cells in a biological sample, comprising: (a) providing a biological sample from a subject; (b) isolating regulatory T-cells from the biological sample; (c) contacting the isolated regulatory T-cells with an effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), under conditions wherein the T-regulatory cells are stimulated to alter one or more biological function, thereby stimulating the regulatory T-cells in the biological sample; and, additionally, (d) returning cells to the subject in need of treatment.

In aspects, the present disclosure is directed to a method for repressing/suppressing an immune response in a subject, comprising administering a therapeutically effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), wherein the Tregitope compound or composition represses/suppresses the immune response. In aspects, the Tregitope compound or composition represses/suppresses an innate immune response. In aspects, the Tregitope compound or composition represses/suppresses an adaptive immune response. In aspects, the Tregitope compound or composition represses/suppresses an effector T cell response. In aspects, the Tregitope compound or composition represses/suppresses a memory T cell response. In aspects, the Tregitope compound or composition represses/suppresses helper T cell response. In aspects, the Tregitope compound or composition represses/suppresses B cell response. In aspects, the Tregitope compound or composition represses/suppresses an NKT cell (natural killer T cell) response. In another aspect, the administration of a Tregitope compound or composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method of suppressing an immune response, specifically an antigen specific immune response in a subject, through the administration of a therapeutically effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), wherein said Tregitope compound or composition activates naturally occurring TRegs (in aspects, including natural TRegs and/or adaptive TRegs, and in aspects CD4+/CD25+/FoxP3+ regulatory T-cells) or suppresses the activation of CD4+ T-cells, the proliferation of CD4+ and/or CD8+ T-cells, and/or suppresses the activation or proliferation of β-cells or NKT Cells. In aspects, a Tregitope compound or composition of the present disclosure may be covalently bound, non-covalently bound, or in admixture with a specific target antigen. In aspects, an administered Tregitope compound or composition of the present disclosure that is covalently bound, non-covalently bound, or in admixture with a specific target antigen results in the diminution of immune response against the target antigen.

In aspects, the target antigen may be an autologous protein or protein fragment. In aspects, the target antigen may be an allergen. In aspects, the target antigen may allogenic protein or protein fragments. In aspects, the target antigen may be a biologic medicine or fragments thereof. In aspects, the target antigen is a human Factor VIII or a Coagulation Factor VIII replacement protein or supplement. In aspects, the suppressive effect is mediated by natural T_{Regs}. In aspects, the suppressive effect is mediated by an adaptive T_{Regs}. In aspects, the one or more Tregitope included in the Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) suppresses an effector T cell response. In aspects, the one or more Tregitopes of the presently disclosed Tregitope compounds or compositions suppresses an innate immune response. In aspects, the one or more Tregitopes of the presently disclosed Tregitope compounds and compositions suppresses an adaptive immune response. In aspects, the one or more Tregitopes of the presently disclosed Tregitope compounds and compositions suppresses helper T cell response. In aspects, the one or more Tregitopes of the presently disclosed Tregitope compounds and compositions suppresses a memory T cell response. In aspects, the one or more Tregitopes of the presently disclosed Tregitope compounds and compositions suppresses B cell response. In aspects, the one or more Tregitopes of the presently disclosed Tregitope compounds and compositions suppresses NKT cell response.

In aspects, the present disclosure is directed to a kit for preventing or treating a medical condition, in particular, for the suppression of an immune response in a subject, wherein the kit comprises a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein). In aspects, the kit may further comprise an effective amount of an antigen or allergen or therapeutic agent, such as a replacement protein or peptide.

Additionally, the present disclosure is directed to a method for decreasing the immunogenicity and/or increasing tolerogenicity of a polypeptide, which may be particularly useful when a polypeptide (such as human Factor VIII or a Factor VIII replacement protein or supplement (or fragments thereof), or human Factor V or a Factor V replacement protein or supplement (or fragments thereof)) serves as a therapeutic protein. In aspects, said method comprises insertion of one or more regulatory T cell epitopes (e.g., a peptide or polypeptide comprising, consisting of, or consisting essentially of one or more peptides or polypeptides having an amino acid sequence of SEQ ID NOS: 1-14 and 74-116 (and/or fragments or variants thereof), and optionally 1 to 12 additional amino acids distributed in any ratio on the N-terminus and/or C-terminus of the polypeptide of SEQ ID NOS: 1-14 and 74-116) into said polypeptide (e.g., human Factor VIII or a Factor VIII replacement protein or supplement (or fragments thereof), or human Factor V or a Factor V replacement protein or supplement (or fragments thereof)). In aspects, the one or more regulatory T cell epitopes inserted into the human Factor V or Factor VIII molecule or replacement protein/supplement (or fragments thereof) can suppress an antigen-specific immune response against the human Factor V or Factor VIII molecule or replacement protein/supplement (or fragments thereof). In aspects, said one or more regulatory T cell epitopes may be fused to or inserted internally within (e.g., but not limited to, site directed mutagenesis or other recombinant techniques) a human Factor V or Factor VIII molecule or replacement protein/supplement (or fragments thereof), such as in instances where the Tregitope is not located in its natural position within the Factor V or Factor VIII molecule or replacement protein/supplement (or fragments thereof) or wherein the Factor V or Factor VIII molecule or replacement protein/supplement (or fragments thereof) thereof is missing such a Tregitope (e.g., if a particular Factor V or Factor VIII molecule or replacement protein/supplement (or fragments thereof) has a mutated or missing corresponding section). In aspects, said insertion of the one or more regulatory T cell epitopes into the Factor V or Factor VIII molecule or replacement protein/supplement (or fragments thereof) thereof comprises insertion of all or some of the amino acids of the one or more regulatory T cell epitopes. In aspects, said insertion of the one or more regulatory T cell epitopes into the Factor V or Factor VIII molecule or replacement protein/supplement (or fragments thereof) thereof comprises insertion of some or all of the amino acids of the one or more regulatory T cell epitopes and removing one or more amino acids at the site of insertion of the regulatory T cell epitope amino acids. In aspects, said insertion of the one or more regulatory T cell epitopes into the Factor V or Factor VIII molecule or replacement protein/supplement (or fragments thereof) comprises mutating the sequence of the Factor V or Factor VIII molecule or replacement protein/supplement (or fragments thereof) thereof to include the one or more regulatory T cell epitopes (for example, but not limited to, introduction one or more point mutations into the antibody or fragment thereof by site-directed mutagenesis or other recombinant techniques). In aspects, said insertion of the one or more regulatory T cell epitopes into the Factor V or Factor VIII molecule or replacement protein/supplement (or fragments thereof), which in aspects will introduce the one or more regulatory T cell epitope sequences, such that the previous immunogenicity of the sequence is decreased and the tolerogenicity of the new sequence is enhanced. In aspects, the number of said added one or more amino acids at the site of insertion of the regulatory T cell epitope amino acids need not correspond to the number of amino acids deleted from the sequence of the Factor V or Factor VIII molecule or replacement protein/supplement (or fragments thereof). In aspects, said insertion of one or more regulatory T cell epitopes into the Factor V or Factor VIII molecule or replacement protein/supplement (or fragments thereof) results in decreasing the immunogenicity of the antibody or fragment thereof. In aspects, said insertion of one or more regulatory T cell epitopes into the Factor V or Factor VIII molecule or replacement protein/supplement (or fragments thereof) thereof results in a increasing the tolerogenicity of the Factor V or Factor VIII molecule or replacement protein/supplement (or fragments thereof). In aspects, the one or more regulatory T cell epitopes have a sequence comprising, consisting of, or consisting essentially of one or more of SEQ ID NOS: 1-14 and 74-116.

For example, in aspects, one or more Tregitopes of the instant disclosure having a sequence comprising one or more of SEQ ID NOS: 1, 3-8, 10-14, 74-78, and 85-114 (and fragments or variants thereof), and optionally 1 to 12 additional amino acids distributed in any ratio on the N-terminus and/or C-terminus of the polypeptide of SEQ ID NOS: 1, 3-8, 10-14, 74-78, and 85-114), may be fused to or inserted internally within (e.g., but not limited to, site directed mutagenesis or other recombinant techniques) a human coagulation Factor V molecule or replacement protein/supplement (or a fragments thereof), such as in instances where the Tregitope is not located in its natural position within the human coagulation Factor V molecule or replacement protein/supplement (or a fragments thereof) or where the human coagulation Factor V molecule or replacement protein/supplement (or a fragments thereof) is missing such a Tregitope (e.g., if a particular human coagulation Factor V molecule or replacement protein/supplement (or a fragments thereof) has a mutated or missing corresponding section). As described previously, a Tregitope comprising one or more of SEQ ID NOS: 1, 3-8, 10-14, 74-78, and 85-114 may also be fused to or inserted internally within a polypeptide, e.g., a polypeptide that does not comprise human coagulation Factor V molecule or replacement protein/supplement (or a fragments thereof).

Additionally, in aspects, one or more Tregitopes of the instant disclosure having a sequence comprising one or more of SEQ ID NOS: 2, 9, and 79-84 (and fragments or variants thereof), and optionally 1 to 12 additional amino acids distributed in any ratio on the N-terminus and/or C-terminus of the polypeptide of SEQ ID NOS: 2, 9, and 79-84, may be fused to or inserted internally within (e.g., but not limited to, site directed mutagenesis or other recombinant techniques) a human coagulation Factor VIII molecule or replacement protein/supplement (or a fragments thereof), such as in instances where the Tregitope is not located in its natural position within the human coagulation Factor VIII molecule or replacement protein/supplement (or a fragments thereof) or where the human coagulation Factor VIII molecule or replacement protein/supplement (or a fragments thereof) is missing such a Tregitope (e.g., if a particular human coagulation Factor VIII molecule or replacement protein/supplement (or a fragments thereof) has a mutated or missing corresponding section). As described previously, a Tregitope comprising one or more of SEQ ID NOS: 2, 9, and 79-84 may also be fused to or inserted internally within a polypeptide, e.g., a polypeptide that does not comprise human coagulation Factor VIII molecule or replacement protein/supplement (or a fragments thereof).

### BRIEF DESCRIPTION OF THE DRAWINGS

The present disclosure may be better understood with reference to the following figures. The patent or application file contains at least one drawing executed in color. Copies of this patent or patent application publication with color drawing(s) will be provided by the Office upon request and payment of the necessary fee.

FIGS. 1A-E are a series of graphs reporting HLA DRB1 competition binding curves. In particular, FIG. 1A summarizes the results for HLA DRB1 *0101 assay (left panel shows SEQ ID NOS: 1, 3, 4, and 6, right panel shows SEQ ID NOS: 5 and 7), FIG. 1B summarizes the results for the HLA DRB1 *0301 assay. FIG. 1C summarizes the results for the HLA DRB1 *0701 assay for the selected FV peptides (left panel shows SEQ ID NOS: 1, 3, 4, and 6, right panel shows SEQ ID NOS: 5 and 7), FIG. 1D summarizes the results for HLA DRB1 *1101 assay for selected FV peptides (left panel shows SEQ ID NOS: 1, 3, 4, and 6, right panel shows SEQ ID NOS: 5 and 7), and FIG. 1E report summarizes the results for HLA DRB1* 1501 (left panel shows SEQ ID NOS: 1, 3, 4, and 6, right panel shows SEQ ID NOS: 5 and 7).

FIGS. 2A-C demonstrates the effects of exposure to putative Tregitopes on the phenotypes of dendritic cells. FIG. 2A depicts an overlay of HLA-DR/CD11c dot plots for SEQ ID NO: 15 (dark gray, which is described in the figure as 167) and media control (light gray) highlights the shift in observed HLA-DR expression. FIG. 2B is the equation used to calculate % Δ in HLA-DR MFI where % is relative to media control. FIG. 2C is a bar graph showing the % Δ in HLA-DR MFI for each peptide stimulant (wherein SEQ ID NO: 15 is Treg 167).

FIGS. 3A-C depicts the % Δ change in the HLA negative population calculated for each peptide stimulant relative to the media control. In particular, FIG. 3A is a plot of HLA-DR versus CD11c for SEQ ID NO: 15 (which is described in the figure as Tregitope 167) and the media control. FIG. 3B is a bar graph that plots the % of HLA+ and HLA− each peptide stimulant where the vehicle is media. T_{Reg} 167 was used as a control and has the sequence PAVLQSSGLYSLSLSSVVTVPSSSLGTQ (SEQ ID NO: 15). FIG. 3C is the equation used to calculate the % Δ change in the HLA negative population.

FIG. 4 is a series of dot plots representing the surface expression of CD11 vs HLA-DR analyzed on assay day 7 across the five donors in the presence of various peptide stimulants. T_{Reg} 167 (shown as 167 in the figure) was used as a control and has the sequence PAVLQSSGLYSLSLSSVVTVPSSSLGTQ (SEQ ID NO: 15).

FIG. 5 is a series of dot plots representing the surface expression of CD11c vs CD86 analyzed on assay day 7 across the five donors in the presence of various peptide stimulants. T_{Reg} 167 (shown as 167 in the figure) was used as a control and has the sequence PAVLQSSGLYSLSLSSVVTVPSSSLGTQ (SEQ ID NO: 15).

FIGS. 6A-B depict the gating strategy for highly activated regulatory T cells. CD4+ Tcells are gated for elevated CD25, Ganzyme B, Foxp3, and low CFSE (proliferation). FIG. 6A shows the gating strategy on the assay with no addition of Tetanus Toxoid (TT), while FIG. 6B shows the gating strategy on the assay with the addition of 0.5 μg/mL of TT.

FIG. 7 demonstrates that gating of highly activated Granzyme B positive CD4⁺ T cells (CD25⁺ Granzyme B⁺) shows that a subset of these cells is composed of highly proliferative regulatory T cells (CFSE low FoxP3⁺⁺). SEQ ID NO: 1 (shown as FV621) increases the relative proportion of this population (FIG. 7, top row), while SEQ ID NO: 3 (shown as FV432) has no significant effect (FIG. 7, bottom row). Data in the figure corresponds to Donor 135.

FIGS. 8A-B depicts the gating strategy for two measures of CD4+ T cell activation. FIG. 8A demonstrates that activated T effector cells (T_{eff}) are characterized by CD25 and FoxP3 where activated T_{eff} are CD25^{hi}/FoxP3^{int} population, while FIG. 8B shows proliferation of characterized CD4 T cells where cells are first gated on CD4+ and percent proliferation of the CD4+/CD25+ T cells is determined by the CFSE depletion.

FIG. 9 depicts the activation of CD4+ T cells upon stimulation with tetanus toxoid (TT) and suppression by peptide SEQ ID NO: 1 (bottom row) and control peptide SEQ ID NO: 3 (top row).

FIG. 10 reports the proliferation of CD4+ T cells upon stimulation with TT and suppression of same by peptide SEQ ID NO: 1 (bottom row) and control peptide SEQ ID NO: 3 (top row).

FIG. 11 depicts the activation of CD4+ T cells upon stimulation with tetanus toxoid (TT) and suppression by peptide SEQ ID NO: 1 (bottom row) and control peptide SEQ ID NO: 3 (top row).

FIG. 12 aligns the Tregitopes (SEQ ID NO: 8 (start peptide) and SEQ ID NO: 1 (extended peptide)) with its Factor VIII homologues (SEQ ID NO: 9 (start peptide) and SEQ ID NO 2 (extended peptide), respectively), displaying the homology between the peptides. Anchor residues are shown as bolded; conserved TCR contacts are shown as bolded and underlined; and mismatched TCR contacts are shown as bolded and italicized.

FIGS. 13A-B depicts the comparative inhibition of CD4+ T cell activation (FIG. 13A) and CD4+ T cell proliferation (FIG. 13B) by peptides SEQ ID NO: 1 (top row of both FIG. 13A and FIG. 13B) and SEQ ID NO: 2 (bottom row of FIG. 13A and FIG. 13B) across two donors.

FIG. 14 illustrates the effect of SEQ ID NO: 1 on CD8+ T cell proliferation (top row) and activation (bottom row) for Donor 121.

FIG. 15 shows the effect of peptide SEQ ID NO: 1 on CD8+ T cell proliferation (top row) and activation (bottom row) for Donors 097 and 121.

FIGS. 16A-B summarize the survival plots for peptide SEQ ID NO: 1 showing a favorable survival profile over both negative controls (FIG. 16A) and positive controls (FIG. 16B).

FIGS. 17A-B demonstrate that the inhibitory effect of the Tregitopes of the instant disclosure on activated (proliferating) CD4⁺ effector T cells responding to Tetanus Toxoid (FIG. 17A) mirrors that of highly activated (Granzyme B+) regulatory T cells (FIG. 17B) across donors covering a broad range of HLA-DRB1 haplotypes. For each donor, values are normalized to 100%=TT only, no Tregitope of the instant disclosure. As shown in both FIG. 17A and FIG. 17B, SEQ ID NO: 1 is shown as FV621, SEQ ID NO: 3 is shown as FV432, SEQ ID NO: 4 is shown as FV548, SEQ ID NO: 5 is shown as FV582, SEQ ID NO: 6 is shown as FV1737, and SEQ ID NO: 7 is shown as FV1802.

FIG. 18 shows that Tetanus Toxoid stimulated a population of activated (CD25 high) CD4 T cells to proliferate (CFSE low) (with approximately 90% of the activated cells also proliferating (data not shown)) and further shows that this population of highly activated cells is actively suppressed by SEQ ID NO:1 (shown as FV621) in a dose-dependent manner (FIG. 18, top row), while SEQ ID NO: 3 (shown as FV432) shows no significant inhibitory effect on activated CD4 cells (FIG. 18, bottom row).

FIG. 19 depicts an example of an immunogenic influenza HA peptide that contains an EpiBar and the EpiMatrix analysis of the promiscuous influenza epitope. The influenza HA peptide scores extremely high for all eight alleles in EpiMatrix and has a cluster score of 18. Cluster scores of 10 are considered significant. The band-like EpiBar pattern is characteristic of promiscuous epitopes. Results are shown for PRYVKQNTL (SEQ ID NO: 60), RYVKQNTLK (SEQ ID NO: 61), YVKQNTLKL (SEQ ID NO: 62), VKQNTLKLA (SEQ ID NO: 63) and KQNTLKLAT (SEQ ID NO: 64). Z score indicates the potential of a 9-mer frame to bind to a given HLA allele. All scores in the top 5% are considered “hits”, while non hits (*) below 10% are masked in FIG. 19 for simplicity.

FIG. 20 shows putative epitopes shared between Human Factor V and Human Factor VIII, with >80% TCR contacts preserved (matched on DR15), as well as their EpiMatrix Score. Anchor residues are shown in black, conserved TCR contacts are shown in green, and mis-matched TCR contacts are shown in red. Results are shown for: IHFTGHSFI (SEQ ID NO: 8) and extended peptide ILTIHFTGHSFIYGK (SEQ ID NO: 1), selected; IHFSGHVFT (SEQ ID NO: 9) and extended peptide IHSIHFSGHVFTVRK (SEQ ID NO: 2), selected; FKNMASRPY (SEQ ID NO: 10) and extended peptide KIVFKNMASRPYSIY (SEQ ID NO: 3), selected; IMSTINGYV (SEQ ID NO: 12) and extended peptide ESNIMSTINGYVPES (SEQ ID NO: 5), selected; FHAINGMIY (SEQ ID NO: 14) and extended peptide SHEFHAINGMIYSLP (SEQ ID NO: 7), selected; IEDFNSGLI (SEQ ID NO: 16) and extended peptide ENLIEDFNSGLIGPL (SEQ ID NO: 17); VKDLNSGLI (SEQ ID NO: 18) and extended peptide VDLVKDLNSGLIGAL (SEQ ID NO: 19); KIVFKNMAS (SEQ ID NO: 20) and extended peptide DTLKIVFKNMASRPY (SEQ ID NO: 21); VITLKNMAS (SEQ ID NO: 22) and extended peptide DTVVITLKNMASHPV (SEQ ID NO: 23); FKNQASRPY (SEQ ID NO: 24) and extended peptide LTIFKNQASRPYNIY (SEQ ID NO: 25); LKNMASHPV(SEQ ID NO: 26) and extended peptide VITLKNMASHPVSLH (SEQ ID NO: 27); FRNQASRPY (SEQ ID NO: 28) and extended peptide MVTFRNQASRPYSFY (SEQ ID NO: 29); IMHSINGYV (SEQ ID NO: 30) and extended peptide ASNIMSSINGYVFDS (SEQ ID NO: 31); LLLKQSNSS (SEQ ID NO: 32) and extended peptide SDLLLLKQSNSSKIL (SEQ ID NO: 33); RVLFQDNSS (SEQ ID NO: 34) and extended peptide YLTRVLFQDNSSHLP (SEQ ID NO: 35); FKNLASRPY (SEQ ID NO: 36) and extended peptide QVRFKNLASRPYSLH (SEQ ID NO: 37); LKNMASHPV (SEQ ID NO: 38) and extended peptide VITLKNMASHPVSLH (SEQ ID NO: 39); FKNQASRPY (SEQ ID NO: 40) and extended peptide LIIFKNQASRPYNIY (SEQ ID NO: 41); FRNQASRPY (SEQ ID NO: 42) and extended peptide MVTFRNQASRPYSFY (SEQ ID NO: 43); FHAINGYIM (SEQ ID NO: 44) and extended peptide NYRFHAINGYIMDTL (SEQ ID NO: 45); IKKITAIIT (SEQ ID NO: 46) and extended peptide LLKIKKITAIITQGC (SEQ ID NO: 47); FKKVTPLIH (SEQ ID NO: 48) and extended peptide DTEFKKVTPLIHDRM (SEQ ID NO: 49); VKNFFNPPI (SEQ ID NO: 50) and extended peptide KGHVKNFFNPPIISR (SEQ ID NO: 51); and KHNIFNPPI (SEQ ID NO: 52) and extended peptide SGIKHNIFNPPIIAR (SEQ ID NO: 53).

FIG. 21 shows putative epitopes in which Human Factor V and Human Factor VIII peptide pairs matched at all five TCR contact positions and their putative EpiMatrix scores. Anchor residues are shown in black, conserved TCR contacts are shown in green, and mis-matched TCR contacts are shown in red. Results are shown for: FDENLSWYL (SEQ ID NO: 11) and extended peptide FAVFDENKSWYLEDN (SEQ ID NO: 4); IHSGLIGPL (SEQ ID NO: 13) and extended peptide EKDIHSGLIGPLLI (SEQ ID NO: 6); FDETKSWYF (SEQ ID NO: 54) and extended peptide FTIFDETKSWYFTEN (SEQ ID NO: 55); FDENRSWYL (SEQ ID NO: 56) and extended peptide FSVFDENRSWYLTEN (SEQ ID NO: 57); and VHSGLIGPL (SEQ ID NO: 58) and extended peptide EKDVHSGLIGPLIV (SEQ ID NO: 59).

FIG. 22 summarizes the results obtained through the dendritic cells phenotyping assays of FIG. 5. As presented in FIG. 22, exposure to claimed Tregitope SEQ ID NO: 1, decreased expression of HLA-DR in all five subjects tested. Further, in four out of five subjects, exposure to Tregitope SEQ ID NO: 1 increased the percent of CD86-low present among the CD11c-high cohort. Both trends indicated a shift towards an acquired regulatory phenotype. The analyses of the ΔMFI and % cell population calculations are relative to vehicle control analysis.

FIG. 23 shows the HLA binding results for SEQ ID NO: 1 across five HLA-DR1 types, which indicates a high affinity of binding for SEQ ID NO: 1 across multiple HLA Class II types.

FIGS. 24A-D demonstrate the gating strategy employed for the Bystander Suppression Assay and highlights the regulatory and activation markers in CD4 T cells stimulated by TT, and demonstrating that the major proliferations population corresponds to the T effector memory phenotype (CD45RA-low/CCR7-low). FIG. 24A shows the response to TT by donor 108 in typifies a trend observed across donors and shows typical distinct populations. FIG. 24B shows the gating strategy for the FoxP3 and CD25 markers showing degree of proliferation for each population. FIG. 24C shows the regulatory phenotype of total CD4 T cells and proliferating CD4 T cells showing the high preponderance of activated effector T cells in the latter. FIG. 24D shows that most of the activated T cells (CD25^{hi} FoxP3^{int}, Q2) show an effector memory phenotype (CD45RA-low/CCR7-low).

FIG. 25 is a summary of HLA binding results for the binding curves for certain Tregitopes against the selected Class II HLA alleles as shown in FIGS. 1A-C.

FIG. 26 is the overview of JanusMatrix results for identified the Tregitopes of SEQ ID NOS: 1-7 of the instant disclosure.

FIG. 27 is the JanusMatrix report for the Tregitope of SEQ ID NO: 1 and the 9-mers contained within SEQ ID NO: 1, including SEQ ID NOS: 74-78, 8, and 115.

FIG. 28 is the JanusMatrix report for the Tregitope of SEQ ID NO: 2 and the 9-mers contained within SEQ ID NO: 2, including SEQ ID NOS: 19-84, and 116.

FIG. 29 is the JanusMatrix report for the Tregitope of SEQ ID NO: 3 and the 9-mers contained within SEQ ID NO: 3, including SEQ ID NOS: 85-90 and 10.

FIG. 30 is the JanusMatrix report for the Tregitope of SEQ ID NO: 4 and the 9-mers contained within SEQ ID NO: 4, including SEQ ID NOS: 91-97.

FIG. 31 is the JanusMatrix report for the Tregitope of SEQ ID NO: 5 and the 9-mers contained within SEQ ID NO: 5, including SEQ ID NOS: 98-103 and 12.

FIG. 32 is the JanusMatrix report for the Tregitope of SEQ ID NO: 6 and the 9-mers contained within SEQ ID NO: 6, including SEQ ID NOS: 104-108 and 13.

FIG. 33 is the JanusMatrix report for the Tregitope of SEQ ID NO: 7 and the 9-mers contained within SEQ ID NO: 7, including SEQ ID NOS: 109-114 and 14.

FIG. 34 shows an experimental design for a TTBSA assay evaluating the efficacy of Tregitope-albumin delivery vehicles.

FIGS. 35A-B show HLA DRB1 binding of candidate FV peptides and comparing with IgG Tregitopes. FIG. 35A) Selected Factor V peptides were evaluated for their ability to bind HLA DRB1 in vitro and IC50 values were calculated. FV432, FV621 and FV1802 peptides bind to multiple HLA DRB1 alleles (DRB1*0101, *0301, *0401, *0701, *1101, *1301 and *1501) whereas FV548, FV582 and FV1737 peptides had more limited binding to HLA DR. A seven-point competition assay using a validated control peptide was performed; binding affinity, IC50 were determined by interpolation. FV621 binds with strong affinity binder all HLA DR alleles tested; variation in binding affinity varied less than 10-20% in repeat assays. FIG. 35B) Comparison of FV621 to previously published Tregitope peptides (084, 167, and 289) for binding to HLA DRB1 alleles. Binding assays were performed as described above for FV peptides. Tregitopes 167 and 289 are Fc-derived: 084 is Fab-derived.

FIGS. 36A-D are directed to evaluating the FV peptides and HSA-FV621 for inhibition of CD4+ T cell recall response in the Tetanus Toxoid Bystander Suppression Assay (TTBSA). PBMCs from healthy donors were stimulated with 0.5 μg/ml of TT and increasing concentrations of FV peptides (5, 10, 15 and 20 μg/ml). Proliferation of CD4+ T cells was measured six days post-stimulation by flow cytometry by CFSE dilution. FIG. 36A) Representative flow and histogram plots indicate a dose dependent effect on the proliferation of CD4+ T cells with increasing concentrations of FV621. FIG. 36B) Graphs showing the percent inhibition of CD4+ T cell proliferation for each of the FV-derived peptides compared to TT stimulation alone for donors 157, 135 and 143. FV621 demonstrated consistent suppression of the TT response across donors as compared to the other FV peptides. FIG. 36C) Inhibition of the CD4+ T cell recall response by HSA-conjugates in TTBSA. PBMCs from healthy donors were stimulated with 0.5 μg/ml of TT with or without FV621 or HSA-FV621 and analyzed at six days post-stimulation by flow cytometry for inhibition of CD4+ T cell proliferation. Data shown represents the average 3 donors. P values ***≤0.0002 and ****≤0.0001 represents the statistical significance between TT and FV621 stimulation using a two-tailed t-test. FIG. 36D) FV621 Treatment inhibited OVA-induced immune response in vivo. C57BL/6 mice were immunized with OVA in CFA/IFA at day 0 and day 14 and treated with FV621 in CFA at day 0. Serum and splenocytes were collected at day 21 and anti-OVA antibody titers in serum were measured by ELISA (left) and IFNγ production by splenocytes was measured in a FluoroSpot assay (right).

FIGS. 37A-E show the comparison of the inhibition of CD4+ T cell recall response by the FV621 peptide with previously validated IgG derived Tregitopes 289, 084 and 167 in the TTBSA. PBMCs from healthy donors were stimulated with 0.5 μg/ml of TT with or without the addition of the indicated concentrations of FV621 peptide or Tregitope 289, 084 or 167 and analyzed at six days post-stimulation by flow cytometry. TT induced memory CD4+ T cell proliferation was normalized to 100% and the effect of Tregitope costimulation with TT on the inhibition of CD4+ T cell proliferation by Tregitope 084 FIG. 37A), Tregitope 289 FIG. 37B), Tregitope 167 FIG. 37C) and FV peptide FV621 FIG. 37D) were plotted. Data were compiled from 6 to 10 donors from 4 to 5 independent experiments. FV621 exhibited a similar effect on CD4+ T cell proliferation as compared to the established IgG derived Tregitopes. FIG. 37E) PBMCs were stimulated with 0.5 μg/ml of TT with or without the indicated concentrations of FV621 or 289 and analyzed at six days post-stimulation by flow cytometry. Data are combined from 3 donors. P values *≤0.05, P values **≤0.01, P values ***≤0.0002 and ****≤0.0001 represents statistical significance vs. TT using a two-tailed t-test.

FIGS. 38A-B show FV621 peptide effect on effector and regulatory CD4 T cell response in the bystander assay. FIG. 38A) The upper left panel shows the gating strategy and the bottom panel summarizes the effect of FV621 on the effector CD4+ T (CD25hiFoxP3int) cell population in this example donor. The bar graph on the right shows the inhibition of T effector cell percentage with increasing concentrations of FV621. FIG. 38B) To further discriminate T regulatory cells, CD3+ cells were gated for CD4+/CD127low followed by CD25hi/FoxP3hi as shown. The lower panel shows the effect of FV621 on regulatory CD4+ T cells. The bar graph on the right in Fig. B shows that FV621 increased the percentage (%) of T regulatory cells upon stimulation with FV621. PBMCs from healthy donor were stimulated with TT in the presence or absence of FV621 and analyzed six days post stimulation by flow cytometry. These data are representative of 6-8 individual donors.

FIGS. 39A-C show the dffect of FV621 on CD8+ and CD4+ T cell response in PBMCs from healthy donors stimulated with CEF peptides. FIG. 39A) Effect of increasing FV621 concentrations on CD8+ T cell proliferation (CFSE low) and activation (CD25 high) in PBMCs stimulated with 2 μg/ml CEF peptides. Lower panels show the effect of increasing concentration of FV621 peptide on the proliferative response of CD8+ T cells FIG. 39B) or CD4 T+ cells FIG. 39C) in the same assay, to increasing concentrations of CEF peptides.

FIG. 40A-C show Factor V peptides on the downregulation of HLA DR in antigen presenting cells (APCs). Healthy donor PBMCs were incubated with Tregitope (Treg167), flu peptide (HA306-318) and different factor V peptides for three days and analyzed by flow cytometry for the expression of CD11c and HLA DR. FIG. 40A) Shows the representative FACS plot for expression of HLA DR vs CD11c. FIG. 40B) Comparing MFI of HLA DR expression in CD11c+ cells and FIG. 40C) Graph of (%) change in MFI of HLA DR expression for the total CD11c + relative to media and peptide stimulation. Data are representative plots for one donor from three individual experiments.

FIGS. 41A-B show the effect of FV621 stimulation on the level of granzyme B in T regulatory (square) and T effector (circle) compartment of T cells. PBMCs were stimulated with TT and sorted into granzyme B positive CD4+ T cell populations FIG. 41A) Flow cytometry gating strategy for the activated Treg (GrB+CD25hiFoxP3hi) and Teff (GrB+CD25hiFoxP3int) cells granzyme B production. FIG. 41B) Comparing the effect of FV621 on the (%) of granzyme B positive Treg and Teff cells. Data are representative of 5 individual donors.

### DETAILED DESCRIPTION OF THE INVENTION

### General

The adaptive immune cascade begins when soluble protein antigens are taken up by Antigen Presenting Cells (APCs) and processed through the Class II antigen presentation pathway. Protein antigens in the Class II presentation pathway are degraded by various proteases found in the Endoplasmic Reticulum. Some of the resulting protein fragments are bound to Class II MHC molecules. Peptide-loaded MHC molecules are trafficked to the cell surface where they are interrogated by CD4+ T cells. Peptide fragments that are capable of binding to an MHC molecule and mediating the cell to cell interaction between APC and circulating T cells are referred to as T cell epitopes. Recognition of these peptide-MHC complexes by CD4+ T cells can lead to either an immune activating or immune suppressive response based on the phenotype of the responding T cells and the local cytokine/chemokine milieu. In general, engagement between the MHC/peptide complex and the T cell receptor (TCR) of T effector cells leads to activation and the subsequent secretion of pro-inflammatory cytokines such as IL-4, and IFN-γ. On the other hand, the activation of natural T regulatory cells (T_{Regs}) leads to the expression of the immune suppressive cytokines IL-10 and TGF-β, among others (Shevach E, (2002), Nat Rev Immunol, 2(6):389-400). These cytokines act directly on nearby effector T cells leading in some cases to anergy or apoptosis. In other cases, regulatory cytokines and chemokines convert effector T cells to T regulatory phenotypes; this process is referred here as “induced” or “adaptive” tolerance. T cell epitopes that are capable of binding to MHC molecules and engaging and/or activating circulating naturally occurring T_{Regs} (in aspects, including natural T_{Regs} and/or adaptive T_{Regs}), are referred to as Tregitopes. In aspects, the instantly-disclosed Tregitopes are T cell epitope clusters, which are epitopes capable of binding to multiple MHC alleles and multiple TCRs.

Initial self/non-self discrimination occurs in the thymus during neonatal development where cortical and medullary epithelial cells express specific self-protein epitopes to immature T cells. T cells recognizing self-antigens with high affinity are deleted, but autoreactive T cells with moderate affinity sometimes avoid deletion and can be converted to so called natural regulatory T cells (T_{Reg}) cells. These natural T_{Reg} cells are exported to the periphery and help to control a latent autoimmune response. Natural regulatory T cells are a critical component of immune regulation and self-tolerance.

Self-tolerance is regulated by a complex interplay between T cells, B cells, cytokines and surface receptors. T regulatory immune responses counterbalance T effector immune response to protein antigens (whether self or foreign). A tilt of the balance toward the autoreactive side, either by increasing the number or function of autoreactive T effector cells or by diminishing the number or function of T regulatory cells, is manifested as autoimmunity.

A second form of tolerance occurs in the periphery where mature T cells are converted to an ‘adaptive’ T_{Reg} phenotype upon activation via their T cell receptor in the presence of IL-10 and TGF-β, usually supplied by bystander T regulatory cells. The possible roles for these ‘adaptive’ T_{Reg} cells include dampening immune response following the successful clearance of an invading pathogen, controlling excessive inflammation caused by an allergic reaction, controlling excessive inflammation caused by low level or chronic infection, or possibly controlling inflammatory response targeting beneficial symbiotic bacteria and viruses. ‘Adaptive’ T_{Regs} may also play a role in suppressing immune response targeting human antibodies that have undergone somatic hypermutation (Chaudhry A et al., (2011), Immunity, 34(4):566-78).

T_{Reg} cells are also instrumental in B cell tolerance. B cells express a single low affinity Fc receptor, FcyRIIB on their cell surface (Ravetch JV et al., (1986), Science, 234(4777):718-25). This receptor contains the immunoreceptor tyrosine-based inhibition motif sequence (ITIM) in its cytoplasmic domain. Co-ligation of FcγRIIB and the B-cell receptor (BCR) by immune complexes act to trigger the tyrosine phosphorylation of the ITIM leading to the recruitment of the inositol phosphatase, SHIP, which inhibits BCR-triggered proliferation by interfering with the activation of MAP kinases and blocks phagocytosis by the dissociation of Burton's tyrosine kinase (Btk) from the cell membrane, which inhibits calcium influx into the cell. FcγRIIB can also induce apoptosis independent of the ITIM. Upon homo-aggregation of FcγRIIB by ICs, the association of Btk with the cell membrane is enhanced, thereby triggering an apoptotic response (Pearse R, et al., (1999), Immunity, 10(6):753-60). Expression of FcγRIIB is highly variable and cytokine dependent. IL-4 and IL-10, which are expressed by activated Th2 and T_{Reg} cells, have been shown to act synergistically to enhance FcγRIIB expression (Joshi T et al., (2006), Mol Immuno., 43(7):839-50), thus aiding in the suppression of a humoral response.

It is possible to exploit Tregitope specific T_{Reg} cells to suppress unwanted immune responses and also to induce adaptive T_{Reg} to co-delivered proteins. This discovery has implications for the design of therapeutic regimens and antigen-specific therapies for transplantation, protein therapeutics, allergy, chronic infection, autoimmunity and vaccine design. Administration of a drug, a protein, or an allergen in conjunction with Tregitopes, including a Tregitope compound or composition of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) can suppress an effector immune response. Tregitopes, including Tregitope compounds and compositions of the present disclosure, can be used to deliberately manipulate the immune system toward tolerance.

The Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) are useful in the selective engagement and activation of regulatory T cells. It is demonstrated herein that certain naturally occurring T_{Regs} (in aspects, including natural T_{Regs} and/or adaptive T_{Regs}), can be engaged, activated, and/or applied to the suppression of unwanted immune responses in both systemic and limited, disease specific, contexts. In aspects, the Tregitope compounds and compositions of the present disclosure can be used to engage and activate pre-existing populations of regulatory T cells to suppress an immune response caused by Factor VIII supplements that are used to prevent or stop bleeding in patients suffering from Hemophilia A

Despite extensive efforts, with few exceptions, the antigen specificity of natural T_{Regs}, and more importantly natural T_{Regs} circulating in clinically significant volumes, is unknown. Presented herein is a demonstration that certain human proteins circulating in the blood steam, such as immunoglobulins or the serum proteins coagulation Factor V (“FV”) and coagulation Factor VIII (“FVIII”), contain T cell epitopes that relate to naturally occurring populations of regulatory T cells (in aspects, including natural T_{Regs} and/or adaptive T_{Regs}). In the course of normal immune surveillance, these proteins are taken up by professional APCs, such as dendritic cells or macrophages, and degraded. During the degradation process, some of the epitopes contained in these proteins are bound to MHC molecules, transported to the cell surface presented to regulatory T cells. Those cells, once activated by the APC, release cytokines and chemokines help to suppress autoimmune responses that would otherwise hinder the function of the extra cellular proteins.

By using the Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) to selectively activate naturally occurring T_{Regs} (in aspects, including natural T_{Regs} and/or adaptive T_{Regs}), it is herein shown that the Tregitope compounds and compositions of the present disclosure can be used to suppress a variety of unwanted immune responses. In its simplest form, systemic application of the Tregitope compounds and compositions of the present disclosure can be used as a generalized immune suppressant useful for controlling severe autoimmune reactions such as, for example, MS flare-ups, allergic reactions, transplant reactions, or uncontrolled response to infection.

In a more controlled application, for example but not limited to, topically applied to joints affected by rheumatoid arthritis (RA), the Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) can be used to suppress localized autoimmune responses. In a targeted application, such as might be achieved through the fusion, bonding or admixture of the Tregitope compounds and compositions of the present disclosure to certain other T cell epitopes, the Tregitope compounds and compositions can suppress highly specific immune reactions to the fused, bonded, or admixed T cell epitopes while leaving the balance of the immune system intact. For example, through the delivery of a Tregitope compound or composition of the present disclosure fused to an autoimmune antigen such as insulin, an allergen such as Brazil nut antigen, or an antigenic protein such as an antibody (which can be IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody) or replacement enzyme (such as Factor V or Factor VIII replacement protein), the immune system can be trained to “tolerate” the co-delivered antigen by, e.g., inducing naturally occurring T_{Regs} (in aspects, including natural T_{Regs} and/or adaptive T_{Regs}) and/or converting the phenotype of responding effector T cells to that of adaptive regulatory T cells.

In certain aspects, the Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) can be used to suppress an immune response caused by Factor V supplements or Factor VIII supplements (such as those that are used to prevent or stop bleeding in patients suffering from Hemophilia A). For example, Factor VIII supplements can be administered with the Tregitope compounds and compositions of the present disclosure (e.g. but not limited to, through fusion, bonding, or admixture of the Factor VIII supplement(s) with the Tregitope compounds and compositions of the present disclosure, or insertion and/or linkage of the Tregitope(s) of the invention into the Factor VIII supplement(s)), with the Tregitope compounds and compositions of the present disclosure suppressing an immune response targeting the Factor VIII supplement(s). Such immune reprogramming could reduce or eliminate immune response targeting the Factor VIII supplements used to prevent or stop bleeding in patients suffering from Hemophilia A, while leaving the balance of the immune system intact.

As stated above, the Tregitopes of the present disclosure are derived from circulating extracellular proteins. To be useful, these Tregitopes should be true T cell epitopes (i.e., capable of binding to both MHC molecules and TCRs). In aspects, the Tregitopes should be related to a pre-existing population of regulatory T cells that is sufficiently large to have a therapeutic effect. T cell epitope clusters, which are epitopes capable of binding to multiple MHC alleles and multiple TCRs, are key to satisfying this latter qualification.

More particularly, the Tregitopes of the present disclosure are components of coagulation Factor V or Factor VIII. In their natural state, the Tregitopes of the present disclosure are capable of engaging and activating naturally occurring T_{Regs} (in aspects, including natural T_{Regs} and/or adaptive T_{Regs}) which prevent or terminate immune responses. These coagulation Factor V or VIII peptides are highly related to fragments of coagulation Factor VIII. In aspects, exposure of patients with Hemophilia A to the Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) can prevent an immune response to coagulation Factor VIII allowing treated patients the full benefit of Factor VIII supplements. Additionally, treatment with Tregitope compounds and compositions of the present disclosure can expand corresponding naturally occurring T_{Reg} populations (in aspects, including natural T_{Regs} and/or adaptive T_{Regs}), making them available to be activated by homologous peptides derived from Factor VIII, thereby suppressing effector response targeting Factor VIII. The instantly-disclosed treatments provides the following advantages:

1. Treatment with the Tregitope compounds and compositions of the present disclosure is highly antigen specific (e.g., treatment with the Tregitope compounds and compositions can, e.g., expand and/or stimulate corresponding naturally occurring T_{Reg} populations (in aspects, including natural T_{Regs} and/or adaptive T_{Regs}) in a highly antigen specific manner);

2. An efficient and less expensive treatment regimen when compared to current antigen specific therapies wherein patients are treated over a prolonged period of time with frequent high dose Factor VIII infusions; and

3. A second line of defense treatment when high dose Factor VIII treatment fails to induce immune tolerance in the treated patient.

In aspects, the present disclosure is directed to therapeutic Tregitope compounds and compositions (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) that are safely administered to a patient experiencing an autoimmune response. The mechanism of action of the claimed Tregitopes (and Tregitopes within said Tregitope compounds and compositions) is natural, supporting their efficacy and safety. In aspects, the present disclosure is directed to therapeutic Tregitope compounds and compositions that are safely administered to a Hemophiliac A patient in need of treatment, given that the Tregitopes of the instant disclosure are natural components of coagulation Factor V and Factor VIII, and as such, are naturally present in all humans.

In aspects, the present is directed to Tregitope compounds and compositions (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) that include one or more of the regulatory Tregitopes disclosed in Table 1, as well as fragments thereof, variants thereof, and fragments of such variants thereof, provided said fragments and/or variants retain MHC binding propensity and/or TCR specificity and/or regulatory T cell stimulating or suppressive activity. In certain aspects, the Tregitopes can be capped with an n-terminal acetyl and/or c-terminal amino group.

TABLE 1

| SEQUENCE | SEQUENCE ID NO: |
|---|---|
| ILTIHFTGHSFIYGK | (SEQ ID NO: 1) |
| IHSIHFSGHVFTVRK | (SEQ ID NO: 2) |
| KIVFKNMASRPYSIY | (SEQ ID NO: 3) |
| FAVFDENKSWYLEDN | (SEQ ID NO: 4) |
| ESNIMSTINGYVPES | (SEQ ID NO: 5) |
| EKDIHSGLIGPLLI | (SEQ ID NO: 6) |
| SHEFHAINGMIYSLP | (SEQ ID NO: 7) |
| IHFTGHSFI | (SEQ ID NO: 8) |
| IHFSGHVFT | (SEQ ID NO: 9) |
| FKNMASRPY | (SEQ ID NO: 10) |
| FDENLSWYL | (SEQ ID NO: 11) |
| IMSTINGYV | (SEQ ID NO: 12) |
| IHSGLIGPL | (SEQ ID NO: 13) |
| FHAINGMIY | (SEQ ID NO: 14) |
| ILTIHFTGH | (SEQ ID NO: 74) |
| LTIHFTGHS | (SEQ ID NO: 75) |
| TIHFTGHSF | (SEQ ID NO: 76) |
| FTGHSFIYG | (SEQ ID NO: 77) |
| TGHSFIYGK | (SEQ ID NO: 78) |
| IHSIHFSGH | (SEQ ID NO: 79) |
| HSIHFSGHV | (SEQ ID NO: 80) |
| SIHFSGHVF | (SEQ ID NO: 81) |
| HFSGHVFTV | (SEQ ID NO: 82) |
| FSGHVFTVR | (SEQ ID NO: 83) |
| SGHVFTVRK | (SEQ ID NO: 84) |
| KIVFKNMAS | (SEQ ID NO: 85) |
| IVFKNMASR | (SEQ ID NO: 86) |
| VFKNMASRP | (SEQ ID NO: 87) |
| KNMASRPYS | (SEQ ID NO: 88) |
| NMASRPYSI | (SEQ ID NO: 89) |
| MASRPYSIY | (SEQ ID NO: 90) |
| FAVFDENKS | (SEQ ID NO: 91) |
| AVFDENKSW | (SEQ ID NO: 92) |
| VFDENKSWY | (SEQ ID NO: 93) |
| FDENKSWYL | (SEQ ID NO: 94) |
| DENKSWYLE | (SEQ ID NO: 95) |
| ENKSWYLED | (SEQ ID NO: 96) |
| NKSWYLEDN | (SEQ ID NO: 97) |
| ESNIMSTIN | (SEQ ID NO: 98) |
| SNIMSTING | (SEQ ID NO: 99) |
| NIMSTINGY | (SEQ ID NO: 100) |
| MSTINGYVP | (SEQ ID NO: 101) |
| STINGYVPE | (SEQ ID NO: 102) |
| TINGYVPES | (SEQ ID NO: 103) |
| EKDIHSGLI | (SEQ ID NO: 104) |
| KDIHSGLIG | (SEQ ID NO: 105) |
| DIHSGLIGP | (SEQ ID NO: 106) |
| HSGLIGPLL | (SEQ ID NO: 107) |
| SGLIGPLLI | (SEQ ID NO: 108) |
| SHEFHAING | (SEQ ID NO: 109) |
| HEFHAINGM | (SEQ ID NO: 110) |
| EFHAINGMI | (SEQ ID NO: 111) |
| HAINGMIYS | (SEQ ID NO: 112) |
| AINGMIYSL | (SEQ ID NO: 113) |
| INGMIYSLP | (SEQ ID NO: 114) |
| HFTGHSFIYG | (SEQ ID NO: 115) |
| IHFSGHVFTV | (SEQ ID NO: 116) |

### Definitions

To further facilitate an understanding of the present invention, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the term “biological sample” as refers to any sample of tissue, cells, or secretions from an organism.

As used herein, the term “transplantation” refers to the process of taking a cell, tissue, or organ, called a “transplant” or “graft” from one subject and placing it or them into a (usually) different subject. The subject who provides the transplant is called the “donor”, and the subject who received the transplant is called the “recipient”. An organ or graft transplanted between two genetically different subjects of the same species is called an “allograft”. A graft transplanted between subjects of different species is called a “xenograft”.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens (including a virus), cells or tissues infected with pathogens, or, in cases of autoimmunity or pathological inflammation, normal human cells or tissues. In aspects, an immune response includes a measurable cytotoxic T lymphocyte (CTL) response (e.g., against a virus expressing an immunogenic polypeptide) or a measurable B cell response, such as the production of antibodies, (e.g., against an immunogenic polypeptide). One of ordinary skill would know various assays to determine whether an immune response against a peptide, polypeptide, or related composition was generated, including use of the experiments and assays as disclosed in the Examples herein. Various B lymphocyte and T lymphocyte assays are well known, such as ELISAs, EliSpot assays, cytotoxic T lymphocyte CTL assays, such as chromium release assays, proliferation assays using peripheral blood lymphocytes (PBL), tetramer assays, and other cytokine production assays. See Benjamini et al. (1991), hereby incorporated by reference.

As used herein, the term “effective amount”, “therapeutically effective amount”, or the like of a composition, including Tregitope compounds and compositions of the present disclosure (including one or more of e.g., polypeptides (which may be termed herein as “T_{reg} activating regulatory T-cell epitope”, “Tregitope”, or “T-cell epitope polypeptide”) having a sequence comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) is a quantity sufficient to achieve a desired therapeutic and/or prophylactic effect, e.g., an amount that results in the prevention of, or a decrease in, the symptoms associated with a disease that is being treated. The amount of a composition of the present disclosure administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present invention can also be administered in combination with each other or with one or more additional therapeutic compounds.

As used herein, the term “regulatory T cell”, “Treg” or the like, means a subpopulation of T cells that suppress immune effector function, including the suppression or down regulation of CD4+ and/or CD8+ effector T cell (Teff) induction, proliferation, and/or cytokine production, through a variety of different mechanisms including cell-cell contact and suppressive cytokine production. In aspects, CD4+ Tregs are characterized by the presence of certain cell surface markers including but not limited to CD4, CD25, and FoxP3. In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, CD8+ Tregs may also exert immune suppressive effects through direct killing of target cells, characterized by the expression upon activation of effector molecules including but not limited to granzyme B and/or perforin.

As used herein, the term “T cell epitope” means an MHC ligand or protein determinant, 7 to 30 amino acids in length, and capable of specific binding to human leukocyte antigen (HLA) molecules and interacting with specific T cell receptors (TCRs). As used herein, in the context of a T cell epitope that is known or determined (e.g. predicted) to engage a T cell, the terms “engage”, “engagement” or the like means that when bound to a MHC molecule (e.g. human leukocyte antigen (HLA) molecules), the T cell epitope is capable of interacting with the TCR of the T cell and activating the T cell. Generally, T cell epitopes are linear and do not express specific three-dimensional characteristics. T cell epitopes are not affected by the presence of denaturing solvents. The ability to interact with T cell epitopes can be predicted by in silico methods (De Groot AS et al., (1997), AIDS Res Hum Retroviruses, 13(7):539-41; Schafer JR et al., (1998), Vaccine, 16(19):1880-4; De Groot AS et al., (2001), Vaccine, 19(31):4385-95; De Groot AR et al.,(2003), Vaccine, 21(27-30):4486-504, all of which are herein incorporated by reference in their entirety.

As used herein, the term “T-cell epitope cluster” refers to polypeptide that contains between about 4 to about 40 MHC binding motifs. In particular embodiments, the T-cell epitope cluster contains between about 5 to about 35 MHC binding motifs, between about 8 and about 30 MHC binding motifs, or between about 10 and 20 MHC binding motifs.

As used herein, the term “regulatory T cell epitope” (“Tregitope”) refers to a “T cell epitope” that causes a tolerogenic response (Weber CA et al., (2009), Adv Drug Deliv, 61(11):965-76) and is capable of binding to MHC molecules and engaging (i.e. interacting with and activating) circulating naturally occurring Tregs (in aspects, including natural Tregs and/or adaptive Tregs). In aspects, upon activation, CD4+regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin, leads to the expression of the immune suppressive cytokines including, but not limited to, IL-10 and TGF-β and TNF-α. In aspects, upon activation, regulatory CD8+ T cells secrete immune suppressive cytokines and chemokines including but not limited to IFNγ, IL-10, and/or TGFβ. In aspects, CD8+ Tregs may also exert immune suppressive effects through direct killing of target cells, characterized by the expression upon activation of effector molecules including but not limited to granzyme B and/or perforin. In aspects, the instantly disclosed Tregitopes are T cell epitope clusters, which are epitopes capable of binding to multiple MHC alleles and multiple TCRs.

As used herein, the term “immune stimulating T-cell epitope polypeptide” refers to a molecule capable of inducing an immune response, e.g., e.g., a humoral, T cell-based, or innate immune response. In aspects, an immune stimulating T-cell epitope polypeptide is human Coagulation Factor V (or Factor V replacement protein or supplement, or human Coagulation Factor VIII (or Factor VIII replacement protein or supplement).

As used herein, the term “B cell epitope” means a protein determinant capable of specific binding to an antibody. B cell epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the terms “the major histocompatibility complex (MHC)”, “MHC molecules”, “MHC proteins” or “HLA proteins” are to be understood as meaning, in particular, proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin. MHC molecules of class II consist of an α-chain and a β-chain and are capable of binding a peptide of about 12 to 25 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

As used herein, the term “MHC complex” refers to a protein complex capable of binding with a specific repertoire of polypeptides known as HLA ligands and transporting said ligands to the cell surface.

As used herein, the term “MHC Ligand” means a polypeptide capable of binding to one or more specific MHC alleles. The term “HLA ligand” is interchangeable with the term “MHC Ligand”. Cells expressing MHC/Ligand complexes on their surface are referred to as “Antigen Presenting Cells” (APCs). Similarly, as used herein, the term “MHC binding peptide” relates to a peptide which binds to an MHC class I and/or an MHC class II molecule. In the case of MHC class I/peptide complexes, the binding peptides are typically 8-10 amino acids long although longer or shorter peptides may be effective. In the case of MHC class II/peptide complexes, the binding peptides are typically 10-25 amino acids long and are in particular 13-18 amino acids long, whereas longer and shorter peptides may also be effective.

As used herein, the term “T Cell Receptor” or “TCR” refers to a protein complex expressed by T cells that is capable of engaging a specific repertoire of MHC/Ligand complexes as presented on the surface of cells, such as antigen presenting cells (APCs).

As used herein, the term “MHC Binding Motif” refers to a pattern of amino acids in a protein sequence that predicts binding to a particular MHC allele.

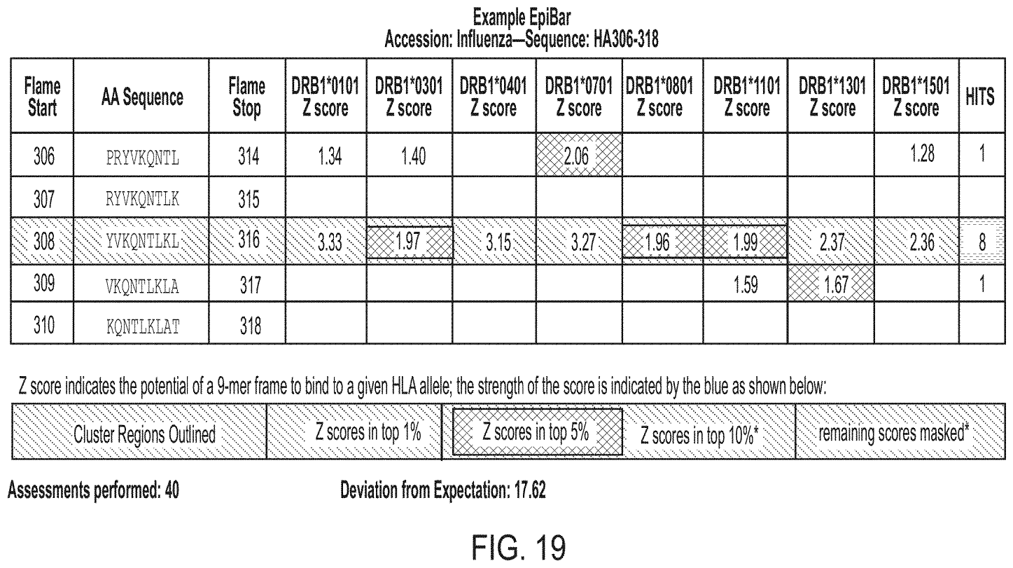

As used herein, the term “EpiBar™” refers to a single 9-mer frame that is predicted to bind to at least four different HLA alleles. A representative example of an immunogenic peptide that contains an EpiBar™ is shown below in FIG. 19. FIG. 19 depicts an example of an EpiBar and the EpiMatrix analysis of a promiscuous influenza epitope. Consider the influenza HA peptide, an epitope known to be promiscuously immunogenic. It scores extremely high for all eight alleles in EpiMatrix. Its cluster score is 18. Cluster scores higher than 10 are considered to be significant. The band-like EpiBar pattern is characteristic of promiscuous epitopes. Results are shown in FIG. 19 for PRYVKQNTL (SEQ ID NO: 60), RYVKQNTLK (SEQ ID NO: 61), YVKQNTLKL (SEQ ID NO: 62), VKQNTLKLA (SEQ ID NO: 63) and KQNTLKLAT (SEQ ID NO: 64). Z score indicates the potential of a 9-mer frame to bind to a given HLA allele. All scores in the top 5% are considered “hits”, while non hits (*) beow 10% are masked in FIG. 19 for simplicity.

As used herein, the term “native Fc” refers to a molecule or sequence comprising the sequence of a non-antigen-binding fragment resulting from digestion of whole antibody, whether in monomeric or multimeric form, into which a peptide sequence may be added by insertion into or replacement of a loop region. The original immunoglobulin source of the native Fc is preferably of human origin and may be any of the immunoglobulins, including but not limited to IgG1 and IgG2. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

As used herein, the term “Immune Synapse” means the protein complex formed by the simultaneous engagement of a given T cell epitope to both a cell surface MHC complex and TCR.

The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide (e.g., a polypeptide comprising, consisting of, or consisting essentially of one or more of SEQ ID NOS: 1-14 and 74-116 or variants and fragments thereof, which in aspects may be isolated, synthetic, or recombinant) of the present disclosure, however, can be joined to, linked to, or inserted into another polypeptide (e.g., a heterologous polypeptide) with which it is not normally associated in a cell and still be “isolated” or “purified.” As used herein with respect to the one or more Tregitopes of the instant disclosure, the term “heterologous polypeptide” is intended to mean that the one or more Tregitopes is heterologous to, or not included naturally, in the heterologous polypeptide. For example, one or more Tregitopes of the present disclosure (and/or one or more other Tregitopes, such as an IgG derived Tregitope as disclosed in U.S. Pat. No. 7.884,184, which is incorporated by reference in its entirety) can be linked to (e.g., fused in-frame, chemically-linked, or otherwise bound) and/or inserted into a heterologous polypeptide (e.g., but not limited to, an antibody (which, in aspects, may be monoclonal, polyclonal, mouse, human, humanized, monospecific, bispecific, glycosylated (e.g., sugar chain-modified), Fc-modified, or antibody-drug conjugate; or an antibody fragment thereof (e.g., Fab, scFv, diabody, sdAb, or tandem scFv); or an antibody of different class or subclass (e.g., IgG (e.g., IgG1, IgG2, IgG3, or IgG4), IgM, IgE, or IgA)). Additionally, one or more Tregitopes of the present disclosure can be joined to, linked to, or inserted into another polypeptide wherein said one or more Tregitopes of the present disclosure is not naturally included in the polypeptide and/or said one or more Tregitopes of the present disclosure is not located at its natural position in the polypeptide. For example, in aspects, the one or more Tregitopes may be inserted into or replace amino acids in a Fc domain as disclosed in U.S. Pat. No. 7,442,778, U.S. Pat. No. 7,645,861, U.S. Pat. No. 7,655,764, U.S. Pat. No. 7,655,765, and/or U.S. Pat. No. 7,750,128 (each of which are herein incorporated by reference in their entirety). In aspects, the one or more Tregitopes may be covalently bound to one or more internal conjugation site(s) in a Fc domain as disclosed in U.S. Pat. No. 8,008,453, U.S. Pat. No. 9,114,175, and/or U.S. Pat. No. 10,188740 (each of which are herein incorporated by reference in their entirety). When a polypeptide is recombinantly produced, it can also be substantially free of culture medium, for example, culture medium represents less than about 20%, less than about 10%, or less than about 5% of the volume of the polypeptide preparation.

As used herein, a “concatemeric” peptide or polypeptide refers to a series of at least two peptides or polypeptides linked together. Such linkages may form of string-of-beads design. In aspects, each of the peptides or polypeptides of concatemeric polypeptide may optionally be spaced by one or more linkers, and in further aspects neutral linkers. The term “linker” may refer to a peptide added between two peptide domains such as epitopes or vaccine sequences to connect said peptide domains. In aspects, a linker sequence is used to reduce steric hindrance between each one or more identified peptides of the instant disclosure, is well translated, and supports or allows processing of the each one or more identified polypeptides of the instant disclosure. In aspects, the linker should have little or no immunogenic sequence elements. In aspects, each peptide or polypeptide of the concatemeric polypeptide may optionally have one or more linkers, which may optionally be cleavage sensitive sites, adjacent to their N and/or C terminal end. In such a concatemeric peptide, two or more of the peptides may have a cleavage sensitive site between them. Alternatively two or more of the peptides may be connected directly to one another or through a linker that is not a cleavage sensitive site.

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier, or diluent” or the like refer to an excipient, carrier, or diluent that can be administered to a subject, together with an agent, and which does not destroy the pharmacological activity thereof and is nontoxic when administered in doses sufficient to deliver a therapeutic amount of the agent

As used herein, a “free thiol” refers to a thiol side chain of an amino acid optionally in a polypeptide and/or protein, wherein the thiol contains a sulfhydryl group. For example, free thiols are not bound to the side chains of other amino acids through intramolecular or intermolecular disulfide bonds.

As used herein, “functionalities” are groups on blood components, including mobile and fixed proteins, to which reactive groups on modified therapeutic peptides react to form covalent bonds. Functionalities may include hydroxyl groups for bonding to ester reactive groups, thiol groups for bonding to maleimides, imidates and thioester groups; amino groups for bonding to activated carboxyl, phosphoryl or any other acyl groups on reactive groups.

As used herein, “blood components” may be either fixed or mobile. Fixed blood components are non-mobile blood components and include tissues, membrane receptors, interstitial proteins, fibrin proteins, collagens, platelets, endothelial cells, epithelial cells and their associated membrane and membranous receptors, somatic body cells, skeletal and smooth muscle cells, neuronal components, osteocytes and osteoclasts and all body tissues especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components may be at least about 12 hours.

As used herein, the term “purpose built computer program” refers to a computer program designed to fulfill a specific purpose; typically to analyze a specific set of raw data and answer a specific scientific question.

As used herein, the term “z-score” indicates how many standard deviations an element is from the mean. A z-score can be calculated from the following formula. z=(X−μ)/σ where z is the z-score, X is the value of the element, μ is the population mean, and σ is the standard deviation.

As used herein, the singular forms “a,” “an,” and “the” are intended to include the plural forms, including “at least one,” unless the content clearly indicates otherwise. “Or” means “and/or.” As used herein, the term “and/or” and “one or more” includes any and all combinations of the associated listed items. For example, the term “one or more” with respect to the “one or more of SEQ ID NOS: 1-14 and 74-116 of the present disclosure” includes any and all combinations of SEQ ID NOS: 1-14 and 74-116. The term “or a combination thereof” means a combination including at least one of the foregoing elements.

The following abbreviations and/or acronyms are used throughout this application:
APC antigen presenting cells
CEF cytomegalovirus, Epstein-Barr virus and influenza virus
CFSE dye carboxyfluorescein succinimidyl ester dye
DMSO dimethyl sulfoxide
DR antibody antigen D related antibody
ELISA enzyme-linked immunosorbent assay
FACS fluorescence-activated cell sortings
Fmoc 9-fluoronyl methoxy carbonyl
FV human coagulation Factor V
FVIII human coagulation Factor VIII
HLA human leukocyte antigen
HPLC high-performance liquid chromatography
IVIG intravenous purified Immunoglobulin G antibody
MFI mean fluorescence index
MHC major histocompatibility complex
PBMC peripheral blood mononuclear cell
PI proliferation index
RPMI Roswell Park Memorial Institute medium
T_{eff} effector T cell
T_{Reg} regulatory T cell
TT tetanus toxoid
UV ultraviolet

As used herein, a “variant” peptide or polypeptide (including a variant Tregitope) can differ in amino acid sequence by one or more substitutions, deletions, insertions, inversions, fusions, and truncations or a combination of any of these. In aspects, a variant peptide or polypeptide (including a variant T-cell epitope) can differ in amino acid sequence by one or more substitutions, deletions, insertions, inversions, fusions, and truncations or a combination of any of these provided said variants retain MHC binding propensity and/or TCR specificity, and/or regulatory T cell stimulating or suppressive activity.

As used herein, an “antibody” can take various forms, including, but not limited to, one or more of the following: monoclonal or polyclonal; mouse, human, or humanized; monospecific or bispecific; glycosylated; Fc-modified; antibody-drug conjugate; antibody of different class or subclass, such as IgG (e.g., IgG1, IgG2, IgG3, or IgG4), IgM, IgE, or IgA; and/or antibody fragments or derivatives thereof (e.g., Fab, scFv, diabody, sdAb, or tandem sccFv.

The present disclosure also includes polypeptide fragments of the Tregitopes of the invention. The invention also encompasses fragments of the variants of the Tregitopes described herein, provided said fragments and/or variants retain MHC binding propensity and/or TCR specificity, and/or regulatory T cell stimulating or suppressive activity.

The present disclosure also provides chimeric or fusion polypeptides (which in aspects may be isolated, synthetic, or recombinant) wherein one or more of the instantly disclosed Tregitopes is a part thereof. In aspects, a chimeric or fusion polypeptide composition comprises one or more polypeptides (Treg activating regulatory T-cell epitope, Tregitope, Tregitope peptide, or T-cell epitope polypeptide) of the instant disclosure linked to a heterologous polypeptide (e.g. but not limited to, a Factor V or Factor VIII molecule or replacement protein/supplement (or fragments thereof), a monoclonal antibody, polyclonal antibody, mouse antibody, human antibody, humanized antibody, mono specific antibody, bispecific antibody, glycosylated antibody, Fc-modified antibody, or antibody-drug conjugates; an antibody of different class or subclass (e.g., IgG (e.g., IgG1, IgG2, IgG3, IgG4), IgM, IgA, IgD or IgE molecules) or antigen-specific antibody fragments thereof (including, but not limited to, a Fab, F(ab′)2, Fv, disulfide linked Fv, scFv, single domain antibody, closed conformation multispecific antibody, disulfide-linked scFv, diabody)). As previously stated, the term “heterologous polypeptide” is intended to mean that the one or more Tregitopes of the instant disclosure (e.g., one or more of SEQ ID NOS: 1-14 and 74-116) are heterologous to, or not included naturally, in the heterologous polypeptide. In aspects, the one or more Tregitopes may be inserted into the heterologous polypeptide (e.g., through mutagenesis or other known means in the art), may be added to the C-terminus (with or without the use of linkers, as is known in the art), and/or added to the N-terminus (with or without the use of linkers, as is known in the art) of the heterologous polypeptide. In aspects, the one or more Tregitopes may be inserted into or replace amino acids in a Fc domain as disclosed in U.S. Pat. No. 7,442,778, U.S. Pat. No. 7,645,861, U.S. Pat. No. 7,655,764, U.S. Pat. No. 7,655,765, and/or U.S. Pat. No. 7,750,128 (each of which are herein incorporated by reference in their entirety). For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, chimeric or fusion polypeptides comprise one or more Tregitope of the present disclosure operatively linked to a heterologous polypeptide. “Operatively linked” indicates that the polypeptide (e.g., the one or more Treg activating regulatory T-cell epitope, Tregitope, Tregitope peptide, or T-cell epitope polypeptide of the present disclosure) and the heterologous protein are fused in-frame or chemically linked or otherwise bound. For example, in aspects, the one or more Tregitopes may be covalently bound to one or more internal conjugation site(s) in an Fc domain as disclosed in U.S. Pat. No. 8,008,453, U.S. Pat. No. 9,114,175, and/or U.S. Pat. No. 10,188,740 (each of which are herein incorporated by reference in their entirety). In aspects, an isolated, synthetic, or recombinant chimeric or fusion polypeptide composition comprises a polypeptide, said polypeptide having a sequence comprising one or more of SEQ ID NOS: 1-14 and 74-116 of the present disclosure, wherein said one or more of SEQ ID NOS: 1-14 and 74-116 is not naturally included in the polypeptide and/or said of one or more of SEQ ID NOS: 1-14 and 74-116 is not located at its natural position in the polypeptide. In aspects, the one or more of SEQ ID NOS: 1-14 and 74-116 of the present disclosure can be joined, linked to (e.g., fused in-frame, chemically linked, or otherwise bound), and/or inserted into the polypeptide. In aspects, the one or more of SEQ ID NOS: 1-14 and 74-116 of the present disclosure can be joined or linked to (e.g., fused in-frame, chemically-linked, or otherwise bound) to a small molecule, drug, or drag fragment, for example but not limited to, a drug or drug fragment that is binds with high affinity to defined HLAs. In aspects of the above chimeric or fusion polypeptide compositions, the one or more polypeptides (Treg activating regulatory T-cell epitope, Tregitope, Tregitope peptide, or T-cell epitope polypeptide) of the present disclosure have a sequence comprising, consisting of, or consisting essentially of one or more of SEQ ID NOS: 1-14 and 74-116, and optionally 1 to 12 additional amino acids distributed in any ratio on the N terminus and/or C-terminus of the polypeptide of SEQ ID NOS: 1-14 and 74-116. In aspects of the above chimeric or fusion polypeptide compositions, the one or more polypeptides (Treg activating regulatory T-cell epitope, Tregitope, Tregitope peptide, or T-cell epitope polypeptide) of the present disclosure comprises, consists of, or consists essentially of a sequence one or more of SEQ ID NOS: 1-2. In aspects of the above chimeric or fusion polypeptide compositions, the one or more polypeptides (Treg activating regulatory T-cell epitope, Tregitope, Tregitope peptide, or T-cell epitope polypeptide) of the present disclosure comprises, consists of, or consists essentially the amino acid sequence of SEQ ID NO: 1.

An “isolated” polypeptide (e.g., an isolated Treg activating regulatory T-cell epitope, Tregitope, Tregitope peptide, or T-cell epitope polypeptide) polypeptide, concatemeric peptide, or chimeric or fusion polypeptide can be purified from cells that naturally express it, purified from cells that have been altered to express it (recombinant), or synthesized using known protein synthesis methods. In one embodiment, a peptide, polypeptide, concatemeric peptide, or chimeric or fusion polypeptide is produced by recombinant DNA or RNA techniques. For example, a nucleic acid molecule encoding the Tregitope is cloned into an expression vector, the expression vector introduced into a host cell and the polypeptide expressed in the host cell. The Tregitope can then be isolated from the cells by an appropriate purification scheme using standard protein purification techniques.

For the purposes of the present disclosure, peptides, polypeptides, concatemeric peptides, or chimeric or fusion polypeptides of the instant disclosure can include, for example, modified forms of naturally occurring amino acids such as D-stereoisomers, non-naturally occurring amino acids; amino acid analogs; and mimetics. Further, in aspects, peptides, polypeptides, concatemeric peptides, or chimeric or fusion polypeptides of the instant disclosure can include retro-inverso peptides of the instantly disclosed peptides, polypeptides, concatemeric peptides, or chimeric or fusion polypeptides of the instant disclosure, provided said retro-inverso peptides, polypeptides, concatemeric peptides, or chimeric or fusion polypeptides of the instant disclosure at least in part retain MHC binding propensity and/or TCR specificity, and/or regulatory T cell stimulating or suppressive activity.

Although any methods and materials similar or equivalent to those described herein can be used in the practice or testing of the present disclosure, the preferred methods and materials are described. Other features, objects, and advantages of the present disclosure will be apparent from the description and the Claims. In the Specification and the appended Claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

### Polypeptides, Concatemeric Polypeptides, and Chimeric or Fusion Polypeptides

In aspects, the present disclosure provides Tregitope compounds and compositions, including (Treg activating regulatory T-cell epitope, Tregitope, Tregitope peptide, or T-cell epitope polypeptide, which in aspects may be isolated, synthetic, or recombinant) as disclosed herein; nucleic acids, expression cassettes, plasmids, expression vectors, recombinant viruses, or cells (all of which in aspects may be isolated, synthetic, or recombinant) as disclosed herein; isolated, synthetic, or recombinant chimeric or fusion polypeptide compositions as disclosed herein; and/or pharmaceutical compositions or formulations as disclosed herein. In aspects, the Tregitope compounds and compositions include one or more of the regulatory Tregitopes of Table 1 (including fragments thereof, variants thereof, and fragments of such variants thereof, provided said fragments and/or variants retain MHC binding propensity and/or TCR specificity, and/or regulatory T cell stimulating or suppressive activity). In certain aspects, the Tregitopes can be capped with an n-terminal acetyl and/or c-terminal amino group.

In one aspect, the present disclosure provides a novel class of T cell epitopes (which may be isolated, synthetic, or recombinant), termed ‘Tregitopes’, which comprise a peptide or polypeptide chain derived from common human proteins. Tregitopes of the present disclosure are highly conserved among known variants of their source proteins (e.g., present in more than 10% of known variants). Tregitopes of the present disclosure comprise at least one putative T cell epitope as identified by EpiMatrix™ analysis. EpiMatrix™ is a proprietary computer algorithm developed by EpiVax (Providence, R.I.), which is used to screen protein sequences for the presence of putative T cell epitopes. Input sequences are parsed into overlapping 9-mer frames where each frame overlaps the last by 8 amino acids. Each of the resulting frames is then scored for predicted binding affinity with respect to a panel of eight common Class II HLA alleles (DRB1*0101, DRB1*0301, DRB1*0401, DRB1*0701, DRB1*0801, DRB1*1101, DRB1*1301, and DRB1*1501). Raw scores are normalized against the scores of a large sample of randomly generated peptides. The resulting “Z” score is reported. In aspects, any 9-mer peptide with an allele-specific EpiMatrix™ Z-score in excess of 1.64, theoretically the top 5% of any given sample, is considered a putative T cell epitope.

Peptides containing clusters of putative T cell epitopes are more likely to test positive in validating in vitro and in vivo assays. The results of the initial EpiMatrix™ analysis are further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly 30 amino acids in length and, considering their affinity to multiple alleles and across multiple 9-mer frames, can contain anywhere from about 4 to about 40 putative T cell epitopes. Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, the Tregitopes of the instant disclosure contain several putative T cell epitopes forming a pattern known as a T cell epitope cluster.

Many of the most reactive T cell epitope clusters contain a feature referred to as an “EpiBar™”. As previously described, an EpiBar™ is a single 9-mer frame that is predicted to be reactive to at least four different HLA alleles. In aspects, the Tregitopes of the present disclosure can comprise one or more EpiBars™.

The JanusMatrix system (EpiVax, Providence, R.I.) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and as such may be very useful Tregitopes of the present disclosure.

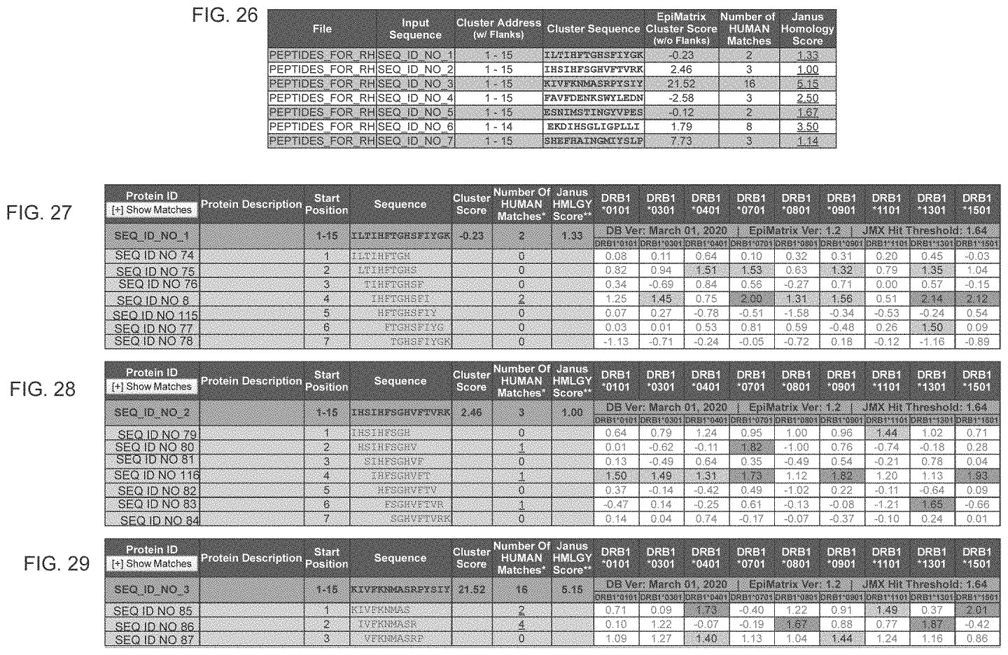

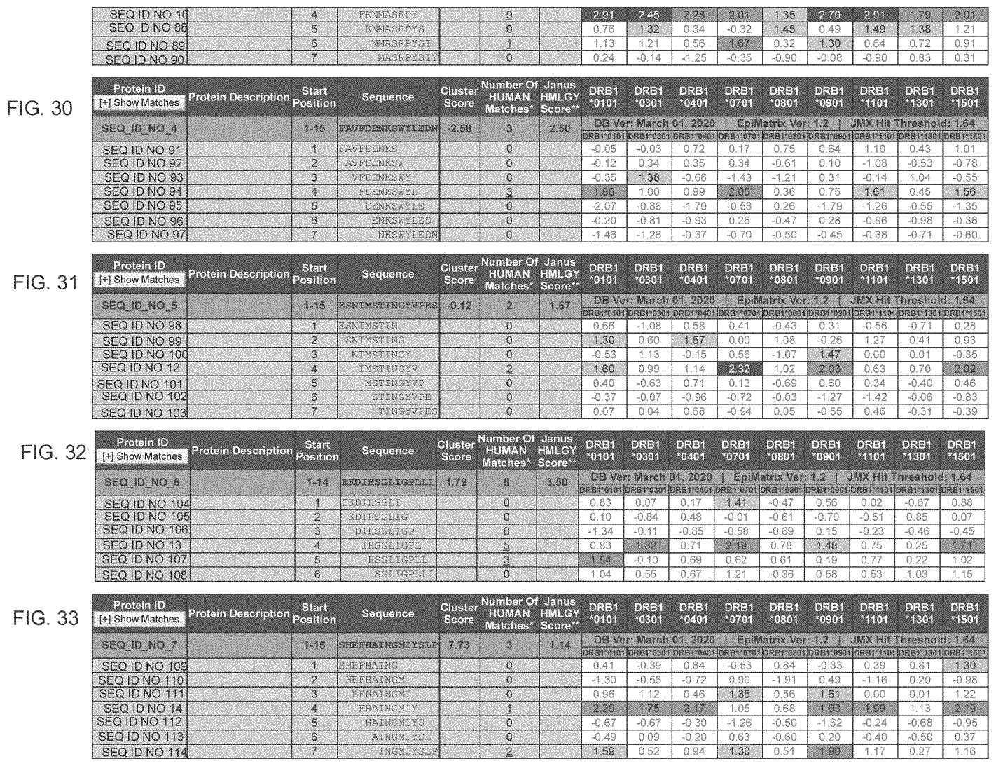

FIG. 26 is the overview of JanusMatrix results for identified the Tregitopes of SEQ ID NOS: 1-7 of the instant disclosure. FIG. 27 is the JanusMatrix report for the Tregitope of SEQ ID NO: 1 and the 9-mers contained within SEQ ID NO: 1, including SEQ ID NOS: 74-78, 8, and 115. FIG. 28 is the JanusMatrix report for the Tregitope of SEQ ID NO: 2 and the 9-mers contained within SEQ ID NO: 2, including SEQ ID NOS: 19-84, and 116. FIG. 29 is the JanusMatrix report for the Tregitope of SEQ ID NO: 3 and the 9-mers contained within SEQ ID NO: 3, including SEQ ID NOS: 85-90 and 10. FIG. 30 is the JanusMatrix report for the Tregitope of SEQ ID NO: 4 and the 9-mers contained within SEQ ID NO: 4, including SEQ ID NOS: 91-97. FIG. 31 is the JanusMatrix report for the Tregitope of SEQ ID NO: 5 and the 9-mers contained within SEQ ID NO: 5, including SEQ ID NOS: 98-103 and 12. FIG. 32 is the JanusMatrix report for the Tregitope of SEQ ID NO: 6 and the 9-mers contained within SEQ ID NO: 6, including SEQ ID NOS: 104-108 and 13. FIG. 33 is the JanusMatrix report for the Tregitope of SEQ ID NO: 7 and the 9-mers contained within SEQ ID NO: 7, including SEQ ID NOS: 109-114 and 14. For each of FIGS. 26-33, * is the count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. Further, the Janus Homology Score** represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

In aspects, Tregitopes of the present disclosure bind to at least one and preferably two or more common HLA class II molecules with at least a moderate affinity (e.g., in aspects, <1000 μM IC₅₀, <500 μM IC₅₀, <400 μM IC₅₀, <300 μM IC₅₀, or <200 μM IC₅₀ in HLA binding assays based on soluble HLA molecules). In aspects, Tregitopes of the present disclosure are capable of being presented at the cell surface by APCs in the context of at least one and, in other aspects, two or more alleles of the HLA. In this context, the Tregitope HLA complex can be recognized by naturally occurring TRegs (in aspects, including natural TRegs and/or adaptive TRegs) having TCRs that are specific for the Tregitope HLA complex and circulating in normal control subjects. In aspects, the recognition of the Tregitope-HLA complex can cause the matching regulatory T cell to be activated and to secrete regulatory cytokines and chemokines.

In aspects, the present disclosure is directed to a polypeptide (which may be termed herein as “Treg activating regulatory T-cell epitope”, “Tregitope”, “Tregitope peptide”, or “T-cell epitope polypeptide”) having a sequence comprising, consisting of, or consisting essentially of one or more of SEQ ID NOS: 1-14 and 74-116 (and fragments and variants thereof). The phrase “consisting essentially of” is intended to mean that a polypeptide according to the present disclosure, in addition to having the sequence according to any of SEQ ID NOS: 1-14 and 74-116 or a variant thereof, contains additional amino acids or residues that may be present at either terminus of the peptide and/or on a side chain that are not necessarily forming part of the peptide that functions as an MHC ligand and provided they do not substantially impair the activity of the peptide to function as a Tregitope. In aspects, a polypeptide comprises, consists of, or consists essentially of one or more of SEQ ID NOS: 1-2. In aspects, a polypeptide comprises, consists of, or consists essentially of the amino acid sequence of SEQ ID NO: 1. In aspects, the peptides or polypeptides of the instant disclosure can be either in neutral (uncharged) or salt forms, and may be either free of or include modifications such as glycosylation, side chain oxidation, or phosphorylation. In certain aspects, such polypeptides can be capped with an N-terminal acetyl and/or C-terminal amino group.

In aspects, the instant disclosure is directed to a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116. In aspects, the instant disclosure is directed to a peptide or polypeptide have a core amino acid sequence comprising, consisting of, or consisting essentially of one or more peptides or polypeptides having an amino acid sequence of SEQ ID NOS: 1-14 and 74-116, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide having a core sequence comprising, consisting of, or consisting essentially of one or more peptides or polypeptides having an amino acid sequence of SEQ ID NOS: 1-14 and 74-116 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to a same HLA molecule (i.e., retain MHC binding propensity) as said polypeptide core sequence without said flanking amino acids. In aspects, said polypeptide with the flanking amino acids is still able to bind to a same HLA molecule (i.e., retain MHC binding propensity) and/or retain the same TCR specificity, and/or retain regulatory T cell stimulating or suppressive activity, as said polypeptide core sequence without said flanking amino acids. In aspects, said flanking amino acid sequences are those that also flank the peptides or polypeptides included therein in the naturally occurring protein (e.g., in human Factor V or human Factor VIII). In aspects, said flanking amino acid sequences as described herein may serve as a MHC stabilizing region. The use of a longer peptide may allow endogenous processing by patient cells and may lead to more effective antigen presentation and induction of T cell responses. In aspects, the peptides or polypeptides can be either in neutral (uncharged) or salt forms, and may be either free of or include modifications such as glycosylation, side chain oxidation, or phosphorylation. In certain aspects, the peptides or polypeptides can be capped with an n-terminal acetyl and/or c-terminal amino group.

In aspects, the instant disclosure is directed to a polypeptide comprising an amino acid sequence having at least 75%, 80%, 85%, 90%, or 95% homology to any one of SEQ ID NOS: 1-14 and 74-116 (and/or fragments thereof), wherein said polypeptide is still able to bind to a same HLA molecule (i.e., retain MHC binding propensity) and/or retain the same TCR specificity, and/or retain regulatory T cell stimulating or suppressive activity.

In aspects, the present disclosure is directed to a concatemeric polypeptide or peptide that comprises at one or more of the instantly-disclosed polypeptides or peptides (e.g., but not limited to, a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects a concatemeric peptide is composed of 1 or more, 2 or more, 3 or more, 4 or more, 5 or more 6 or more 7 or more, 8 or more, 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. It should be understood that the present disclosure also relates to nucleic acids (e.g., RNA mRNA, DNA, cDNA) encoding such concatemeric peptides. Each peptide or polypeptide of the concatemeric polypeptide may optionally have one or more linkers, which may optionally be cleavage sensitive sites, adjacent to their N and/or C terminal end. Such suitable linkers and cleavage sensitive sites, including AAY cleavage motifs or a poly GS linker which may be include on the N terminus of the C-terminal element, are known in the art. In such a concatemeric peptide, two or more of the peptide epitopes may have a cleavage sensitive site between them. Alternatively, two or more of the peptide epitopes may be connected directly to one another or through a linker that is not a cleavage sensitive site. In aspects, such linker is antigenically neutral, and the liker is preferably less than the length of a peptidyl backbone of 9 amino acids linearly arranged. In aspects, linker length is the length of a peptidyl backbone of between 2 and 8 amino acids, linearly arranged. In aspects, the spacer is unable to hydrogen bond in any spatially distinct manner to other distinct elements of the enhancing hybrid peptide.

In aspects, and with respect to antigenically neutral linker elements, various chemical groups may be incorporated as linkers instead of amino acids. Examples are described in U.S. Pat. No. 5,910,300, the contents of which are incorporated herein by reference. In aspects, a linker may be comprised of an aliphatic chain optimally interrupted by heteroatoms, for example a C2-C6 alkylene, or ═N—(CH2)2-6—N═. Alternatively, a spacer may be composed of alternating units, for example of hydrophobic, lipophilic, aliphatic and aryl-aliphatic sequences, optionally interrupted by heteroatoms such as O, N, or S. Such components of a spacer are preferably chosen from the following classes of compounds: sterols, alkyl alcohols, polyglycerides with varying alkyl functions, alkyl-phenols, alkyl-amines, amides, hydroxyphobic polyoxyalkylenes, and the like. Other examples are hydrophobic polyanhydrides, polyorthoesters, polyphosphazenes, polyhydroxy acids, polycaprolactones, polylactic, polyglycolic polyhydroxy-butyric acids. A linker may also contain repeating short aliphatic chains, such as polypropylene, isopropylene, butylene, isobutylene, pentamethlyene, and the like, separated by oxygen atoms.

Additional peptidyl sequences which can be used in as possible linkers are described in U.S. Pat. No. 5,856,456, the contents of which are incorporated herein by reference. In one embodiment, a linker has a chemical group incorporated within which is subject to cleavage. Without limitation, such a chemical group may be designed for cleavage catalyzed by a protease, by a chemical group, or by a catalytic monoclonal antibody. In the case of a protease-sensitive chemical group, tryptic targets (two amino acids with cationic side chains), chymotryptic targets (with a hydrophobic side chain), and cathepsin sensitivity (B, D or S) are favored. The term ‘tryptic target’ is used herein to describe sequences of amino acids which are recognized by trypsin and trypsin-like enzymes. The term ‘chymotryptic target’ is used herein to describe sequences of amino acids which are recognized by chymotrypsin and chymotrypsin-like enzymes. In addition, chemical targets of catalytic monoclonal antibodies, and other chemically cleaved groups are well known to persons skilled in the art of peptide synthesis, enzymatic catalysis, and organic chemistry in general, and can be designed into the hybrid structure and synthesized, using routine experimental methods.

In aspects, a concatemeric polypeptide of the instant disclosure is produced using the EpiAssembler System (EpiVax). The EpiAssembler system is useful for assembling overlapping epitopes to Immunogenic Consensus Sequences (ICS). EpiAssembler is an algorithm that optimizes the balance between pathogen and population coverage. EpiAssembler uses the information from the sequences produced by Conservatrix and EpiMatrix to form highly immunogenic consensus sequences.

In aspects of above-described concatemeric peptides or polypeptides, the concatemeric peptides or polypeptides may be isolated, synthetic, or recombinant. In aspects, the concatemeric peptides or polypeptides can be in either neutral (uncharged) or salt forms, and may be either free of or include modifications such as glycosylation, side chain oxidation, or phosphorylation. In certain aspects, the concatemeric polypeptides can be capped with an N-terminal acetyl and/or C-terminal amino group.

As used herein, two polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software. As used herein, amino acid or nucleic acid “homology” is equivalent to amino acid or nucleic acid “identity”. In aspects, the percent homology between the two sequences is a function of the number of identical positions shared by the sequences (e.g., percent homology equals the number of identical positions/total number of positions×100).

In aspects, the present disclosure also encompasses polypeptides having a lower degree of identity but having sufficient similarity so as to perform one or more of the same functions performed by a polypeptide of the instant disclosure (e.g., a polypeptide having a sequence comprising, consisting of, or consisting essentially of one or more of SEQ ID NOS: 1-14 and 74-116 and/or fragments and variants thereof, and optionally 1 to 12 additional amino acids distributed in any ratio on the N terminus and/or C-terminus of the polypeptide of SEQ ID NOS: 1-14 and 74-116; and concatemeric peptides as disclosed herein). Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and Ile; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr. Guidance concerning which amino acid changes are likely to be phenotypically silent are found (Bowie JU et al., (1990), Science, 247(4948):130610, which is herein incorporated by reference in its entirety).

In aspects, a variant polypeptide can differ in amino acid sequence by one or more substitutions, deletions, insertions, inversions, fusions, and truncations or a combination of any of these. Variant polypeptides can be fully functional (e.g., retain MHC binding propensity and/or TCR specificity, and/or retain regulatory T cell stimulating or suppressive activity) or can lack function in one or more activities. Fully functional variants typically contain only conservative variation or variation in non-critical residues or in non-critical regions; in this case, typically MHC contact residues provided MHC binding is preserved. Functional variants can also contain substitution of similar amino acids that result in no change or an insignificant change in function (e.g., retain MHC binding propensity and/or TCR specificity, and/or retain regulatory T cell stimulating or suppressive activity). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues. In aspects, a variant and/or a homologous polypeptide retains the desired regulatory T cell stimulating or suppressive activity of the instant disclosure. Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues. In aspects, functional variants of a polypeptide having a sequence (or a core sequence) comprising, consisting of, or consisting essentially of one or more of SEQ ID NOS: 1-14 and 74-116 as disclosed herein may contain one or more conservative substitutions, and in aspects one or more non-conservative substitutions, at amino acid residues which are not believed to be essential for functioning (with amino acid residues considered being essential for functioning, including, e.g., retain MHC binding propensity and/or TCR specificity, and/or retain regulatory T cell stimulating or suppressive activity) of the instantly-disclosed polypeptides. For example, in aspects, a variant polypeptide having a sequence (or a core sequence) comprising, consisting of, or consisting essentially of one or more of SEQ ID NOS: 1-14 and 74-116, or fragments thereof as disclosed herein, or a concatemeric peptide as disclosed herein, may contain one or more conservative substitutions (and in aspects, a nonconservative substitution) in one or more HLA contact residues, provided HLA binding is preserved. MHC binding assays are well known in the art. In aspects, such assays may include the testing of binding affinity with respect to MHC class I and class II alleles in in vitro binding assays, with such binding assays as are known in the art. Examples include, e.g., the soluble binding assays as disclosed in U.S. Pat. No. 7,884,184 or PCT/US2020/020089, both of which are herein incorporated by reference in their entireties. Additionally, in aspects, a fully functional variant polypeptide having a sequence (or a core sequence) comprising, consisting of, or consisting essentially of one or more of SEQ ID NOS: 1-14 and 74-116 as disclosed herein do not contain mutations at one or more critical residues or regions, such as TCR contact residues.

In aspects, the TCR-binding epitope (which can be referred to as TCR binding residues, TCR facing epitope, TCR facing residues, or TCR contacts) for a 9-mer identified epitope (which may be a 9-mer fragment of one or more of SEQ ID NOS: 1-14 and 74-116 as disclosed herein or a 9-mer fragment of a concatemeric peptide as disclosed herein) that bind to a MHC class II molecule are at position 2, 3, 5, 7, and 8 of the identified epitope, while the MHC-binding agretope (which can be referred to as MHC contacts, MHC facing residues, MHC-binding residues, or MHC-binding face) for a 9-mer identified epitope (which may be a 9-mer fragment of one or more of SEQ ID NOS: 1-14 and 74-116 as disclosed herein or a 9-mer fragment of a concatemeric peptide as disclosed herein) that bind to a MHC class II molecule are at position 1, 4, 6, and 9, both as counted from the amino terminal.

In aspects, the TCR binding epitope for a 9-mer identified epitope (which may be a 9-mer fragment of one or more of SEQ ID NOS: 1-14 and 74-116 or as disclosed herein or a 9-mer fragment of a concatemeric peptide) that binds to a MHC class I molecule are at position 4, 5, 6, 7, and 8 of the identified epitope, while the MHC binding agretope for a 9-mer identified epitope (which may be a 9-mer fragment of one or more of SEQ ID NOS: 1-14 and 74-116 as disclosed herein or a 9-mer fragment of a concatemeric peptide as disclosed herein) that bind to a MHC class I molecule are at position 1, 2, 3, and 9, both as counted from the amino terminal.

In aspects, the TCR binding epitope for a 10-mer identified epitope that bind to a MHC class I molecule are at position 4, 5, 6, 7, 8, and 9 of the identified epitope (which may be a 10-mer fragment of one or more of SEQ ID NOS: 1-14 and 74-116 as disclosed herein, or a 10-mer fragment of a concatemeric peptide as disclosed herein, or a 10-mer peptide containing a 9-mer of one or more of SEQ ID NOS: 1-14 and 74-116), while the MHC binding agretope for a 10-mer identified epitope (which may be a 10-mer fragment of one or more of SEQ ID NOS: 1-14 and 74-116 as disclosed herein or a 10-mer fragment of a concatemeric peptide as disclosed herein, or a 10-mer peptide containing a 9-mer of one or more of SEQ ID NOS: 1-14 and 74-116) that bind to a MHC class I molecule are at position 1, 2, 3, 9, and 10, both as counted from the amino terminal.

In aspects, the TCR-binding epitope for a 9-mer identified epitope (which may be a 9-mer fragment of one or more of SEQ ID NOS: 1-14 and 74-116 as disclosed herein or a 9-mer fragment of a concatemeric peptide as disclosed herein) that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified epitope, while the MHC binding agretope for a 9-mer identified epitope (which may be a 9-mer fragment of one or more of SEQ ID NOS: 1-14 and 74-116 as disclosed herein or a 9-mer fragment of a concatemeric peptide as disclosed herein) is the complementary face to the TCR facing residues, both as counted from the amino terminal.

In aspects, the TCR binding epitope for 9-mer identified epitope (which may be a 9-mer fragment of one or more of SEQ ID NOS: 1-14 and 74-116 as disclosed herein or a 9-mer fragment of a concatemeric peptide as disclosed herein) that bind to a MHC class I molecule are at positions 4, 5, 6, 7, and 8; 1, 4, 5, 6, 7 and 8; or 1, 3, 4, 5, 6, 7, and 8 of the identified epitope, while the MHC binding agretope for a 9-mer identified epitope (which may be a 9-mer fragment of one or more of SEQ ID NOS: 1-14 and 74-116 as disclosed herein or a 9-mer fragment of a concatemeric peptide as disclosed herein) is the complementary face to the TCR facing residues, both as counted from the amino terminal.

In aspects, the TCR-binding epitope for a 10-mer identified epitope (which may be a 10-mer fragment of one or more of SEQ ID NOS: 1-14 and 74-116 as disclosed herein, or a 10-mer fragment of a concatemeric peptide as disclosed herein, or a 10-mer peptide containing a 9-mer of one or more of SEQ ID NOS: 1-14 and 74-116) that bind to a MHC class I molecule are at any combination of residues at positions 1, 3, 4, 5, 6, 7, 8, and 9 of the identified epitope, while the MHC binding agretope for a 10-mer identified epitope (which may be a 10-mer fragment of one or more of SEQ ID NOS: 1-14 and 74-116 as disclosed herein or a 10-mer fragment of a concatemeric peptide as disclosed herein, or a 10-mer peptide containing a 9-mer of one or more of SEQ ID NOS: 1-14 and 74-116) is the complementary face to the TCR facing residues, both as counted from the amino terminal.

Based on the above, it should be understood that in aspects in which one or more 9-mers and/or 10-mer epitopes are contained within a longer polypeptide and are predicted to bind one or more Class I or Class II MHC molecules and are occurring in close proximity to each other in a naturally occurring sequence (e.g., wherein position 1 of each pair of binding 9-mers and/or 10-mers fall within, e.g., 3 amino acids of each other), such epitopes may be combined to form an epitope cluster. In a given cluster, any given amino acid may be, with respect to a given 9-mer epitope or 10-mer epitope, MHC facing and, with respect to another 9-mer epitope, TCR facing.

In aspects, the present disclosure also includes fragments of the instantly-disclosed polypeptides and concatemeric polypeptides. In aspects, the present disclosure also encompasses fragments of the variants of the instantly-disclosed polypeptides and concatemeric polypeptides as described herein. In aspects, as used herein, a fragment comprises at least about nine contiguous amino acids. In aspects, the present disclosure also encompasses fragments of the variants of the T-cell epitopes described herein. Useful fragments (and fragments of the variants of the polypeptides and concatemeric polypeptides described herein) include those that retain one or more of the biological activities, particularly: MHC binding propensity and/or TCR specificity, and/or retain regulatory T cell stimulating or suppressive activity. Biologically active fragments are, for example, about 9, 10, 11, 12, 1, 14, 15, 16, 20, 25, 30, 40, 50, 60, 70, 80, 90, 100 or more amino acids in length, including any value or range therebetween. Fragments can be discrete (not fused to other amino acids or polypeptides) or can be within a larger polypeptide. Several fragments can be comprised within a single larger polypeptide. In aspects, a fragment designed for expression in a host can have heterologous pre- and pro-polypeptide regions fused to the amino terminus of the polypeptide fragment and an additional region fused to the carboxyl terminus of the fragment.

In aspects, the instantly disclosed polypeptides and concatemeric polypeptides of the present disclosure can include allelic or sequence variants (“mutants”) or analogs thereof, or can include chemical modifications (e.g., pegylation, glycosylation). In aspects, a mutant retains the same function, particularly MHC binding propensity and/or TCR specificity, and/or retain regulatory T cell stimulating or suppressive activity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

The manner of producing the polypeptides of the present disclosure will vary widely, depending upon the nature of the various elements comprising the molecule. For example, an isolated polypeptide can be purified from cells that naturally express it, purified from cells that have been altered to express it (recombinant), or synthesized using known protein synthesis methods. The synthetic procedures may be selected so as to be simple, provide for high yields, and allow for a highly purified stable product. For example, polypeptides of the instant disclosure can be produced either from a nucleic acid disclosed herein, or by the use of standard molecular biology techniques, such as recombinant techniques, mutagenesis, or other known means in the art. An isolated polypeptide can be purified from cells that naturally express it, purified from cells that have been altered to express it (recombinant), or synthesized using known protein synthesis techniques. In aspects, a polypeptide of the instant disclosure is produced by recombinant DNA or RNA techniques. In aspects, a polypeptide of the instant disclosure can be produced by expression of a recombinant nucleic acid of the instant disclosure in an appropriate host cell. For example, a nucleic acid molecule encoding the polypeptide is cloned into an expression cassette or expression vector, the expression cassette or expression vector introduced into a host cell and the polypeptide expressed in the host cell. The polypeptide can then be isolated from the cells by an appropriate purification scheme using standard protein purification techniques. Alternatively a polypeptide can be produced by a combination of ex vivo procedures, such as protease digestion and purification. Further, polypeptides of the instant disclosure can be produced using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, one or more peptides or polypeptides of the instant disclosure (e.g., but not limited to, a peptide or polypeptide comprising, consisting, or consisting essentially of an amino acid sequence of SEQ ID NOS. 1-14 and 74-116 (and/or fragments or variants thereof), and optionally 1 to 12 additional amino acids distributed in any ratio on the N terminus and/or C-terminus of the polypeptide of SEQ ID NOS. 1-14 and 74-116) may be joined to, linked to (e.g., fused in-frame, chemically-linked, or otherwise bound), and/or inserted into a heterologous polypeptide. As previously described, with respect to the one or more Tregitopes of the instant disclosure, the term “heterologous polypeptide” is intended to mean that the one or more Tregitopes of the instant disclosure are heterologous to, or not included naturally, in the heterologous polypeptide. In aspects, one or more of the instantly-disclosed polypeptides (Treg activating regulatory T-cell epitopes, Tregitopes, or T-cell epitope polypeptides) may be inserted into the heterologous polypeptide (e.g., through recombinant techniques, mutagenesis, or other known means in the art), may be added to the C-terminus (with or without the use of linkers, as is known in the art), and/or added to the N-terminus (with or without the use of linkers, as is known in the art) of the heterologous polypeptide. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, the one or more peptides or polypeptides of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, the peptides or polypeptides can be either in neutral (uncharged) or salt forms, and may be either free of or include modifications such as glycosylation, side chain oxidation, or phosphorylation. In certain aspects, the peptides or polypeptides can be capped with an n-terminal acetyl and/or c-terminal amino group.

In aspects, a polypeptide comprises one or more of SEQ ID NOS: 1-14 and 74-116 of the present disclosure (in aspects, including fragments and variants thereof of SEQ ID NOS: 1-14 and 74-116, provided said fragments and/or variants retain MHC binding propensity and TCR specificity, and/or retain regulatory T cell stimulating or suppressive activity) joined to, linked to (e.g., fused in-frame, chemically-linked, or otherwise bound), and/or inserted into a heterologous polypeptide, such as an antibody (which can be IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody)). In aspects, the one or more Tregitopes may be inserted into or replace amino acids in a Fc domain as disclosed in U.S. Pat. No. 7,442,778, U.S. Pat. No. 7,645,861, U.S. Pat. No. 7,655,764, U.S. Pat. No. 7,655,765, and/or U.S. Pat. No. 7,750,128 (each of which are herein incorporated by reference in their entirety). In aspects, the one or more of SEQ ID NOS: 1-14 and 74-116 may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, the peptides or polypeptides can be either in neutral (uncharged) or salt forms, and may be either free of or include modifications such as glycosylation, side chain oxidation, or phosphorylation. In certain aspects, the peptides or polypeptides can be capped with an n-terminal acetyl and/or c-terminal amino group.

In aspects, the Tregitope compounds and compositions of the present disclosure comprise one or more Tregitopes incorporated as an internal sequence into an Fc domain as disclosed in U.S. Pat. No. 7,442,778, U.S. Pat. No. 7,645,861, U.S. Pat. No. 7,655,764, U.S. Pat. No. 7,655,765, and/or U.S. Pat. No. 7,750,128 (each of which are herein incorporated by reference in their entirety). Such an internal sequence may be added by insertion (i.e., between amino acids in the previously existing Fc domain) or by replacement of amino acids in the previously existing Fc domain (i.e., removing amino acids in the previously existing Fc domain and adding peptide amino acids). In the latter case, the number of peptide amino acids added need not correspond to the number of amino acids removed from the previously existing Fc domain; for example, in aspects, the compositions may comprise an added internal sequence of 9-15 amino acids, with a sequence of 1-21 amino acids removed from the native Fc domain. In aspects, the one or more Tregitopes are inserted at or replace (e.g., full or partial replacement) one or more preferred internal sites in the Fc domain as disclosed in U.S. Pat. No. 7,442,778, U.S. Pat. No. 7,645,861, U.S. Pat. No. 7,655,764, U.S. Pat. No. 7,655,765, and/or U.S. Pat. No. 7,750,128.

In aspects, the present disclosure is directed to polypeptide (which, in aspects, may be an isolated, synthetic, or recombinant) having a sequence comprising one or more of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116), wherein said one or more of SEQ ID NOS: 1-14 and 74-116 is not naturally included in the polypeptide and/or said one or more of SEQ ID NOS: 1-14 and 74-116 is not located at its natural position in the polypeptide. In aspects, one or more Tregitopes of the instant disclosure (which, in aspects, may be an isolated, synthetic, or recombinant) having a sequence comprising one or more of SEQ ID NOS: 1-14 and 74-116 (and fragments or variants thereof), and optionally 1 to 12 additional amino acids distributed in any ratio on the N terminus and/or C-terminus of the polypeptide of SEQ ID NOS: 1-14 and 74-116, may also be fused to or inserted internally within (e.g., but not limited to, site directed mutagenesis or other recombinant techniques) a Factor V or Factor VIII molecule or replacement protein/supplement (or fragments thereof), such as in instances where the Tregitope is not located in its natural position within the Factor V or Factor VIII molecule or replacement protein/supplement (or fragments thereof) or wherein the Factor V or Factor VIII molecule or replacement protein/supplement (or fragments thereof) is missing such a Tregitope (e.g., if a particular Factor V or Factor VIII molecule or replacement protein/supplement (or fragments thereof) has a mutated or missing corresponding section). In aspects, such polypeptides of the present disclosure, which are further described below, may be isolated, synthetic, and/or recombinant, and may comprise post-transcriptional modifications such as glycosylation, added chemical groups, etc. In aspects, such polypeptides can be either in neutral (uncharged) or salt forms, and may be either free of or include modifications such as glycosylation, side chain oxidation, or phosphorylation. In certain aspects, such polypeptides can be capped with an n-terminal acetyl and/or c-terminal amino group.

In aspects, a polypeptide (which, in aspects, may be an isolated, synthetic, or recombinant) comprises one or more of SEQ ID NOS: 1, 3-8, 10-14, 74-78, and 85-114 (and fragments or variants thereof), and optionally 1 to 12 additional amino acids distributed in any ratio on the N terminus and/or C-terminus of the polypeptide of SEQ ID NOS: 1, 3-8, 10-14, 74-78, and 85-114, wherein said polypeptide does not comprise human coagulation Factor V molecule or replacement protein/supplement (or a fragments thereof). In aspects, if a polypeptide does comprise human coagulation Factor V molecule or replacement protein/supplement (or a fragments thereof), then said one or more of SEQ ID NOS: 1, 3-8, 10-14, 74-78, and 85-114 (and fragments or variants thereof), and optionally 1 to 12 additional amino acids distributed in any ratio on the N terminus and/or C-terminus of the polypeptide of SEQ ID NOS: 1, 3-8, 10-14, 74-78, and 85-114, is not located in its natural position in the human coagulation Factor V molecule or replacement protein/supplement (or a fragments thereof). In aspects, one or more Tregitopes having a sequence comprising SEQ ID NOS: 1, 3-8, 10-14, 74-78, and 85-114 (and fragments or variants thereof), and optionally 1 to 12 additional amino acids distributed in any ratio on the N terminus and/or C-terminus of the polypeptide of SEQ ID NOS: 1, 3-8, 10-14, 74-78, and 85-114, may also be fused to or inserted internally within (e.g., but not limited to, using immune engineering techniques such as but not limited to, site directed mutagenesis or other recombinant techniques) a Factor V molecule or replacement protein/supplement (or a fragments thereof), such as in instances where the Tregitope is not located in its natural position within the Factor V molecule or replacement protein/supplement (or a fragments thereof) or where the Factor V molecule or replacement protein/supplement (or a fragments thereof is missing such a Tregitope (e.g., if a particular human coagulation Factor V molecule or replacement protein/supplement (or a fragments thereof) has a mutated or missing corresponding section).

In aspects, a polypeptide (which, in aspects, may be an isolated, synthetic, or recombinant) comprises one or more of SEQ ID NOS: 2, 9, and 79-84 (and fragments or variants thereof), and optionally 1 to 12 additional amino acids distributed in any ratio on the N terminus and/or C-terminus of the polypeptide of SEQ ID NOS: 2, 9, and 79-84, wherein said polypeptide does not comprise human coagulation Factor VIII molecule or replacement protein/supplement (or a fragments thereof). In aspects, if a polypeptide does comprise human coagulation Factor VIII molecule or a fragment thereof, then said one or more of SEQ ID NOS: 2, 9, and 79-84 (and fragments or variants thereof), and optionally 1 to 12 additional amino acids distributed in any ratio on the N terminus and/or C-terminus of the polypeptide of SEQ ID NOS: 2, 9, and 79-84, is not located in its natural position in the human coagulation Factor VIII molecule or replacement protein/supplement (or a fragments thereof). In aspects, one or more Tregitopes having a sequence comprising SEQ ID NOS: 2, 9, and 79-84 (and fragments or variants thereof), and optionally 1 to 12 additional amino acids distributed in any ratio on the N terminus and/or C-terminus of the polypeptide of SEQ ID NOS: 2, 9, and 79-84, may also be fused to or inserted internally within (e.g., but not limited to, using immune engineering techniques such as but not limited to, site directed mutagenesis or other recombinant techniques) an Factor VIII molecule or replacement protein/supplement (or a fragments thereof), such as in instances where the Tregitope is not located in its natural position within the human coagulation Factor VIII molecule or replacement protein/supplement (or a fragments thereof) or where the human coagulation Factor VIII molecule or replacement protein/supplement (or a fragments thereof is missing such a Tregitope (e.g., if a particular human coagulation Factor VIII molecule or replacement protein/supplement (or a fragments thereof) has a mutated or missing corresponding section).

In aspects, said insertion of the one or more regulatory T cell epitopes into the heterologous polypeptide (e.g., an antibody or fragment thereof as described above, the human coagulation Factor V molecule or replacement protein/supplement (or a fragments thereof) as described above, or the human coagulation Factor VIII molecule or replacement protein/supplement (or a fragments thereof) as described above, comprises insertion of all or some of the amino acids of the one or more regulatory T cell epitopes (e.g., inserting the entire sequence of the Tregitope or a fragment thereof). In aspects, said insertion of the one or more regulatory T cell epitopes into the polypeptide comprises insertion of some or all of the amino acids of the one or more regulatory T cell epitopes and removing one or more amino acids at the site of insertion of the regulatory T cell epitope amino acids. In aspects, said insertion of the one or more regulatory T cell epitopes into the polypeptide comprises mutating the sequence of the polypeptide thereof to include the one or more regulatory T cell epitopes (for example, but not limited to, introduction one or more point mutations into the polypeptide by site-directed mutagenesis or other recombinant techniques). In aspects, said insertion of the one or more regulatory T cell epitopes into the polypeptide, which in aspects will introduce the one or more regulatory T cell epitope sequences, such that the previous immunogenicity of the polypeptide sequence is decreased and the tolerogenicity of the new polypeptide sequence is enhanced. In aspects, the number of said added one or more amino acids at the site of insertion of the regulatory T cell epitope amino acids need not correspond to the number of amino acids deleted from the sequence of the polypeptide. In aspects in which the one or more regulatory T cell epitopes are inserted or fused into a particular polypeptide (e.g., a human coagulation Factor V or Factor VIII molecule or replacement protein/supplement (or a fragments thereof)) that has a mutated or missing corresponding section for which the Tregitope might be normally found, said insertion or fusion is at the site within the polypeptide where the Tregitope would normally be present. In aspects, said insertion of one or more regulatory T cell epitopes into the polypeptide thereof results in decreasing the immunogenicity of the polypeptide.

In aspects, isolated, synthetic, or recombinant Tregitopes of the present disclosure include a Tregitope of Table 1 (including fragments thereof, variants thereof, and fragments of such variants thereof, provided said fragments and/or variants retain MHC binding propensity and/or TCR specificity). In aspects, isolated, synthetic, or recombinant Tregitopes of the present disclosure include one or more of:

(SEQ ID NO: 1)
ILTIHFTGHSFIYGK;
(SEQ ID NO: 2)
IHSIHFSGHVFTVRK;
(SEQ ID NO: 3)
KIVFKNMASRPYSIY;
(SEQ ID NO: 4)
FAVFDENKSWYLEDN;
(SEQ ID NO: 5)
ESNIMSTINGYVPES;
(SEQ ID NO: 6)
EKDIHSGLIGPLLI;
(SEQ ID NO: 7)
SHEFHAINGMIYSLP;
(SEQ ID NO: 8)
IHFTGHSFI;
(SEQ ID NO: 9)
IHFSGHVFT;
(SEQ ID NO: 10)
FKNMASRPY;
(SEQ ID NO: 11)
FDENLSWYL;
(SEQ ID NO: 12)
IMSTINGYV;
(SEQ ID NO: 13)
IHSGLIGPL;
(SEQ ID NO: 14)
FHAINGMIY;

and fragments thereof, variants thereof, and fragments of such variants thereof, provided said fragments and/or variants retain MHC binding propensity and/or TCR specificity. In certain aspects, such polypeptides can be capped with an n-terminal acetyl and/or c-terminal amino group.

In aspects, the Tregitope compounds and compositions of the present disclosure comprise a Tregitope peptide as described herein (e.g., but not limited to, a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) that is modified by attaching a reactive moiety to the Tregitope peptide to create a modified Tregitope peptide, wherein the reactive moiety of the modified Tregitope peptide is capable of forming a bond with a reactive functionality on a blood component, wherein upon formation of a bond between the reactive moiety of the Tregitope peptide and the reactive functionality on the blood component, a Tregitope-blood component conjugate is formed, as disclosed in U.S. Pat. No. 6,849,714, U.S. Pat. No. 6,887,470, U.S. Pat. No. 7,256,253, and U.S. Pat. No. 7,307,148 (each of which are herein incorporated by reference in their entirety). In aspects, the Tregitope in the Tregitope-blood component conjugate retains all or most of its original biologic activity. In aspects, the bond formed between the reactive moiety of the one or more modified Tregitope peptides and the blood component is a covalent bond. In aspects, the Tregitope peptide sequence is independently selected from SEQ ID NOS: 1-14 and 74-116, and optionally 1 to 12 additional amino acids distributed in any ratio on the N terminus and/or C-terminus of the polypeptide of SEQ ID NOS: 1-14 and 74-116.

Tregitope-blood component conjugates can extend the half-life of the modified polypeptides comprising Tregitopes in vivo, protect the modified polypeptides comprising Tregitopes from rapid proteolytic degradation, protect the modified polypeptides comprising Tregitopes from rapid clearance from circulation and/or rapid kidney excretion, allow for wide distribution of Tregitope-blood component conjugates throughout the body of a subject, aid in delivery of modified polypeptides comprising Tregitopes to appropriate immune cells (such as macrophages and APCs), allow the modified polypeptides comprising Tregitopes to be processed by the endocytic pathway of certain immune cells (such as macrophages and APCs), and aid in the presentation of modified polypeptides comprising Tregitopes as an antigen by said immune cells.

In aspects, the Tregitope-blood component conjugates comprise a blood component which acts as a carrier protein (e.g., albumin), and further comprise a modified polypeptide, said modified polypeptide comprising one or more regulatory T cell epitopes (termed “Tregitopes”). The modified polypeptide comprises a reactive moiety that is attached to the polypeptide, with the reactive moiety being capable of forming a bond (e.g., a covalent linkage) with a reactive functionality on the blood component. Tregitope-blood component conjugates may be formed by modifying a polypeptide comprising a Tregitope by attaching a reactive moiety to the polypeptide to create a modified polypeptide, then forming a bond between reactive moiety of the modified polypeptide with a reactive functionality on a blood component, as disclosed in U.S. Pat. No. 6,849,714, U.S. Pat. No. 6,887,470, U.S. Pat. No. 7,256,253, and U.S. Pat. No. 7,307,148, herein incorporated by reference in their entireties. In aspects of above-described Tregitope-blood component conjugates and modified polypeptides comprising Tregitopes, the Tregitope-blood component conjugates and modified polypeptides comprising Tregitopes may be isolated, synthetic, or recombinant.

In aspects, the blood components of the Tregitope-blood component conjugates may be either fixed or mobile, as disclosed in U.S. Pat. No. 6,849,714, U.S. Pat. No. 6,887,470, U.S. Pat. No. 7,256,253, and U.S. Pat. No. 7,307,148. Fixed blood components are non-mobile blood components and include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues, especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components is at least about 12 hours. In aspects of the Tregitope-blood component conjugates, the blood component is albumin, such as serum albumin, human serum albumin, recombinant albumin, and recombinant human serum albumin. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys34) (the location of the amino acid in the amino acid sequence of human serine albumin), containing a free thiol with a pKa of approximately 5, which may serve as a preferred reactive functionality of albumin. Cys34 of albumin is capable of forming a stable thioester bond with maleimidopropionamido (MPA), which is a preferred reactive moiety of a modified Tregitope peptide.

In aspects, reactive functionalities on the blood component of the Tregitope-blood component conjugates or on the blood components that are capable of forming a conjugate with the instantly-disclosed modified polypeptides are groups on blood components, including mobile and fixed proteins, to which reactive groups on modified therapeutic peptides react to form covalent bonds. As disclosed in U.S. Pat. No. 6,849,714, U.S. Pat. No. 6,887,470, U.S. Pat. No. 7,256,253, and U.S. Pat. No. 7,307,148, such functionalities usually include hydroxyl groups for bonding to ester reactive groups, thiol groups for bonding to maleimides, imidates and thioester groups; amino groups for bonding to activated carboxyl, phosphoryl or any other acyl groups on reactive groups. In aspects, the reactive functionality of the blood component is an amino group, a hydroxyl group, or a thiol group. In aspects, the reactive functionality of the blood component is a component of a side group of an amino acid in a polypeptide and/or protein, wherein the reactive functionality is near the surface of the polypeptide and/or protein. In aspects, the reactive functionality of the blood component is a thiol group of a free cysteine residue of a proteinaceous blood component. In aspects, the reactive functionality is a free thiol group of the cysteine at amino acid 34 (Cys³⁴) of serine albumin. In aspects, the reactive functionality of the blood component is a thiol with a pKa of approximately 5 in a physiological environment, such as plasma. In aspects, the reactive functionality of the blood component is a thiol with a pKa of approximately 5.5 in a physiological environment, such as plasma. In aspects, the reactive functionality of the blood component is a thiol with a pKa of 3-7 in a physiological environment, such as plasma. In aspects, the reactive functionality of the blood component is a thiolate anion. In aspects, the reactive functionality is a thiolate anion of the cysteine at amino acid 34 (Cys³⁴) of serine albumin.

In aspects, the modified polypeptides of the Tregitope-blood component conjugates and the modified polypeptides used to form the Tregitope-blood component conjugates comprise a reactive moiety that is attached to the polypeptide, with the reactive moiety being capable of forming a bond (e.g., a covalent linakge) with a reactive functionality on the blood component. In aspects, the reactive group is capable of reacting with an amino group, a hydroxyl group, or a thiol group on blood component to form a covalent bond therewith. In aspects, the reactive moiety is placed at a site such that when the modified polypeptide is bonded to the blood component, the modified peptide retains a substantial proportion of the parent compound's activity. In aspects, the reactive moiety may be a succinimidyl or maleimido group. In aspects, the reactive moiety may be attached to an amino acid positioned in the less therapeutically active region of amino acids of the polypeptide to be modified. In aspects, the reactive moiety is attached to the amino terminal amino acid of the modified polypeptide. In aspects, the reactive moiety is attached to the carboxy terminal amino acid of the modified polypeptide. In aspects, the reactive moiety is attached to an amino acid positioned between the amino terminal amino acid and the carboxy terminal amino acid of the modified polypeptide. In aspects, the reactive group may be attached to the polypeptide (to be modified) either via a linking group, or optionally without using a linking group. Further, one or more additional amino acids (e.g., one or more lysines) may be added to the polypeptide to facilitate the attachment of the reactive group. Linking groups are chemical moieties that link or connect reactive groups of blood components to polypeptides comprising one or more Tregitopes. Linking groups may comprise one or more alkyl groups, alkoxy group, alkenyl group, alkynyl group or amino group substituted by alkyl groups, cycloalkyl group, polycyclic group, aryl groups, polyaryl groups, substituted aryl groups, heterocyclic groups, and substituted heterocyclic groups. Linking groups may also comprise poly ethoxy aminoacids such as AEA ((2-amino)ethoxy acetic acid) or a preferred linking group AEEA ([2-(2-amino)ethoxy)]ethoxy acetic acid). In aspects, linking groups may comprise a polyethyleneglycol linker (e.g. but not limited to, PEG2 or PEG12).

As should be understood, modified polypeptides may be administered in vivo such that conjugation with blood components occurs in vivo, or they may be first conjugated to blood components in vitro and the resulting peptidase stabilized polypeptide administered in vivo. Further, as disclosed in in U.S. Pat. No. 6,849,714, U.S. Pat. No. 6,887,470, U.S. Pat. No. 7,256,253, and U.S. Pat. No. 7,307,148, a peptidase stabilized polypeptide is a modified polypeptide that has been conjugated to a blood component via a covalent bond formed between the reactive group of the modified peptide and the functionalities of the blood component, with or without a linking group. Such reaction is preferably established by covalent bonding of a polypeptide modified with a maleimide link (e.g. prepared from GMBS, MPA or other maleimides) to a thiol group on a mobile blood protein such as serum albumin or IgG. Peptidase stabilized polypeptides are more stable in the presence of peptidases in vivo than a non-stabilized peptide. A peptidase-stabilized therapeutic peptide generally has an increased half-life of at least 10-50% as compared to a non-stabilized peptide of identical sequence. Peptidase-stability is determined by comparing the half-life of the unmodified therapeutic peptide in serum or blood to the half-life of a modified counterpart therapeutic peptide in serum or blood. Half-life is determined by sampling the serum or blood after administration of the modified and non-modified peptides and determining the activity of the peptide. In addition to determining the activity, the length of the therapeutic peptide may also be measured.

In aspects, the modified polypeptides of the Tregitope-blood component conjugates and the modified polypeptides used to form the Tregitope-blood component conjugates comprise one or more Tregitopes as disclosed herein. In aspects, the one or more Tregitopes of the modified polypeptides have a sequence comprising, consisting of, or consisting essentially of one or more of SEQ ID NOS: 1-14 and 74-116 (and fragments and variants thereof) as essentially disclosed herein. In aspects, the one or more Tregitopes of the modified polypeptide may optionally have one or more linkers, which may optionally be cleavage sensitive sites, adjacent to their N and/or C terminal end. In such a modified polypeptide, two or more of the Tregitopes may have a cleavage sensitive site between them. Alternatively, two or more of the Tregitopes may be connected directly to one another or through a linker that is not a cleavage sensitive site. In aspects, the modified polypeptide comprising the one or more Tregitopes and/or the Tregitopes contained therein can be either in neutral (uncharged) or salt forms, and may be either free of or include modifications such as glycosylation, side chain oxidation, or phosphorylation. In certain aspects, the modified polypeptide comprising the one or more Tregitopes peptides or polypeptides can be capped with an n-terminal acetyl and/or c-terminal amino group. In aspects, the one or more Tregitopes included in the modified polypeptide can be capped with an n-terminal acetyl and/or c-terminal amino group.

In aspects, the blood component that forms the Tregitope-blood component conjugate with the modified Tregitope is albumin. In aspects, the reactive functionality of the blood component is an amino group, a hydroxyl group, or a thiol group. In aspects, the reactive functionality of the blood component is a component of a side group of an amino acid in a polypeptide and/or protein, wherein the reactive functionality is near the surface of the polypeptide and/or protein. In aspects, the reactive functionality of the blood component is a thiol group of a free cysteine residue of a proteinaceous blood component. In aspects, the reactive functionality is a free thiol group of the cysteine at amino acid 34 (Cys³⁴) of serine albumin. In aspects, the reactive functionality of the blood component is a thiol with a pKa of approximately 5 in a physiological environment, such as plasma. In aspects, the reactive functionality of the blood component is a thiol with a pKa of approximately 5.5 in a physiological environment, such as plasma. In aspects, the reactive functionality of the blood component is a thiol with a pKa of 3-7 in a physiological environment, such as plasma. In aspects, the reactive functionality of the blood component is a thiolate anion. In aspects, the reactive functionality is a thiolate anion of the cysteine at amino acid 34 (Cys³⁴) of serine albumin.

In aspects, the reactive moiety of the modified Tregitope peptide is a soft electrophile. In aspects, the reactive moiety of the modified Tregitope peptide is an electrophile selective for thiols. In a preferred embodiment, the reactive moiety attached to the Tregitope to create the modified Tregitope peptide is maleimide. In aspects, the reactive moiety is maleimide propionic acid. In a preferred embodiment, the reactive moiety attached of the modified Tregitope peptide is maleimide, the blood component is albumin, and the reactive functionality on the albumin is a free thiol or thiolate anion of Cys³⁴ of albumin. When the reactive moiety of the modified Tregitope peptide a maleimide, the blood component is albumin, and the reactive functionality of the albumin is a free thiol or thiolate anion of Cys³⁴ of albumin, a stable thioester linkage between the maleimide group and the sulfhydryl is formed which cannot be cleaved under physiological conditions. In aspects, the modified Tregitope peptide contains a linker, wherein the reactive moiety is attached to the Tregitope peptide through the linker. In aspects, the modified Tregitope peptide binds to the blood component in a 1:1 molar ratio.

The manner of producing the modified Tregitope peptides of the present disclosure will vary widely, depending upon the nature of the various elements comprising the molecule. The synthetic procedures may be selected so as to be simple, provide for high yields, and allow for a highly purified stable product. Normally, the reactive moiety will be created as the last stage, for example, with a carboxyl group, esterification to form an active ester will be the last step of the synthesis.

In aspects, the present disclosure is also directed to a method of synthesizing the modified Tregitope peptide, as disclosed in U.S. Pat. No. 6,849,714, U.S. Pat. No. 6,887,470, U.S. Pat. No. 7,256,253, and U.S. Pat. No. 7,307,148. In aspects, the method comprises the following steps. In the first step, the one or more Tregitope sequence of the polypeptide can be as essentially disclosed herein. In the second step, if the polypeptide does not contain a cysteine, then the polypeptide may be synthesized from the carboxy terminal amino acid and the reactive moiety is added to the carboxy terminal amino acid. Alternatively, a terminal lysine (or one or more lysines) may added to the carboxy terminal amino acid and the reactive moiety is added to the terminal lysine. In the third step, if the polypeptide contains only one cysteine, then the cysteine is reacted with a protective group prior to addition of the reactive moiety to an amino acid in a less therapeutically active region of the polypeptide. In the fourth step, if the polypeptide contains two cysteines as a disulfide bridge, then the two cysteines are oxidized and the reactive moiety is added to the amino terminal amino acid, or to the carboxy terminal amino acid, or to an amino acid positioned between the carboxy terminal amino acid and the amino terminal amino acid of the polypeptide. In the fifth step, if the polypeptide contains more than two cysteines as disulfide bridges, the cysteines are sequentially oxidized in the disulfide bridges and the peptide is purified prior to the addition of the reactive moieties to the carboxy terminal amino acid.

In aspects, the present disclosure is also directed to a method of synthesizing the Tregitope-blood component conjugate. In a first step, reactive maleimidopropionamido (MPA) is added via an N-terminal lysine on the polypeptide comprising one or more Tregitopes to create a modified polypeptide. In aspects, one or more lysines are present on the N-terminus of the polypeptide, optionally present at the N-terminus of a Tregitope sequence selected from the group of SEQ. ID NOS: 1-14 and 74-116 as disclosed herein. Optionally, polyethyleneglycol linker, such as PEG2 or PEG12, is present between the one or more lysines and a Tregitope sequence, or at the N-terminus of a Tregitope sequence. In aspects, a lysosomal cleavage site, such as a Cathepsin B site, optionally consisting (sequentially from N-terminus to C-terminus) of valine and citrulline, is present between the PEG2 or PEG12 moiety and the Tregitope sequence. The lysosomal cleavage site (such as Cathepsin B site) may be incorporated to provide a lysosomal protease site, allowing the Tregitope to be released into the lysosomal compartment. In aspects, lysosomal cleavage site (such as Cathepsin B site) is present to provide a lysosomal protease site, allowing the Tregitope to be released into cells, preferably into the early endosome. In a preferred embodiment, the lysosomal cleavage site (such as Cathepsin B site) is present to provide a lysosomal protease site, allowing the Tregitope to be released into cells, such as into a membrane-enclosed vesicle (such as the early endosome, late endosome, or lysosome), such that the Tregitope may be processed for antigen presentation. In aspects, the Tregitope is presented as antigen by immune cells, such as macrophages or antigen-presenting cells, preferably presented as an MHC class II antigen. In aspects, a lysosomal cleavage site, such as a Cathepsin B site, optionally consisting (sequentially from N-terminus to C-terminus) of valine and citrulline, is present between the PEG2 moiety and the Tregitope sequence, and/or between one or more Tregitopes. In aspects, one or more Tregitopes may be present on the construct, optionally more proximate to the C-terminus than the linker. In aspects, one or more lysosomal cleavage sites are present between multiple Tregitopes (for example, such that a single lysosomal cleavage site separates two Tregitopes, or such that one lysosomal cleavage site is present between a first and second Tregitope, and another lysosomal cleavage site is present between a second and third Tregitope, and so on). In aspects, a norleucine (Nle) residue is present at the C-terminus as a means to quantitate the amount of Tregitope peptide incorporated into the final Tregitope-blood component conjugate, for example for evaluation by mass spectrometry. In aspects, the C-terminus of the polypeptide is capped with a c-terminal amino group. In a second step, a maleimide-based chemistry is used to covalently link the modified polypeptide to a blood component, preferably serum albumin, in a 1:1 molar ratio. The second step may be performed in vivo or ex vivo, as described further below and in the examples of the present disclosure.

In aspects, the formation of the Tregitope-blood component conjugate protects the Tregitope, when present in vivo, from rapid degradation by peptidases, rapid clearance from circulation, and/or rapid kidney excretion. In aspects, the formation of the Tregitope-blood component conjugate significantly extends the half-life of the Tregitope in vivo. In aspects, the formation of the Tregitope-blood component conjugate allows wide distribution of the Tregitope-blood component conjugate throughout the body of a subject. In aspects, the Tregitope-blood component conjugate does not cross the blood-brain barrier when present in the plasma of a subject. In aspects, the Tregitope-blood component conjugate aid in delivery of Tregitopes to appropriate immune cells, such as macrophages and/or antigen-presenting cells (APCs). In aspects, upon delivery of Tregitopes to appropriate immune cells, such as macrophages and/or APCs, the Tregitopes are encompassed in a membrane-bound vesicle, preferably a vesicle in the endocytic pathway such as an early endosome, late endosome, or lysosome. In aspects, the Tregitopes, once processed by the appropriate immune cells, such as macrophages and/or APCs, are presented as MHC class II antigens.

In aspects, the Tregitope in the Tregitope-blood component conjugate has a plasma half-life in vivo of up to 12 hours. In aspects, the Tregitope in the Tregitope-blood component conjugate has a plasma half-life in vivo of up to 1 day. In aspects, the Tregitope in the Tregitope-blood component conjugate has a plasma half-life in vivo of up to 40-48 hours. In aspects, the Tregitope in the Tregitope-blood component conjugate has a plasma half-life in vivo of up to 60 hours. In aspects, the Tregitope in the Tregitope-blood component conjugate has a plasma half-life in vivo of up to 15 days.

In aspects, the modified polypeptide comprising one or more Tregitopes is administered to a subject, wherein upon administration, the modified polypeptide reacts in vivo with a reactive functionality of a circulating blood component. In aspects, the peptide is administered to a human subject, and the blood component is human albumin, preferably the circulating albumin of the human subject.

In aspects, the modified polypeptides used to form the Tregitope-blood component conjugates is capable of forming a bond ex vivo with a reactive functionality on a blood component, wherein upon formation of a bond between the reactive moiety of the modified polypeptide and the reactive functionality on the blood component, a Tregitope-blood component conjugate is formed, as disclosed in U.S. Pat. No. 6,849,714, U.S. Pat. No. 6,887,470, U.S. Pat. No. 7,256,253, and U.S. Pat. No. 7,307,148. In aspects, the modified polypeptide as disclosed herein is configured to covalently attach to a reactive functionality of a blood component outside of the body. In aspects, the blood component is albumin. In aspects, the blood component is selected from the group of recombinant albumin, human recombinant albumin, and albumin from a genomic source.

In aspects, the present disclosure is also directed to an ex vivo method of synthesizing the modified Tregitope peptide and the Tregitope-blood component conjugate, as disclosed in U.S. Pat. No. 6,849,714, U.S. Pat. No. 6,887,470, U.S. Pat. No. 7,256,253, and U.S. Pat. No. 7,307,148. In aspects, the modified polypeptide as disclosed herein is added to blood, serum or saline solution containing human serum albumin to permit covalent bond formation between the modified therapeutic peptide and the blood component. In aspects, the polypeptide comprising one or more Tregitopes as disclosed herein is modified with maleimide and it is reacted with serum albumin in saline solution. In aspects, once the modified polypeptide has reacted with the blood component, to form a Tregitope-blood component conjugate, the conjugate may be administered to the subject. In aspects, after the modified polypeptide has reacted with the blood component to form the conjugate, but before the conjugate is administered to the subject, the conjugate may be separated from non-conjugated blood components in the reaction mixture. In aspects, conjugate may be separated from non-conjugated blood components in the reaction mixture by separating substances on the basis of their varying strengths of hydrophobic interactions with hydrophobic ligands immobilized to an uncharged matrix. In aspects, the uncharged matrix may be a hydrophobic solid support, wherein the support comprises a column containing a hydrophobic resin such as, but not limited to, octyl sepharose, phenyl sepharose and butyl sepharose. In aspects, this technique may be performed with moderately high concentrations of salts (≈1M) in the start buffer (salt promoted adsorption). Elution is achieved by a linear or stepwise decrease in salt concentration. The type of ligand, the degree of substitution, the pH and the type and concentration of salt used during the adsorption stage have a profound effect on the overall performance (e.g. selectivity and capacity) of a HIC matrix (Hydrophobic Interaction Chromatography matrix).

The solvent is one of the most important parameters which influence capacity and selectivity in HIC (Hydrophobic Interaction Chromatography). In general, the adsorption process is more selective than the desorption process. It is therefore important to optimize the start buffer with respect to pH, type of solvent, type of salt and concentration of salt. The addition of various “salting-out” salts to the sample promotes ligand-protein interactions in HIC. As the concentration of salt is increased, the amount of bound protein increases up to the precipitation point for the protein. Each type of salt differs in its ability to promote hydrophobic interactions.

Increasing the salting-out effect strengthens the hydrophobic interactions, whereas increasing the chaotropic effect weakens them. Examples of salts with high salting-out effects, in order from greater salting-out effect to smaller salting-out effect, include: PO₄³⁻, SO₄²⁻, CH₃COO⁻, Cl⁻, Br⁻, NO₃⁻, ClO₄⁻, I⁻, and SCN⁻. Examples of salts with high chaotropic effects, in order from greater chaotropic effect to smaller chaotropic effect, include: NH₄⁺, Rb⁺, K⁺, Na⁺, Cs⁺, Li⁺, Mg²⁺, and Ba²⁺. The most commonly used salts for HIC are ammonium sulfate ((NH₄)₂SO₄), sodium sulfate ((Na)₂SO₄)), magnesium sulfate (MgSO₄), sodium chloride (NaCl), potassium chloride (KCl), and ammonium acetate (CH₃COONH₄).

Protein binding to HIC adsorbents is promoted by moderate to high concentrations of “salting-out” salts, most of which also have a stabilizing influence on protein structure due to their preferential exclusion from native globular proteins, i.e. the interaction between the salt and the protein surface is thermodynamically unfavorable. The salt concentration should be high enough (e.g. 500-1000 mM) to promote ligand-protein interactions yet below that which causes precipitation of the protein in the sample. In the case of albumin, the salt concentration should be kept below 3M (moles per liter). The principle mechanism of salting-out consists of the salt-induced increase of the surface tension of water (Melander and Horvath, 1977). Thus, a compact structure becomes energetically more favorable because it corresponds to smaller protein-solution interfacial area. Under these conditions, for example buffer composed of SO₄²⁻, PO₄²⁻ or CH₃COO⁻ with any counter ion, these salts exhibit their salting-out effect upon essentially all conjugated albumin described herein in a manner different to non-conjugated albumin (i.e. mercaptalbumin and albumin capped with cysteine), thus enabling a consistent chromatographic separation between conjugated albumin versus non-conjugated albumin. Thus, lower concentrations of salt are required to promote interactions between ligand and conjugated albumin than between ligand and non-conjugated albumin. This chromatographic separation is essentially independent of (a) the sequence of albumin (e.g. human, mouse, rat, etc.) (b) the source of albumin (i.e. plasma derived or recombinant) (c) the molecular weight of the conjugated modified Tregitope, (d) the position of the reactive moiety within the structure of the molecule, (e) the peptide sequence or chemical structure of the molecule, and (f) the three-dimensional structure of the conjugated molecule, e.g. linear versus loop structure.

In aspects, the salt of the aqueous buffer has a sufficient salting-out effect. In aspects, for providing a sufficient salting out effect, the salt may be phosphate, sulfate and acetate. In aspects, the selection of the cation of the buffer is can be selected, without limitation, from the group consisting of NH₄⁺, Rb⁺, K⁺, Na⁺, Cs⁺, Li⁺, Mg²⁺ and Ba²⁺. In aspects, the aqueous buffer may be selected from the group of ammonium phosphate, ammonium sulfate and magnesium phosphate. In aspects, the buffer pH is between 3.0 and 9.0; more preferably between 6.0 and 8.0, and even more preferably, the pH is 7.0. In aspects, the buffer and the hydrophobic solid support are at room temperature (about 25° C.) or at 4° C. or in between.

In aspects, the present disclosure also provides chimeric or fusion polypeptide compositions. In aspects, the present disclosure provides isolated, synthetic, or recombinant chimeric or fusion polypeptide compositions wherein one or more of the instantly-disclosed Tregitopes is a part thereof. In aspects, a chimeric or fusion polypeptide composition comprises one or more polypeptides (Treg activating regulatory T-cell epitope, Tregitope, Tregitope peptide, or T-cell epitope polypeptide) of the present disclosure joined to, linked to (e.g., fused in-frame, chemically-linked, or otherwise bound), and/or inserted into a heterologous polypeptide (e.g. but not limited to, a human coagulation Factor V molecule or replacement protein/supplement (or a fragments thereof), a human coagulation Factor VIII molecule or replacement protein/supplement (or a fragments thereof), or an antibody (which can be IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody)). As previously described, with respect to the one or more Tregitopes of the instant disclosure, the term “heterologous polypeptide” is intended to mean that the one or more Tregitopes of the instant disclosure are heterologous to, or not included naturally, in the heterologous polypeptide. In aspects, one or more of the instantly-disclosed polypeptides (Treg activating regulatory T-cell epitopes, Tregitopes, or T-cell epitope polypeptides) may be inserted into the heterologous polypeptide (e.g., through recombinant techniques, mutagenesis techniques, or other known means in the art), may be added to the C-terminus, and/or added to the N-terminus of the heterologous polypeptide. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, the one or more Tregitopes may be inserted into or replace amino acids in a Fc domain as disclosed in U.S. Pat. No. 7,442,778, U.S. Pat. No. 7,645,861, U.S. Pat. No. 7,655,764, U.S. Pat. No. 7,655,765, and/or U.S. Pat. No. 7,750,128 (each of which are herein incorporated by reference in their entirety). In aspects, chimeric or fusion polypeptides comprise one or more of the instantly-disclosed polypeptides (Treg activating regulatory T-cell epitopes, Tregitopes, or T-cell epitope polypeptides) operatively linked to a heterologous polypeptide. “Operatively linked” indicates that the one or more of the instantly-disclosed polypeptides (Treg activating regulatory T-cell epitopes, Tregitopes, or T-cell epitope polypeptides) and the heterologous polypeptide are fused in-frame or chemically-linked or otherwise bound.

In aspects of the above isolated, synthetic, or recombinant chimeric or fusion polypeptide compositions, the one or more polypeptides (Treg activating regulatory T-cell epitope, Tregitope, Tregitope peptide, or T-cell epitope polypeptide) of the present disclosure have a sequence comprising, consisting of, or consisting essentially of one or more of SEQ ID NOS: 1-14 and 74-116. In aspects of the instantly-disclosed chimeric or fusion polypeptide compositions, the one or more polypeptides comprise, consist, or consist essentially of an amino acid sequence of SEQ ID NOS. 1-14 and 74-116 (and/or fragments or variants thereof), and optionally 1 to 12 additional amino acids distributed in any ratio on the N terminus and/or C-terminus of the polypeptide of SEQ ID NOS. 1-14 and 74-116. In aspects of the chimeric or fusion polypeptide compositions, the one or more Tregitopes as disclosed herein may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition comprises a polypeptide, said polypeptide having a sequence comprising one or more of SEQ ID NOS. 1-14 and 74-116 (and/or fragments or variants thereof), and optionally 1 to 12 additional amino acids distributed in any ratio on the N terminus and/or C-terminus of the polypeptide of SEQ ID NOS. 1-14 and 74-116 of the present disclosure, wherein said one or more of SEQ ID NOS: 1-14 and 74-116 is not naturally included in the polypeptide and/or said of one or more of SEQ ID NOS: 1-14 and 74-116 is not located at its natural position in the polypeptide. In aspects, the one or more Tregitopes of the present disclosure can be joined, linked to (e.g., fused in-frame, chemically-linked, or otherwise bound), and/or inserted into the polypeptide. In aspects, chimeric or fusion polypeptide compositions comprise one or more of the instantly-disclosed Tregitopes operatively linked to a heterologous polypeptide having an amino acid sequence not substantially homologous to the Tregitope. In aspects, the chimeric or fusion polypeptide does not affect function of the Tregitope per se. For example, the fusion polypeptide can be a GST-fusion polypeptide in which the Tregitope sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Ig fusions. Such fusion polypeptides, particularly poly-His fusions or affinity tag fusions, can facilitate the purification of recombinant polypeptide. In certain host cells (e.g., mammalian host cells), expression and/or secretion of a polypeptide can be increased by using a heterologous signal sequence. Therefore, in aspects, the fusion polypeptide contains a heterologous signal sequence at its N-terminus. In aspects of the above recombinant chimeric or fusion polypeptide compositions, the heterologous polypeptide or polypeptide comprises a biologically active molecule. In aspects, the biologically active molecule is selected from the group consisting of an immunogenic molecule, a T cell epitope, a viral protein, and a bacterial protein. In aspects, the biologically active molecule is a human Coagulation Factor VIII molecule or replacement protein/supplement. In aspects, the biologically active molecule is a human Coagulation Factor V molecule or replacement protein/supplement. In aspects, the one or more of SEQ ID NOS: 1-14 and 74-116 of the present disclosure can be joined or linked to (e.g., fused in-frame, chemically-linked, or otherwise bound) to a small molecule, drug, or drug fragment. For example, one or more of SEQ ID NOS. 1-14 and 74-116 (and/or fragments or variants thereof, and optionally 1 to 12 additional amino acids distributed in any ratio on the N terminus and/or C-terminus of the polypeptide of SEQ ID NOS. 1-14 and 74-116) of the present disclosure can be joined or linked to (e.g., fused in-frame, chemically-linked, or otherwise bound) a drug or drug fragment that is binds with high affinity to defined HLAs. In aspects of the above chimeric or fusion polypeptide compositions or fusion products, the one or more polypeptides (Treg activating regulatory T-cell epitope, Tregitope, Tregitope peptide, or T-cell epitope polypeptide) of the present disclosure included therein have a sequence comprising, consisting of, or consisting essentially of one or more of SEQ ID NOS: 1-14 and 74-116 (or fragments or variants thereof). In aspects of the above chimeric or fusion polypeptide compositions or fusion products, the one or more polypeptides (Treg activating regulatory T-cell epitope, Tregitope, Tregitope peptide, or T-cell epitope polypeptide) of the present disclosure included therein have a sequence comprising, consisting of, or consisting essentially of one or more of SEQ ID NOS: 1-2 (or fragments or variants thereof). In aspects of the above chimeric or fusion polypeptide compositions or fusion products, the one or more polypeptides (Treg activating regulatory T-cell epitope, Tregitope, Tregitope peptide, or T-cell epitope polypeptide) of the present disclosure included therein have a sequence comprising, consisting of, or consisting essentially of SEQ ID NOS: 1 (or fragments or variants thereof). In aspects of the above-described chimeric or fusion polypeptide compositions or fusion products, the chimeric or fusion polypeptide compositions or fusion products can be recombinant, isolated, and/or synthetic.

A chimeric or fusion polypeptide composition can be produced by standard recombinant DNA or RNA techniques as are known in the art. For example, DNA or RNA fragments coding for the different polypeptide sequences may be ligated together in-frame in accordance with conventional techniques. In another embodiment, the fusion gene can be synthesized by conventional techniques including automated DNA synthesizers. Alternatively, polymerase chain reaction (PCR) amplification of nucleic acid fragments can be carried out using anchor primers which give rise to complementary overhangs between two consecutive nucleic acid fragments which can subsequently be annealed and re-amplified to generate a chimeric nucleic acid sequence. (Short Protocols in Molecular Biology: A Compendium of Methods from Current Protocols in Molecular Biology, (2ND, 1992), FM Asubel et al. (eds), Green Publication Associates, New York, N.Y. (Publ), ISBN: 9780471566355, which are herein incorporated by reference in their entirety). Further, one or more polypeptides (Treg activating regulatory T-cell epitope, Tregitope, Tregitope peptide, or T-cell epitope polypeptide) of the present disclosure (e.g., one or more Tregitopes of the present disclosure having a sequence comprising, consisting of, or consisting essentially of one or more of SEQ ID NOS: 1-14 and 74-116) can be inserted into a heterologous polypeptide or inserted into a non-naturally occurring position of a polypeptide through recombinant techniques, synthetic polymerization techniques, mutagenesis techniques, or other standard techniques known in the art. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, the one or more Tregitopes may be inserted into or replace amino acids in a Fc domain as disclosed in U.S. Pat. No. 7,442,778, U.S. Pat. No. 7,645,861, U.S. Pat. No. 7,655,764, U.S. Pat. No. 7,655,765, and/or U.S. Pat. No. 7,750,128 (each of which are herein incorporated by reference in their entirety). In aspects, the one or more Tregitopes may be covalently bound to one or more internal conjugation site(s) in a Fc domain as disclosed in U.S. Pat. No. 8,008,453, U.S. Pat. No. 9,114,175, and/or U.S. Pat. No. 10,188740 (each of which are herein incorporated by reference in their entirety). Moreover, many expression vectors are commercially available that already encode a fusion moiety (e.g., a GST protein). A nucleic acid molecule encoding a Tregitope of the invention can be cloned into such an expression vector such that the fusion moiety is linked in-frame to the at least one Tregitope.

In aspects, the polypeptides, concatemeric polypeptides, and chimeric or fusion polypeptides can be purified to homogeneity or partially purified. It is understood, however, that preparations in which the Tregitope compounds and compositions are not purified to homogeneity are useful. The critical feature is that the preparation allows for the desired function of the Tregitope, even in the presence of considerable amounts of other components. Thus, the present disclosure encompasses various degrees of purity. In one embodiment, the language “substantially free of cellular material” includes preparations of the Tregitope having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a polypeptide, concatemeric polypeptide, and chimeric or fusion polypeptide of the present disclosure is recombinantly produced, the composition can also be substantially free of culture medium, for example, culture medium represents less than about 20%, less than about 10%, or less than about 5% of the volume of the polypeptides, concatemeric polypeptides, and chimeric or fusion polypeptides preparation. The language “substantially free of chemical precursors or other chemicals” includes preparations of the polypeptides, concatemeric polypeptides, and chimeric or fusion polypeptides in which it is separated from chemical precursors or other chemicals that are involved in the T-cell epitope's synthesis. The language “substantially free of chemical precursors or other chemicals” can include, for example, preparations of the polypeptides, concatemeric polypeptides, and chimeric or fusion polypeptides having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

In aspects, the present disclosure also includes pharmaceutically acceptable salts of the Regulatory T-cell epitope compounds and compositions (including one or more of e.g., peptides or polypeptides as disclosed herein; concatemeric peptides as disclosed herein; chimeric or fusion polypeptide compositions as disclosed herein (which in aspects may be isolated, synthetic, and/or recombinant). “Pharmaceutically acceptable salt” means a salt that is pharmaceutically acceptable and that possesses the desired pharmacological activity of the parent peptide or polypeptide (e.g., peptides, polypeptides, concatemeric peptides, and/or chimeric or fusion polypeptides as disclosed herein). As used herein, “pharmaceutically acceptable salt” refers to derivative of the instantly-disclosed polypeptides, concatemeric polypeptides, and/or chimeric or fusion polypeptides, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

### Nucleic Acids

In aspects, the present disclosure also provides for nucleic acids (e.g., DNAs (including cDNA, RNAs (such as, but limited to mRNA), vectors, viruses, or hybrids thereof, all of which may be isolated, synthetic, or recombinant) that encode in whole or in part one or more one or more peptides, polypeptides, concatemeric peptides, and/or chimeric or fusion polypeptides of the present disclosure as described herein. In aspects, the nucleic acid further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a non-human cell, and optionally the cell is transformed with the nucleic acid, or the expression cassette, plasmid, expression vector, or recombinant virus. In aspects, cells are transduced, transfected, or otherwise engineered to contain within one or more of e.g., polypeptides of the present disclosure; isolated, synthetic, or recombinant nucleic acids, expression cassettes, plasmids, expression vectors, or recombinant viruses as disclosed herein; and/or isolated, synthetic, or recombinant chimeric or fusion polypeptide compositions as disclosed herein. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen SH et al., (1994), Proc Natl Acad Sci USA, 91(8):3054-7, which are herein incorporated by reference in their entirety). Similarly, the nucleic acid molecules of the present disclosure can be inserted into plasmids. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. Such pharmaceutical compositions can be included in a container, pack, or dispenser together with instructions for administration. In aspects of the above nucleic acids (e.g., DNAs, RNAs, vectors, viruses, or hybrids thereof) that encode in whole or in part at least one or more peptides, polypeptides, concatemeric peptides, and/or chimeric or fusion polypeptides as described herein, the nucleic acids encode one or more peptides or polypeptides of the instant disclosure as described above (e.g., but not limited to, a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116; as well as the concatemeric peptides as disclosed herein. In aspects, the present disclosure is directed to a vector comprising a nucleic acid of the present disclosure encoding one or more polypeptides of the present disclosure or chimeric or fusion polypeptide composition of the present disclosure. In aspects, the present disclosure is directed to a cell comprising a vector of the present disclosure. In aspects, the cell can be a mammalian cell, bacterial cell, insect cell, or yeast cell.

The nucleic acid of the instant disclosure may be DNAs (including but not limited to cDNA) or RNAs (including but not limited to mRNA), single- or double-stranded. The nucleic acid is typically DNA or RNA (including mRNA). The nucleic acid may be produced by techniques well known in the art, such as synthesis, or cloning, or amplification of the sequence encoding the immunogenic polypeptide; synthesis, or cloning, or amplification of the sequence encoding the cell membrane addressing sequence; ligation of the sequences and their cloning/amplification in appropriate vectors and cells. The nucleic acids provided herein (whether RNAs, DNAs, vectors, viruses or hybrids thereof) that encode in whole or in part one or more peptides, polypeptides, concatemeric peptides, and/or chimeric or fusion polypeptides as described herein can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including e.g. in vitro, bacterial, fungal, mammalian, yeast, insect or plant cell expression systems. In aspects nucleic acids provided herein are synthesized in vitro by well-known chemical synthesis techniques (as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of nucleic acids provided herein, such as, e.g., subcloning, labeling probes (e.g., random-primer labeling using Klenow polymerase, nick translation, amplification), sequencing, hybridization and the like are well described in the scientific and patent literature (see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993), all of which are herein incorporated by reference in their entirety).

A further object of the present disclosure relates to a nucleic acid molecule encoding one or more peptides, polypeptides, concatemeric peptides, and/or chimeric or fusion polypeptides as described herein. The nucleic acid may be used to produce the one or more peptides, polypeptides, concatemeric peptides, and/or chimeric or fusion polypeptides as described herein in vitro or in vivo, or to produce cells expressing the polypeptide on their surface, or to produce vaccines wherein the active agent is the nucleic acid or a vector containing the nucleic acid. The nucleic acid may be, e.g., DNA, cDNA, PNA, CNA, RNA, either single- and/or double-stranded, or native or stabilized forms of polynucleotides as are known in the art.

As previously mentioned, the nucleic acid molecules according to the present disclosure may be provided in the form of a nucleic acid molecule per se such as naked nucleic acid molecules; a plasmid, a vector; virus or host cell, etc., either from prokaryotic or eukaryotic origin. Vectors include expression vectors that contain a nucleic acid molecule of the invention. An expression vector capable of expressing a polypeptide can be prepared. Expression vectors for different cell types are well known in the art and can be selected without undue experimentation. Generally, the (e.g., cDNA, or RNA, including mRNA) is inserted into an expression vector, such as a plasmid, in proper orientation and correct reading frame for expression. If necessary, the DNA (e.g., cDNA, or RNA, including mRNA) may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. The vectors of the present invention may, for example, comprise a transcriptional promoter, and/or a transcriptional terminator, wherein the promoter is operably linked with the nucleic acid molecule, and wherein the nucleic acid molecule is operably linked with the transcription terminator. One or more peptides or polypeptides of the present disclosure may be encoded by a single expression vector. Such nucleic acid molecules may act as vehicles for delivering peptides/polypeptides to the subject in need thereof, in vivo, in the form of, e.g., DNA/RNA vaccines.

In aspects, the vector may be a viral vector comprising a nucleic acid as defined above. The viral vector may be derived from different types of viruses, such as, Swinepox, Fowlpox, Pseudorabies, Aujezky's virus, salmonella, vaccinia virus, BHV (Bovine Herpes Virus), HVT (Herpes Virus of Turkey), adenovirus, TGEV (Transmissible Gastroenteritidis Coronavirus), Erythrovirus, and SIV (Simian Immunodeficiency Virus). Other expression systems and vectors may be used as well, such as plasmids that replicate and/or integrate in yeast cells.

The instant disclosure also relates to a method for preparing a peptide, polypeptide, concatemeric peptide, and/or chimeric or fusion polypeptide of the instant disclosure, the method comprising culturing a host cell containing a nucleic acid or vector as defined above under conditions suitable for expression of the nucleic acid and recovering the polypeptide. As indicated above, the proteins and peptides may be purified according to techniques known per se in the art.

### Pharmaceutical Compositions and Formulations

In aspects, the Tregitope compounds and compositions of the present disclosure (including one or more of e.g., polypeptides as disclosed herein; concatemeric peptides as disclosed herein; chimeric of fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, and recombinant viruses, or cells as disclosed herein; hereafter referred to as “T-cell epitope compounds and compositions of the present disclosure”) may be comprised in a pharmaceutical composition or formulation. In aspects, the instantly-disclosed pharmaceutical compositions or formulations generally comprise a Tregitope compound or composition of the present disclosure and a pharmaceutically acceptable carrier, excipient, and/or adjuvant. In aspects, the instantly-disclosed pharmaceutical compositions or formulations may further comprise diluents, adjuvants, freeze drying stabilizers, wetting or emulsifying agents, pH buffering agents, gelling or viscosity enhancing additives, and preservatives, depending on the route of administration. In aspects, said pharmaceutical compositions are suitable for administration. Pharmaceutically acceptable carriers and/or excipients are determined in part by the particular composition being administered, as well as by the particular method used to administer the composition. Accordingly, there is a wide variety of suitable formulations of pharmaceutical compositions for administering the instantly disclosed Tregitope compositions (see, e.g., Remington's Pharmaceutical Sciences, (18TH Ed, 1990), Mack Publishing Co., Easton, Pa. Publ)). In aspects, the pharmaceutical compositions are generally formulated as sterile, substantially isotonic, and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art would be able to determine the appropriate timing, sequence and dosages of administration for particular Tregitope compounds and compositions of the present disclosure. The dosage of the Tregitope compounds and compositions of the present disclosure will depend on the species, breed, age, size, treatment history, and health status of the animal (e.g., human) to be treated, as well as the route of administration, e.g., subcutaneous, intradermal, oral intramuscular or intravenous administration. The Tregitope compounds and compositions of the instant disclosure can be administered as single doses or in repeated doses. The Tregitope compounds and compositions of the instant disclosure can be administered alone, or can be administered simultaneously or sequentially administered with one or more further compositions, such as other porcine immunogenic or vaccine compositions. Where the compositions are administered at different times, the administrations may be separate from one another or overlapping in time.

Examples of pharmaceutically acceptable carriers, excipients or diluents include, but are not limited to demineralized or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

In aspects, preferred examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the Tregitope compounds and compositions of the present disclosure and as previously described above, use thereof in the compositions is contemplated. Supplementary active compounds can also be incorporated into the compositions.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde. Further adjuvants may include, but are not limited to, poly-ICLC, 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRTX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In aspects of the pharmaceutical compositions or vaccines as disclosed herein, the adjuvant comprises poly-ICLC. The TLR9 agonist CpG and the synthetic double-stranded RNA (dsRNA) TLR3 ligand poly-ICLC are two of the most promising vaccine adjuvants currently in clinical development. In preclinical studies, poly-ICLC appears to be the most potent TLR adjuvant when compared to LPS and CpG. This appears due to its induction of pro-inflammatory cytokines and lack of stimulation of IL-10, as well as maintenance of high levels of co-stimulatory molecules in DCs. Poly-ICLC is a synthetically prepared double-stranded RNA consisting of polyI and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and mixed production of type I interferons, cytokines, and chemokines.

Examples of freeze-drying stabilizer may be for example carbohydrates such as sorbitol, mannitol, starch, sucrose, dextran or glucose, proteins such as albumin or casein, and derivatives thereof.

In aspects, Tregitope compounds and compositions of the present disclosure are formulated to be compatible with its intended route of administration. The Tregitope compounds and compositions of the present disclosure can be administered by parenteral, topical, intravenous, oral, subcutaneous, intra-arterial, intradermal, transdermal, rectal, intracranial, intrathecal, intraperitoneal, intranasal; vaginally; intramuscular route or as inhalants. In aspects, Tregitope compositions of the present disclosure can be injected directly into a particular tissue where deposits have accumulated, e.g., intracranial injection. In other aspects, intramuscular injection or intravenous infusion may be used for administration of Tregitope compounds and compositions of the present disclosure. In some methods, T-cell epitope compounds and compositions of the present disclosure are administered as a sustained release composition or device, such as but not limited to a Medipad™ device. In aspects, T-cell epitope compounds and compositions of the present disclosure are administered intradermally, e.g., by using a commercial needle-free high-pressure device such as Pulse NeedleFree technology (Pulse 50TM Micro Dose Injection System, Pulse NeedleFree Systems; Lenexa, Kans., USA). In aspects, said commercial needle-free high-pressure device (e.g., Pulse NeedleFree technology) confers one or more of the following benefits: non-invasive, reduces tissue trauma, reduces pain, requires a smaller opening in the dermal layer to deposit the composition in the subject (e.g., only requires a micro skin opening), instant dispersion of the composition, better absorption of the composition, greater dermal exposure to the composition, and/or reduced risk of sharps injury.

In aspects, Tregitope compounds and compositions of the present disclosure can optionally be administered in combination with other agents that are at least partly effective in treating various medical conditions as described herein. For example, in the case of administration into the central nervous system of a subject, Tregitope compositions of the present disclosure can also be administered in conjunction with other agents that increase passage of the agents of the invention across the blood-brain barrier.

In aspects, solutions or suspensions used for parenteral, intradermal, or subcutaneous application can include, but are not limited to, the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents.

In aspects, the parenteral preparation can be enclosed in ampoules, disposable syringes or multiple dose vials made of glass or plastic.

In aspects, pharmaceutical compositions or formulations suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition is sterile and should be fluid to the extent that easy syringeability exists. It is stable under the conditions of manufacture and storage and is preserved against the contaminating action of microorganisms such as bacteria and fungi. In aspects, formulations including a Tregitope compound and composition of the present disclosure may include aggregates, fragments, breakdown products and post-translational modifications, to the extent these impurities bind HLA and present the same TCR face to cognate T cells they are expected to function in a similar fashion to pure Tregitopes. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, and sodium chloride in the composition. Prolonged absorption of the injectable compositions can be brought about by including in the composition a compound that delays absorption, e.g., aluminum monostearate and gelatin.

In aspects, sterile injectable solutions can be prepared by incorporating the Tregitope compounds and compositions of the present disclosure in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Further, Tregitope compounds and compositions of the present disclosure can be administered in the form of a depot injection or implant preparation that can be formulated in such a manner as to permit a sustained or pulsatile release of the active ingredient.

In aspects, oral compositions generally include an inert diluent or an edible carrier and can be enclosed in gelatin capsules or compressed into tablets. In aspects, for the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. In aspects, the tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel or corn starch; a lubricant such as magnesium stearate or Sterotes; a glidant such as colloidal silicon dioxide; a sweetening compound such as sucrose or saccharin; or a flavoring compound such as peppermint, methyl salicylate or orange flavoring.

For administration by inhalation, Tregitope compounds and compositions of the present disclosure can be delivered in the form of an aerosol spray from pressured container or dispenser that contains a suitable propellant, e.g., a gas such as carbon dioxide, or a nebulizer.

In aspects, systemic administration of the Tregitope compounds and compositions of the present disclosure can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the Tregitope compounds and compositions may be formulated into ointments, salves, gels, or creams, and applied either topically or through transdermal patch technology as generally known in the art.

In aspects, the Tregitope compounds and compositions of the present disclosure can also be prepared in the form of suppositories (e.g., with conventional suppository bases such as cocoa butter and other glycerides) or retention enemas for rectal delivery.

In aspects, the Tregitope compounds and compositions of the present disclosure are prepared with carriers that protect the Tregitope compounds and compositions against rapid elimination from the body, such as a controlled-release formulation, including implants and microencapsulated delivery systems. Biodegradable, biocompatible polymers can be used, such as, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art (U.S. Pat. No. 4,522,811, which is herein incorporated by reference in its entirety). In aspects, the Tregitope compounds and compositions of the present disclosure can be implanted within or linked to a biopolymer solid support that allows for the slow release of the Tregitope compounds and compositions to the desired site.

In aspects, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the instant disclosure are dictated by and directly dependent on the unique characteristics of the binding agent and the particular therapeutic effect to be achieved, and the limitations inherent in the art of compounding such Tregitope compounds and compositions for the treatment of a subject.

In aspects, the one or more of the Tregitope compounds and compositions as disclosed herein can also be administered to the patient by ex vivo pulsing of isolated dendritic cells (DC) with Tregitopes, followed by reinfusion of the pulsed cells into the patient. These can be prepared according to methods known to those skilled in the art (Butterfield, (2013), Front Immunol, 4:454 and Dissanayake et al., (2014), PLoS One, 9(3)1-10). These reinfusions may be administered by the above methods and compositions.

In aspects of a pharmaceutical composition as described herein, the composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the instantly-disclosed peptides or polypeptides (including concatemeric polypeptides) or nucleic acids encoding such peptides or polypeptides (including concatemeric polypeptides). For example, in aspects, a pharmaceutical composition can comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypeptides (including up to 40 peptides or polypeptides), including any value or range therebetween, comprising, consisting of, or consisting essentially of one or more peptides or polypeptides having an amino acid sequence comprising, consisting of, or consisting essentially of one or more of SEQ ID NOS: 1-14 and 74-116 and/or fragments and variants thereof, and optionally 1 to 12 additional amino acids distributed in any ratio on the N terminus and/or C-terminus of the polypeptide of SEQ ID NOS: 1-14 and 74-116; concatemeric peptides as disclosed herein; and nucleic acids (e.g., RNA mRNA, DNA, cDNA) encoding such peptides, polypeptides, or concatemeric peptides, and/or fragments and variants thereof, as described herein.

In aspects, the Tregitope compounds and compositions of the present disclosure (including one or more of e.g., polypeptides (which may be termed herein as “Treg activating regulatory T-cell epitope”, “Tregitope”, or “T-cell epitope polypeptide”) having a sequence comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) are combined in admixture with an antigen, allergen, or a therapeutic protein. Such compositions are useful in methods of inducing tolerance to the antigen, allergen, or a therapeutic protein in a subject in need thereof, wherein local delivery of the admixture with an antigen, allergen, or a therapeutic protein results in increased tolerance to the antigen or allergen in the subject, and delivered with an appropriate excipient resulting in induced tolerance to the antigen, allergen, or a therapeutic protein. This combination may be administered with the Tregitope compounds and compositions of the present disclosure bound either covalently or non-covalently, or they may be administered as an admixture, or a branched or chemically-link preparation. Such compositions are useful in methods of inducing tolerance to an antigen or allergen or a therapeutic protein (e.g., but not limited to, Insulin, coagulation Factor VIII (FVIII) and/or coagulation Factor VIII supplements). For example, such composition are useful in a subject in need thereof, wherein local delivery of the admixture with an antigen or allergen or therapeutic protein results in increased tolerance to the antigen or allergen or therapeutic protein in the subject, and delivered with an appropriate excipient resulting in induced tolerance to the antigen or allergen or therapeutic protein. In aspects, the Tregitope compounds and compositions of the present disclosure are in combination with a therapeutic blood clotting protein for the purpose of suppressing an immune response against the therapeutic blood clotting protein in a T-cell dependent manner. This combination may be administered with the Tregitope compounds and compositions of the present disclosure bound either covalently or non-covalently, or they may be administered as an admixture. Such compositions are useful in methods of inducing tolerance to the therapeutic blood clotting protein in a subject in need thereof, wherein local delivery of the admixture with the therapeutic blood clotting protein results in increased tolerance to the therapeutic blood clotting protein in the subject, and delivered with an appropriate excipient resulting in induced tolerance to the therapeutic blood clotting protein. In aspects of the above Tregitope compounds and compositions combined in admixture with an antigen or allergen or a therapeutic protein or bound either covalently or non-covalently to with an antigen or allergen or a therapeutic protein, the one or more polypeptides (Treg activating regulatory T-cell epitope, Tregitope, Tregitope peptide, or T-cell epitope polypeptide) of the present disclosure included therein have a sequence: comprising, consisting of, or consisting essentially of one or more of SEQ ID NOS: 1-14 and 74-116; comprising, consisting of, or consisting essentially of one or more of SEQ ID NOS: 1-2, and more particularly SEQ ID NO: 1.

### Methods of Use

Stimulating regulatory T cells with Tregitope compounds and compositions of the present disclosure (including one or more of e.g., polypeptides (which may be termed herein as “Treg activating regulatory T-cell epitope”, “Tregitope”, or “T-cell epitope polypeptide”) having a sequence comprising, consisting of, or consisting essentially of one or more of SEQ ID NOS: 1-14 and 74-116 (and/or fragments and variants thereof), and optionally 1 to 12 additional amino acids distributed in any ratio on the N-terminus and/or C-terminus of the polypeptide of SEQ ID NOS: 1-14 and 74-116 as disclosed herein; concatemeric peptides as disclosed herein; nucleic acids, chimeric or fusion polypeptide compositions as disclosed herein; nucleic acids as disclosed herein, expression cassettes, plasmids, expression vectors, recombinant viruses, or cells as disclosed herein; and/or pharmaceutical compositions or formulations as disclosed herein) can stimulate, induce, and/or expand corresponding naturally occurring T_{Reg} populations (in aspects, including natural T_{Regs} and/or adaptive T_{Regs}) and in aspects results in increased secretion of one or more of the following cytokines and chemokines: IL-10, IL-35, TGF-β, TNF-α and MCP1. This increased secretion of regulatory cytokines and chemokines is a hallmark of regulatory T cells. In aspects, stimulation can result in the increased expression of IL-2Rα by corresponding naturally occurring T_{Regs} populations (in aspects, including natural T_{Regs} and/or adaptive T_{Regs}) and deprivation of IL-2 to effector T cells. In further aspects, stimulation can result in increased perforin granzyme by corresponding naturally occurring T_{Reg} populations (in aspects, including natural T_{Regs} and/or adaptive T_{Regs}), which allows for such Treg populations to kill T effector cells and other immune stimulatory cells. In even further aspects, such stimulation can result in the generation of immune suppressive adenosine by corresponding naturally occurring T_{Reg} populations (in aspects, including natural T_{Regs} and/or adaptive T_{Regs}). In other aspects, such stimulation can result in corresponding naturally occurring T_{Reg} populations (in aspects, including natural T_{Regs} and/or adaptive T_{Regs}) binding to and removing costimulatory molecules on dendritic cells, resulting the inhibition of dendritic cell function. Further, in aspects, such stimulation can result in T_{Reg} induced upregulation of checkpoint molecules on dendritic cells and other cell populations, e.g., but not limited to endothelial cells, by corresponding naturally occurring T_{Reg} populations (in aspects, including natural T_{Regs} and/or adaptive T_{Regs}). In additional aspects, such stimulation can result in T_{reg} stimulation of B-regulatory cells. B-regulatory cells (“B-regs”) are cells that are responsible for the anti-inflammatory effect, which is characterized by the expression of CD1d, CD5, and the secretion of IL-10. B-regs are also identified by expression of Tim-1 and can be induced through Tim-1 ligation to promote tolerance. The ability of being B-regs was shown to be driven by many stimulatory factors such as toll-like receptors, CD40-ligand and others. However, full characterization of B-regs is ongoing. B-regs also express high levels of CD25, CD86, and TGF-β. The increased secretion of such regulatory cytokines and chemokines by regulatory T cells, as well as other activities described above, are hallmarks of regulatory T cells. In aspects, regulatory T cells activated by the Tregitope compositions of the present disclosure may express a CD4+CD25+FOXP3 phenotype. In aspects, regulatory T cells activated by the Tregitope compositions of the present disclosure may express a CD4+CD25+Foxp3+ phenotype. Regulatory T cells activated by the Tregitope compounds and compositions of the present disclosure directly suppress T-effector immune responses ex vivo as measured by decreased antigen-specific Th1- or Th2-associated cytokine levels, principally INF-γ, IL-4, and IL-5, and by decreased proliferation and/or effector function of antigen-specific T effector cells as measured by CFSE dilution and/or cytolytic activity. In aspects, regulatory T cells activated by the Tregitope compounds and compositions of the present disclosure directly suppress T effector immune responses in vivo, as measured by decreased antigen-specific Th1- or Th2-associated cytokine levels (as measured by Elisa assay), decreased antigen-specific T effector cell levels (as measured by EliSpot assay), decreased cytolytic activity, and/or decreased antibody titers for protein antigens.

In aspects, natural regulatory T cells activated by the Tregitope compounds and compositions of the present disclosure (stimulate the development of adaptive T_{Reg} cells. In aspects, co-incubating peripheral T cells with the Tregitope compounds and compositions of the present disclosure in the presence of antigen results in the expansion of antigen-specific CD4+/CD25+ T cells, upregulates the expression of the Foxp3 gene or Foxp3 protein in those cells and suppresses the activation of antigen-specific T effector cells in vitro. In aspects, the Tregitope compounds and compositions of the present disclosure may result in the activation and/or expansion of T regulatory type 1 (Tr1) cells. Tr1 cells have strong immunosuppressive capacity in several immune-mediated diseases (Roncarolo and Battaglia, 2007, Nat Rev Immunol 7, 585-598; Roncarolo et al., 2011, Immunol Rev 241, 145-163; Pot et al., 2011, Semin Immunol 23, 202-208). The secretion of high levels of IL-10, and the killing of myeloid antigen-presenting cells (APCs) via Granzyme B are the main mechanisms of Tr1-mediated suppression (Groux et al., 1997, Nature 389, 737-742; Magnani et al., 2011 Eur J Immunol 41, 1652-1662). Tr1 cells are distinguished from T helper (T_{H})1, T_{H}2, and T_{H}17 cells by their unique cytokine profile and the regulatory function. Tr1 cells have been shown secrete higher levels of IL-10 than IL-4 and IL-17, the hallmark cytokines of T_{H}2 and T_{H}17 cells, respectively. Tr1 cells can also secrete low levels of IL-2 and, depending on the local cytokine milieu, can produce variable levels of IFN-γ, together, the key T_{H}1 cytokines (Roncarolo et al., 2011, Immunol Rev 241, 145-163). FOXP3 is not a biomarker for Tr1 cells since its expression is low and transient upon activation. IL-10-producing Tr1 cells express ICOS (Haringer et al., 2009, J Exp Med 206, 1009-1017) and PD-1 (Akdis et al., 2004, J Exp Med 199, 1567-1575), but these markers are not specific (Maynard et al., 2007, Nat Immunol 8, 931-941). CD49b, the α2 integrin subunit of the very-late-activation antigen (VLA)-2, has been proposed as a marker for IL-10-producing T cells (Charbonnier et al., 2006, J Immunol 177, 3806-3813); but it is also expressed by human T_{H}17 cells (Boisvert et al., 2010, Eur J Immunol 40, 2710-2719). Moreover, murine CD49b⁺ T cells secrete IL-10 (Charbonnier et al., 2006, J Immunol 177, 3806-3813) but also pro-inflammatory cytokines (Kassiotis et al., 2006, J Immunol 177, 968-975). Lymphocyte activation gene-3 (LAG-3), a CD4 homolog that binds with high affinity to MHC class II molecules, is expressed by murine IL-10-producing CD4⁺ T cells (Okamura et al., 2009, Proc Natl Acad Sci USA 106, 13974-13979), but also by activated effector T cells (Workman and Vignali, 2005, J Immunol 174, 688-695; Bettini et al., 2011, J Immunol 187, 3493-3498; Bruniquel et al., 1998, Immunogenetics 48, 116-124; Lee et al., 2012, Nat Immunol 13, 991-999) and by FOXP3⁺ regulatory T cells (Tregs) (Camisaschi et al., 2010, J Immunol 184, 6545-6551). It was recently shown that human Tr1 cells express CD226 (DNAM-1), which is involved in the specific killing of myeloid APCs (Magnani et al., 2011 Eur J Immunol 41, 1652-1662). In further aspects, Tregitope compounds and compositions of the present disclosure may result in the activation and/or expansion of TGF-β secreting Th3 cells, regulatory NKT cells, regulatory CD8⁺ T cells, double negative regulatory T cells. “Th3 cells” refer to cells having the following phenotype CD4⁺ FoxP3⁺ and capable of secreting high levels TGF-β upon activation, amounts of IL-4 and IL-10 and no IFN-γ or IL-2. These cells are TGF-β derived. “Regulatory NKT cells” refers to cells having the following phenotype at rest CD161⁺CD56⁺CD16+ and a Vα24/Vβ11 TCR. “Regulatory CD8+ T cells” refers to cells having the following phenotype at rest CD8⁺CD122⁺ and capable of secreting highs levels of IL-10 upon activation. “Double negative regulatory T cells” refers to cells having the following phenotype at rest TCRαβ⁺CD4⁻CD8⁻.

In aspects, the Tregitope compounds and compositions of the present disclosure (including one or more of e.g., polypeptides are useful for regulating immune response to monoclonal antibodies, protein therapeutics, self-antigens promoting autoimmune response, allergens, transplanted tissues and in other applications where tolerance is the desired outcome. In aspects, the Tregitope compounds and compositions of the present disclosure are useful for regulating an immune response caused by Factor VIII supplements used to prevent or stop bleeding in patients suffering from Hemophilia A.

In aspects, the Tregitopes of the present disclosure can bind MHC class II molecules, engage TCR in context of MHC class II molecules and activate naturally occurring T_{Regs} (in aspects, including natural T_{Regs} and/or adaptive T_{Regs}s).

Suppressing an Immune Response in a Subject in Need Thereof. In aspects, the present disclosure is directed to a method of stimulating, inducing, and/or expanding regulatory T-cells by administering or introducing or contacting with an amino acid sequence of SEQ ID NOS: 1-14 and 74-116 or a fragment or variant thereof, either directly or through introduction of a nucleic acid encoding such and providing or allowing for transcription and translation thereof.

In aspects, the present disclosure is directed to a method of stimulating, inducing, and/or expanding regulatory T-cells (e.g., naturally occurring T_{Regs} (in aspects, including natural T_{Regs} and/or adaptive T_{Regs})) to suppress an immune response in a subject in need thereof by administering to the subject a therapeutically effect amount of a Tregitope compound or composition of the present disclosure. In aspects, the immune response is the result of one or more therapeutic treatments with at least one therapeutic protein, treatment with a vaccine (particularly in situations in which an adverse event results from the vaccination), or treatment with at least one antigen. In a particular embodiment, the immune response is the result of one or more therapeutic treatments with a Coagulation Factor VIII protein or supplement or a Coagulation Factor V protein or supplement. Thus, the administration of one or more Tregitope compounds and compositions of the present disclosure can be used to prevent the development of, or terminate, and already-established immune response to establish tolerance induction to Factor VIII (and Coagulation Factor VIII supplements) in patients suffering from Hemophilia A. In aspects, the instant disclosure provides methods of using a Tregitope compound or composition of the present disclosure in combination with a therapeutic blood clotting protein (e.g., a Coagulation Factor VIII supplement) for the purpose of suppressing an immune response against the therapeutic blood clotting protein in a T-cell dependent manner. This combination may be administered with the Tregitope compounds and compositions of the present disclosure bound either covalently or non-covalently, or they may be administered as an admixture. In another aspect, the administration of a Tregitope compound or composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method for repressing/suppressing an immune response in a subject, comprising administering a therapeutically effective amount of Tregitope compound or composition of the present disclosure, wherein the Tregitope compound or composition represses/suppresses the immune response. In aspects, the Tregitope compound or composition represses/suppresses an innate immune response. In aspects, the Tregitope compound or composition represses/suppresses an adaptive immune response. In aspects, the Tregitope compound or composition represses/suppresses an effector T cell response. In aspects, the Tregitope compound or composition represses/suppresses a memory T cell response. In aspects, the Tregitope compound or composition represses/suppresses helper T cell response. In aspects, the Tregitope compound or composition represses/suppresses B cell response. In aspects, the Tregitope compound or composition represses/suppresses a nkT cell response.

In aspects, the present invention is directed to a method of suppressing an immune response, specifically an antigen specific immune response in a subject, through the administration of a therapeutically effective amount of a Tregitope compound or composition of the present disclosure, wherein said Tregitope compound or composition activates naturally occurring T_{Regs} (in aspects, including natural T_{Regs} and/or adaptive T_{Regs}, and in aspects CD4⁺/CD25⁺/FoxP3⁺ regulatory T-cells) or suppresses the activation of CD4+ T-cells, the proliferation of CD4⁺ and/or CD8+ T-cells, and/or suppresses the activation or proliferation of β-cells or nkT Cells. In aspects, a Tregitope compound or composition of the present disclosure may be either covalently bound, non-covalently bound, or in admixture with a specific target antigen. In particular aspects, one or more of e.g., isolated, synthetic, or recombinant isolated, synthetic, or recombinant polypeptides (Treg activating regulatory T-cell epitope, Tregitope, Tregitope peptide, or T-cell epitope polypeptide) and/or chimeric or fusion polypeptide compositions of the presently disclosed Tregitope compounds and compositions may be either covalently bound, non-covalently bound, or in admixture with a specific target antigen. In aspects, an administered Tregitope compound or composition of the present disclosure that is covalently bound, non-covalently bound, or in admixture with a specific target antigen results in the diminution of immune response against the target antigen.

In aspects, the target antigen may be an autologous protein or protein fragment. In aspects, the target antigen may be an allergen. In aspects, the target antigen may be an allogenic protein or protein fragments. In aspects, the target antigen may be a biologic medicine or fragments thereof. In aspects, the target antigen is a coagulation Factor VIII replacement protein or supplement or Factor V replacement protein or supplement. In aspects, the suppressive effect is mediated by natural T_{Regs}. In aspects, the suppressive effect is mediated by adaptive T_{Regs}. In aspects, the one or more Tregitopes included in the Tregitope compound or composition of the present disclosure suppresses an effector T cell response. In aspects, the one or more Tregitopes of the presently-disclosed Tregitope compound or composition suppresses an innate immune response. In aspects, the one or more Tregitopes of the presently-disclosed Tregitope compound or composition suppresses an adaptive immune response. In aspects, the one or more Tregitopes of the presently-disclosed Tregitope compound or composition suppresses helper T cell response. In aspects, the one or more Tregitopes of the presently-disclosed Tregitope compound or composition suppresses a memory T cell response. In aspects, the one or more Tregitopes of the presently-disclosed Tregitope compound or composition suppresses β cell response. In aspects, the one or more Tregitopes of the presently-disclosed Tregitope compound or composition suppresses nkT cell response.

Designing Small Molecule Therapeutics. In one aspect, the present disclosure provides methods of using a Tregitope compound or composition of the present disclosure for the purpose of designing small molecule therapeutics. In one aspect, Tregitope-specific T cells are stimulated three times with pools of small molecule mixtures at a concentration of 1 μg/ml and autologous dendritic cells (DC) at 2-week intervals, followed by stimulation with heterologous DC and antigens. T cells (1.25×10⁵) and DC (0.25×10⁵) are added per well in round-bottom, 96-well plates. T cell medium is made by supplementing 500 ml of RPMI medium 1640 with 50 ml of FCS (HyClone Laboratories, Inc., Logan, Utah), penicillin, and streptomycin (GIBCO Laboratories, Gaithersburg, Md.); 20 mM Hepes (GIBCO); and 4 ml 1 N NaOH solution. The IL-2 concentration is initially 0.1 nM and gradually is increased to 1 nM during subsequent rounds of stimulation. T cell clones are derived by limiting dilution by using 0.6×10⁵ Epstein-Barr virus-transformed B cells (100 Gray) and 1.3×10⁵ heterologous peripheral blood mononuclear cells (33 Gray) as feeder cells and 1 μg/ml Difco™ phytohemagglutinin (Bacterius Ltd, Houston, Tex.) in medium containing 2 nM IL-2. Small molecules pools that stimulate the Tregitope specific T cells are then tested as individual molecules.

Cloning T Cell Receptors. In aspects, the present disclosure provides methods of using a Tregitope compound or composition of the present disclosure for the purpose of cloning T cell receptors. Cloning of Tregitope specific T cells can be conducted by techniques known to one of skill in the art. For example, isolated PBMCs are stimulated with Tregitopes at 10 μg/ml RPMI media containing 20% HSA. IL-2 is added (10 U/ml final concentration) every other day starting on day 5. T cells are stained with tetramer pools on day 11 or 12. For each pool, 2−3×10⁵ cells are incubated with 0.5 mg of PE-labeled tetramer in 50 ml of culture medium (10 mg/ml) at 37° C. for 1 to 2 h, and then stained with anti-CD4-FITC (BD PharMingen, San Diego, Calif.) for 15 min at room temperature. Cells are washed and analyzed with a Becton Dickinson FACSCalibur flow cytometer (Becton Dickinson, San Jose, Calif.). Tetramers loaded with the corresponding single peptides are generated for those pools that give positive staining, and analysis is done on day 14 or 15. Cells that are positive for a particular tetramer are single-cell sorted into 96-well U-bottom plates by using a Becton Dickinson FACSVantage (San Jose, Calif.) on the same or following day. Sorted cells are expanded with 1.5−3×10⁵ unmatched, irradiated (5000 rad) PBMC per well as feeders with 2.5 mg/ml PHA and 10 U/ml IL-2 added 24 h later. Specificity of cloned T cells is confirmed by staining with tetramers (loaded with cognate peptide or control peptide, HA307-319) and T cell proliferation assays with 10 mg/ml of specific peptide (Novak EJ et al., J Immunol, 166(11):6665-70, which is herein incorporated by reference in its entirety). In aspects, total RNA is extracted with an RNeasy Mini Kit (Qiagene, Hilden, Del.) from the Tregitope specific T cell lines generated as described above. One microgram of total RNA is used to clone the TCR cDNAs by a rapid amplification of cDNA end (RACE) method using aGeneRacer® kit (Invitrogen, Carlsbad, Calif.). Before synthesizing the single-strand cDNA, the RNA is de-phosphorylated, de-capped, and ligated with an RNA oligonucleotide according to the instruction manual of 5′ RACE GeneRacer® kit. SuperScript II RT® (Life Technologies Corp, Carlebad, Calif.) and GeneRacer® Oligo-dT are used for reverse transcription of the RNA Oligo-ligated mRNA to single-strand cDNAs. 5′ RACE is performed by using GeneRacer® 5′ (GeneRacer® Kit) as 5′ primer and gene-specific primer TCRCAR (5′-GTT AAC TAG TTC AGC TGG ACC ACA GCC GCA GC-3′; SEQ ID NO: 65) or TCRCB1R (5′- CGG GTT AAC TAG TTC AGA AAT CCT TTC TCT TGA CCA TGG C -3′; SEQ ID NO: 66), or TCRCBR2 (5′-CTA GCC TCT GGA ATC CTT TCT CTT G-3′; SEQ ID NO: 67) as 3′ primers for TCR α, β1, or β2 chains, respectively. The polymerase chain reaction (PCR) products are cloned into pCR2.1 TOPO vector (Invitrogen, Carlsbad, Calif.) and then transformed into One Shot TOP10 Competent Escherichia coli (Invitrogen, Carlsbad, Calif.). Plasmid DNAs are prepared from 96 individual clones from each construct for TCRα, β1, and β2 chains. Full-length insert of all the plasmids is sequenced to determine the vα/vβ usage (Zhao Y et al., (2006), J Immunother, 29(4):398-406, herein incorporated by reference in its entirety).

Methods of Preventing or Treating a Medical Condition. The present disclosure is directed to, for example methods of preventing or treating one or more medical conditions in a subject comprising administering a Tregitope compound or composition of the present disclosure, and preventing or treating the medical condition in a subject by said step of administering. The medical condition can be, for example, primary immunodeficiencies (such as autoimmunity associated with primary immune deficiency disorders); immune-mediated thrombocytopenia, Kawasaki disease, hematopoietic stem cell transplantation in patients older than 20 years, chronic B-cell lymphocytic leukemia and pediatric HIV type 1 infections. Specific examples include: (Hematology) aplastic anemia, pure red cell aplasia, Diamond-Blackfan anemia, autoimmune hemolytic anemia, hemolytic disease of the newborn, acquired factor VIII inhibitors, acquired von Willebrand disease, immune-mediated neutropenia, refractoriness to platelet transfusion, neonatal alloimmune/autoimmune thrombocytopenia, posttransfusion purpura, thrombotic thrombocytopenia purpura/hemolytic uremic syndrome; (Infectious diseases), solid organ transplantation, surgery, trauma, burns, and HIV infection; (Neurology) epilepsy and pediatric intractable Guillain-Barré syndrome, chronic inflammatory demyelinating polyneuropathy, myasthenia gravis, Lambert-Eaton myasthenic syndrome, multifocal motor neuropathy, multiple sclerosis; (Obstetrics) recurrent pregnancy loss; (Pulmonology) asthma, chronic chest symptoms, rheumatology, rheumatoid arthritis (adult and juvenile), systemic lupus erythematosus, systemic vasculitides, dermatomyositis, polymyositis, inclusion-body myositis, wegener granulomatosis; (Miscellaneous) adrenoleukodystrophy, amyotrophic lateral sclerosis, Behcet syndrome, acute cardiomyopathy, chronic fatigue syndrome, congential heart block, cystic fibrosis, autoimmune blistering dermatosis, diabetes mellitus, acute idiopathic dysautonomia, acute disseminated encephalomyelitis, endotoxemia, hemolytic transfusion reaction, hemophagocytic syndrome, acute lymphoblastic leukemia, lower motor neuron syndrome, multiple myeloma, human T-cell lymphotrophic virus-1-associated myelopathy, nephritic syndrome, membranous nephropathy, nephrotic syndrome, euthyroid ophthalmopathy, opsoclonus-myoclonus, recurrent otitis media, paraneoplastic cerebellar degeneration, paraproteinemic neuropathy, parvovirus infection (general), polyneuropathy, organomegaly, endocrinopathy, M-protein, and skin changes (POEMS) syndrome, progressive lumbosacral plexopathy, lyme radiculoneuritis, Rasmussen syndrome, Reiter syndrome, acute renal failure, thrombocytopenia (nonimmune), streptococcal toxic shock syndrome, uveitis and Vogt-Koyanagi-Harada syndrome.

In a particular aspect, the present invention is directed to, for example, methods of treating allergy, autoimmune disease, transplant-related disorders such as graft versus host disease, enzyme or protein deficiency disorders, hemostatic disorders (e.g., Hemophilia A, B, or C), cancers (particularly tumor associated autoimmunity), infertility, or infections (viral, bacterial, or parasitic). The Tregitope compound or composition of the present disclosure can be used with in conjunction with other proteins or compounds used for treating a subject with a medical condition in order to reduce adverse events or enhance the efficacy of the co-administered compound.

Application to Preventing or Treating an Antibody and/or CD4+ T cell Response Caused by Factor VIII Supplements used to Prevent or Stop Bleeding in Patients Suffering from Hemophilia A. Hemophilia is a disease characterized by excessive bleeding after a cut or injury. The cause of this excessive bleeding is failure for blood to clot properly in patients with hemophilia. Hemophilia is a genetic disorder, which means it's the result of a change in genes that was either inherited (passed on from parent to child) or happened during development in the womb. Bleeding can be external or internal. Internal bleeding of the joints (like the knees or hips) is common in children with hemophilia. Hemophilia mostly affects boys—about 1 in every 5,000-10,000. Girls who inherit the gene rarely get the condition, but as carriers of the gene they can pass it to their children.

When an injury results in bleeding, platelets begin the clotting process. Platelets release chemicals that attract additional platelets as well as activating proteins in the blood known as clotting factors, in humans, these are known as human coagulation Factors I through XIII. These proteins mix with the platelets to form fibers, which strengthen the clot and stop the bleeding. Patients suffering from hemophilia fail to produce sufficient amounts of the clotting factor that work together to clot blood. Hemophilia results from a patient being unable to produce enough Factor VIII or Factor IX to clot blood.

There are two major kinds of hemophilia: hemophilia A and hemophilia B. About 80% of cases are hemophilia A, which is a Factor VIII deficiency, hemophilia B and C occur when the body produces too little Factor IX. Hemophilia can be mild, moderate, or severe, based on the amount of the clotting factor in the blood:

Mild hemophilia: the body makes 6% to 50% of the affected clotting factor

Moderate hemophilia: the body makes 2% to 5% of the affected clotting factor

Severe hemophilia: the body makes less than 1% of the affected clotting factor

In general, a patient with milder hemophilia may bleed too much only once in a while. A patient with severe hemophilia is at risk for bleeding problems much more often.

Hemophilia is a lifelong condition with no cure other than liver transplantation, a procedure that can sometimes cause health problems more serious than hemophilia itself.

Factor VIII replacement therapy helps blood to clot and prevents long-term joint damage due to bleeding. It may be administered while a bleeding episode is happening to promote clotting, or in regularly scheduled treatments to keep the blood healthy. The therapy is “infused” in the blood—given through an intravenous (IV) line either at a clinic or at home by a visiting nurse or by parents (and patients themselves) who have had training to administer this therapy. Once the clotting factor is in the blood, it begins to work quickly.

Human patients suffering from Hemophilia A, however, frequently mount an antibody response against the coagulation Factor VIII supplements prescribed to prevent or stop bleeding episodes. A patient's body views the new clotting factor as foreign and develops antibodies that block its clotting action. About a quarter of children with severe Hemophilia A develop antibodies to the clotting factor. These antibodies can make the hemophilia difficult to treat because these antibody responses diminish the efficacy of the Factor VIII treatments leading to further complications, such as severe adverse bleeding episodes and arthritis.

One strategy both for the prevention and therapy of an unwanted antibody and/or CD4+ T cell response caused by Factor VIII supplements used to prevent or stop bleeding in patients suffering from Hemophilia A is the induction of regulatory T cells. Hemophiliacs can be protected from developing an unwanted immune response when receiving Factor VIII supplements. In aspects, the present invention is directed to preventing or diminishing an autoimmune response caused by human coagulation Factor VIII supplements used to prevent or stop bleeding in patients suffering from Hemophilia A comprising administering a Tregitope compound or composition of the present disclosure, thereby treating the medical condition. The Tregitope compounds and compositions of the invention can be used with or in conjunction with other proteins or compounds (e.g., but not limited to, human coagulation Factor VIII supplements) used for treating a subject with a medical condition (e.g., but not limited to Hemophilia A) in order to reduce adverse events or enhance the efficacy of the co-administered compound.

Application to Allergy. Allergen-specific regulatory T cells play an important role in controlling the development of allergy and asthma. Naturally occurring T_{Regs} (in aspects, including natural T_{Regs} and/or adaptive T_{Regs}, and in aspects CD4⁺/CD25⁺/FoxP3⁺ regulatory T-cells) have been shown to inhibit the inappropriate immune responses involved in allergic diseases. A number of recent studies indicate that regulatory T cells play an important role in controlling the overdevelopment of T-helper type 2 biased immune responses in susceptible individuals, not only in animal models, but in humans as well. Recent studies indicate that T_{regs} also suppress T cell co-stimulation by the secretion of TGF-β and IL-10, suggesting an important role of T_{regs} in the regulation of allergic disorders. Impaired expansion of natural or adaptive regulatory T cells leads to the development of allergy, and treatment to induce allergen-specific T_{regs} would provide curative therapies for allergy and asthma. One strategy both for the prevention and therapy of asthma is the induction of T_{regs}. Animals can be protected from developing asthma by immune stimulation leading to Th1 or T_{reg} responses. Accordingly, Tregitope compounds and compositions of the present disclosure are useful in methods for the prevention or treatment of allergy and/or asthma. As such, in aspects, the present disclosure is directed to a method of preventing or treating allergy and/or asthma in a subject, the method comprising administering a therapeutically-effective amount of a Tregitope compound or composition of the present disclosure, and preventing or treating allergy and/or asthma in a subject by said step of administering.

Application to Transplantation. The Tregitope compounds and compositions of the present disclosure are useful to induce tolerance during the transplantation process, by promoting the development of cells that specifically down regulate immune responses against donor cells. Induction of Ag-specific T_{Reg} cells for treating organ-specific autoimmunity is an important therapeutic development, avoiding generalized immune suppression. In murine models of bone marrow transplantation, T_{Regs} promote donor bone marrow engraftment and decrease the incidence and severity of graft versus host disease without abrogating the beneficial graft versus tumor immunologic effect. These findings, in concert with observations that T_{Regs} in mice and humans share phenotypic and functional characteristics, have led to active investigations into the use of these cells to decrease complications associated with human hematopoietic cell transplantation. An imbalance of T_{Regs} and effector T cells contributes to the development of graft versus host disease, however, the mechanisms of immunoregulation, in particular, the allorecognition properties of T_{Regs}, their effects on and interaction with other immune cells, and their sites of suppressive activity, are not well understood.

Accumulating evidence from both humans and experimental animal models has implicated the involvement of T_{Regs} in the development of graft versus host disease (GVHD). The demonstration that T_{Regs} can separate GVHD from graft versus tumor (GVT) activity suggests that their immunosuppressive potential could be manipulated to reduce GVHD without detrimental consequence on GVT effect. Although a variety of T lymphocytes with suppressive capabilities have been reported, the two best-characterized subsets are the naturally arising, intrathymic-generated T_{Regs} (natural T_{Regs}) and the peripherally generated, adaptive T_{Regs} (adaptive T_{Regs}). Accordingly, Tregitope compounds and compositions of the present disclosure are useful in methods for inducing tolerance during the transplantation process. As such, in aspects, the present disclosure is directed to a method of inducing tolerance during the transplantation process in a subject, the method comprising administering a therapeutically-effective amount of a Tregitope compound or composition of the present disclosure, and inducing tolerance during the transplantation process in a subject by said step of administering.

Application as a Tolerizing Agent and to Autoimmunity. In aspects, Tregitope compounds and compositions of the present disclosure can be used as a tolerizing agents for immunogenic compounds (protein therapeutics) (Weber CA et al., (2009), Adv Drug Deliv, 61(11):965-76). This discovery has implications for the design of protein therapeutics. Thus, administration of a monoclonal antibody, autologous cytokine, or foreign protein in conjunction with a Tregitope compound or composition of the present disclosure suppresses adverse T effector immune responses. In vivo, T_{Regs} act through dendritic cells to limit autoreactive T-cell activation, thus preventing their differentiation and acquisition of effector functions. By limiting the supply of activated pathogenic cells, T_{Regs} prevent or slow down the progression of autoimmune diseases. This protective mechanism appears, however, insufficient in autoimmune individuals, likely because of a shortage of T_{Regs} cells and/or the development and accumulation of T_{Reg}-resistant pathogenic T cells over the long disease course. Thus, restoration of self-tolerance in these patients may require purging of pathogenic T cells along with infusion of T_{Regs} with increased ability to control ongoing tissue injury. Organ-specific autoimmune conditions, such as thyroiditis and insulin-dependent diabetes mellitus have been attributed to a breakdown of this tolerance mechanism (Mudd PA et al., (2006), Scand J Immunol, 64(3):211-8). Accordingly, Tregitope compounds and compositions of the present disclosure are useful in methods for the prevention or treatment of autoimmunity. As such, in aspects, the present disclosure is directed to a method of preventing or treating autoimmunity in a subject, the method comprising administering a therapeutically-effective amount of a Tregitope compound or composition of the present disclosure, and preventing or treating autoimmunity in a subject by said step of administering.

Application to Diabetes. Type 1 (juvenile) diabetes is an organ-specific autoimmune disease resulting from destruction of insulin-producing pancreatic beta-cells. In non-diabetics, islet cell antigen-specific T cells are either deleted in thymic development or are converted to T regulatory cells that actively suppress effector responses to islet cell antigens. In juvenile diabetics and in the NOD mouse model of juvenile diabetes, these tolerance mechanisms are missing. In their absence, islet cell antigens are presented by human leukocyte antigen (HLA) class I and II molecules and are recognized by CD8(+) and CD4(+) auto-reactive T cells. Destruction of islet cells by these auto-reactive cells eventually leads to glucose intolerance. Co-administration of Tregitopes and islet cell antigens leads to the activation of naturally occurring T regulatory cells and the conversion of existing antigen specific effector T cell to a regulatory phenotype. In this way, deleterious autoimmune response is redirected leading to the induction of antigen-specific adaptive tolerance. Modulation of auto-immune responses to autologous epitopes by induction of antigen-specific tolerance can prevent ongoing beta-cell destruction. Accordingly, Tregitope compounds and compositions of the present disclosure are useful in methods for the prevention or treatment of diabetes. As such, in aspects, the present disclosure is directed to a method of preventing or treating diabetes in a subject, the method comprising administering a therapeutically-effective amount of Tregitope compound or composition of the present disclosure, and preventing or treating diabetes in a subject by said step of administering.

Application to Hepatitus B (HBV) infection. Chronic HBV is usually either acquired (by maternal fetal transmission) or can be a rare outcome of acute HBV infection in adults. Acute exacerbations of chronic hepatitis B (CH-B) are accompanied by increased cytotoxic T cell responses to hepatitis B core and e antigens (HBcAg/HBeAg). In a recent study, the SYFPEITHI T cell epitope mapping system was used to predict MHC class II-restricted epitope peptides from the HBcAg and HbeAg (Feng IC et al., (2007), J Biomed Sci, 14(1):43-57). MHC class II tetramers using the high scoring peptides were constructed and used to measure T_{Reg} and CTL frequencies. The results showed that T_{Reg} cells specific for HBcAg declined during exacerbations accompanied by an increase in HBcAg peptide-specific cytotoxic T cells. During the tolerance phase, FOXp3-expressing T_{Reg} cell clones were identified. These data suggest that the decline of HbcAg T_{Reg} T cells accounts for the spontaneous exacerbations on the natural history of chronic hepatitis B virus infection. Accordingly, Tregitope compounds and compositions of the present disclosure are useful in methods for the prevention or treatment of viral infections. As such, in aspects, the present disclosure is directed to a method of preventing or treating a viral infection (e.g., HBV infection) in a subject, the method comprising administering a therapeutically-effective amount of a Tregitope compound or composition of the present disclosure, and preventing or treating said viral infection in a subject by said step of administering.

Application to SLE. A T_{Reg} epitope that plays a role in Systemic Lupus Erythematosis (SLE) or Sjogren's syndrome has been defined. This peptide encompasses residues 131-151 (RIHMVYSKRSGKPRGYAFIEY; SEQ ID NO: 68) of the spliceosome protein. Binding assays with soluble HLA class II molecules and molecular modeling experiments indicated that the epitope behaves as promiscuous epitope and binds to a large panel of human DR molecules. In contrast to normal T cells and T cells from non-lupus autoimmune patients, PBMCs from 40% of randomly selected lupus patients contain T cells that proliferate in response to peptide 131-151. Alteration of the ligand modified the T cell response, suggesting that several populations of T cells responding to this peptide exist, among which may be T_{Reg} cells. T regulatory epitopes have also been defined in Sjogren's syndrome. Accordingly, Tregitope compounds and compositions of the present disclosure administered in combination with the Tregitope of SEQ ID NO: 68 are useful in methods for the prevention or treatment of SLE. As such, in aspects, the present disclosure is directed to a method of preventing or treating SLE in a subject, the method comprising administering a therapeutically-effective amount of a Tregitope compound or composition of the present disclosure in combination with the Tregitope of SEQ ID NO: 68, and preventing or treating SLE in a subject by said step of administering.

Application to Autoimmune Thyroiditis. Autoimmune Thyroiditis is a condition that occurs when antibodies arise to self-thyroid peroxidase and/or thyroglobulin, which cause the gradual destruction of follicles in the thyroid gland. HLA DR5 is closely associated with the disease. Accordingly, Tregitope compounds and compositions of the present disclosure administered in combination with thyroid peroxidase and/or thyroglobulin TSHR or portions thereof are useful in methods for the prevention or treatment of autoimmune thyroiditis. As such, in aspects, the present disclosure is directed to a method of preventing or treating autoimmune thyroiditis in a subject, the method comprising administering a therapeutically-effective amount of a Tregitope compound or composition of the present disclosure in combination with thyroid peroxidase and/or thyroglobulin TSHR or portions thereof, and preventing or treating autoimmune thyroiditis in a subject by said step of administering. In further aspects, Tregitope compounds and compositions of the present disclosure administered in combination with TSHR or other Graves' disease antigens or portions thereof are useful in methods for the prevention or treatment of Grave's disease. Graves' disease is an autoimmune disorder that is characterized by antibodies to self-thyroid stimulating hormone receptor (TSHR) leading to leading to hyperthyroidism, or an abnormally strong release of hormones from the thyroid gland. Several genetic factors can influence susceptibility to Graves' disease. Females are much more likely to contract the disease than males; White and Asian populations are at higher risk than black populations and HLA DRB1-0301 is closely associated with the disease. As such, in aspects, the present disclosure is directed to a method of preventing or treating Grave's disease in a subject, the method comprising administering a therapeutically-effective amount of a Tregitope compound or composition of the present disclosure in combination with TSHR or other Graves' disease antigens or portions thereof, and preventing or treating Grave's disease in a subject by said step of administering.

Ex Vivo Expansion and/or Stimulation of T-Regulatory Cells Using Tregitope compounds and compositions. In aspects, the present disclosure provides ex vivo methods for the expansion of regulatory T-cells. In one embodiment, the invention provides a method of expanding regulatory T-cells in a biological sample, the method comprising: (a) providing a biological sample from a subject; (b) isolating regulatory T-cells from the biological sample; and contacting the isolated regulatory T-cells with an effective amount of a Tregitope compound or composition of the present disclosure under conditions wherein the T-regulatory cells increase in number to yield an expanded regulatory T-cells, thereby expanding the regulatory T-cells in the biological sample. In aspects, the method further comprises the step of administration of the expanded regulatory T-cells to a subject. In aspects, the subject administered the expanded regulatory T-cells is the same individual from which the original biological sample was obtained, e.g., by autologous transplantation of the expanded Tregitope (Ruitenberg JJ et al., (2006), BMC Immunol, 7:11).

In aspects, the present disclosure provides ex vivo methods for stimulation of regulatory T-cells in a biological sample, the method comprising: (a) providing a biological sample from a subject; (b) isolating regulatory T-cells from the biological sample; and contacting the isolated regulatory T-cells with an effective amount of a Tregitope compound or composition of the present disclosure under conditions wherein the T-regulatory cells are stimulated to alter one or more biological function, thereby stimulating the regulatory T-cells in the biological sample. In aspects, the method further comprises the step of administration of the stimulated regulatory T-cells to a subject. In aspects, the subject administered the stimulated regulatory T-cells is the same individual from which the original biological sample was obtained, e.g., by autologous transplantation of the expanded Tregitope.

Ex Vivo Pulsing of Antigen Presenting Cells using Tregitope compounds and compositions. In aspects, the present disclosure provides ex vivo methods for antigen presenting cells (e.g., dendritic cells, macrophages, etc.) in a biological sample, the method comprising: (a) providing a biological sample from a subject; (b) isolating antigen presenting cells from the biological sample; and contacting the isolated antigen presenting with an effective amount of a Tregitope compound or composition of the present disclosure under conditions wherein the antigen presenting cells are stimulated to alter one or more biological function (e.g., to present the Tregitopes and/or skew the antigen presenting cells to a be tolerogenic (which in aspects can further include cytokine treatment of the antigen presenting cells to induce such a tolerogenic state), thereby stimulating the antigen presenting cells in the biological sample. In aspects, the method further comprises the step of administration of the stimulated antigen presenting cells to a subject. In aspects, the subject administered the stimulated antigen presenting cells is the same individual from which the original biological sample was obtained, e.g., by autologous transplantation of the stimulated antigen presenting cells.

In Vitro Uses of Tregitope compounds and compositions. In aspects, the present disclosure provides the use of a Tregitope compound or composition of the present disclosure as reagents in the study of regulatory T-cell function in in vitro studies and experimental models.

Methods of Immune Engineering. In aspects, the present disclosure is directed to a methods of immune engineering, including removal or insertion of one or more Tregitopes of the instant disclosure, from or into a polypeptide.

For example, in aspects the present disclosure is directed to a method for decreasing the immunogenicity and/or increasing tolerogenicity of a polypeptide, which may be particularly useful when a polypeptide (such as human Factor VIII or a Factor VIII replacement protein or supplement (or fragments thereof), or human Factor V or a Factor V replacement protein or supplement (or fragments thereof)) serves as a therapeutic protein. In aspects, said method comprises insertion of one or more regulatory T cell epitopes (e.g., a peptide or polypeptide comprising, consisting of, or consisting essentially of one or more peptides or polypeptides having an amino acid sequence of SEQ ID NOS: 1-14 and 74-116 (and/or fragments or variants thereof), and optionally 1 to 12 additional amino acids distributed in any ratio on the N-terminus and/or C-terminus of the polypeptide of SEQ ID NOS: 1-14 and 74-116) into said polypeptide (e.g., human Factor VIII or a Factor VIII replacement protein or supplement (or fragments thereof), or human Factor V or a Factor V replacement protein or supplement (or fragments thereof)). In aspects, the one or more regulatory T cell epitopes inserted into the human Factor V or Factor VIII molecule or replacement protein/supplement (or fragments thereof) can suppress an antigen-specific immune response against the human Factor V or Factor VIII molecule or replacement protein/supplement (or fragments thereof). In aspects, said one or more regulatory T cell epitopes may be fused to or inserted internally within (e.g., but not limited to, site directed mutagenesis or other recombinant techniques) a human Factor V or Factor VIII molecule or replacement protein/supplement (or fragments thereof), such as in instances where the Tregitope is not located in its natural position within the Factor V or Factor VIII molecule or replacement protein/supplement (or fragments thereof) or wherein the Factor V or Factor VIII molecule or replacement protein/supplement (or fragments thereof) thereof is missing such a Tregitope (e.g., if a particular Factor V or Factor VIII molecule or replacement protein/supplement (or fragments thereof) has a mutated or missing corresponding section). In aspects, said insertion of the one or more regulatory T cell epitopes into the Factor V or Factor VIII molecule or replacement protein/supplement (or fragments thereof) thereof comprises insertion of all or some of the amino acids of the one or more regulatory T cell epitopes. In aspects, said insertion of the one or more regulatory T cell epitopes into the Factor V or Factor VIII molecule or replacement protein/supplement (or fragments thereof) thereof comprises insertion of some or all of the amino acids of the one or more regulatory T cell epitopes and removing one or more amino acids at the site of insertion of the regulatory T cell epitope amino acids. In aspects, said insertion of the one or more regulatory T cell epitopes into the Factor V or Factor VIII molecule or replacement protein/supplement (or fragments thereof) comprises mutating the sequence of the Factor V or Factor VIII molecule or replacement protein/supplement (or fragments thereof) thereof to include the one or more regulatory T cell epitopes (for example, but not limited to, introduction one or more point mutations into the antibody or fragment thereof by site-directed mutagenesis or other recombinant techniques). In aspects, said insertion of the one or more regulatory T cell epitopes into the Factor V or Factor VIII molecule or replacement protein/supplement (or fragments thereof), which in aspects will introduce the one or more regulatory T cell epitope sequences, such that the previous immunogenicity of the sequence is decreased and the tolerogenicity of the new sequence is enhanced. In aspects, the number of said added one or more amino acids at the site of insertion of the regulatory T cell epitope amino acids need not correspond to the number of amino acids deleted from the sequence of the Factor V or Factor VIII molecule or replacement protein/supplement (or fragments thereof). In aspects, said insertion of one or more regulatory T cell epitopes into the Factor V or Factor VIII molecule or replacement protein/supplement (or fragments thereof) results in decreasing the immunogenicity of the antibody or fragment thereof. In aspects, said insertion of one or more regulatory T cell epitopes into the Factor V or Factor VIII molecule or replacement protein/supplement (or fragments thereof) thereof results in a increasing the tolerogenicity of the Factor V or Factor VIII molecule or replacement protein/supplement (or fragments thereof). In aspects, the one or more regulatory T cell epitopes have a sequence comprising, consisting of, or consisting essentially of one or more of SEQ ID NOS: 1-14 and 74-116.

For example, in aspects, one or more Tregitopes of the instant disclosure having a sequence comprising one or more of SEQ ID NOS: 1, 3-8, 10-14, 74-78, and 85-114 (and fragments or variants thereof), and optionally 1 to 12 additional amino acids distributed in any ratio on the N-terminus and/or C-terminus of the polypeptide of SEQ ID NOS: 1, 3-8, 10-14, 74-78, and 85-114), may be fused to or inserted internally within (e.g., but not limited to, site directed mutagenesis or other recombinant techniques) a human coagulation Factor V molecule or replacement protein/supplement (or a fragments thereof), such as in instances where the Tregitope is not located in its natural position within the human coagulation Factor V molecule or replacement protein/supplement (or a fragments thereof) or where the human coagulation Factor V molecule or replacement protein/supplement (or a fragments thereof) is missing such a Tregitope (e.g., if a particular human coagulation Factor V molecule or replacement protein/supplement (or a fragments thereof) has a mutated or missing corresponding section). As described previously, a Tregitope comprising one or more of SEQ ID NOS: 1, 3-8, 10-14, 74-78, and 85-114 may also be fused to or inserted internally within a polypeptide, e.g., a polypeptide that does not comprise human coagulation Factor V molecule or replacement protein/supplement (or a fragments thereof).

Additionally, in aspects, one or more Tregitopes of the instant disclosure having a sequence comprising one or more of SEQ ID NOS: 2, 9, and 79-84 (and fragments or variants thereof), and optionally 1 to 12 additional amino acids distributed in any ratio on the N-terminus and/or C-terminus of the polypeptide of SEQ ID NOS: 2, 9, and 79-84, may be fused to or inserted internally within (e.g., but not limited to, site directed mutagenesis or other recombinant techniques) a human coagulation Factor VIII molecule or replacement protein/supplement (or a fragments thereof), such as in instances where the Tregitope is not located in its natural position within the human coagulation Factor VIII molecule or replacement protein/supplement (or a fragments thereof) or where the human coagulation Factor VIII molecule or replacement protein/supplement (or a fragments thereof) is missing such a Tregitope (e.g., if a particular human coagulation Factor VIII molecule or replacement protein/supplement (or a fragments thereof) has a mutated or missing corresponding section). As described previously, a Tregitope comprising one or more of SEQ ID NOS: 2, 9, and 79-84 may also be fused to or inserted internally within a polypeptide, e.g., a polypeptide that does not comprise human coagulation Factor VIII molecule or replacement protein/supplement (or a fragments thereof).

Kits. The methods described herein can be performed, e.g., by utilizing pre-packaged kits comprising at least one Tregitope compound or composition of the present disclosure, which can be conveniently used, e.g., in clinical settings to treat subjects exhibiting symptoms or family history of a medical condition described herein. In one embodiment, the kit further comprises instructions for use of the at least one Tregitope compound or composition of the instant disclosure to treat subjects exhibiting symptoms or family history of a medical condition described herein.

### Aspects

A 1st aspect is directed to a polypeptide consisting of an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS: 1-14 and 74-116.

A 2nd aspect is directed to a polypeptide consisting essentially of an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS: 1-14 and 74-116.

A 3rd aspect is directed to a polypeptide comprising an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS: 1-14 and 74-116.

A 4th aspect is directed to a polypeptide according to any one of aspects 1-3, wherein said variant or fragment of an amino acid sequence selected from the group consisting of SEQ ID NOS: 1-14 and 74-116 retains MHC binding propensity and TCR specificity, and/or retains regulatory T cell stimulating or suppressive activity.

A 5^{th} aspect is directed to a polypeptide consisting of an amino acid sequence having at least 75%, 80%, 85%, 90%, or 95% homology to any one of SEQ ID NOS: 1-14 and 74-116, and fragments thereof, wherein said polypeptide retains MHC binding propensity and the same TCR specificity, and/or retains regulatory T cell stimulating or suppressive activity.

A 6^{th} aspect is directed to a polypeptide consisting essentially of an amino acid sequence having at least 75%, 80%, 85%, 90%, or 95% homology to any one of SEQ ID NOS: 1-14 and 74-116, and fragments thereof, wherein said polypeptide retains MHC binding propensity and the same TCR specificity, and/or retains regulatory T cell stimulating or suppressive activity.

A 7^{th} aspect is directed to a polypeptide comprising an amino acid sequence having at least 75%, 80%, 85%, 90%, or 95% homology to any one of SEQ ID NOS: 1-14 and 74-116, and fragments thereof, wherein said polypeptide retains MHC binding propensity and the same TCR specificity, and/or retains regulatory T cell stimulating or suppressive activity.

An 8th aspect is directed to a polypeptide according to any one of aspects 1-7, wherein said polypeptide has one or more conservative substitutions compared to the polypeptide.

A 9th aspect is directed to a polypeptide according to aspect 8, wherein said polypeptide retains MHC binding propensity and TCR specificity, and/or retains regulatory T cell stimulating or suppressive activity.

A 10^{th} aspect is directed to a polypeptide composition comprising one or more T-cell epitope polypeptides linked to a heterologous polypeptide, wherein the T-cell epitope polypeptide is a polypeptide according to any one of aspects 1-9.

An 11^{th} aspect is directed to a polypeptide composition according to aspect 10, wherein the T-cell epitope polypeptide is linked to the N-terminus of the heterologous polypeptide.

An 12th aspect is directed to a polypeptide composition according to any one or aspects 10-11, wherein the T-cell epitope polypeptide is linked to the C-terminus of the heterologous polypeptide.

A 13^{th} aspect is directed to a polypeptide composition according to any one or aspects 10-12, wherein the heterologous polypeptide comprises a biologically active molecule and wherein the biologically active molecule is selected from the group consisting of an immunogenic molecule, a T-cell epitope, a viral protein, and a bacterial protein.

A 14th aspect is directed to a polypeptide composition according to any one or aspects 10-13, wherein the heterologous polypeptide is operatively linked to the T-cell epitope polypeptide.

A 15^{th} aspect is directed to a nucleic acid encoding a polypeptide consisting of an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS: 1-14 and 74-116.

A 16^{th} aspect is directed to a nucleic acid encoding a polypeptide consisting essentially of an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS: 1-14 and 74-116.

A 17^{th} aspect is directed to a nucleic acid encoding a polypeptide comprising an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS: 1-14 and 74-116.

A 18^{th} aspect is directed to a nucleic acid of any one of aspects 8-10, wherein said fragment or variant of the nucleic acid encoding a polypeptide comprising an amino acid sequence selected from the group consisting of SEQ ID NOS: 1-14 and 74-116 encodes a polypeptide that retains regulatory T cell stimulating or suppressive activity.

A 19th aspect is directed to a plasmid comprising a nucleic acid of any one of aspects 15-18.

A 20^{th} aspect is directed to a vector comprising a nucleic acid according to any one of aspects 15-18.

A 21^{st} aspect is directed to a pharmaceutical composition comprising a polypeptide according to any one of aspects 1-14 and a pharmaceutically-acceptable carrier and/or excipient.

A 22^{nd} aspect is directed to a pharmaceutical composition comprising a nucleic acid according to any one of aspects 15-18 and a pharmaceutically-acceptable carrier and/or excipient.

A 23^{rd} aspect is directed to a pharmaceutical composition comprising a plasmid according to aspect 19 and a pharmaceutically-acceptable carrier and/or excipient.

A 24^{th} aspect is directed to a pharmaceutical composition comprising a vector according to aspect 20 and a pharmaceutically-acceptable carrier and/or excipient.

A 25^{th} aspect is directed to a method for suppressing an immune response in a subject in need thereof, the method comprising administering to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 26^{th} aspect is directed to a method of inducing regulatory T-cells to suppress immune response in a subject comprising administrating to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 27^{th} aspect is directed to a method for stimulating regulatory T-cells in a subject in need thereof, the method comprising administering to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 28^{th} aspect is directed to a method suppressing an antigen-specific immune response in a subject in need thereof, the method comprising administering to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 29th aspect is directed to a method according to any one of aspects 25-28, wherein the administration of the Tregitope composition activates CD4⁺/CD25⁺/FoxP3⁺ regulatory T-cells.

A 30^{th} aspect is directed to a method according to any one of aspects 25-28, wherein the administration of the Tregitope composition suppresses activation of CD4⁺ T-cells.

A 31^{st} aspect is directed to a method according to any one of aspects 25-28, wherein the administration of the Tregitope composition suppresses activation or proliferation of CD4⁺ effector T-cells and/or CD8⁺ effector T-cells.

A 32^{nd} aspect is directed to a method according to any one of aspects 25-28, wherein the administration of the Tregitope composition suppresses activation or proliferation of B-cells.

A 33^{rd} aspect is directed to a method according to any one of aspects 25-28, wherein the subject suffers from an allergy, an autoimmune disease, a transplant related disorder, an enzyme or protein deficiency disorder, or a blood clotting disorder.

A 34^{th} aspect is directed to a method according to any one of aspects 25-28, wherein the immune response is a result of one or more therapeutic treatments select from the group consisting of, treatment with at least one therapeutic protein, treatment with a vaccine, and treatment with at least one antigen

A 35^{th} aspect is directed to a method according to any one of aspects 25-28, wherein the administration of the pharmaceutical Tregitope composition shifts one or more antigen presenting cells to a regulatory phenotype.

A 36^{th} aspect is directed to a method according to any one of aspects 25-28, wherein the administration of the regulatory T-cell epitope shifts one or more dendritic cells to a regulatory phenotype.

A 37^{th} aspect is directed to a method according to any one of aspects 25-28, wherein the administration of the pharmaceutical Tregitope composition shifts one or more dendritic cells to a regulatory phenotype.

A 38^{th} aspect is directed to a method according to any one of aspects 25-28, wherein the regulatory phenotype is characterized by a decrease in CD11c and HLA-DR expression in the dendritic cells or other antigen presenting cells.

A 39^{th} aspect is directed to a method according to any one of aspects 25-28, wherein the administration of the regulatory T-cell epitope shifts one or more T cells to a regulatory phenotype.

A 40^{th} aspect is directed to a method according to any one of aspects 25-28, wherein the administration of the regulatory T-cell epitope shifts one or more CD4+ T cells to a regulatory phenotype.

A 41^{st} aspect is directed to a method according to any one of aspects 25-28, wherein the administration of the regulatory T-cell epitope shifts one or more CD8+ T cells to a regulatory phenotype.

A 42^{nd} aspect is directed to a method according to any one of aspects 25-28, wherein the administration of the regulatory T-cell epitope shifts one or more B cells to a regulatory phenotype.

A 43^{rd} aspect is directed to a method for expanding a population of regulatory T cells of a patient, comprising:

(a) providing a biological sample obtained from a subject; and

(b) isolating regulatory T-cells from the biological sample; and contacting the isolated regulatory T-cells with an effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24 under conditions wherein the T-regulatory cells increase in number to yield an expanded regulatory T-cell composition, thereby expanding the regulatory T-cells in the biological sample; and

(c) returning said increased number of regulatory T cells to said patient.

A 44^{th} aspect is directed to a method for stimulating regulatory T cells in a biological sample, comprising:

(a) providing a biological sample obtained from a subject;

(b) isolating regulatory T-cells from the biological sample; and contacting the isolated regulatory T-cells with an effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24 under conditions wherein the T-regulatory cells are stimulated to alter one or more biological function, thereby stimulating the regulatory T-cells in the biological sample.

### Exemplification

The examples that follow are not to be construed as limiting the scope of the invention in any manner. In light of the present disclosure, numerous embodiments within the scope of the claims will be apparent to those of ordinary skill in the art. While certain examples pertain to particular Tregitopes of the instant disclosure, it will be understood that examples and methods can be used for any Tregitope of the present disclosure (e.g., one or more polypeptides comprising, consisting, or consisting essentially of an amino acid sequence of SEQ ID NOS. 1-14 and 74-116 (and/or fragments or variants thereof), and optionally 1 to 12 additional amino acids distributed in any ratio on the N terminus and/or C-terminus of the polypeptide of SEQ ID NOS. 1-14 and 74-116).

### (1) In-Silico Identification of a Tregitope Compound or Composition

T cells specifically recognize epitopes presented by antigen presenting cells (APCs) in the context of MHC (Major Histocompatibility Complex) Class II molecules. These T-helper epitopes can be represented as linear sequences comprising 7 to 30 contiguous amino acids that fit into the MHC Class II binding groove. A number of computer algorithms have been developed and used for detecting Class II epitopes within protein molecules of various origins (De Groot AS et al., (1997), AIDS Res Hum Retroviruses,13(7):539-41; Schafer JR et al., (1998), Vaccine,16(19):1880-4; De Groot AS et al., (2001), Vaccine, 19(31):4385-95; De Groot AS et al., (2003), Vaccine, 21(27-30):4486-504). These “in silico” predictions of T-helper epitopes have been successfully applied to the design of vaccines and the de-immunization of therapeutic proteins, i.e. antibody-based drugs, Fc fusion proteins, anticoagulants, blood factors, bone morphogenetic proteins, engineered protein scaffolds, enzymes, growth factors, hormones, interferons, interleukins, and thrombolytics (Dimitrov DS, (2012), Methods Mol Biol, 899:1-26). The preferred “therapeutic protein” of the instant invention is human Coagulation Factor VII or Factor VIII.

The EpiMatrix™ system (EpiVax, Providence, R.I.) is a set of predictive algorithms encoded into computer programs useful for predicting class I and class II HLA ligands and T cell epitopes. The EpiMatrix™ system uses 20×9 coefficient matrices in order to model the interaction between specific amino acids (20) and binding positions within the HLA molecule (9). In order to identify putative T cell epitopes resident within any given input protein, the EpiMatrix™ System first parses the input protein into a set of overlapping 9-mer frames where each frame overlaps the last by eight amino acids. Each frame is then scored for predicted affinity to one or more common alleles of the human HLA molecule; typically DRB1*0101, DRB1*0301, DRB1*0401, DRB1*0701, DRB1*0801, DRB1*1101, DRB1*1301, and DRB1*1501 (Mack et al., (2013), Tiss Antig, 81(4):194-203). Briefly, for any given 9-mer peptide specific amino acid codes (one for each of 20 naturally occurring amino acids) and relative binding positions (1-9) are used to select coefficients from the predictive matrix. Individual coefficients are derived using a proprietary method similar to, but not identical to, the pocket profile method first developed by Sturniolo (Sturniolo T et al., 1999, Nat Biotechnol, 17(6):555-61, herein incorporated by reference in its entirety). Individual coefficients are then summed to produce a raw score. EpiMatrix™ raw scores are then normalized with respect to a score distribution derived from a very large set of randomly generated peptide sequences. The resulting “Z” scores are normally distributed and directly comparable across alleles.

EpiMatrix™ peptide scoring. It was determined that any peptide scoring above 1.64 on the EpiMatrix™ “Z” scale (approximately the top 5% of any given peptide set) has a significant chance of binding to the MHC molecule for which it was predicted. Peptides scoring above 2.32 on the scale (the top 1%) are extremely likely to bind; most published T cell epitopes fall within this range of scores. Previous studies have also demonstrated that EpiMatrix™ accurately predicts published MHC ligands and T cell epitopes (De Groot AS, Martin W. Reducing risk, improving outcomes: bioengineering less immunogenic protein therapeutics. Clin Immunol. 2009 May;131(2):189-201.doi: 10.1016/j.clim.2009.01.009. Epub 2009 Mar. 6., herein incorporated by reference in its entirety).

Identification of promiscuous T cell Epitope Clusters. Potential T cell epitopes are not randomly distributed throughout protein sequences but instead tend to “cluster.” T cell epitope “clusters” range from 9 to roughly 30 amino acids in length and, considering their affinity to multiple alleles and across multiple frames, contain anywhere from 4 to 40 binding motifs. Following epitope mapping, the result set produced by the EpiMatrix™ algorithm is screened for the presence of T cell epitope clusters and EpiBarr™ by using a proprietary algorithm known as Clustimer™. Briefly, the EpiMatrix™ scores of each 9-mer peptide analyzed are aggregated and checked against a statistically derived threshold value. High scoring 9mers are then extended one amino acid at a time. The scores of the extended sequences are then re-aggregated and compared to a revised threshold value. The process is repeated until the proposed extension no longer improves the overall score of the cluster. Tregitope(s) identified in the present studies were identified by the Clustimer-™ algorithm as T cell epitope clusters. They contain significant numbers of putative T cell epitopes and EpiBars™ indicating a high potential for MHC binding and T cell reactivity.

Identification of tolerogenic T cell Epitope Clusters. The JanusMatrix system (EpiVax, Providence, R.I.) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and as such may be very useful Tregitopes of the present disclosure

### EXAMPLE 1

### Identification of a Tregitope Compound or Composition

Hemophiliac patients produce truncated or mutated Factor VIII (FVIII) proteins or no FVIII protein at all. As a result, therapeutic FVIII is sometimes recognized as “foreign” by the immune system of patients suffering from hemophilia; resulting in the activation of FVIII specific CD4+ T cells and the development of anti-FVIII antibodies. This “Anti-Drug Antibody” response occurs in 25-30% of Hemophilia A patients treated with recombinant FVIII, increasing morbidity and lowering the quality of life (Waters B and Lillicrap D, (2009), J Thromb Haemost, 7(9):1446-56, herein incorporated by reference in its entirety).

FVIII and Factor V (FV) are homologous glycoproteins that are cofactors for proteolytic activation in the coagulation cascade. They share a conserved domain structure of (A1-A2-B-A3-C1-02) and also share 35% amino acid identity in the A and C domains (Pipe SW et al., J Biol Chem, 273(14):8537-44, herein incorporated by reference in its entirety). Regulatory T cells (T_{Reg}), recognizing regulatory T cell epitopes (Tregitopes) present in the amino acid sequence of FV, cross react with homologous regulatory T cell epitopes present in the amino acid sequence of FVIII. Tregitopes derived from FV may be useful for the purpose of suppressing anti-therapeutic immune response targeting FVIII, particularly in FVIII deficient hemophiliacs.

9-mer peptides present in FV which are ligands to HLA DRB1*1501 (a known risk factor for hemophilia) and which can be related to homologous sequences present in FVIII provided said FVIII-derived homologues are also putative ligands to DRB1*1501 and at least four of the five T cell receptor (TCR) contact residues present in those peptides (relative positions 2, 3, 5, 7, and 8) are shared between the FV-derived putative epitope and its FVIII-derived homologue were identified. The complete amino acid sequence of human FV (Genbank accession: NP_000121.2) was parsed into overlapping 9-mer frames and scored using the EpiMatrix™ system as described previously. The complete amino acid sequence of human FVIII (Genbank accession: NP_000123.1) was parsed into overlapping 9-mer frames and scored using the EpiMatrix™ system as described previously.

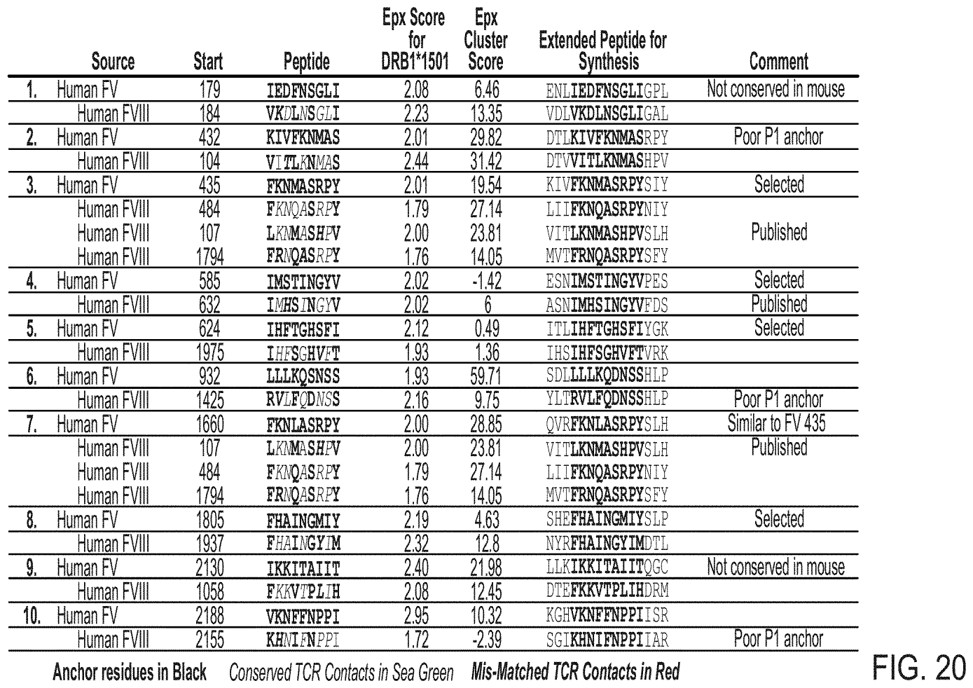

A purpose built computer program was used to screen putative DRB1*1501 ligands derived from FV against a set of putative DRB1*1501 ligands derived from FVIII. Ten of the putative epitopes identified in FV were matched to TCR homologues in FVIII. Each of the FV-derived ligands and its FVIII-derived homologue(s) were then screened against the Immune Epitope database (IEDB) (National Institute of Allergy and Infectious Diseases (NIAID) Bethesda, Md.) by conventional means in order to identify any previously validated epitopes present in the experimental set. The results of in-silico analysis are presented in FIG. 20. Three peptides were eliminated from further consideration due to the presence of a poor P1 binding anchor in either the FV derived peptide or the FVIII-derived homologue: SEQ ID NO: 3, FV 932 (SEQ ID NO: 32 and extended peptide SEQ ID NO: 33), and FV 2188 (SEQ ID NO: 50 and extended peptide SEQ ID NO 51). Two additional peptides were eliminated due to poor conservation within the murine versions of FV and/or FVIII; suggesting use in animal based models would be problematic: FV 179 (SEQ ID NO: 16 and extended peptide SEQ ID NO 17) and FV 2130 (SEQ ID NO: 46 and extended peptide SEQ ID NO 47). Peptide FV 1660 (SEQ ID NO: 36 and extended peptide SEQ ID NO 37) was eliminated due to its close homology to a previously selected peptide FV 435 (SEQ ID NO: 3 and extended peptide SEQ ID NO: 10). The remaining four peptides were selected for further testing. In order to ensure a high affinity bond between peptide ligands and Class II HLA, 9-mer peptides must be extended to include n- and c-terminal “flanks” of at least 2 to 3 amino acids in length. Three amino acids to the n- and c-terminal flanks of each of the four peptides were selected for further analysis. The synthesized peptides are designated SEQ ID NO: 1 (ILTIHFTGHSFIYGK); SEQ ID NO: 2 (IHSIHFSGHVFTVRK); SEQ ID NO: 3 (KIVFKNMASRPYSIY); SEQ ID NO: 5 (ESNIMSTINGYVPES); and SEQ ID NO: 7 (SHEFHAI NGMIYSLP).

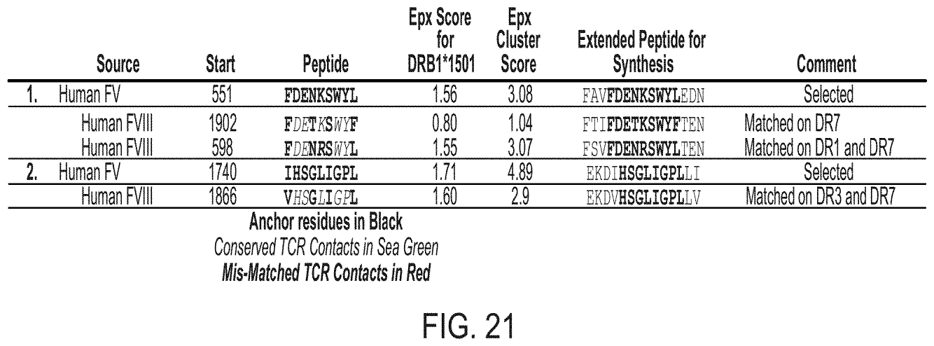

In a second analysis, FV-FVIII peptide pairs matched at all five TCR contact positions were identified. High EpiMatrix™ Z-scores for HLA DRB1*1501 were not required. The only requirement was that both peptides in a given pair score high for at least one matched DRB1 allele. The returned peptides sets are presented in FIG. 21. Based on this analysis, two additional peptides were selected for further testing: SEQ ID NO: 4 (FAVFDENKSWYLEDN) and SEQ ID NO: 6 (EKDIHSGLIGPLLI).

FIG. 26 is the overview of JanusMatrix results for identified the Tregitopes of SEQ ID NOS: 1-7 of the instant disclosure. FIG. 27 is the JanusMatrix report for the Tregitope of SEQ ID NO: 1 and the 9-mers contained within SEQ ID NO: 1, including SEQ ID NOS: 74-78, 8, and 115. FIG. 28 is the JanusMatrix report for the Tregitope of SEQ ID NO: 2 and the 9-mers contained within SEQ ID NO: 2, including SEQ ID NOS: 19-84, and 116. FIG. 29 is the JanusMatrix report for the Tregitope of SEQ ID NO: 3 and the 9-mers contained within SEQ ID NO: 3, including SEQ ID NOS: 85-90 and 10. FIG. 30 is the JanusMatrix report for the Tregitope of SEQ ID NO: 4 and the 9-mers contained within SEQ ID NO: 4, including SEQ ID NOS: 91-97. FIG. 31 is the JanusMatrix report for the Tregitope of SEQ ID NO: 5 and the 9-mers contained within SEQ ID NO: 5, including SEQ ID NOS: 98-103 and 12. FIG. 32 is the JanusMatrix report for the Tregitope of SEQ ID NO: 6 and the 9-mers contained within SEQ ID NO: 6, including SEQ ID NOS: 104-108 and 13. FIG. 33 is the JanusMatrix report for the Tregitope of SEQ ID NO: 7 and the 9-mers contained within SEQ ID NO: 7, including SEQ ID NOS: 109-114 and 14. For each of FIGS. 26-33, * is the count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. Further, the Janus Homology Score** represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

### (2) Methods for the Assessment of Tregitope Binding to Soluble MHC.

Synthesis of peptides. The Tregitopes of the invention can be produced by direct chemical synthesis or by recombinant methods (J Sambrook et al., Molecular Cloning: A Laboratory Manual, (2ED, 1989), Cold Spring Harbor Laboratory Press, Cold Springs Harbor, N.Y. (Publ), herein incorporated by reference in its entirety). Sample Tregitopes were prepared using Fmoc-chemical (9-fluoronylmethoxycarbonyl synthesis, under the guidance and direction of the Inventors of the present invention at 21^{st} Century Biochemicals (Marlborough, Mass.). In certain aspects, the Tregitopes were capped with an n-terminal acetyl and c-terminal amino group. HPLC, mass spectrometry and UV scan (ensuring purity, mass and spectrum, respectively) analysis of the selected Tregitopes indicated >80% purity.

An amino acid analysis of SEQ ID NO:1 ILTIHFTGHSFIYGK was conducted by a third-party contractor (New England Peptide, Inc., Gardner, Mass.) confirming the predicted composition (data not shown).

Mass Spectrum and Analytical HPLC analysis was performed by a second independent contractor (21^{st} Century Biochemicals, Inc., Marlboro, Mass.) further confirming the composition of the Tregitope (data not shown).

HLA Binding Assay. Binding activity was analyzed at EpiVax (Providence, R.I.). The binding assay used (Steere AC et al., (2006), J Exp Med, 2003(4):961-71) yielded an indirect measure of peptide-MHC affinity. Soluble HLA molecules were loaded onto a 96-well plate with the unlabeled experimental Tregitopes and labeled control peptide. Once the binding mixture reached steady equilibrium (at 24 hours), the HLA-Tregitope complexes were captured on an ELISA plate coated with anti-human DR antibody and detected with a Europium-linked probe for the label (PerkinElmer, Waltham, Mass.). Time-resolved fluorescence measuring bound labeled control peptide is assessed by a SpectraMax® M5 unit (Spectramax, Radnor, Pa.). Binding of experimental Tregitopes was expressed as the percent inhibition of the labeled control peptide (experimental fluorescence/control fluorescence multiplied by 100). The percent inhibition values for each experimental Tregitope (across a range of molar concentrations) were used to calculate the concentration at which it inhibits 50% of the labeled control Tregitope's specific binding, i.e. the Tregitope's IC₅₀.

Selected experimental Tregitopes were solvated in DMSO. The diluted Tregitopes were then mixed with binding reagents in aqueous buffering solution, yielding a range of final concentrations from 100,000 nM down to 100 nM. Tregitopes were then assayed against a panel of five common Class II HLA alleles: HLA-DRB1*0101, HLA-DRB1*0301, HLA-DRB1*0701, HLA-DRB1*1101, and HLA-DRB1*1501. From the percent inhibition of labeled control peptide at each concentration, IC₅₀ values were derived for each Tregitope/allele combination using linear regression analysis.

In this assay, the experimental Tregitopes are considered to bind with very high affinity if they inhibit 50% of control peptide binding at a concentration of 100 nM or less, high affinity if they inhibit 50% of control peptide binding at a concentration between 100 nM and 1,000 nM, and moderate affinity if they inhibit 50% of control peptide binding at a concentration between 1,000 nM and 10,000 nM. Low affinity peptides inhibit 50% of control peptide binding at concentrations between 10,000 nM and 100,000 nM. Peptides that fail to inhibit at least 50% of control peptide binding at any concentration below 100,000 nM and do not show a dose response are considered non-binders (NB).

### EXAMPLE 2

### Peptide Characterization by Binding to HLA Class II Molecules

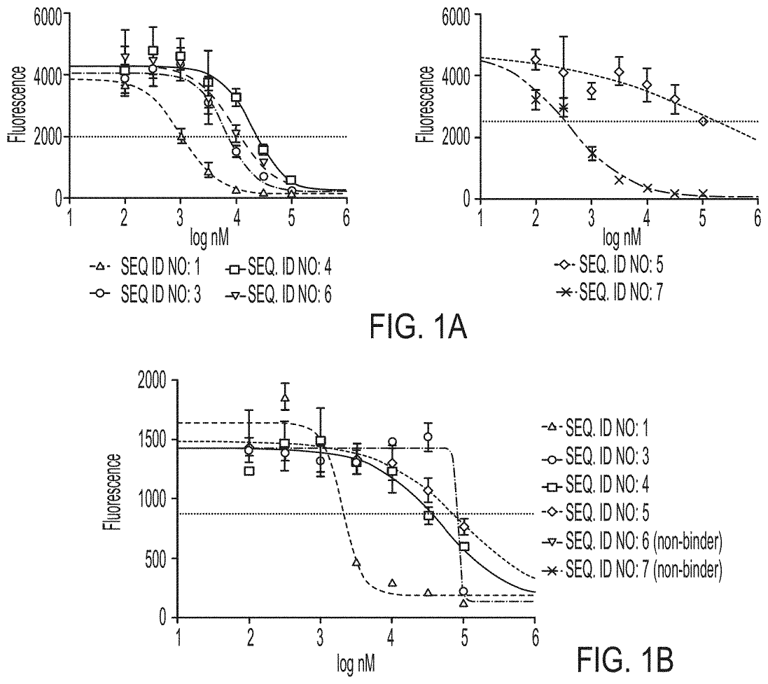

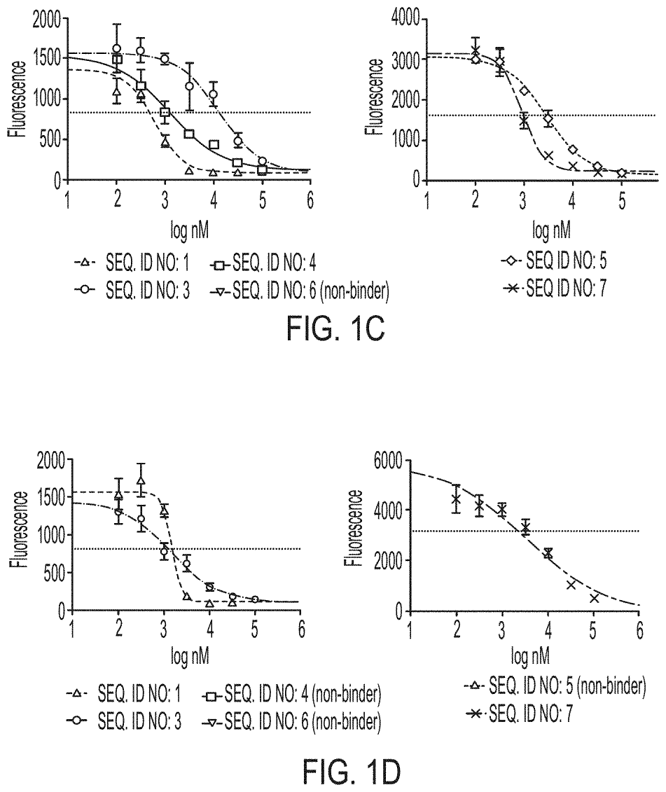

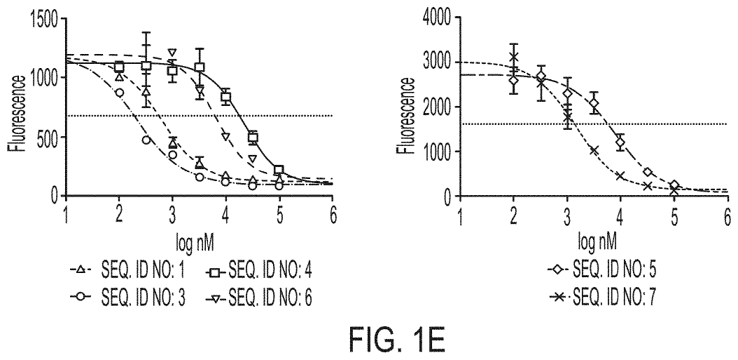

Soluble MHC binding assays were performed on the Tregitopes of the invention according to the methods described previously. IC₅₀ values (nM) were derived from a six-point inhibition curve. The list of synthesized Tregitopes used in binding assays is presented in Table 2. FIGS. 1A-C shows the binding curves for certain Tregitopes against the selected Class II HLA alleles. FIG. 1A summarizes the results for HLA DRB1 *0101 assay, FIG. 1B summarizes the results for the HLA DRB1 *0301 assay. FIG. 1C summarizes the results for the HLA DRB1 *0701 assay for the selected FV peptides, FIG. 1D summarizes the results for HLA DRB1 *1101 assay for selected FV peptides, and FIG. 1E report summarizes the results for HLA DRB1* 1501.

TABLE 2
Peptides Selected for HLA Binding

| | | Max EPX Score for HLA DRB1 | | | | |
|---|---|---|---|---|---|---|
| Peptide ID | Peptide Sequence | *0101 | *0301 | *0701 | *1101 | *1501 |
| SEQ. ID NO: 1 | ILTIHFTGHSFIYGK | [symbol] | 1.45 | 2.00 | [symbol] | 2.12 |
| SEQ. ID NO: 3 | KIVFKNMA [symbol] RPY [symbol] IY | 2.91 | 2.45 | 2.01 | 2. [symbol] 1 | 2.01 |
| SEQ. ID NO: 4 | FAVFDENK [symbol] WYLEDN | 1.86 | 1. [symbol] | 2.05 | 1.61 | 1.56 |
| SEQ. ID NO: 5 | ESNIMSTINGYVPES | 1.60 | [symbol] | 2.32 | 1. [symbol] | 2.02 |
| SEQ. ID NO: 6 | EKDIHSGLIGPLLI | 1.64 | 1.82 | 2.19 | 0.77 | 1.71 |
| SEQ. ID NO: 7 | [symbol] HEFHAINGMIYSLP | 2.29 | 1.75 | 1. [symbol] | 1. [symbol] | 2.1 [symbol] |

[symbol]

indicates data missing or illegible when filed

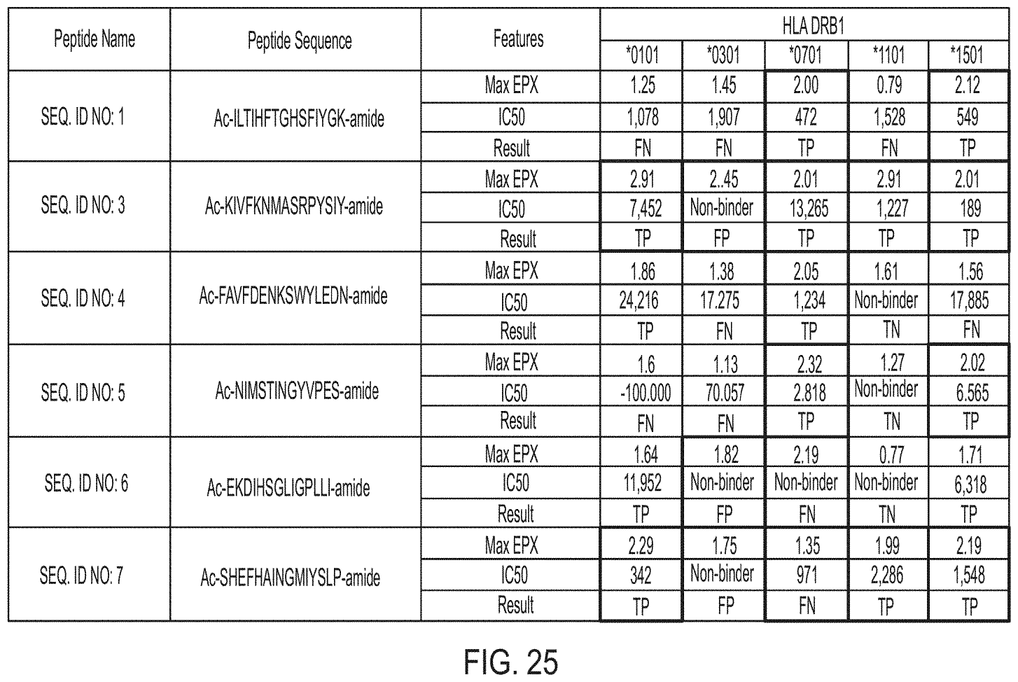

A summary of HLA binding results is presented in FIG. 25. EpiMatrix™ Predictions, calculated IC₅₀ values, and results classifications are reported for each Tregitope and HLA allele. Tregitope-allele combinations predicted to be cross-reactive between FV and FVIII are indicated. Of these Tregitope-allele interactions, 14/16 were shown to bind HLA indicating that these Tregitopes will generate measurable responses in human PBMC assays. Based on these findings, four Tregitopes were selected for further testing: SEQ ID NO: 3, SEQ ID NO: 5, SEQ ID NO: 1, and SEQ ID NO: 7. Tregitopes SEQ ID NO: 4 and SEQ ID NO: 6 were set aside due to their highly-restricted HLA binding profiles.

### (3) Methods for Assessing the Phenotype of Peptide-Exposed APC

Surface expression of Class II HLA (HLA-DR) and CD86 by professional antigen presenting cells (APCs) is one way APCs modulate T cell response. Expression of Class II HLA surface marker is down-regulated in response to Tregitopes, and in particular to, the control Tregitope 167 (21^{st} Century Biochemicals, Marlboro, Mass.). Additionally, reduced expression of surface marker CD86 correlates positively with enhanced T_{Reg} function (Zheng Y et al., J Immunol, 2004, 172(5):2778-84). In this assay, candidate Tregitopes, including the selected Tregitopes, were tested for their ability to down-regulate the expression of Class II HLA and the co-stimulatory molecule CD86 on the surface of professional APCs, specifically dendritic cells.

Each of the four Tregitopes selected for further testing were individually tested for regulatory potential using a proprietary APC phenotyping assay previously developed at EpiVax (EpiVax, Providence, R.I.). Previously harvested and frozen PBMC were thawed and suspended in chRPMI by conventional means. Under the direction and guidance of the Inventors from EpiVax, HLA typing was conducted on small, extracted samples of cellular material, provided to EpiVax, by Hartford Hospital (Hartford, Conn.). On assay day 0, 0.5×10⁶ cells were extracted, screened for the presence of surface marker CD11c (a marker specific to dendritic cells) and analyzed for the presence of surface markers H LA-DR and CD86 by flow cytometry. The remaining cells were plated (4.0×10⁶ cell per ml in chRPMI plus 800 ul media) and stimulated (50 μg/mL) with one of the four selected peptides or positive and negative controls including buffer only (negative control), Tregitope 167 (positive control, SEQ ID NO: 15) (21^{st} Century Biochemicals, Marlboro, Mass.), Flu-HA 306-318 (negative control) (21^{ST} Century Biochemicals, Marlboro, Mass.) and Ova 323-339 (negative control) (21^{st} Century Biochemicals, Marlboro, Mass.). Plated cells were incubated for seven days at 37° C. On assay day 7, incubated cells were screened by flow cytometry for the presence of surface marker CD11c. CD11c positive cells were then analyzed for the presence of surface markers HLA-DR and CD86. The experimental peptides were tested in samples drawn from five different human donors.

Prior to March 2015, all whole blood samples used in the experiments were sourced from healthy donors under IRB 07115 protocol (Clinical Partners, Johnston, R.I.). Leukocytes were isolated using a conventional ficoll separation gradient (Noble PB and Cutts JH, Can Vet J, 1967, 8(5):110-11). After April 2015, Leukocyte Reduction Filters were obtained from the Rhode Island Blood Center (Providence, R.I.) to filter the white blood cells from whole blood obtained from healthy donors. After the whole blood was run through the filters, the filters were flushed in the opposite direction to push collected white blood cells out of the filter. The white blood cells were then isolated using a conventional ficoll separation gradient. The collected white blood cells were thereafter frozen for future use. When needed for use in an assay, the frozen white blood cells were thawed using conventional methods. For the GvHD studies discussed below, PBMCs were obtained from HemaCare, Van Nuys, Calif. and the experiments were performed at Lifespan Hospital (Providence, R.I.).

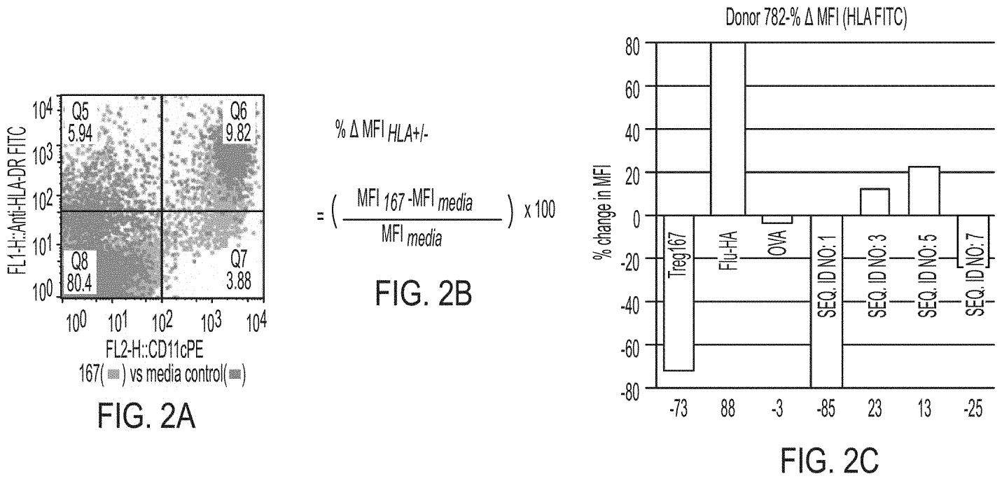

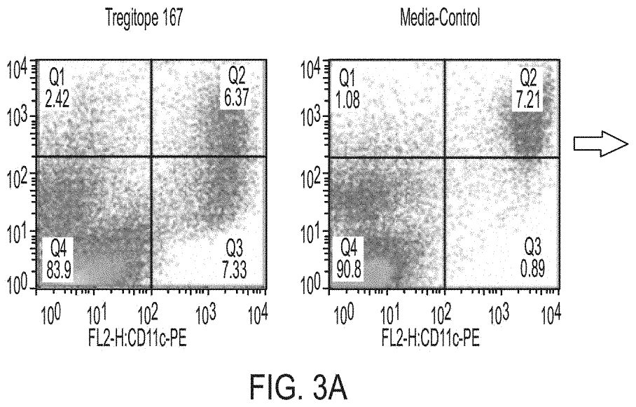

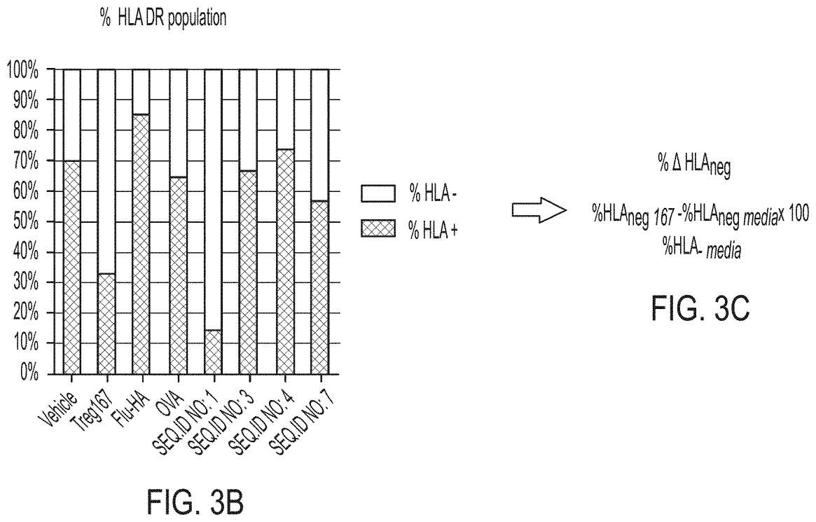

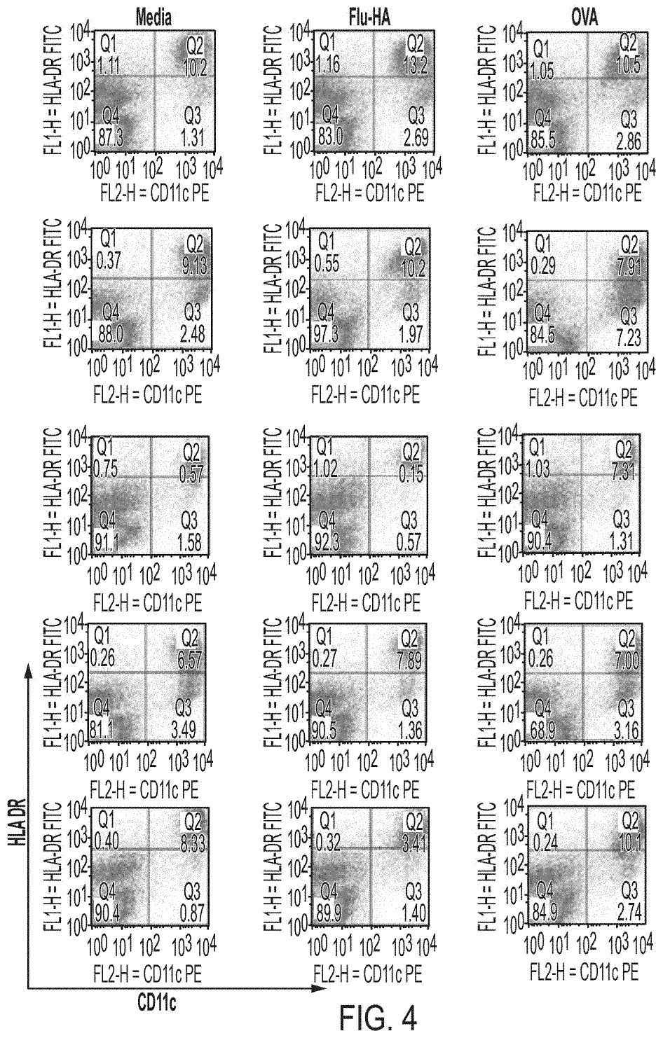

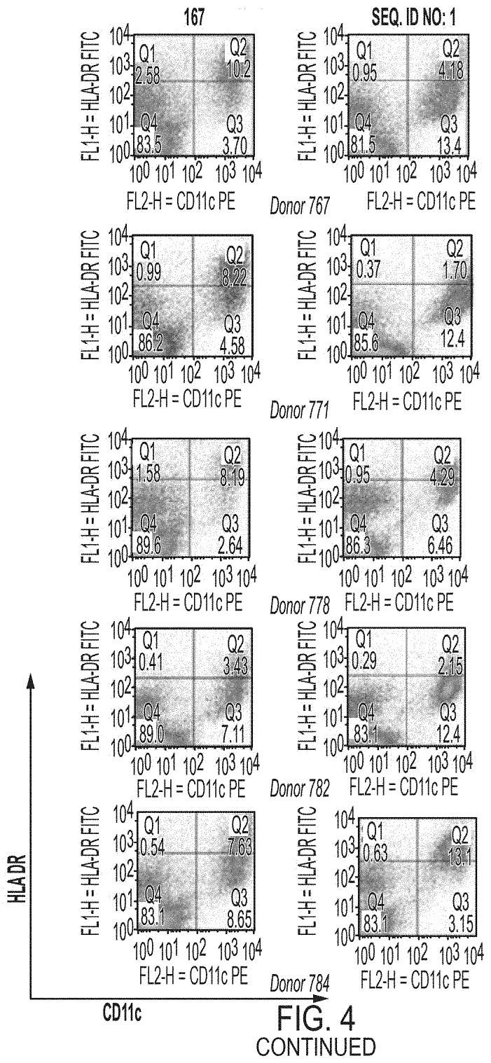

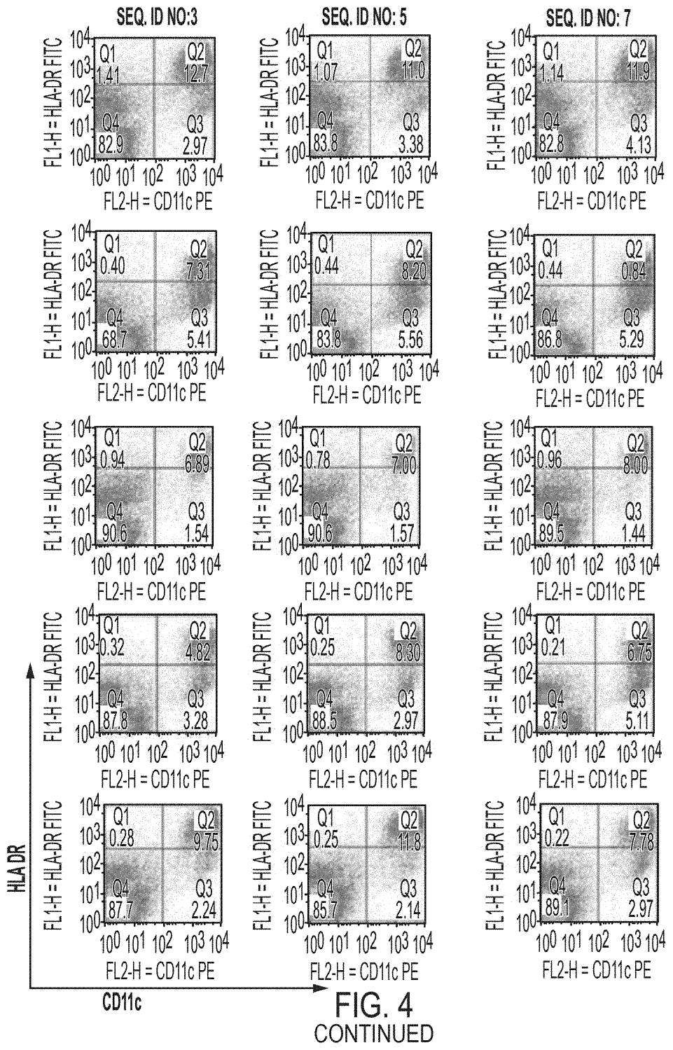

Exposure to putative Tregitopes on the phenotypes of dendritic cells was measured by multiple means. First, for each experimental condition, dot-plots, contrasting surface expression of CD11c and HLA-DR, were produced (FIG. 4). Dot-plots of cells exposed to all control and experimental peptides were overlaid onto dot-plots produced from control cells exposed to only the culture media. The overlay provided an effective method to visually observe shifts in H LA-DR distribution between Tregitope stimulated and unstimulated CD11c-high cells (see FIG. 2A, depicts an overlay of HLA-DR/CD11c dot plots for SEQ ID NO. 15 (Tregitope 167, dark gray) and media control (light gray) highlights the shift in observed HLA-DR expression). Observed shifts in the distribution of H LA-DR were reported as a qualitative measure. Next, the change in intensity of HLA-DR expression for the CD11c-high segment of each dot-plot was calculated using the equation of FIG. 2B. Percent change in intensity of HLA-DR expression equals Mean Florescence Index (MFI) of HLA-DR expression for peptide exposed cells minus MFI of HLA-DR expression for media exposed cells divided by MFI of HLA-DR expression for media exposed cells, times 100 (^{HLA-DR}MFI_{peptide}−^{HLA-DR}MFI_{media}/^{HLA-DR}MFI_{media}*100). FIG. 2C is a bar graph showing the % Δ in HLA-DR MFI for each peptide stimulant. Next, the percent change in the percentage of HLA-DR-low cells present among the CD11c high population was calculated for each peptide relative to media control (FIG. 3A-C). Percent change in the percentage of HLA-DR-low cells was calculated using the equation of FIG. 3C, and equals the percent of HLA-DR-low for peptide exposed cells minus the percent of HLA-DR-low for media exposed cells divided by percent of HLA-DR-low for media exposed cells times 100 (^{HLA-DR-low}%_{peptide}−^{HLA-DR-low}%_{media}/^{HLA-DR-low}%_{media}*100). In this assay, a negative change in observed HLA-DR MFI and a positive change in percentage of HLA-DR-low cells present in the CD11c-high population indicated reduced expression of HLA and a shift to a regulatory APC phenotype. FIG. 3A is a plot of HLA-DR versus CD11c for SEQ ID NO. 15 (Tregitope 167, which was used as a control and has the formula PAVLQSSGLYSLSLSSVVTVPSSSLGTQ) and the media control. FIG. 3B is a bar graph that plots the % of HLA+ and HLA− each peptide stimulant where the vehicle is media.

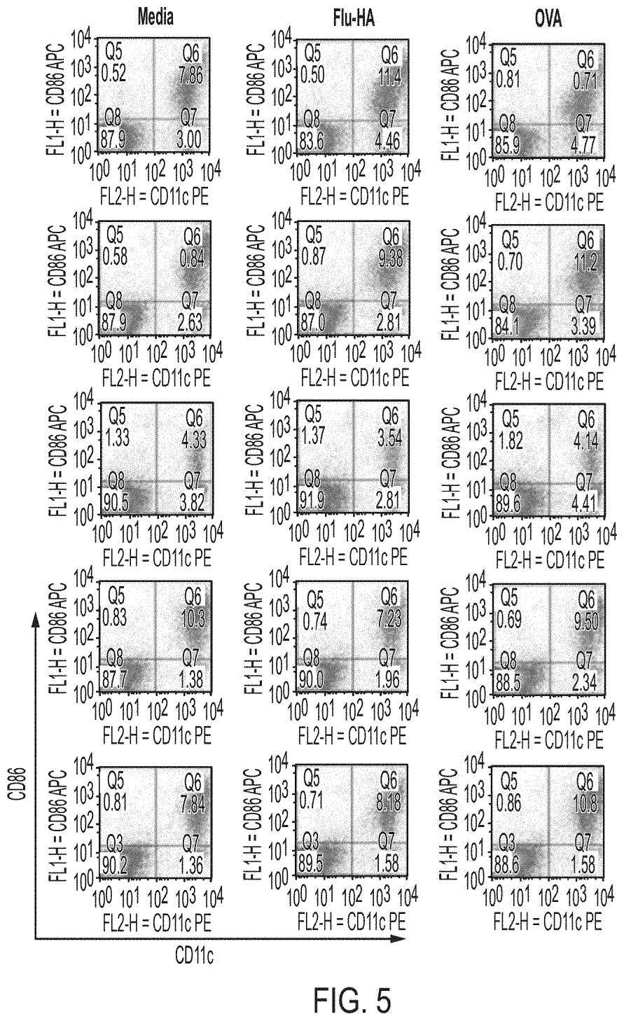

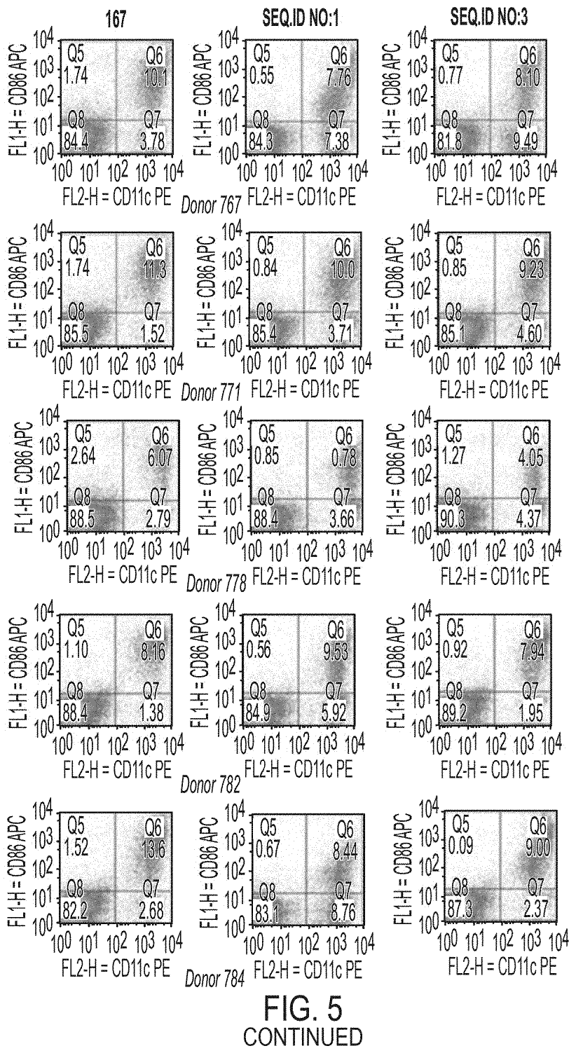

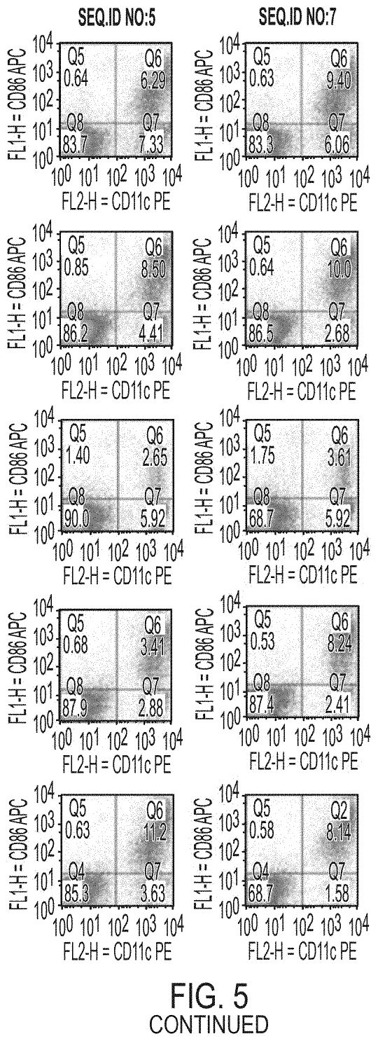

A similar process was used to assess the impact of select Tregitope exposure on surface expression of CD86; a costimulatory molecule known to promote T cell activation. First, for each experimental condition, dot-plots contrasting surface expression of CD11c and CD86 were produced (FIG. 5). Dot-plots of cells exposed to all control and experimental Tregitopes were overlaid onto dots-plots produced from control cells exposed to only the culture media. The overlay provided an effective method to visually observe shifts in CD86 distribution between Tregitope stimulated and un-stimulated CD11c-high cells. (data not shown). Observed shifts in the distribution of CD86 were reported as a qualitative measure. Next, the change in intensity of CD86-high expression for the CD11c-high segment of each dot-plot was calculated. Percent change in intensity of CD86-high expression equals Mean Florescence Index (MFI) of CD86 expression for peptide exposed cells minus MFI of CD86-high expression for media exposed cells divided by MFI of CD86 expression for media exposed cells, times 100 (^{CD86-high}MFI_{peptide}−^{CD86-high}MFI_{media}^{CD86-high}MFI_{media}*100). (data not shown). Next, the percent change in the percentage of CD86-low cells present among the CD11c high population was calculated. Percent change in the percentage of CD86-high cells equals the percent of CD86-high for peptide exposed cells minus the percent of CD86-high for media exposed cells divided by percent of CD86-high for media exposed cells, times 100 (^{CD86-low}%|_{peptide}−^{CD86-low}%_{media}/^{CD86-low}%_{media}* 100). In this assay, a negative change in observed CD86 MFI and a positive change in percentage of CD86-low cells present in the CD11c-high population indicates reduced expression of CD86 and a shift to a regulatory APC phenotype.

### EXAMPLE 3

### Characterization of Peptide Exposed APC

Dendritic cell phenotyping assays were performed on the selected Tregitopes according to the methods described previously. Dot-plots corresponding to each experimental condition tested in each of five human donors are presented in FIG. 4 and FIG. 5.

FIG. 4 is a series of dot plots representing the surface expression of CD11 vs H LA-DR analyzed on assay day 7 across the five donors in the presence of various peptide stimulants. The downward movement of the CD11c+/HLA-DR+ population (upper right quadrant of FIG. 4) was apparent in the samples treated with SEQ ID NO: 1 as compared to media control indicating an acquired regulatory phenotype. Tregitope 167 (SEQ ID NO. 15, positive control) and some of the other FV peptides responded similarly, but the observed shift is more prominent with SEQ ID NO: 1.

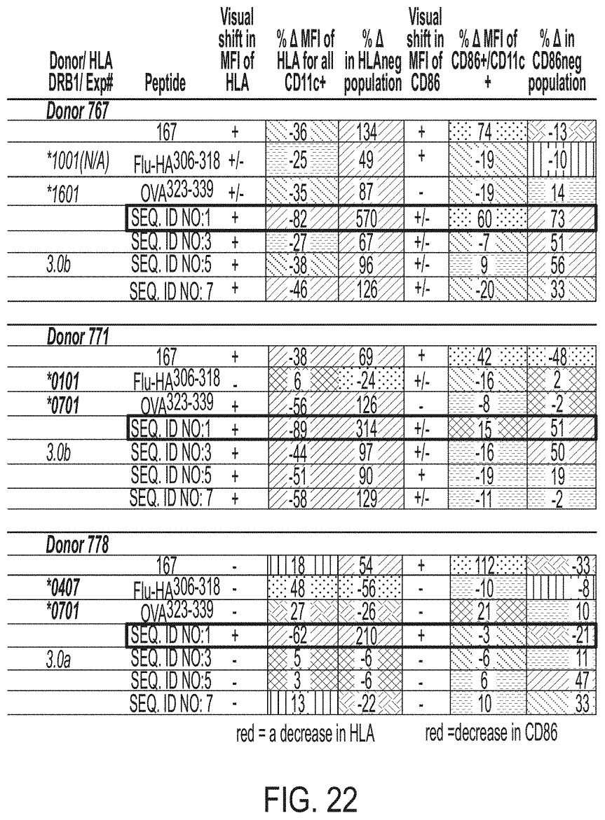

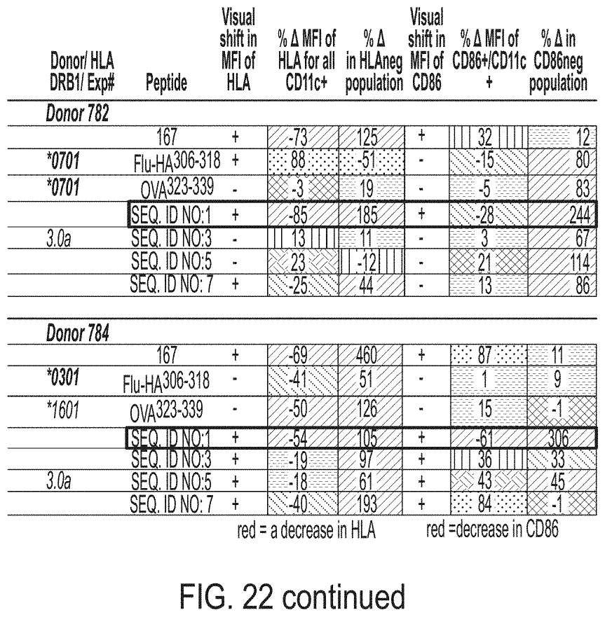

FIG. 5 is a series of dot plots representing the surface expression of CD11c vs CD86 analyzed on assay day 7 across the five donors in the presence of various peptide stimulants. An increase in CD86-low cells present in the samples treated with SEQ ID NO: 1, when compared to media control, indicated a shift to the acquired regulatory phenotype. FIG. 22 summarizes the results obtained through the dendritic cells phenotyping assays described previously. As presented in FIG. 22, exposure to claimed Tregitope SEQ ID NO: 1, decreased expression of HLA-DR in all five subjects tested. Further, in four out of five subjects, exposure to Tregitope SEQ ID NO: 1 increased the percent of CD86-low present among the CD11c-high cohort. Both trends indicated a shift towards an acquired regulatory phenotype.

(4) Methods for Assessing Peptide Effects on Proliferation of Regulatory T cells

Previous studies performed by EpiVax (Providence, R.I.) demonstrated increased proliferation of regulatory T cells following exposure to known Tregitope including positive control Tregitope 167 (SEQ ID NO: 15, 21st Century Biochemicals, Marlboro, Mass.). In this assay, candidate Tregitopes, including the Tregitopes of the instant disclosure, were tested for their ability to induce proliferation among CD4+CD25+ FoxP3+ regulatory T cells. Previously harvested and frozen PBMC were thawed and suspended in conditioned chRPMI (3.3×10⁶ cells/mL) by conventional means. Cells were stained with CFSE (Cat#: 65-0850-84, Affymetrix, Santa Clara, Calif.) and plated at 300,000 cells per well. Plates were incubated overnight (37° C. in 5% CO₂). On assay day 1, SEQ ID NO: 1 and SEQ ID NO: 3 (control peptide) were reconstituted in sterile DMSO yielding a final stock concentration of 20 mg/mL. Previous titration experiments performed at EpiVax (EpiVax, Providence, R.I.) have established that stimulation with 0.5 μg/ml Tetanus Toxoid (TT) (Astarte Biologics, Bothell, Wash.) elicits a measurable CD4+ effector memory T cells response in PBMC drawn from healthy control donors (Rhode Island Blood Center, Providence, R.I.). Tetanus Toxoid stock (100 μg/mL) (Astarte Biologics, Bothell, Wash.) was diluted in conditioned chRPMI yielding a working concentration of 1 ug/mL. Plated cells were then stimulated with either 100 μL of conditioned chRPMI (negative control), 100 μL Tetanus Toxoid solution (positive control) (Astarte Biologics, Bothell, Wash.), 100 μL of a dilution of 2991 μL Tetanus Toxoid solution plus 9 μL SEQ ID NO: 1 solution, 100 μL of a dilution of 2997 μL Tetanus Toxoid solution plus 3 μL SEQ ID NO: 1 solution, or 100 μL of a dilution of 6998.2 μL Tetanus Toxoid solution plus 1.8 μL SEQ ID NO: 1 solution. In parallel, control wells with identical number of the same cells were incubated with SEQ ID NO: 3 peptide solutions prepared as described for SEQ ID NO: 1. All plates were then incubated for six additional days. On assay day five, 100 μL of supernatant was removed from each well and replaced with freshly conditioned chRPMI.

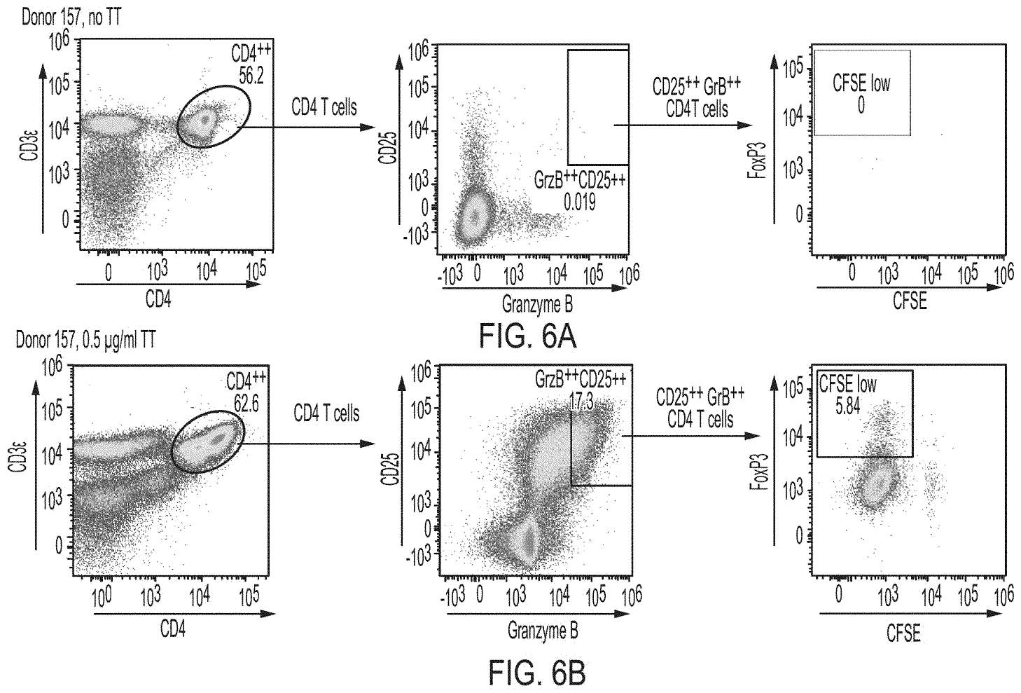

We selected highly activated regulatory T cells displaying elevated levels of FoxP3, CD25, Granzyme B and proliferation. The gating strategy for highly activated regulatory T cells is shown in FIGS. 6A-B (which depict a representative result using Donor 157), in which CD4+ T cells are gated for elevated CD25, Granzyme B, FoxP3, and low CFSE (proliferation). FIG. 6A shows the results of the representative assay with no added TT, while FIG. 6B shows the results of the representative assay with 0.5 μg/ml TT.

### EXAMPLE 4

### SEQ ID NO: 1 Strongly Induces a Population of Highly Proliferative, Activated Regulatory T Cells

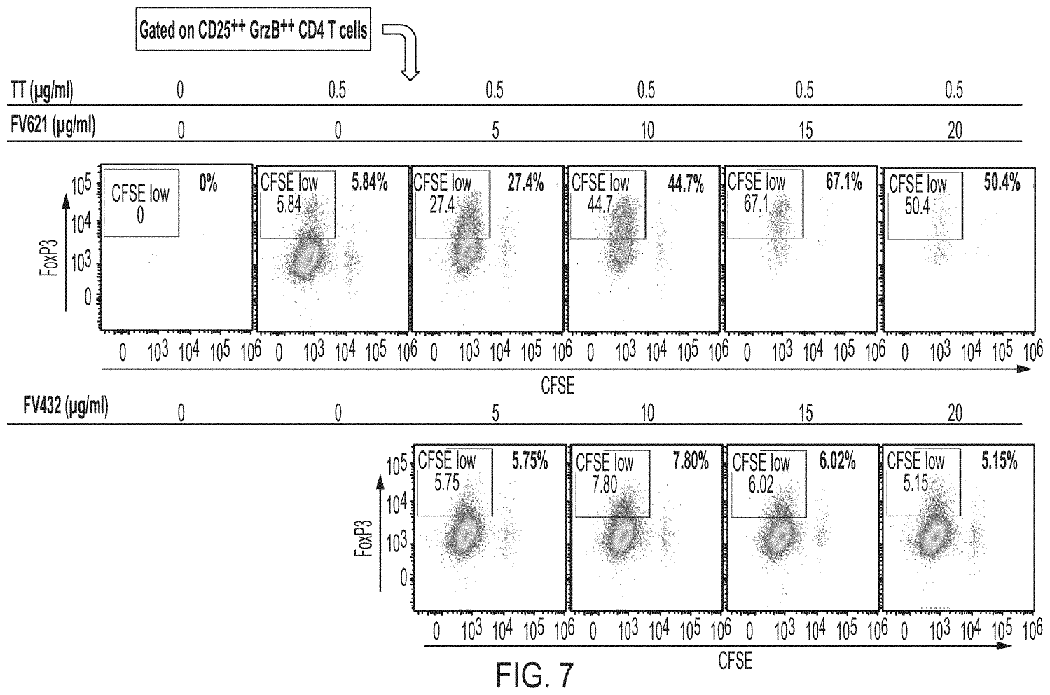

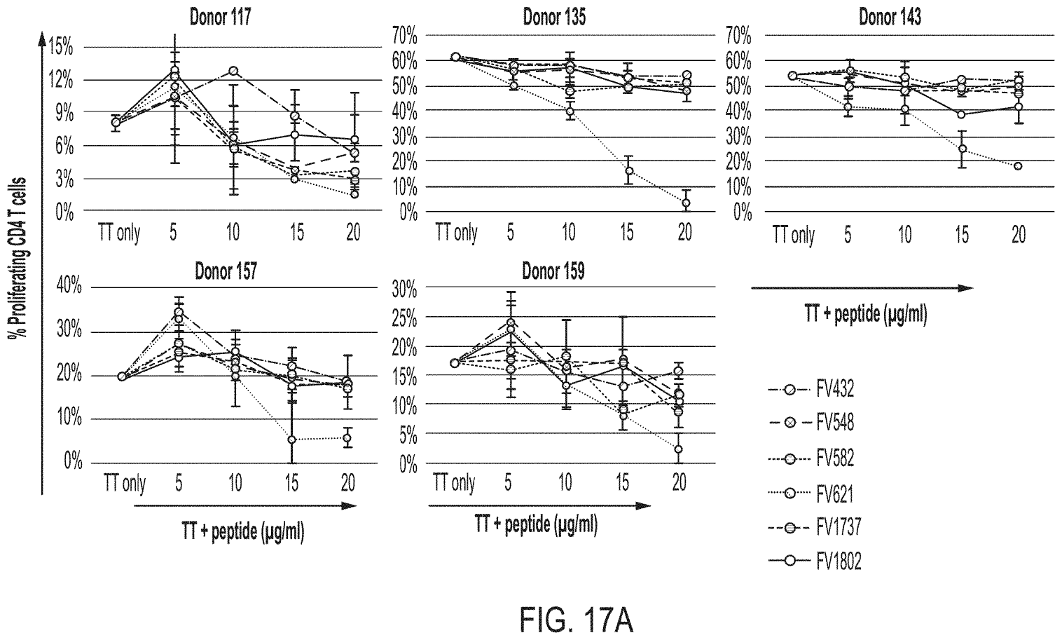

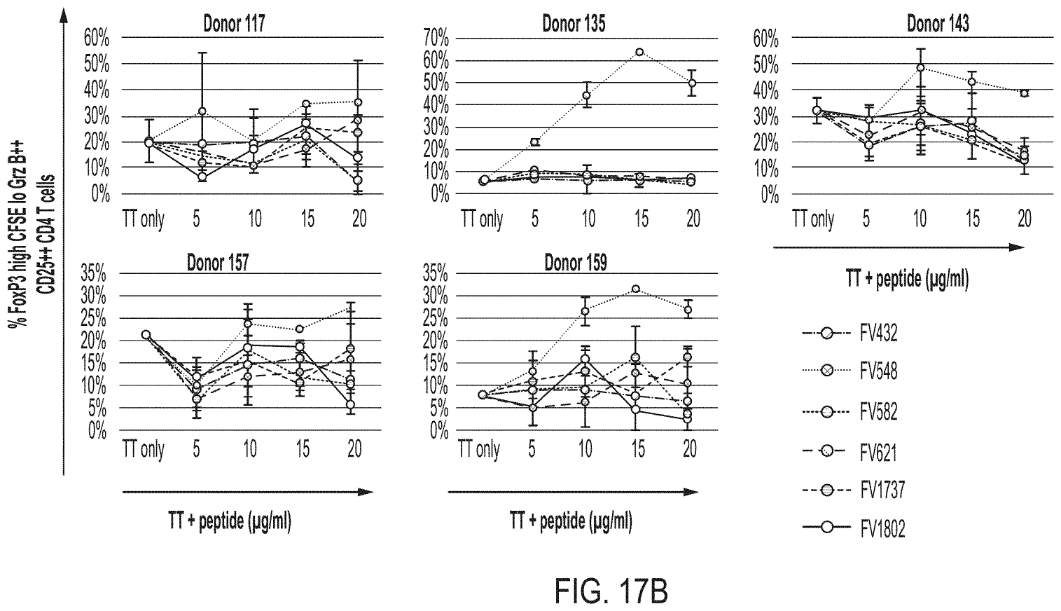

Regulatory T cell proliferation assays were performed on the Tregitopes of the present disclosure according to the methods described previously. As depicted in FIG. 7, gating on highly activated Granzyme B positive CD4⁺ T cells (CD25⁺ Granzyme B⁺) (as shown in FIGS. 6A-B), shows that a subset of them is composed of highly proliferative regulatory T cells (CFSE low FoxP3⁺). SEQ ID NO: 1 increases the relative proportion of this population, while SEQ ID NO: 3 has no significant effect. Data in the figure corresponds to Donor 135. The increase in this population of highly activated, Granzyme B positive regulatory T cells correlates closely with the degree of effector T cell inhibition shown by different Tregitopes of the present disclosure across multiple donors (FIGS. 17A-B), markedly increasing in relative numbers only in those cases where the Tregitopes of the present disclosure have an inhibitory effect on effector CD4⁺ cells. This is suggestive of the involvement of cytotoxic regulatory T cells in the inhibitory mechanism of SEQ ID NO: 1. In total, this data demonstrates that SEQ ID NO: 1 strongly induces a population of highly proliferative, activated regulatory T cells rich in Granzyme B.

(5) Methods for Assessing Peptide Effects on Proliferation of CD4+ Effector T cells

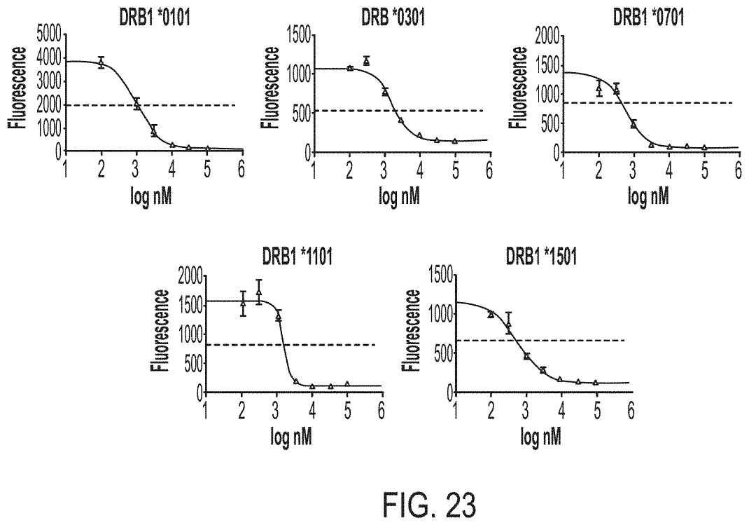

CD4+ effector memory T cells contained within PBMC cell populations can be induced to proliferate in response to stimulation with known T cell epitopes. EpiVax (Providence, R.I.) has demonstrated that SEQ ID NO: 1 binds multiple HLA molecules and that SEQ ID NO: 1 can induce a regulatory phenotype in exposed APC (Clinical Partners, Johnston, R.I.). Results of the competitive inhibition HLA binding assay provides an indirect measure of peptide-MHC affinity (Steere AC et al., J Exp Med, (2006), 203(4):961-71, herein incorporated by reference in its entirety). Binding of experimental Tregitopes is expressed as the percent inhibition of the labeled control peptide (experimental fluorescence/control fluorescence multiplied by 100). The percent inhibition values for each experimental Tregitope (across a range of molar concentrations) are used to calculate the concentration at which it inhibits 50% of the labeled control peptide's specific binding. This value is referred to as the IC₅₀. FIG. 23 shows the HLA binding results while Table 3 shows IC₅₀ for SEQ ID NO: 1 across five HLA-DR1 types, which indicates a high affinity of binding for SEQ ID NO: 1 across multiple HLA Class II types.

TABLE 3
HLA Binding results across multiple HLA alleles,
showing IC₅₀ for SEQ ID NO: 1.

| | | | HLA-DRB1 allele and IC₅₀ | | | | |
|---|---|---|---|---|---|---|---|
| Peptide | Sequence | Features | *0101 | *0301 | *0701 | *1101 | *1501 |
| SEQ ID | Ac- | IC₅₀ | 1.078 | 2.067 | 472 | 1.528 | 549 |
| NO: 1 | ILTIHFTGHSFIYGK- | | | | | | |
| | amide | | | | | | |

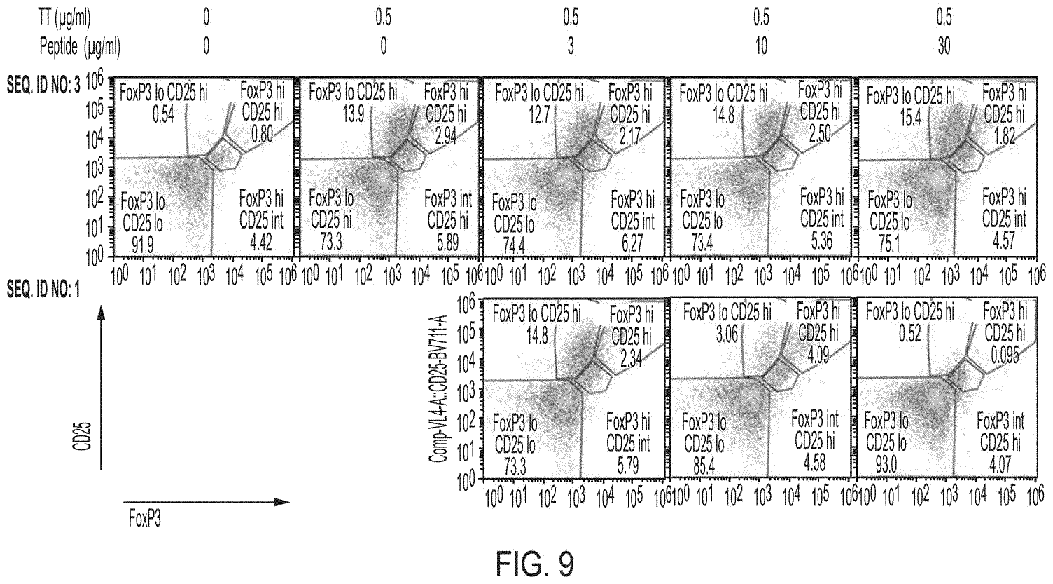

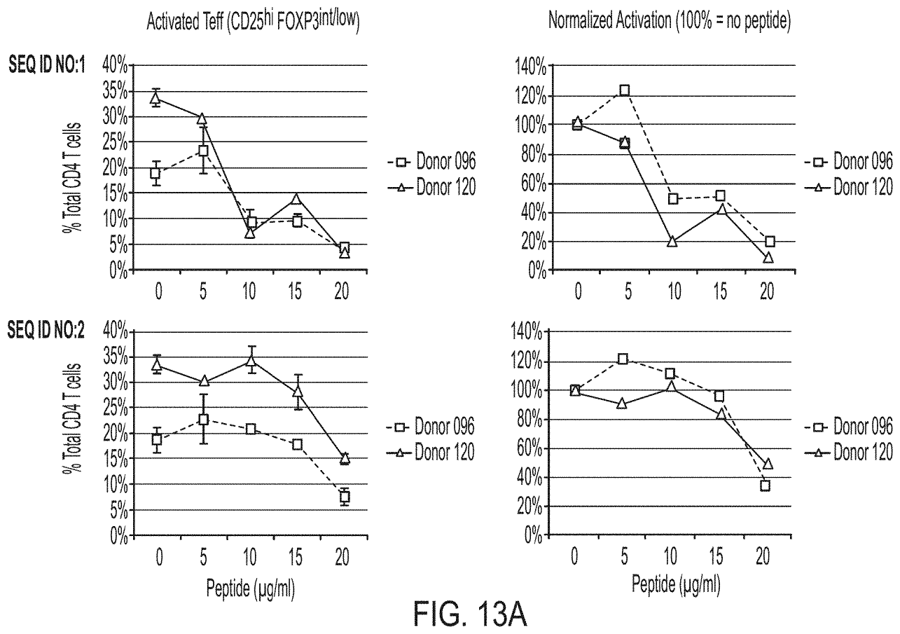

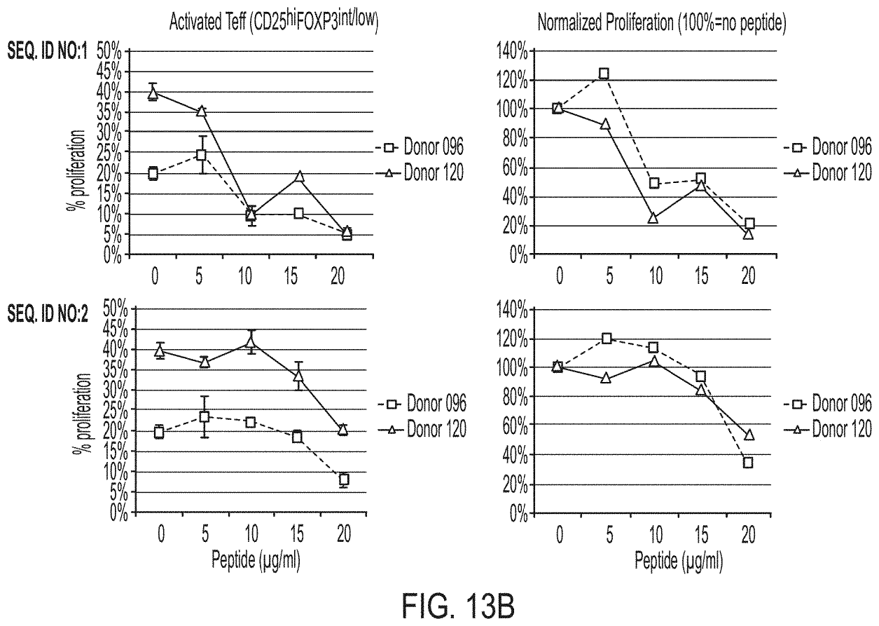

The purpose of this experiment was to establish the ability of SEQ ID NO: 1 to suppress the proliferation of antigen stimulated CD4+ effector memory T cells by either direct (engagement and activation of T_{Reg}) or indirect (modulation of APC phenotype) means. For the initial study, SEQ ID NO: 3 was used as a negative control (FIGS. 9-11). In subsequent studies, the performance of SEQ ID NO: 1 was compared to its FVIII derived homologue SEQ ID NO: 2 (FIG. 13).

Previously harvested and frozen PBMC were thawed and suspended in conditioned chRPMI (3.3×10⁶ cells/mL) by conventional means. Cells were stained with CFSE (Cat#: 65-0850-84, Affymetrix, Santa Clara, Calif.) and plated at 300,000 cells per well. Plates were incubated overnight (37° C. in 5% CO₂). On assay day 1, SEQ ID NO: 1 and SEQ ID NO: 3 (control peptide) were reconstituted in sterile DMSO yielding a final stock concentration of 20 mg/mL. Previous titration experiments performed at EpiVax (EpiVax, Providence, R.I.) have established that stimulation with 0.5 μg/ml Tetanus Toxoid (TT) (Astarte Biologics, Bothell, Wash.) elicits a measurable CD4+ effector memory T cells response in PBMC drawn from healthy control donors (Rhode Island Blood Center, Providence, R.I.). Tetanus Toxoid stock (100 μg/mL) (Astarte Biologics, Bothell, Wash.) was diluted in conditioned chRPMI yielding a working concentration of 1 ug/mL. Plated cells were then stimulated with either 100 μL of conditioned chRPMI (negative control), 100 μL Tetanus Toxoid solution (positive control) (Astarte Biologics, Bothell, Wash.), 100 μL of a dilution of 2991 μL Tetanus Toxoid solution plus 9 μL SEQ ID NO: 1 solution, 100 μL of a dilution of 2997 μL Tetanus Toxoid solution plus 3 μL SEQ ID NO: 1 solution, or 100 μL of a dilution of 6998.2 μL Tetanus Toxoid solution plus 1.8 μL SEQ ID NO: 1 solution. In parallel, control wells with identical number of the same cells were incubated with SEQ ID NO: 3 peptide solutions prepared as described for SEQ ID NO: 1. All plates were then incubated for six additional days. On assay day five, 100 μL of supernatant was removed from each well and replaced with freshly conditioned chRPMI.

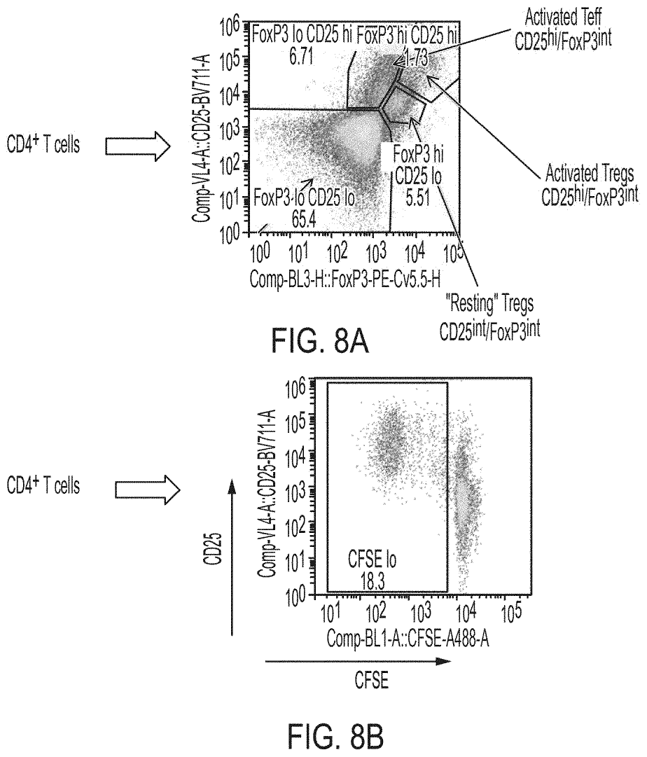

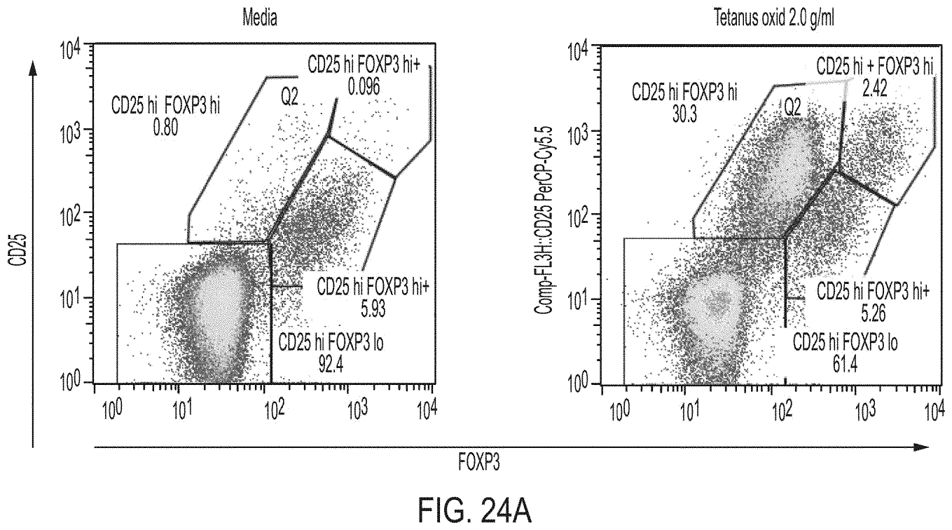

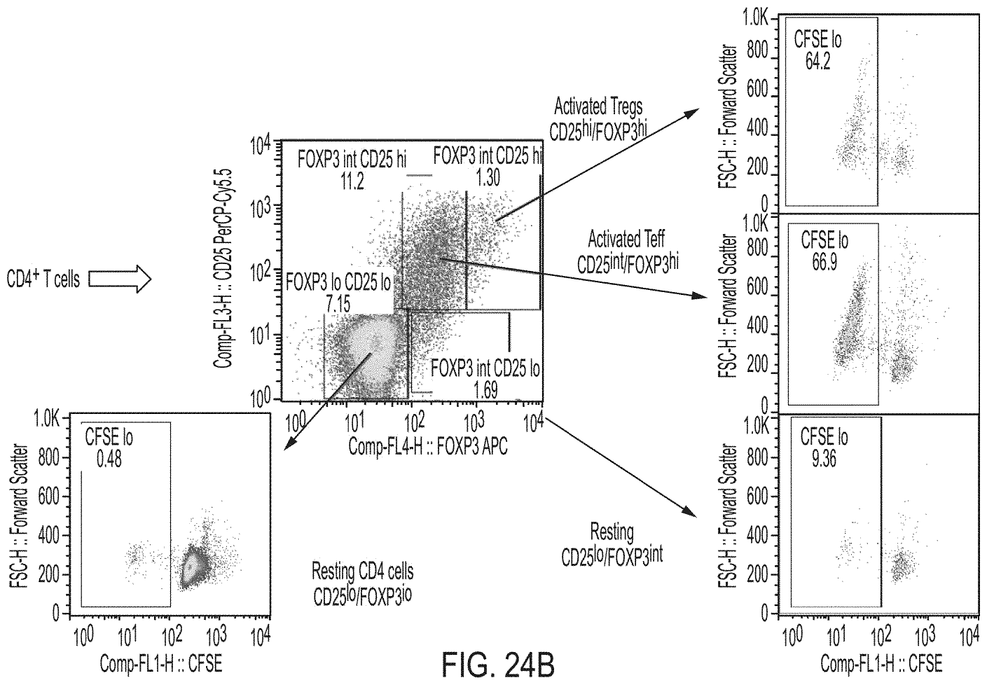

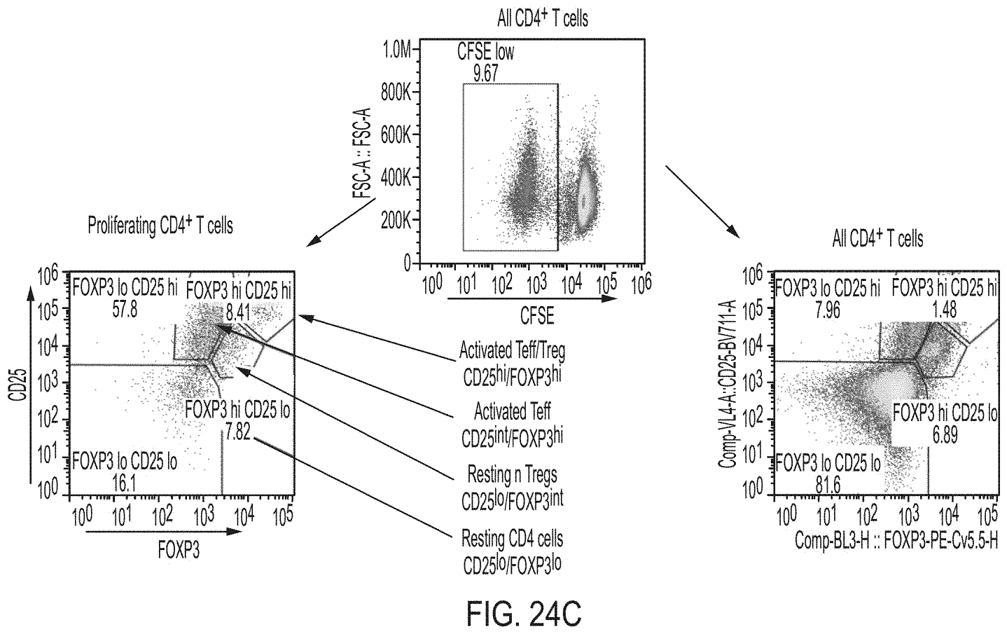

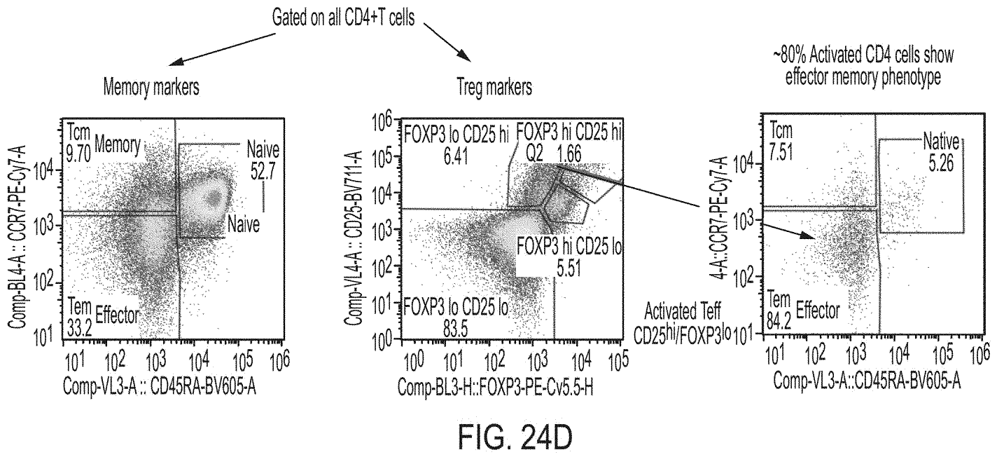

On assay day seven, cells were removed from incubation. Cells were labeled for live/dead discrimination, for surface markers CD127, CCR7, CD4, CD45RA, and CD25 and for intracellular FoxP3. Stained cells were further prepared for FACS analysis by conventional means. Cells were first gated to eliminate aggregates and dead cells. Live cells were gated for CD4 T cells and all subsequent analysis was done on this population. The activated Teffector population was identified as the CD4+/CD25-high/FoxP3-intermediate (CD4⁺/CD25^{hi}/FoxP3^{int}) (FIG. 8A). In a parallel analysis of this identified T effector cell population, it was shown that proliferation of this major population also corresponds to a CD45RA-low and CCR7-low effector memory T cells: FIGS. 24A-D, demonstrate the gating strategy employed for the Bystander Suppression Assay and highlights the regulatory and activation markers in CD4 T cells stimulated by TT, and demonstrating that the major proliferations population corresponds to the T effector memory phenotype (CD45RA-low/CCR7-low).

Proliferation of CD4+/CD25-high (CD4⁺/CD25^{hi}) T cells was estimated from the dilution of the CFSE stain (Cat#: 65-0850-84, Affymetrix, Santa Clara, Calif.) and % proliferation determined by the CFSE-low)(CFSE^{lo}) population (FIG. 8B).

### EXAMPLE 5A

### Peptide SEQ ID NO: 1 Suppressed Proliferation and Activation of CD4+ Effector T Cells

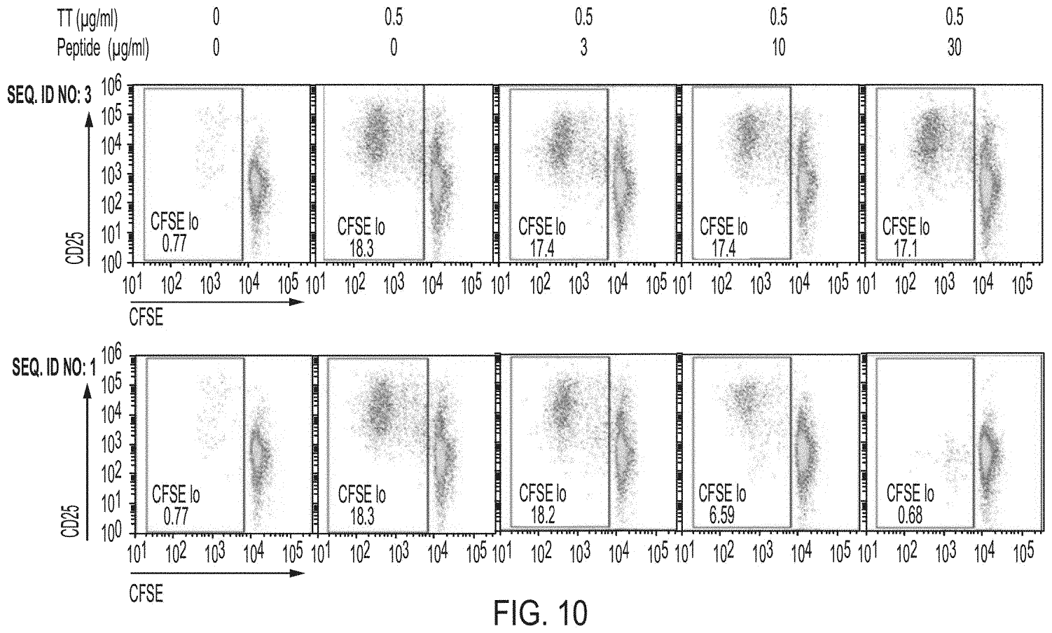

The change in activation (FIG. 9) and proliferation (FIG. 10) of CD4+ effector cells when the proliferation stimulant (Tetanus Toxoid) is co-delivered with SEQ ID NO: 1 was measured and the proliferative response of CD4+ T cells, comprised mainly of T effector memory cells, was characterized.

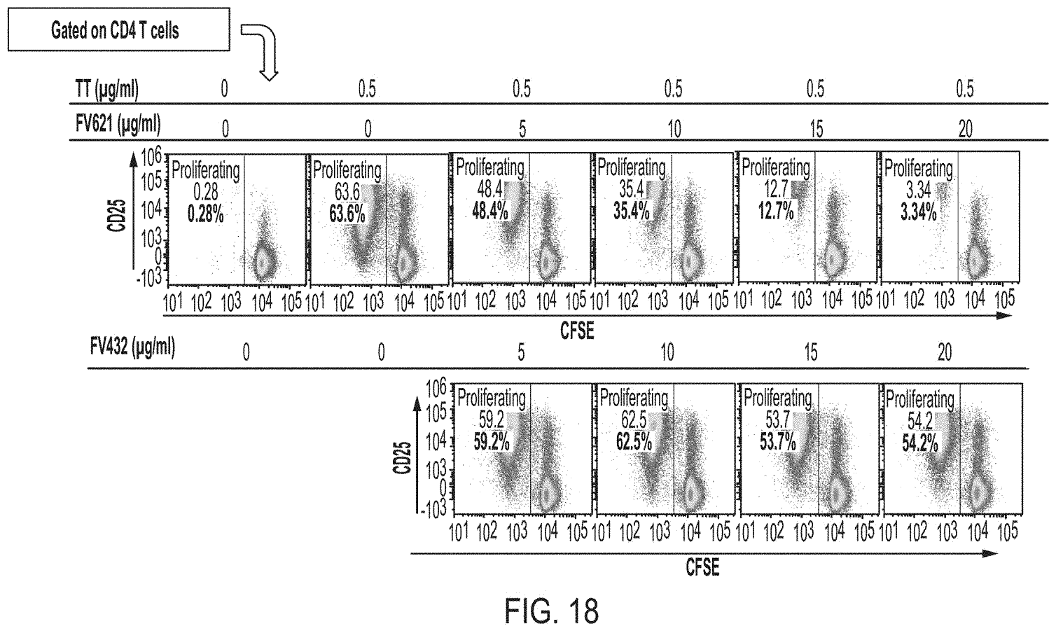

T cell proliferation assays were performed on the Tregitopes of the present disclosure according to the methods described previously. Dot plots corresponding to each experimental condition tested for activation and proliferation are presented in FIG. 9 and FIG. 10, respectively. FIG. 9 shows that, in donor 114, peptide SEQ ID NO: 1 strongly suppressed a population of activated effector CD4+ T cells (CD4+/CD25-high/FoxP3-intermediate, shown as CD4⁺/CD25^{hi}/FoxP3^{int}) reacting to Tetanus Toxoid (FIG. 9, lower row) in a dose-dependent manner, while control peptide SEQ ID NO: 3 had no appreciable effect (FIG. 9, upper row). In the same experiment, the proliferation of total CD4+ T cells activated by peptide SEQ ID NO: 1 (CFSE low cells, shown as CFSE^{lo}, in FIG. 10) was strongly suppressed by peptide SEQ ID NO: 1 (FIG. 10, lower row) in a dose-dependent manner, while control peptide SEQ ID NO: 3 had little to no effect (FIG. 10, upper row). FIG. 18 demonstrates that Tetanus Toxoid stimulated a population of activated (CD25 high) CD4 T cells to proliferate (CFSE low), with approximately 90% of the activated cells also proliferating (data not shown). This population of highly activated cells is actively suppressed by SEQ ID NO:1 (shown as FV621) in a dose-dependent manner (FIG. 18, upper row), while SEQ ID NO: 3 (shown as FV432) shows no significant inhibitory effect on activated CD4 cells (FIG. 18, lower row).

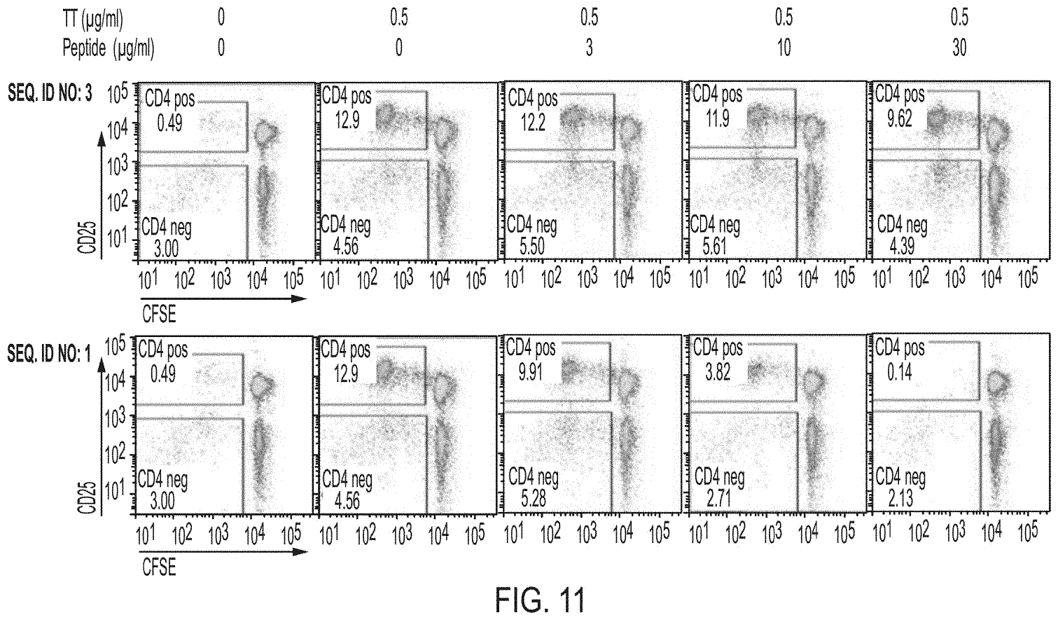

Further, gating on CD4+ and CD4− live cells demonstrated that the inhibitory effect of SEQ ID NO: 1 on cell proliferation was more pronounced on the CD4+ population than on the CD4− population (FIG. 11). SEQ ID NO: 1 suppressed proliferation of CD4− T cells in a dose dependent manner (FIG. 11, lower row). Negative control peptide SEQ ID NO: 3 showed little inhibitory effect on the CD4− populations (FIG. 11, upper row).

### EXAMPLE 5B

### Peptide SEQ ID NO: 1 and its FVIII Homologue Suppressed Proliferation of CD4+ Effector T Cells

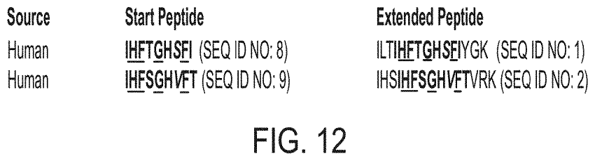

Additionally, the inhibitory effects of the Factor V derived Tregitope (SEQ ID NO: 1) with its Factor VIII homologue (SEQ ID NO: 2) on the CD4+ T cell effector memory response to TT in PBMCs in normal donors was compared, analyzed and evaluated. FIG. 12 shows an alignment of peptides SEQ ID NO: 1 and SEQ ID NO: 2 displaying the homology between both peptide sequences.

A study designed to evaluate the effect of peptides SEQ ID NO: 1 and SEQ ID NO: 2 on the recall response to TT of PBMCs derived from two normal donors was undertaken. The protocol used is previously described. Tregitope concentrations ranged from 5, 10, 15, and 20 ug/ml. SEQ ID NO: 1 inhibited CD4+ T cell activation and proliferation of T_{eff} responding to TT in a concentration-dependent manner (FIG. 13A (upper two panels) and FIG. 13B (upper two panels), respectively). As shown in FIG. 13A, for Donor 096, CD4+ T cell activation was reduced by 80% at 20 ug/ml (11 mM). Also as shown in FIG. 13A, for Donor 120, CD4+ T cell activation was reduced by 90% at 20 ug/ml (11 mM). SEQ ID NO. 2 when added with TT, inhibited both CD4+ T cell memory responses (CD4+ T cell activation and proliferation of T_{eff}) in a concentration-dependent manner (FIG. 13A (lower two panels) and FIG. 13B (lower two panels), respectively). As shown in FIG. 13A, for donor 096, CD4+ T cell activation was reduced by 70% at 20 ug/ml (11 mM). Also as shown in FIG. 13A, for Donor 120, CD4+ T cell activation was reduced by 50% at 20 ug/ml (11 mM). As shown in FIG. 13B, in general, Donor 120 responded with a 5-20% stronger inhibitor effect (for both CD4+ proliferation and T_{eff} activation) for a given peptide concentration of either SEQ ID NO: 1 (upper two panels) or SEQ ID NO. 2 (lower two panels) across the range of concentrations tested. The similarity in peptide sequence between the SEQ ID NO: 1 and its SEQ ID NO: 2 homolog, along with their parallel T cell inhibitory function, evidences that antigen-specific tolerance to FV is a useful method to stimulate tolerance to a homologous replacement protein, such as FVIII in Hemophilia A patients. In this assay, both SEQ ID NO: 1 and SEQ ID NO: 2 suppressed proliferation of CD4+ T cells and activated T effector T cells in a dose dependent manner.

### (6) Methods for Assessing SEQ ID NO: 1 Peptide Effects on CD8+ Effector T Cells.

CD8+ effector memory T cells contained within PBMC cell populations can be induced to proliferate in response to stimulation with known class I T cell epitopes. SEQ ID NO: 1 binds multiple HLA alleles and induces a regulatory phenotype in exposed APC (Clinical Partners, Johnston, R.I.) (the gating strategy employed allows for the identification of the APC fraction in PBMC collected from the whole blood donors). The results of this assay establish the ability of SEQ ID NO: 1 to suppress the proliferation of antigen stimulated CD8+ T effector memory T cells by either direct (engagement and activation of T_{Reg}) or indirect (modulation of APC phenotype) means.

T cell proliferation assays were performed on the Tregitopes of the present disclosure according to the methods described previously. PBMCs from two healthy donors were thawed and suspended in conditioned chRPMI (3.3×10⁶ cells/mL) by conventional means. Cells were stained with CFSE (Cat#: 65-0850-84, Affymetrix, Santa Clara, Calif.) and plated at 300,000 cells per well. Plates were incubated overnight (37° C. in 5% CO₂). On assay day 1, SEQ ID NO: 1 was re-constituted in sterile DMSO yielding a final stock concentration of 20 mg/mL. Intermediate solutions of SEQ ID NO: 1 at twice the final concentration in chRPMI were prepared as described previously. Final concentration of SEQ ID NO: 1 tested from 2.5, 5, 10 and 20 ug/ml. As a CD8+ stimulating antigen, the CEF peptide pool which consists of 23 MHC class I restricted viral epitopes derived from human cytomegalovirus, Epstein-Barr virus and influenza virus was used. CEF peptides were added to the wells (data shown for 2 μg/mL) with cells and media (control) or SEQ ID NO: 1 at 0, 1, 2 or 4 ug/ml. All plates were incubated for six additional days. On assay day 5, 100 uL of supernatant was removed from each well and replaced with freshly conditioned chRPMI.

Conventional methods were used to stain cells for live/dead marker, extracellular markers CD4, CD8α and CD25, CD127, CD45RA and CCR7, and intracellular marker FoxP3. After FACS analysis, cells were gated to eliminate aggregates and dead cells. On the live cells population, CD8α and CD4 cells were gated separately and each population was analyzed for proliferation (CFSE low population) or activation (CD25-high/FoxP3 low/intermediate, shown as FoxP3int_lo CD25hi) as explained previously.

### EXAMPLE 6

### Peptide SEQ ID NO: 1 Suppressed Proliferation of CD8+ Effector T Cells

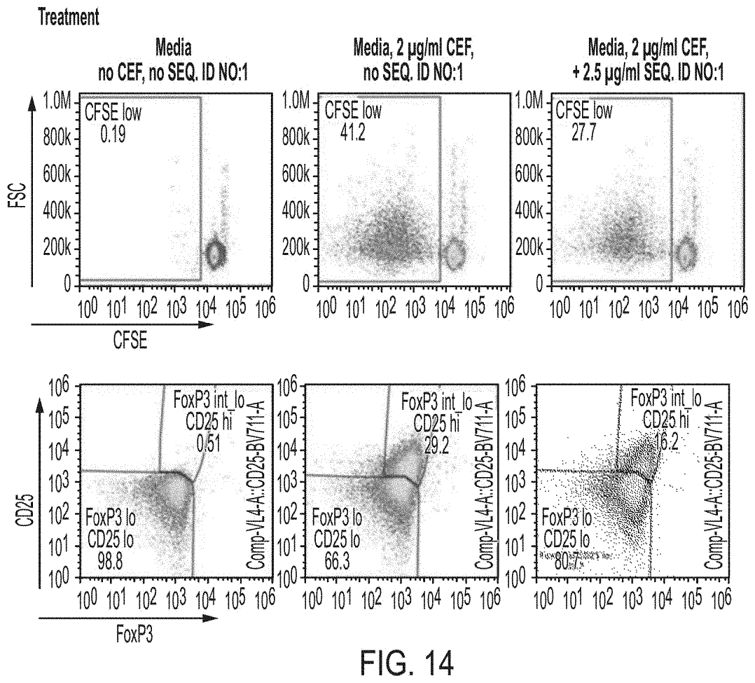

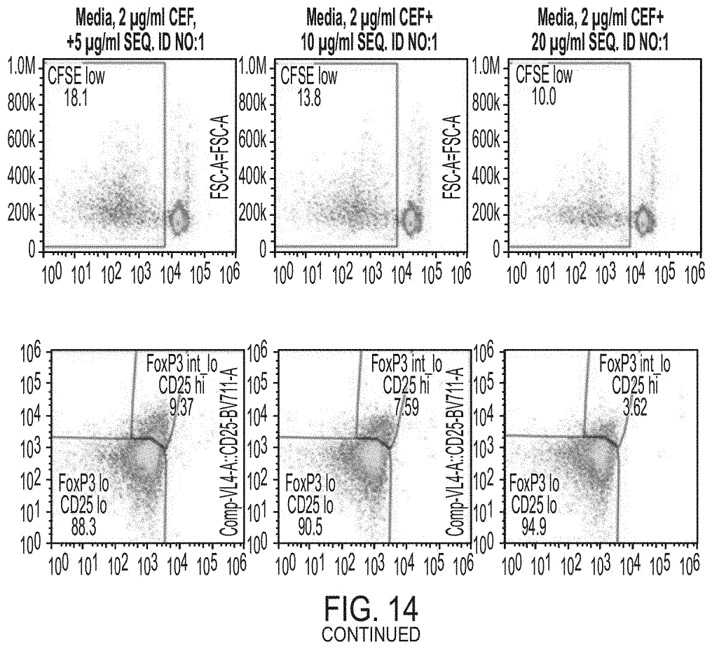

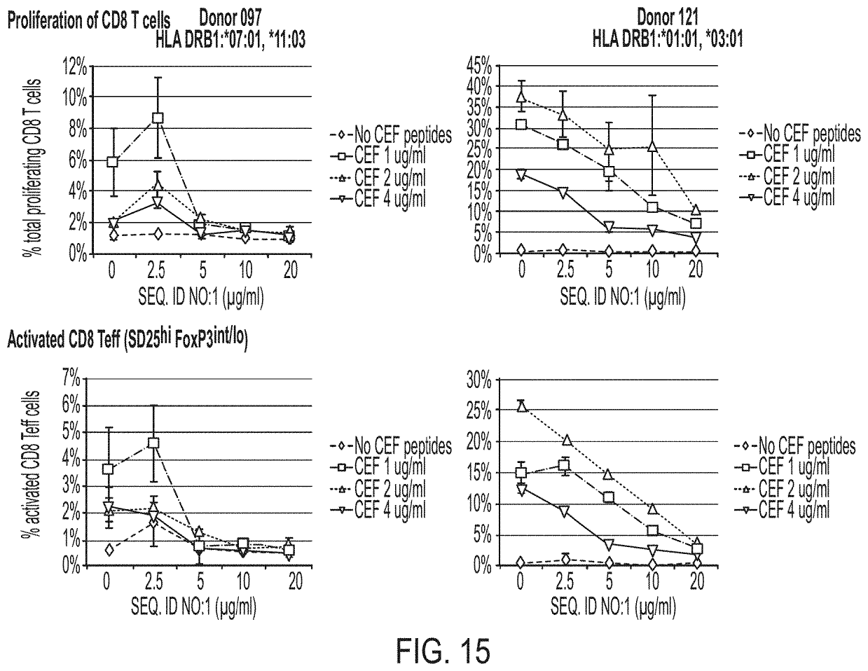

The potential inhibition of CD8+ T cell response by SEQ ID NO: 1 when PBMC from healthy donors are stimulated with CEF peptides mixture was tested. FIG. 14 shows that SEQ ID NO: 1 strongly inhibits the CD8+ T cell proliferative response to CEF peptides (upper row), as well as activation of CD8+ cells (lower row). FIG. 15 shows that the percent of proliferating CD8+ T cells (CFSE low) (upper two panels) and the percent of activated CD8+ T effector cells (CD25^{hi} FoxP3^{int/lo}) (lower two panels), decreases with increasing concentrations of SEQ ID NO: 1, demonstrating that SEQ ID NO: 1 also has an inhibitory effect on the CD8+ T cell population. In both cases, SEQ ID NO: 1 strongly inhibited the response in a dose-dependent manner.

### (7) Methods for Assessing Peptide Effects on Immune Response (GvHD)

Bone marrow transplant is a procedure whereby unhealthy bone marrow is replaced with donated healthy bone marrow. Bone marrow transplants can be used to treat patients with life-threatening blood cancers like leukemia (Vincente D et al., (2007), Bone Marrow Transplant, 40(4):349-54), diseases which result in bone marrow failure like aplastic anemia (Champlin RE et al., (2007), Blood, 109(10):4582-5), and other immune system or genetic diseases (Chinen J and Bucley RH, (2010), J Allergy Clin Immunol, 125(2 Suppl 2):S324-35). Graft versus host disease (GvHD) is known as a major complication in bone marrow transplantation and is characterized by immediate and high mortality after onset (Lee SJ et al., (2003), Biol Blood Marrow Transplant, 9(4):215-33). In GvHD, severe tissue damage is caused by donor lymphocytes as they make their way from transplanted donor tissue to HLA-mismatched recipient tissues. Symptoms include severe damage in various organs such as skin, lungs, liver and intestines caused by infusion in the recipient (Goker H et al., (2001), Exp Hematol, 29(3):259-77).

It was observed by EpiVax (Providence, R.I.) that transplantation of human peripheral blood mononuclear cells (PBMCs) (obtained from leukopaks (Hemacare, Van Nuys, Calif.)) into an immune deficient mouse causes a GvHD-like syndrome resulting in death by 20-50 days. In this model, T cells contained within the transplanted PBMC infiltrate the host mouse's skin, liver, intestine, lungs and kidneys causing severe damage and ultimately death. Immunodeficient mouse strain NOD-scid IL-2Rγ^{null} (NSG—Jax stock #005557) (The Jackson Laboratory, Bar Harbor, Me.) mice and transplants of human PBMC were used to assess the impact of SEQ ID NO: 1 on the progression of GvHD. On assay day—1, mice were grouped by weight into matched treatment and control groups (Table 4) and then irradiated with 100 cGy from an X-ray irradiator source (Lifespan Hospital, Providence, R.I.). After 6 hours of irradiation, mice subjects received 10 million hPBMCs IV via the tail vein. The mice in Group 8 received irradiation, but no PBMCs. Starting on assay day 0 and continuing through assay day 25, subject mice were dosed according to schedule outlined in Table 4.

Clinical observations, including weight loss, posture, activity, and appearance of hair coat and skin, were made three times per week. A subject mouse was euthanized if it exhibited a >20% weight loss from the starting date or exhibited a combination of the following clinical signs: (i) a 10-20% weight loss from the starting date (ii) coldness to touch (iii) lethargy with a hunched posture and scruffy coat.

TABLE 4
Experimental groups and dosing schedule for GVHD study

| Group | Mice | PBMC | Test Articles | Dose amount | Dosing frequency |
|---|---|---|---|---|---|
| 1 | 3 | + | PBS (disease control) | 300 μl PBS | Days 0, 2, 4, 7, 10, 15, 20, 25 |
| 2 | 10 | + | PBS + DMSO (vehicle) | 0.5% DMSO | Days 0, 2, 4, 7, 10, 15, 20, 25 |
| 6 | 10 | + | SEQ ID NO: 1 | 20 μg | Days 0, 2, 4, 7, 10, 15, 20, 25 |
| 7 | 7 | + | IVIG (positive control) | 50 mg | Days 0, 7, 14, 21, 28 |
| 8 | 3 | − | None (control group) | NA | |

### EXAMPLE 7

### SEQ ID NO: 1 Inhibited the Development of GvHD in Xenogenic GvHD Model

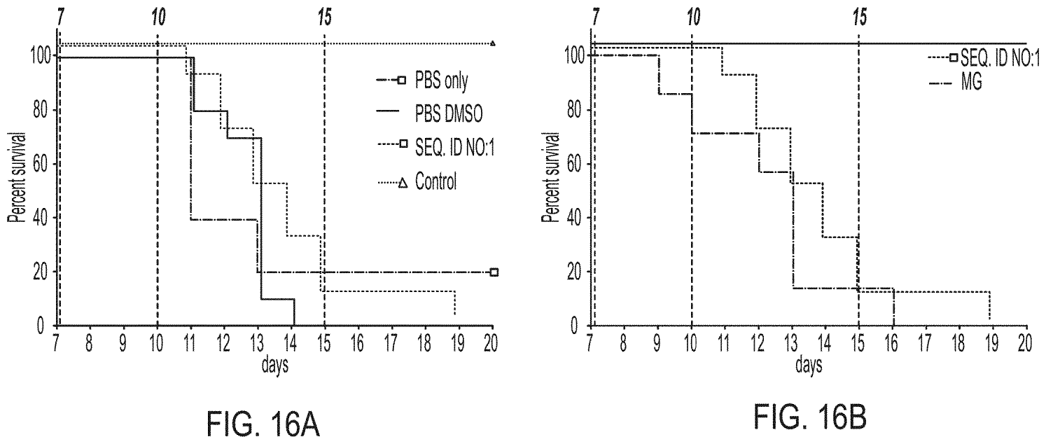

The transplantation of human lymphocytes into immunodeficient mice and subsequent treatment with SEQ ID NO: 1 enabled the assessment of SEQ ID NO: 1 on immune function in this in vivo model. SEQ ID NO: 1 suppressed T-cell activation thus slowing the progression of the disease. The main evaluation criteria used to evaluate was survival of the test subjects. A delay in the development of GvHD for the group treated with SEQ ID NO: 1 was observed as suggested by the Kaplan-Meiers Survival Curve. FIGS. 16A-B evidence that treatment with SEQ ID NO: 1 resulted in extended survival relative to negative controls (FIG. 16A) and the positive control IVIG (FIG. 16B).

### EXAMPLE 8

### Generation of a FVIII-Tregitope Construct

Fusion of Tregitope with an immunogenic protein can lead to the induction of peripheral tolerance of the immunogenic protein. Clotting Factor VIII is immunogenic in people with severe hemophilia A. In one exemplary method of producing such constructs, chimeric constructs comprised of the coding sequence of Factor VIII and Tregitope are produced (Sambrook et al., Molecular Cloning: A Laboratory Manual, 2 ed., Cold Spring Harbor Laboratory Press, (1989)). Briefly, the Factor VIII coding region fused at the carboxy-terminus and/or amino-terminus to a Tregitope is generated by annealing overlapping oligos and sub-cloned into an expression plasmid. A Tregitope may also be inserted into Factor VII, e.g. by mutagenesis (i.e., site-directed mutagenesis). The plasmids are transfected into DG44 CHO cells and stable transfectants selected. The chimeric protein is purified over an immunoaffinity column and evaluated for tolergenicity. Tables 5-8 illustrates exemplary embodiments of such proteins (e.g., a chimeric protein).

TABLE 5
Factor VIII-Tregitope: SEQ ID NO: 69
(Tregitope bold)

MQIELSTCFFLCLLRFCFSATRRYYLGAVELSWDYMQSDLGELPVDARFP
PRVPKSFPFNTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVFDEGKSWHSETKNSLMQDRDAASARAWPKM
HTVNGTVNRSLPGLIGCHRKSVYWHVIGMGTTPEVHSIFLEGHTFLVRNH
RQASLEISPITFLTAQTLLMDLGQFLLFCHISSHQHDGMEAYVKCDSCPE
EPQLRMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQRGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNSRHPSTRQKQFNATTIPENDIEKTDPWFAHRTPMPK
IQNVSSSDLLMLLRQSPTPHGLSLSDLQEAKYETFSDDPSPGAIDSNNSL
SEMTHFRPQLHHSGDMVFTPESGLQLRLNEKLGTTAATELKKLDFKVSST
SNNLISTIPSDNLAAGTDNTSSLGPPSMPVHYDSQLDTTLFGKKSSPLTE
SGGPLSLSEENNDSKLLESGLMNSQESSWGKNVSSTESGRLFKGKRAHGP
ALLTKDNALFKVSISLLKTNKTSNNSATNRKTHIDGPSLLIENSPSVWQN
ILESDTEFKKVTPLIHDRMLMDKNATALRLNHMSNKTTSSKNMEMVQQKK
EGPIPPDAQNPDMSFFKMLFLPESARWIQRTHGKNSLNSGQGPSPKQLVS
LGPEKSVEGQNFLSEKNKVVVGKGEFTKDVGLKEMVFPSSRNLFLTNLDN
LHENNTHNQEKKIQEEIEKKETLIQENVVLPQIHTVTGTKNFMKNLFLLS
TRQNVEGSYDGAYAPVLQDFRSLNDSTNRTKKHTAHFSKKGEEENLEGLG
NQTKQIVEKYACTTRISPNTSQQNFVTQRSKRALKQFRLPLEETELEKRI
IVDDTSTQWSKNMKHLTPSTLTQIDYNEKEKGAITQSPLSDCLTRSESIP
QANRSPLPIAKVSSFPSIRPIYLTRVLFQDNSSNLPAASYRKKDSGVQES
SHFLQGAKKNNLSLAILTLEMTGDQREVGSLGTSATNSVTYKKVENTVLP
KPDLPKTSGKVELLPKVHIYQKDLFPTETSNGSPGHLDLVEGSLLQGTEG
AIKWNEANRPGKVPFLRVATESSAKTPSKLLDPLAWDNHYGTQIPKEEWK
SQEKSPEKTAFKKKDTILSLNACESNHAIAAINEGQNKPEIEVTWAKQGR
TERLCSQNPPVLKRHQREITRTTLQSDQEEIDYDDTISVEMKKEDFDIYD
EDENQSPRSFQKKTRHYFIAAVERLWDYGMSSSPHVLRNRAQSGSVPQFK
KVVFQEFTDGSFTQPLYRGELNEHLGLLGPYIRAEVEDNIMVTFRNQASR
PYSFYSSLISYEEDQRQGAEPRKNFVKPNETKTYFWKVQHHMAPTKDEFD
CKAWAYFSDVDLEKDVHSGLIGPLLVCHTNTLNPAHGRQVTVQEFALFFT
IFDETKSWYFTENMERNCRAPSNIQMEDPTFKENYRFHAINGYIMDTLFG
LVMAQDQRIRWYLLSMGSNENIHSIHFSGHVFTVRKKEEYKMALYNLYPG
VFETVEMLPSKAGIWRVECLIGEHLHAGMSTLFLVYSNKCQTPLGMASGH
IRDFQITASGQYGQWAPKLARLHYSGSINAWSTKEPFSWIKVDLLAPMII
HGIKTQGARQKFSSLYISQFIIMYSLDGKKWQTYRGNSTGTLMVFFGNVD
SSGIKHNIFNPPIIARYIRLHPTHYSIRSTLRMELMGCDLNSCSMPLGME
SKAISDAQITASSYFTNMFATWSPSKARLHLQGRSNAWRPQVNNPKEWLQ
VDFQKTMKVTGVTTQGVKSLLTSMYVKEFLISSSQDGHQWTLFFQNGKVK
VFQGNQDSFTPVVNSLDPPLLTRYLRIHPQSWVHQIALRMEVLGCEAQDL
YILTIHFTGHSFIYGK

TABLE 6
Factor VIII-Tregitope: SEQ ID NO: 70
(Tregitope bold)

ILTIHFTGHSFIYGKMQIELSTCFFLCLLRFCFSATRRYYLGAVELSWDYM
QSDLGELPVDARFPPRVPKSFPFNTSVVYKKTLFVEFTDHLFNIAKPRPPW
MGLLGPTIQAEVYDTVVITLKNMASHPVSLHAVGVSYWKASEGAEYDDQTS
QREKEDDKVFPGGSHTYVWQVLKENGPMASDPLCLTYSYLSHVDLVKDLNS
GLIGALLVCREGSLAKEKTQTLHKFILLFAVFDEGKSWHSETKNSLMQDRD
AASARAWPKMHTVNGTVNRSLPGLIGCHRKSVYWHVIGMGTTPEVHSIFLE
GHTFLVRNHRQASLEISPITFLTAQTLLMDLGQFLLFCHISSHQHDGMEAY
VKCDSCPEEPQLRMKNNEEAEDYDDDLTDSEMDVVRFDDDNSPSFIQIRSV
AKKHPKTWVHYIAAEEEDWDYAPLVLAPDDRSYKSQYLNNGPQRIGRKYKK
VRFMAYTDETFKTREAIQHESGILGPLLYGEVGDTLLIIFKNQASRPYNIY
PHGITDVRPLYSRRLPKGVKHLKDFPILPGEIFKYKWTVTVEDGPTKSDPR
CLTRYYSSFVNMERDLASGLIGPLLICYKESVDQRGNQIMSDKRNVILFSV
FDENRSWYLTENIQRFLPNPAGVQLEDPEFQASNIMHSINGYVFDSLQLSV
CLHEVAYWYILSIGAQTDFLSVFFSGYTFKHKMVYEDTLTLFPFSGETVFM
SMENPGLWILGCHNSDFRNRGMTALLKVSSCDKNTGDYYEDSYEDISAYLL
SKNNAIEPRSFSQNSRHPSTRQKQFNATTIPENDIEKTDPWFAHRTPMPKI
QNVSSSDLLMLLRQSPTPHGLSLSDLQEAKYETFSDDPSPGAIDSNNSLSE
MTHFRPQLHHSGDMVFTPESGLQLRLNEKLGTTAATELKKLDFKVSSTSNN
LISTIPSDNLAAGTDNTSSLGPPSMPVHYDSQLDTTLFGKKSSPLTESGGP
LSLSEENNDSKLLESGLMNSQESSWGKNVSSTESGRLFKGKRAHGPALLTK
DNALFKVSISLLKTNKTSNNSATNRKTHIDGPSLLIENSPSVWQNILESDT
EFKKVTPLIHDRMLMDKNATALRLNHMSNKTTSSKNMEMVQQKKEGPIPPD
AQNPDMSFFKMLFLPESARWIQRTHGKNSLNSGQGPSPKQLVSLGPEKSVE
GQNFLSEKNKVVVGKGEFTKDVGLKEMVFPSSRNLFLTNLDNLHENNTHNQ
EKKIQEEIEKKETLIQENVVLPQIHTVTGTKNFMKNLFLLSTRQNVEGSYD
GAYAPVLQDFRSLNDSTNRTKKHTAHFSKKGEEENLEGLGNQTKQIVEKYA
CTTRISPNTSQQNFVTQRSKRALKQFRLPLEETELEKRIIVDDTSTQWSKN
MKHLTPSTLTQIDYNEKEKGAITQSPLSDCLTRSESIPQANRSPLPIAKVS
SFPSIRPIYLTRVLFQDNSSNLPAASYRKKDSGVQESSHFLQGAKKNNLSL
AILTLEMTGDQREVGSLGTSATNSVTYKKVENTVLPKPDLPKTSGKVELLP
KVHIYQKDLFPTETSNGSPGHLDLVEGSLLQGTEGAIKWNEANRPGKVPFL
RVATESSAKTPSKLLDPLAWDNHYGTQIPKEEWKSQEKSPEKTAFKKKDTI
LSLNACESNHAIAAINEGQNKPEIEVTWAKQGRTERLCSQNPPVLKRHQRE
ITRTTLQSDQEEIDYDDTISVEMKKEDFDIYDEDENQSPRSFQKKTRHYFI
AAVERLWDYGMSSSPHVLRNRAQSGSVPQFKKVVFQEFTDGSFTQPLYRGE
LNEHLGLLGPYIRAEVEDNIMVTFRNQASRPYSFYSSLISYEEDQRQGAEP
RKNFVKPNETKTYFWKVQHHMAPTKDEFDCKAWAYFSDVDLEKDVHSGLIG
PLLVCHTNTLNPAHGRQVTVQEFALFFTIFDETKSWYFTENMERNCRAPSN
IQMEDPTFKENYRFHAINGYIMDTLFGLVMAQDQRIRWYLLSMGSNENIHS
IHFSGHVFTVRKKEEYKMALYNLYPGVFETVEMLPSKAGIWRVECLIGEHL
HAGMSTLFLVYSNKCQTPLGMASGHIRDFQITASGQYGQWAPKLARLHYSG
SINAWSTKEPFSWIKVDLLAPMIIHGIKTQGARQKFSSLYISQFIIMYSLD
GKKWQTYRGNSTGTLMVFFGNVDSSGIKHNIFNPPIIARYIRLHPTHYSIR
STLRMELMGCDLNSCSMPLGMESKAISDAQITASSYFTNMFATWSPSKARL
HLQGRSNAWRPQVNNPKEWLQVDFQKTMKVTGVTTQGVKSLLTSMYVKEFL
ISSSQDGHQWTLFFQNGKVKVFQGNQDSFTPVVNSLDPPLLTRYLRIHPQS
WVHQIALRMEVLGCEAQDLY

TABLE 7
Factor VIII-Tregitope: SEQ ID NO: 71
(Tregitope bold)

MQIELSTCFFLCLLRFCFSATRRYYLGAVELSWDYMQSDLGELPVDARFP
PRVPKSFPFNTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVFDEGKSWHSETKNSLMQDRDAASARAWPKM
HTVNGTVNRSLPGLIGCHRKSVYWHVIGMGTTPEVHSIFLEGHTFLVRNH
RQASLEISPITFLTAQTLLMDLGQFLLFCHISSHQHDGMEAYVKCDSCPE
EPQLRMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQRGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNSRHPSTRQKQFNATTIPENDIEKTDPWFAHRTPMPK
IQNVSSSDLLMLLRQSPTPHGLSLSDLQEAKYETFSDDPSPGAIDSNNSL
SEMTHFRPQLHHSGDMVFTPESGLQLRLNEKLGTTAATELKKLDFKVSST
SNNLISTIPSDNLAAGTDNTSSLGPPSMPVHYDSQLDTTLFGKKSSPLTE
SGGPLSLSEENNDSKLLESGLMNSQESSWGKNVSSTESGRLFKGKRAHGP
ALLTKDNALFKVSISLLKTNKTSNNSATNRKTHIDGPSLLIENSPSVWQN
ILESDTEFKKVTPLIHDRMLMDKNATALRLNHMSNKTTSSKNMEMVQQKK
EGPIPPDAQNPDMSFFKMLFLPESARWIQRTHGKNSLNSGQGPSPKQLVS
LGPEKSVEGQNFLSEKNKVVVGKGEFTKDVGLKEMVFPSSRNLFLTNLDN
LHENNTHNQEKKIQEEIEKKETLIQENVVLPQIHTVTGTKNFMKNLFLLS
TRQNVEGSYDGAYAPVLQDFRSLNDSTNRTKKHTAHFSKKGEEENLEGLG
NQTKQIVEKYACTTRISPNTSQQNFVTQRSKRALKQFRLPLEETELEKRI
IVDDTSTQWSKNMKHLTPSTLTQIDYNEKEKGAITQSPLSDCLTRSESIP
QANRSPLPIAKVSSFPSIRPIYLTRVLFQDNSSNLPAASYRKKDSGVQES
SHFLQGAKKNNLSLAILTLEMTGDQREVGSLGTSATNSVTYKKVENTVLP
KPDLPKTSGKVELLPKVHIYQKDLFPTETSNGSPGHLDLVEGSLLQGTEG
AIKWNEANRPGKVPFLRVATESSAKTPSKLLDPLAWDNHYGTQIPKEEWK
SQEKSPEKTAFKKKDTILSLNACESNHAIAAINEGQNKPEIEVTWAKQGR
TERLCSQNPPVLKRHQREITRTTLQSDQEEIDYDDTISVEMKKEDFDIYD
EDENQSPRSFQKKTRHYFIAAVERLWDYGMSSSPHVLRNRAQSGSVPQFK
KVVFQEFTDGSFTQPLYRGELNEHLGLLGPYIRAEVEDNIMVTFRNQASR
PYSFYSSLISYEEDQRQGAEPRKNFVKPNETKTYFWKVQHHMAPTKDEFD
CKAWAYFSDVDLEKDVHSGLIGPLLVCHTNTLNPAHGRQVTVQEFALFFT
IFDETKSWYFTENMERNCRAPSNIQMEDPTFKENYRFHAINGYIMDTLFG
LVMAQDQRIRWYLLSMGSNENIHSIHFSGHVFTVRKKEEYKMALYNLYPG
VFETVEMLPSKAGIWRVECLIGEHLHAGMSTLFLVYSNKCQTPLGMASGH
IRDFQITASGQYGQWAPKLARLHYSGSINAWSTKEPFSWIKVDLLAPMII
HGIKTQGARQKFSSLYISQFIIMYSLDGKKWQTYRGNSTGTLMVFFGNVD
SSGIKHNIFNPPIIARYIRLHPTHYSIRSTLRMELMGCDLNSCSMPLGME
SKAISDAQITASSYFTNMFATWSPSKARLHLQGRSNAWRPQVNNPKEWLQ
VDFQKTMKVTGVTTQGVKSLLTSMYVKEFLISSSQDGHQWTLFFQNGKVK
VFQGNQDSFTPVVNSLDPPLLTRYLRIHPQSWVHQIALRMEVLGCEAQDL
YIHSIHFSGHVFTVRK

TABLE 8
Factor VIII-Tregitope: SEQ ID NO: 72
(Tregitope bold)

IHSIHFSGHVFTVRKMQIELSTCFFLCLLRFCFSATRRYYLGAVELSWDYM
QSDLGELPVDARFPPRVPKSFPFNTSVVYKKTLFVEFTDHLFNIAKPRPPW
MGLLGPTIQAEVYDTVVITLKNMASHPVSLHAVGVSYWKASEGAEYDDQTS
QREKEDDKVFPGGSHTYVWQVLKENGPMASDPLCLTYSYLSHVDLVKDLNS
GLIGALLVCREGSLAKEKTQTLHKFILLFAVFDEGKSWHSETKNSLMQDRD
AASARAWPKMHTVNGTVNRSLPGLIGCHRKSVYWHVIGMGTTPEVHSIFLE
GHTFLVRNHRQASLEISPITFLTAQTLLMDLGQFLLFCHISSHQHDGMEAY
VKCDSCPEEPQLRMKNNEEAEDYDDDLTDSEMDVVRFDDDNSPSFIQIRSV
AKKHPKTWVHYIAAEEEDWDYAPLVLAPDDRSYKSQYLNNGPQRIGRKYKK
VRFMAYTDETFKTREAIQHESGILGPLLYGEVGDTLLIIFKNQASRPYNIY
PHGITDVRPLYSRRLPKGVKHLKDFPILPGEIFKYKWTVTVEDGPTKSDPR
CLTRYYSSFVNMERDLASGLIGPLLICYKESVDQRGNQIMSDKRNVILFSV
FDENRSWYLTENIQRFLPNPAGVQLEDPEFQASNIMHSINGYVFDSLQLSV
CLHEVAYWYILSIGAQTDFLSVFFSGYTFKHKMVYEDTLTLFPFSGETVFM
SMENPGLWILGCHNSDFRNRGMTALLKVSSCDKNTGDYYEDSYEDISAYLL
SKNNAIEPRSFSQNSRHPSTRQKQFNATTIPENDIEKTDPWFAHRTPMPKI
QNVSSSDLLMLLRQSPTPHGLSLSDLQEAKYETFSDDPSPGAIDSNNSLSE
MTHFRPQLHHSGDMVFTPESGLQLRLNEKLGTTAATELKKLDFKVSSTSNN
LISTIPSDNLAAGTDNTSSLGPPSMPVHYDSQLDTTLFGKKSSPLTESGGP
LSLSEENNDSKLLESGLMNSQESSWGKNVSSTESGRLFKGKRAHGPALLTK
DNALFKVSISLLKTNKTSNNSATNRKTHIDGPSLLIENSPSVWQNILESDT
EFKKVTPLIHDRMLMDKNATALRLNHMSNKTTSSKNMEMVQQKKEGPIPPD
AQNPDMSFFKMLFLPESARWIQRTHGKNSLNSGQGPSPKQLVSLGPEKSVE
GQNFLSEKNKVVVGKGEFTKDVGLKEMVFPSSRNLFLTNLDNLHENNTHNQ
EKKIQEEIEKKETLIQENVVLPQIHTVTGTKNFMKNLFLLSTRQNVEGSYD
GAYAPVLQDFRSLNDSTNRTKKHTAHFSKKGEEENLEGLGNQTKQIVEKYA
CTTRISPNTSQQNFVTQRSKRALKQFRLPLEETELEKRIIVDDTSTQWSKN
MKHLTPSTLTQIDYNEKEKGAITQSPLSDCLTRSESIPQANRSPLPIAKVS
SFPSIRPIYLTRVLFQDNSSNLPAASYRKKDSGVQESSHFLQGAKKNNLSL
AILTLEMTGDQREVGSLGTSATNSVTYKKVENTVLPKPDLPKTSGKVELLP
KVHIYQKDLFPTETSNGSPGHLDLVEGSLLQGTEGAIKWNEANRPGKVPFL
RVATESSAKTPSKLLDPLAWDNHYGTQIPKEEWKSQEKSPEKTAFKKKDTI
LSLNACESNHAIAAINEGQNKPEIEVTWAKQGRTERLCSQNPPVLKRHQRE
ITRTTLQSDQEEIDYDDTISVEMKKEDFDIYDEDENQSPRSFQKKTRHYFI
AAVERLWDYGMSSSPHVLRNRAQSGSVPQFKKVVFQEFTDGSFTQPLYRGE
LNEHLGLLGPYIRAEVEDNIMVTFRNQASRPYSFYSSLISYEEDQRQGAEP
RKNFVKPNETKTYFWKVQHHMAPTKDEFDCKAWAYFSDVDLEKDVHSGLIG
PLLVCHTNTLNPAHGRQVTVQEFALFFTIFDETKSWYFTENMERNCRAPSN
IQMEDPTFKENYRFHAINGYIMDTLFGLVMAQDQRIRWYLLSMGSNENIHS
IHFSGHVFTVRKKEEYKMALYNLYPGVFETVEMLPSKAGIWRVECLIGEHL
HAGMSTLFLVYSNKCQTPLGMASGHIRDFQITASGQYGQWAPKLARLHYSG
SINAWSTKEPFSWIKVDLLAPMIIHGIKTQGARQKFSSLYISQFIIMYSLD
GKKWQTYRGNSTGTLMVFFGNVDSSGIKHNIFNPPIIARYIRLHPTHYSIR
STLRMELMGCDLNSCSMPLGMESKAISDAQITASSYFTNMFATWSPSKARL
HLQGRSNAWRPQVNNPKEWLQVDFQKTMKVTGVTTQGVKSLLTSMYVKEFL
ISSSQDGHQWTLFFQNGKVKVFQGNQDSFTPVVNSLDPPLLTRYLRIHPQS
WVHQIALRMEVLGCEAQDLY

### EXAMPLE 9

### Generation of a FVIII-Multi-Tregitope Construct

Multiple Tregitopes can be present in highly immunogenic proteins to promote adaptive tolerance. In one exemplary method of producing such constructs, chimeric constructs comprised of the coding sequence of clotting Factor VIII and multiple Tregitope(s) are produced (Sambrook et al., Molecular Cloning: A Laboratory Manual, 2 ed., Cold Spring Harbor Laboratory Press, (1989)). Briefly, the Factor VIII coding region fused at the carboxy-terminus and/or to a amino-terminus to Tregitopes is generated by annealing overlapping oligos and sub-cloned into an expression plasmid. Tregitopes may also be inserted into Factor VII, e.g. by mutagenesis (i.e., site-directed mutagenesis). The plasmids are transfected into DG44 CHO cells and stable transfectants selected. The chimeric protein is purified over an immunoaffinity column and evaluated for tolergenicity. Tables 9 illustrates an exemplary embodiments of such proteins (e.g., a chimeric protein).

TABLE 9
Factor VIII-Multi-Tregitope: SEQ ID NO: 73
(Tregitope bold)

MQIELSTCFFLCLLRFCFSATRRYYLGAVELSWDYMQSDLGELPVDARFP
PRVPKSFPFNTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVFDEGKSWHSETKNSLMQDRDAASARAWPKM
HTVNGTVNRSLPGLIGCHRKSVYWHVIGMGTTPEVHSIFLEGHTFLVRNH
RQASLEISPITFLTAQTLLMDLGQFLLFCHISSHQHDGMEAYVKCDSCPE
EPQLRMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQRGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNSRHPSTRQKQFNATTIPENDIEKTDPWFAHRTPMPK
IQNVSSSDLLMLLRQSPTPHGLSLSDLQEAKYETFSDDPSPGAIDSNNSL
SEMTHFRPQLHHSGDMVFTPESGLQLRLNEKLGTTAATELKKLDFKVSST
SNNLISTIPSDNLAAGTDNTSSLGPPSMPVHYDSQLDTTLFGKKSSPLTE
SGGPLSLSEENNDSKLLESGLMNSQESSWGKNVSSTESGRLFKGKRAHGP
ALLTKDNALFKVSISLLKTNKTSNNSATNRKTHIDGPSLLIENSPSVWQN
ILESDTEFKKVTPLIHDRMLMDKNATALRLNHMSNKTTSSKNMEMVQQKK
EGPIPPDAQNPDMSFFKMLFLPESARWIQRTHGKNSLNSGQGPSPKQLVS
LGPEKSVEGQNFLSEKNKVVVGKGEFTKDVGLKEMVFPSSRNLFLTNLDN
LHENNTHNQEKKIQEEIEKKETLIQENVVLPQIHTVTGTKNFMKNLFLLS
TRQNVEGSYDGAYAPVLQDFRSLNDSTNRTKKHTAHFSKKGEEENLEGLG
NQTKQIVEKYACTTRISPNTSQQNFVTQRSKRALKQFRLPLEETELEKRI
IVDDTSTQWSKNMKHLTPSTLTQIDYNEKEKGAITQSPLSDCLTRSESIP
QANRSPLPIAKVSSFPSIRPIYLTRVLFQDNSSNLPAASYRKKDSGVQES
SHFLQGAKKNNLSLAILTLEMTGDQREVGSLGTSATNSVTYKKVENTVLP
KPDLPKTSGKVELLPKVHIYQKDLFPTETSNGSPGHLDLVEGSLLQGTEG
AIKWNEANRPGKVPFLRVATESSAKTPSKLLDPLAWDNHYGTQIPKEEWK
SQEKSPEKTAFKKKDTILSLNACESNHAIAAINEGQNKPEIEVTWAKQGR
TERLCSQNPPVLKRHQREITRTTLQSDQEEIDYDDTISVEMKKEDFDIYD
EDENQSPRSFQKKTRHYFIAAVERLWDYGMSSSPHVLRNRAQSGSVPQFK
KVVFQEFTDGSFTQPLYRGELNEHLGLLGPYIRAEVEDNIMVTFRNQASR
PYSFYSSLISYEEDQRQGAEPRKNFVKPNETKTYFWKVQHHMAPTKDEFD
CKAWAYFSDVDLEKDVHSGLIGPLLVCHTNTLNPAHGRQVTVQEFALFFT
IFDETKSWYFTENMERNCRAPSNIQMEDPTFKENYRFHAINGYIMDTLFG
LVMAQDQRIRWYLLSMGSNENIHSIHFSGHVFTVRKKEEYKMALYNLYPG
VFETVEMLPSKAGIWRVECLIGEHLHAGMSTLFLVYSNKCQTPLGMASGH
IRDFQITASGQYGQWAPKLARLHYSGSINAWSTKEPFSWIKVDLLAPMII
HGIKTQGARQKFSSLYISQFIIMYSLDGKKWQTYRGNSTGTLMVFFGNVD
SSGIKHNIFNPPIIARYIRLHPTHYSIRSTLRMELMGCDLNSCSMPLGME
SKAISDAQITASSYFTNMFATWSPSKARLHLQGRSNAWRPQVNNPKEWLQ
VDFQKTMKVTGVTTQGVKSLLTSMYVKEFLISSSQDGHQWTLFFQNGKVK
VFQGNQDSFTPVVNSLDPPLLTRYLRIHPQSWVHQIALRMEVLGCEAQDL
YILTIHFTGHSFIYGKIHSIHFSGHVFTVRK

### (8) Generation of Tregitope-Blood Component Conjugates

Conjugation of a Tregitope with a blood component conjugate, such as albumin, can be useful as a carrier protein for Tregitope payload. Tregitope-blood component conjugates can extend the half-life of Tregitopes in vivo, protect Tregitopes from rapid proteolytic degradation, protect Tregitopes from rapid clearance from circulation and/or rapid kidney excretion, allow for wide distribution of Tregitope-blood component conjugates throughout the body of a subject, aid in delivery of Tregitopes to appropriate immune cells (such as macrophages and APCs), allow the Tregitopes to be processed by the endocytic pathway of certain immune cells (such as macrophages and APCs), and aid in the presentation of Tregitopes as an antigen by said immune cells.

Tregitope-blood component conjugates may be formed by modifying a Tregitope peptide of the instant disclosure (e.g., but not limited to, a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116)by attaching a reactive moiety to the Tregitope peptide to create a modified Tregitope peptide, then forming a bond between reactive moiety of the modified Tregitope peptide with a reactive functionality on a blood component, as disclosed in U.S. Pat. No. 6,849,714, U.S. Pat. No. 6,887,470, U.S. Pat. No. 7,256,253, and U.S. Pat. No. 7,307,148. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys³⁴) (the location of the amino acid in the amino acid sequence of human serine albumin), containing a free thiol with a pKa of approximately 5, which may serve as a preferred reactive functionality of albumin. Cys³⁴ of albumin is capable of forming a stable thioester bond with maleimidopropionamido (MPA), which is a preferred reactive moiety of a modified Tregitope peptide. The stable thioester bond between albumin and the Tregitope peptide modified with MPA cannot be cleaved under physiological conditions.

The Tregitope peptide may be as disclosed herein, and in certain aspects is preferably selected from SEQ ID NOS: 1-14 and 74-116, or a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116. One or more lysines may be present on the N-terminus of the Tregitope peptide, such as added onto to the N-terminus of peptides selected from SEQ. ID NOS: 1-14 and 74-116. A linker, such as a polyethyleneglycol linker (e.g., PEG2 or PEG12), is present between the one or more lysines and the Tregitope sequence, or at the N-terminus of a Tregitope sequence. In aspects, a lysosomal cleavage site, such as a Cathepsin B site, optionally consisting (sequentially from N-terminus to C-terminus) of valine and citrulline, is present between the PEG2 moiety and the Tregitope sequence. A maleimide-based chemistry may be used to covalently link the modified Tregitope peptide to a blood component, preferably serum albumin, in a 1:1 molar ratio. Linking the modified Tregitope peptide to a blood component may be performed in vivo or ex vivo.

Cathepsin B is the first described member of the family of lysosomal cysteine proteases. Cathepsin B possesses both endopeptidase and exopeptidase activities, in the latter case acting as a peptidyldipeptidase. Cathepsin B was been included in the Tregitope peptide design to facilitate the proper cleavage of the Tregitope from Albumin once it is in the lysosomal compartment in the antigen presenting cells. The Valine-Citrulline is a cathepsin B cleavage site that has been previously used successfully and has been FDA approved in Antibody Drug conjugate (e.g., monomethyl auristatin E (MMAE) conjugate in the drug brentuximab vedotin). Our interest in incorporating the site is to provide cleavage sites that would allow the proper cleavage of the Tregitope from the human serum albumin for efficient MHC class II presentation once it is in the APC. EpiVax saught to determine whether the incorporation of the cathepsin B site is essential to the design of the Tregitope compound or composition.

### EXAMPLE 10

### Generation of a Tregitope-Albumin Conjugate by Ex Vivo Conjugation

Standard Fmoc (9-fluorenylmethoxycarbonyl) solid phase peptide synthesis chemistry was used for peptide synthesis. Synthesis was performed on Intavis™ MultiPep™ automated peptide synthesizers. Amino acids are added stepwise to the growing peptide chain (C-terminus to N-terminus; right to left), while attached to an insoluble polystyrene resin support. Amino acid building blocks, protected at their amino terminus by an Fmoc group, were coupled to the growing chain after activation of the carboxylic acid terminus via one or more condensation reagents (e.g., Hexafluorophosphate Azabenzotriazole Tetramethyl Uronium (HATU), O-(1H-6-Chlorobenzotriazole-1-yl)-1,1,3,3-tetramethyluronium hexafluorophosphate (HCTU)). The reaction by-products at each addition are removed by solvent washing (6×, Dimethylformamide (DMF)). Following each coupling and capping step, the Fmoc is removed via piperidine deprotection of the peptide resin (performed 2×; 20% in DMF volume/volume with 0.1M HOBt to suppress Asp dehydration), the resin washed with DMF 6×, and the next amino acid added. A Cathepsin B cleavage site was incorporated at the N-terminus of the Tregitope sequence.

For a PEG2 construct (“PEG2” or “P2”), after the desired Tregitope peptide was completed a PEG2 moiety was added to the N-terminus, followed by the addition of 4 lysines to the N-terminus. The PEG2 and Lysines were incorporated to provide a potential docking area for the cathepsin B. Additionally, the PEG2 and lysines (via the primary amine on the lysine side-chain) would increase the solubility of the final construct. The composition of the PEG2 construct is shown in Table 10 (below).

TABLE 10
PEG2 construct composition

HSA (Cys 34)- Maleimide linkage- KKKK-Peg2-Val-Cit (CatB cleavage site)_Tregitope_Nle

For a PEG12 construct (“PEG12” or “P12”), two additions of a PEG6 was added after the Tregitope peptide synthesis. In this case, no lysines were added. Increasing the PEG length also provided a docking region for Cathepsin B and improved the solubility of the Tregitope. The composition of the PEG2 construct is shown Table 11 (below).

TABLE 11
PEG12 construct composition

HSA (Cys 34)-Maleimide linkage- Peg12-Val-Cit (CatB cleavage site)_Tregitope_Nle

Subsequently, a small amount of the peptide constructs were removed from the resin and the peptide sample cleaved and deprotected by treatment with trifluoroacetic acid (TFA. 92.5% v/v) in the presence of TIS (triisopropylsilane, 5%) and water (2.5%) to scavenge side-chain protecting groups. Each crude, linear, peptide (˜3-5 mg) was purified by preparative reversed phased HPLC (Gilson) using a 20 mm×50mm YMC C18, 5 um, Hydrosphere column. The peptides were purified to >90% purity (determined via analytical HPLC) and the mass verified utilizing an ABI-SCIEX QSTAR XL Pro Qo-TOF mass spectrometer prior to the Cathepsin B evaluation. The remaining peptides (PEG2-Tregitope and PEG12-Tregitope) was left on the resin for the addition of 3-maleimidoproprionic acid (MPA) at a later time.

Recombinant human cathepsin B (catalog 953-CY of R&D Systems™) was used to evaluate the cleavage of the Val-Cit site engineered into the Tregitope peptide. The activity assay protocol was used according to the R&D Systems™'s recommendations with final assay conditions of 0.01 ug rhCathepsin B and 10 uM of peptide substrate. After incubation of Cathepsin B with purified peptides (at RT for 15min). The peptide was evaluated by mass spec using the Qstar XL Pro™. It was determined that the PEG2 peptide did not have successful cleavage, and further modification of the Cathepsin B protocol did not produce successful cleavage. For the PEG12 product, successful cleavage was demonstrated.

After evaluation of the cleavage of the Val-Cit site by Cathepsin B, the reactive moiety of 3-maleimidoproprionic acid (MPA) was added to the N-terminus of the PEG2 and PEG12 peptides. Similar, to the amino acid building blocks, the MPA is protected by an Fmoc group, and coupled to the growing chain after activation of the carboxylic acid terminus. The final MPA-Tregitope constructs were removed from the resin and the peptide sample was cleaved and deprotected by treatment with trifluoroacetic acid (TFA. 92.5% v/v) in the presence of TIS (triisopropylsilane, 5%) and water (2.5%). Each crude, linear, peptide (˜20 mg) was purified by preparative reversed phased HPLC (Gilson™) using a 20 mm×50mm YMC C18, 5 μm, Hydrosphere column. The MPA-peptides were purified to >90% purity (determined via analytical HPLC) and the mass verified utilizing an ABI-SCIEX QSTAR XL Pro™ Qo-TOF mass spectrometer, as shown in FIGS. 26-29. A total of 15 mg of the MPA-P2 and MPA-P12 Tregitopes was used in the subsequent conjugation to rHSA (Albucult-Novozyme™) to construct the final preformed HSA-Tregitope conjugate.

Ellman's Reagent (5,5′-dithio-bis-[2-nitrobenzoic acid]) was used to estimate sulfhydryl groups in a sample by comparing to a standard curve of a sulfhydryl-containing compound such as cysteine. Ellman's test was performed on rHSA (Sigma™, Albucult®) at multiple concentrations to ensure the accuracy of the analysis. Ellman's reagent (Sigma™), rHSA from Sigma™ lot RF-009 was evaluated for free cysteine that would be available for conjugation with the maleimide. We estimated that 78% of the rHSA had free cysteine available, as shown in Table 12 (below).

TABLE 12
Estimation of free cysteine in rHSA samples

| | | | | | Moles huHSA | |
|---|---|---|---|---|---|---|
| Grams | Moles | O.D. | | | per mole | % free |
| rHSA | huHSA | 412 | Concentration | Moles | free cysteine | cysteine |
| 0.001 | 1.50376E−08 | 0.059 | 4.1696E−06 | 1.16749E−08 | 1.29 | 77.64 |
| 0.002 | 3.00752E−08 | 0.12 | 8.4806E−06 | 2.37456E−08 | 1.27 | 78.95 |

Peptide was solubilized in dH2O, rHSA added (15 mg/mi) and 100 mM Phosphate buffer added to give a final pH of 8. The peptide is added in a 10× molar excess to the HSA. Peptide/HSA was incubated at room temperature for 2 h followed by incubation at 4° C. for approximately 24-30 hours.

After the conjugation step, the HSA-conjugate was then dialyzed into PBS (pH 7.0) first at room temperature for 2 hours, followed by 2 changes to fresh PBS at 4° C. for 18-24 h. This process removed excess peptide from the HSA and HSA-Tregitope conjugate preparation.

The Ellman's test was performed on each conjugate to demonstrate conjugation of the peptide via the rHSA free Cysteine, and determine the efficiency of conjugation in the reaction. The HSA-conjugation preparation did not remove the reduced HSA (mercaptabumin), inherent in the preexisting preparation (˜22% of the HAS pre-conjugation). The remaining unreacted HSA was determined to be 14% for the HSA-MPA_P2-Tregitope construct, meaning after conjugation with the maleimide-Tregitope 14% of the free cysteine remained. Thus, ˜64% of total rHSA preparation was reacted with the MPA_P2-Tregitope peptide.

### (9) Methods for Assessing Effect of Tregitope-Blood Component Conjugates on Immune Cells

A maleimide-based chemistry may be used to covalently link a Tregitope (e.g., but not limited to, a peptide or polypeptide comprising, consisting, or consisting essentially of an amino acid sequence of SEQ ID NOS: 1-14 and 74-116 (and/or fragments or variants thereof), and optionally 1 to 12 additional amino acids distributed in any ratio on the N terminus and/or C-terminus of the polypeptide of SEQ ID NOS: 1-14 and 74-116) payload to recombinant HAS (rHSA) in a 1:1 stoichiometry. Maleimido-propionamido (MPA) forms a stable thiol ester conjugate with the available free Cys34 in HSA. HSA leverages the neonatal receptor (FcRn) recycling pathway, increasing the half-life of any conjugated payload, and potentially decreasing the need for repeat dosing. rHSA is also known to deliver conjugated payloads to the lymph nodes and is endocytosed by dendritic cells and other antigen presenting cells that express FcRn.

EpiVax designed an rHSA-Tregitope conjugate to contain cleavage sites between the Tregitopes. The cleavage sites are specific for an early endosomal protease, which enable the Tregitopes to be liberated from the rHSA molecule, increasing the efficiency of MHC class II presentation on the cell surface. The long and substantiated history of this FDA-Approved rHSA conjugation chemistry approach, as well as its successful manufacturing history support its selection for delivery of our T1D payload.

Once Tregitope-blood component conjugates are formed, for example as described in Example 14 and subsection 9 of the examples section, as well as the detailed description, the Tregitope-blood component conjugates may be evaluated for their effectiveness in inhibiting effector T-cells and activating regulatory T-cells and their proliferation, for example in comparison with Tregitope peptides alone. Further, the Tregitope-blood component conjugates may be evaluated for their capacity to induce immune tolerance against certain antigens

### EXAMPLE 11

### Evaluation of the Inhibitory Effect of Tregitope-Albumin Delivery Vehicle

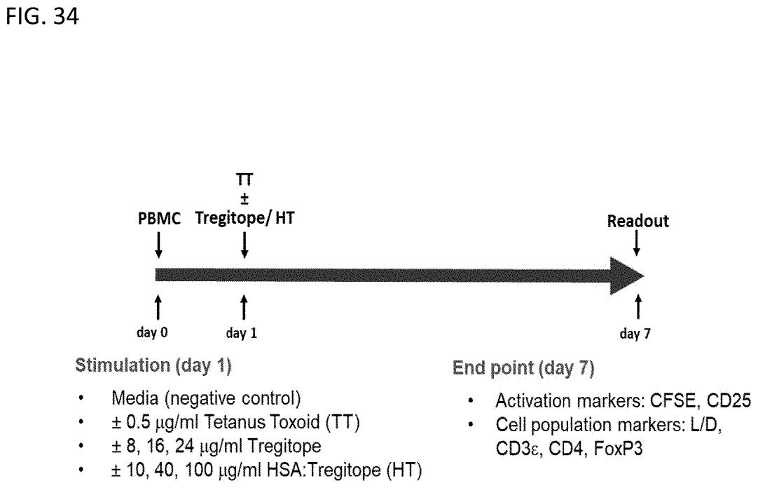

To determine the inhibitory effect of the Tregitope delivery vehicle, healthy donor PBMCs are used in a tetanus toxoid bystander supression assay (TTBSA), and analysis is done on CD4 T-cell proliferation, activation of T cells, frequencies of T effector and T regulatory cells to determine the ratio of Treg/Teff, as is displayed in FIG. 34.

So as to optimize the best combination of Tregitopes for translation to the clinic, the effect of combinations of Tregitopes for their ability to synergistically suppress effector T-cell responses in vitro is analyzed. To facilitate these comparisons, a high throughput in vitro assay is developed using human donor peripheral blood mononuclear cells (PBMCs). This assay, referred to as the Tetanus Toxoid Bystander Suppression Assay, takes advantage of the ability of Tregs to suppress T memory cells specific to Tetanus that are elicited in individuals with a history of Tetanus toxoid (TT) vaccination.

At day 0, PBMCs are incubated and stained with Carboxyfluorescein succinimidyl ester (CFSE) dye. At day 1, cells are stimulated with by adding media, Tetanus Toxoid, and either: 8, 6, or 24 μg/mL of a Tregtiope; or 10, 40, or 100 μg/mL of a Tregtiope-albumin conjugate. Tetanus Toxoid is used at a final concentration of 0.5 μg/ml, where the concentration is methodically titrated and optimized to measure the inhibitory capacity of Tregitopes. Negative controls, including media-only, are included. At day 7, L/D cell population marker, extracellular stain, and intracellular stain are added to the cells. At day 8, a readout is taken. Cell sorting assays for analysis of activation markets (e.g., CFSE, CD25) and cell population markets (e.g., L/D, CD2c, CD4, and FoxP3) are performed.

Incubation of donor PBMCs with TT stimulates expansion of T effector cells. Tregitopes are added to PBMC in vitro with TT, and activate CD25^{hi}FoxP3^{hi} regulatory T cells suppressing expansion of TT-specific T effector cells. Tregitopes significantly inhibit the proliferation (as is measured by CFSE dilution) and activation (as is measured by CD25 expression) of CD4+ T effector cells in a dose dependent manner, and also slightly expand Tregs (CD25⁺/FoxP3⁺/CD127^{lo}), which is suggested by an increase in the ratio of Treg/Teff cells. A reduction of effector T cell proliferation is a direct consequence of the activation of T regulatory cells and/or the conversion of TT-specific T effector to Treg, for example as is supported by the induction of Treg in vivo.

Using the TTBSA, each of a number of available Tregitopes individually and in pairwise combinations is examined for their potential to suppress CD4+ T cell proliferation. The most promising IgG-Tregitope peptides are selected for further testing. A certain Tregitope, Tregitope A, is the single Tregitope has the most suppressive activity in the TTBSA as compared to the other single Tregitopes. Combining Tregitope A with Tregitope C, an even greater suppressive effect on TT-specific T cell proliferation is observed. Conjugating A+C to rHSA improves their efficacy in vitro.

Thus, using TTBSA, it is shown that HSA-Tregitope conjugates inhibit CD 4 T-cell proliferation and activation, and increase the ratio of Treg cells to Teff cells.

### EXAMPLE 12

### Evaluation of the Effectiveness of Preformed Conjugate HSA-Tregitope Therapeutics and Maleimide-Tregitope Peptide Therapeutics

The effect on the response to OVA immunization of preformed conjugate HSA-Tregitope therapeutics and a free-maleimide-Tregitope peptide is evaluated. The latter free-maleimide peptide forms a conjugation in vivo after injection via the reactive maleimide group to the free-Cys34 of the subject's endogenous HSA. 5 mgs of the MPA-P2 and MPA-P12 is used as free-MPA-Tregitope, with the unconjugated HSA in the sample being accounted for by calculating the molar ratio of conjugated to unconjugated HSA.

Mice (female C57BL/6) are immunized s.c. with 50 mg ovalbumin (OVA) on day 0 (CFA) and day 14 (IFA). The preformed HSA conjugate treatments is administered with the OVA in CFA on day 0. Test groups include OVA/HSA-P2-high and OCA/HSA-P2-low. Per injection OVA is 50 μg, and HSA at 800 μg, and HSA-P2H(high) conjugation is at 825 μg (˜20 μg Tregitope). HSA-P2L(low) conjugation is at 100 μg (-3.7 μg Tregitope). Four control groups include PBS only, PBS/OVA, HSA/OVA, and Tregitope/OVA. A last arm is included to evaluate the utility of the free-maleimide Tregitope peptide and is administered by IV into tail vein. There are five mice per group.

Mice are sacrificed on Day 17. Upon sacrifice, cardiac bleeds and spleens are harvested for each animal. IFNγ/IL2 fluorospot assays, IFNγ/IL17 fluorospot assays, CD4 T cell proliferation, and T cell characterization are performed on the splenocytes stimulated with OVA. PHA is used as a positive control stimulation for spleen cell assays. All of the wells in PHA stimulation are confluent. An acceptance criteria is used wherein SFC (spot forming cells) after stimulation must be greater than 50 spots/10⁶ over negative control (media wells) and must also have a stimulation index greater than 2. According to both the IFNγ/IL2 fluorospot and IFNγ/IL17 fluorospot assays, IFNγ production is inhibited by treatment, and the HSA-only control group is inhibited less compared than the treatment groups.

For T-cell proliferation and characterization assays, splenocyte samples are evaluated for induction of FoxP3 expression in TCR Tg cells and for the suppression of OVA specific T cell proliferation (in response to OVA peptide in vitro) by CFSE dilution. To detect FoxP3⁺ Tregs, a single-cell suspension of draining lymph nodes is incubated with 2.4G2 mAb (anti-CD16/32, ATCC) for 15 minutes to block FcR then is stained with anti CD3, CD4, CD25 and anti-clonotypic KJ1-26 for 40 minutes at 4° C. KJ1-26 is specific for clonotypic TCR expressed by DO11.10 transgenic mice. Cells are then be permeabilized and stained for FoxP3 nuclear expression and acquired on a Thermo Attune NxT Autosampler™ for FACS analysis. The CD4⁺CD25⁺FoxP3⁺KJ1-26⁺ live cell gate population is established to determine the number and proportion of OVA-Specific T regulatory cells compared to PBS or HSA alone.

Antigen-specific T cell proliferation is evaluated by CFSE dilution. Draining lymph nodes are harvested, are stained with cell proliferation dye CFSE, and a single-cell suspension is prepared at 2×10⁶ cells/mL. Cells are added to 96-well plates at 100 μL per well in the presence of 10 μg/ml concentration of OVA 323-339 (New England Peptide, Gardner, Mass., USA). Cells are stimulated for 72 hours and harvested for staining with CD3α, CD4, CD8, CD54RA, CCR7, CD25, CD127, IFNγ HLA-II, CD69, CD154, IL-17, IL-21 for 40 minutes at 4° C. Cells are be fixed, permeabilized and stained for FoxP3 expression and analyzed by flow cytometry. An increase of OVA-specific KJ1-26⁺CD4⁺CD25⁺FoxP3⁺ adaptive (converted) T regulatory cells in mice treated with free maleimide-Tregitopes and HSA-Tregitope conjugates as compared to mice treated with rHSA is observed. free maleimide-Tregitopes and HSA-Tregitope conjugates more effectively reduces OVA-specific proliferation of KJ1-26⁺CD4⁺ T effector cells as compared to rHSA alone.

Anti-OVA antibodies in serum from the bleeds harvested on day 17 are evaluated in serum by ELISA, including a serial dilution plot and a standard ELISA to determine antibody concentrations. Mice treated with HSA-conjugates and free maleimide have lower serum antibody titers compared to no treatment, as indicated by absorbance at different dilutions, as well as comparison of absorbance over a standard curve.

### EXAMPLE 13

### Materials and Methods

### Bioinformatics Screening and Selection of Factor V Peptides

Immunoinformatics analysis using the EpiMatrix and JanusMatrix algorithms (J. Schafer, Prediction of well-conserved HIV-1 ligands using a matrix-based algorithm, EpiMatrix, Vaccine. 16 (1998) 1880-1884; and L. Moise, et al., The two-faced T cell epitope, Hum. Vaccin. Immunother. 9 (2013) 1577-1586, both herein incorporated in reference in their entireties), and was used to select Factor V-derived peptides predicted to be regulatory according to the following criteria: the peptides are predicted to bind four or more HLA-DRB1 alleles; and their TCR facing residues share a high degree of homology to sequences with similar HLA-profile in FVIII and/or to other prevalent proteins. Six Factor V-derived peptides that were promiscuous and highly conserved in the human proteome were selected for in vitro screening (HLA binding and T reg assays). And although information on MHC elution and identification by mass spectrometry of these peptides was not a criteria for their selection, subsequently, HLA-DR-restricted sequences that overlap with the more HLA-DR-promiscuous FV621 sequence been reported to be eluted from HLA DR.

In addition, previously described IgG-derived Tregitopes 167 and 289 that are located in the Fc domain and Tregitope 84 located in the kappa domain of IgG, were used as positive control peptides in the HLA binding and T reg assays described below.

### Peptide Synthesis

The peptides used in these studies were synthesized by 21^{st} Century Biochemicals (Marlborough, Mass.). Molecular weight accuracy was verified by mass spectrometry and all peptides were determined to be more than 90% pure by H PLC. Amino acid analysis was performed on all peptide samples so as to normalize net peptide content across assays.

### HLA Binding Assays

The six FV peptides were tested for in vitro binding to HLA-DRB1*0101, *0301, *0401, *0701, *1101, *1301 and *1501 alleles. HLA binding assay results for IgG Fc derived Tregitope peptides have been previously reported (L. P. Cousens, et al., Tregitope update: Mechanism of action parallels IVIg, Autoimmun. Rev. 12 (2013) 436-443, herein incorporated by reference in its entirety), and were repeated for comparison with FV621. The HLA-DRB1 alleles selected for the HLA binding assays represent a wide array of class II alleles, known as “supertype alleles” that share similar binding peptide side-chain preferences for their binding pockets.

The HLA binding assay used for these studies was originally described by Steere et al (A. C. Steere, et al., Antibiotic-refractory Lyme arthritis is associated with HLA-DR molecules that bind a Borrelia burgdorferi peptide, J. Exp. Med. 203 (2006) 961-971, herein incorporated by reference in its entirety) and has been standardized by EpiVax to validate in silico binding predictions. This assay has been described in previous publications in additional detail (e.g., M. Ardito, An Integrated Genomic and Immunoinformatic Approach to H. pylori Vaccine Design, Immunome Res. 7 (2011), herein incorporated by reference in its entirety).

### Human Peripheral Blood Mononuclear Cells (PBMCs)

PBMCs from healthy donors were isolated from leukocyte reduction filters purchased from the Rhode Island Blood Center (RIBC) in Providence, R.I. High resolution HLA class II haplotyping of donor PBMCs was performed at the Transplant Immunology Laboratory at Hartford Hospital in Hartford, Conn. The HLA alleles possessed by individual donors included in this panel represented HLA-DR alleles covering 95% of the allele-specific MHC-binding preferences reported for human populations (data not shown).

For the T reg assays, PBMCs were thawed according standard procedure, labeled with carboxyfluorescein succinimidyl ester (CFSE), and rested overnight in 96 well culture plate prior to use in in vitro assays. To evaluate the dose response of FV621 on the viability, cells were incubated with FV621 for 2, 3, 5 and 7 days in culture and live cells evaluated by flow cytometry using a live-dead viability dye and by counting in a cellometer (Nexcelom) using trypan blue exclusion. All assays were performed in RPMI complete medium: RPMI-1640 + GlutaMax (Life Technologies) containing 10 mM HEPES buffer (Life Technologies), 2 mM L-glutamine (Life Technologies), 50 μg/ml Gentamicin (Life Technologies), 10% Human AB serum (Sigma), MEM Non-essential amino acids (Gibco) and 55 μM β-Mercaptoethanol (Gibco).

Tetanus Toxoid Bystander Suppression (T reg) Assay

To evaluate the regulatory potential of the established and predicted Tregitope peptides and peptide controls, we adapted a previously published Tetanus Toxoid Bystander Suppression Assay (TTBSA) that measures the inhibitory capacity of potential regulatory peptides on the recall response of human CD4 T cells to the tetanus toxoid (TT) antigen [30]. TT vaccination is a routine, nearly universal immunization, such that anonymous blood bank blood donor PBMCs can be used for in vitro assays that require TT-specific memory T cells.

The modified TTBSA is performed as follows: PBMCs were labeled with CFSE (eBioscience) cell proliferation dye (2.5 μM) and then plated at 3×10⁵ cells per well on U-bottom 96-wells plates (Falcon) in RPMI complete media. Labeled cells are rested overnight at 37° C., 5% CO₂ and the following day, the cultures are stimulated with candidate Tregitope peptides or control peptides. Test peptides were solubilized in a minimal amount of DMSO (<1%) and added to the culture medium at a range of concentrations (8, 16, 24, or 5, 10, 15, 20 μg/mL). Tetanus toxoid (TT) (Astarte Biologics, cat no. 1002) at 0.5 μg/ml was then added to all wells including positive control wells without Tregitope peptides. The cells are cultured for 6 days, harvested at day 7 and stained for expression of cell surface and intracellular markers and analyzed by flow cytometry. In addition to testing the effect of FV621 on TT, a whole antigen, we also tested whether the immune response to CEFT peptides (obtained from Panatecs, PA-CEFT-001) would be modified by FV621. This CEFT suppression assay is performed using the same timeline and reagents as the TTBSA, by simply replacing TT with CEFT peptides at 0.5 mg/ml.

### HSA-FV621 Peptide Conjugate in Tetanus Toxoid Bystander Suppression Assay

As peptide drugs are known to have an extremely short half-life in humans, we explored whether human serum albumin (HSA)-conjugated FV621 could prolong the serum half-life of FV621 while also targeting the delivery of the peptide to APCs, with the aim of translating the conjugated product to in vivo pre-clinical studies. The conjugation process involves a maleimide linkage, a process that has been utilized for drug delivery in previous clinical trials. This maleimide-based chemistry can be used to covalently link a payload (here, Tregitope peptide FV621) to HSA in a 1:1 stoichiometry. Maleimidopropionamido (MPA) forms a stable thiol ester conjugate with the available free Cys34 in HSA. We used recombinant HSA (kindly provided by Albumedix, Nottingdam UK) to conjugate FV621 and tested inhibitory capacity comparing to unconjugated FV621 in TTBSA.

### CD8+ T Cell Bystander Assay

To assess whether FV621 also suppressed CD8+ T cell recall responses, we co-incubated PBMCs from healthy donors cultured as described above (in the TTBSA) but substituted a mixture of class I peptides derived from Cytomegalovirus, Epstein-Barr virus and Influenza virus (CEF peptide pool, available from Mabtech, cat no. 3615-1), for Tetanus Toxoid. The cells were incubated with CEF alone or with the addition of FV621 peptide. In a previous experiment performed with IgG Tregitopes [22], MHC class I-restricted CD8+ T cell responses were measured in the presence or absence of IgG Tregitopes after six days of incubation (and epitope-specific CD8+ T cell responses were suppressed). These types of assays serve to demonstrate that the effect of Tregitopes (IgG-derived or FV621) is not due to competition at the MHC binding groove by similar-binding peptides.

### Flow Cytometry and Gating Strategy

Cells were washed with 1×PBS +5% fetal bovine serum (FBS) and stained with Live/Dead Violet fixable stain (Life Technologies) in 1×PBS (Gibco) at 4° C., washed and stained with surface receptor antibodies in 1×PBS +5%FBS in the dark at 4° C. Cells were fixed with Fixation/Permeabilization Buffer (eBioscience) in the dark at room temperature for 30 min and stained for intracellular markers in Permeabilization Buffer (eBioscience) at 4° C. Stained cells were acquired and data collected on an Attune NxT Cytometer with three laser capacity (violet-405 nm, blue-488 nm, and red-637 nm) (Life Technologies) and analyzed using FlowJo software (Treestar, Inc). For gating, after exclusion of doublets, lymphocytes were first identified by a low forward scatter (FSC) and low side scatter (SSC) gate. Dead cells were excluded by Live-Dead staining. The following antibodies were used in this assay: CD3 (OKT3)-PECy7, CD4 (OKT4)-BV410, CD8 (SK1)-APC, CD25 (BC96)-AF700, CD127-PE from Biolegend. Granzyme B (GB11)-AF647, HLA-DR (L243)-FITC, FoxP3 (PCH101)-PECy5.5, CD11c-PE (3.9) and carboxyfluorescein (CFSE) were purchased from eBiosciences.

Proliferation of CD4+ T cells were evaluated by dilution of CFSE with the proliferating population identified as CFSE^{low}. The gating strategy for proliferation of CD4 T cells is illustrated in Supplementary FIG. 1A. CD4+ T effector cells were identified by the expression of CD25^{hi} and FoxP3^{int} and CD4+ T regulatory cells were identified by the expression of CD127^{low}CD25^{hi} and FoxP3^{hi}. Antigen presenting cells (APCs) were identified by the expression of CD11c and HLA-DR. Additional information on gating strategies for cell surface markers (CD8 T cells, CD4 T cells, Tregs, APC) and proliferation are illustrated in the corresponding result sections.

### OVA Immunogenicity Mouse Model

To demonstrate the in vivo effect of FV621, we used a well-established OVA immunogenicity model in C57BL/6 mice. Mice were sensitized with OVA in CFA at day zero and the sensitization repeated with OVA in IFA at day 14. FV621 was emulsified in CFA and delivered with the OVA treatment at day zero to evaluate the inhibitory effect on immune response to OVA. Anti-OVA antibody titers in serum were analyzed at day 17 by ELISA, and IFNg production was also analyzed in spleen cells by ELISpot assay using Mabtech kit.

### RNA Extraction and Gene Expression Analysis

Samples containing PBMCs stimulated with either CD3/CD28 or FV621 for 6 days and total RNA were prepared according to the manufacturer's protocol (RNeasy Mini kit, Qiagen #74104). RNA samples were labeled using the Affymetrix WTPLus kit according to manufacturer's guidelines, and probed using the Clariom S Human Array. Raw data generated from Clariom S Arrays were processed using Affymetrix Expression Console Software. CEL files containing feature intensity values were converted into summarized expression values using by Robust Multi-array Average (RMA) which consists of background adjustment, quantile normalization and summarization across all chips. All samples passed QC thresholds for hybridization, labeling and the expression of housekeeping gene controls.

### Statistical Analysis

Statistical analysis was performed using Prism software (GraphPad version 8.3). The Student's t-test (unless otherwise indicated, unpaired, two-tailed) was used to compare the significance of differences between TT stimulated cells to Tregitope treated cells or the indicated experimental groups. Differences were considered significant when p<0.05 (*), very significant when p<0.01 (**), highly significant when p<0.0002 (***), and extremely significant when p<0.0001 (****). The ordinal test (one-way repeated) ANOVA was used for gene expression analysis. This gene array featured 1,458 genes that showed contrasting gene expression in cells incubated with FV621 or CD3+CD28 stimulation.

### Results

FV621 Binds with High Affinity to Multiple HLA class II HLA DR Alleles

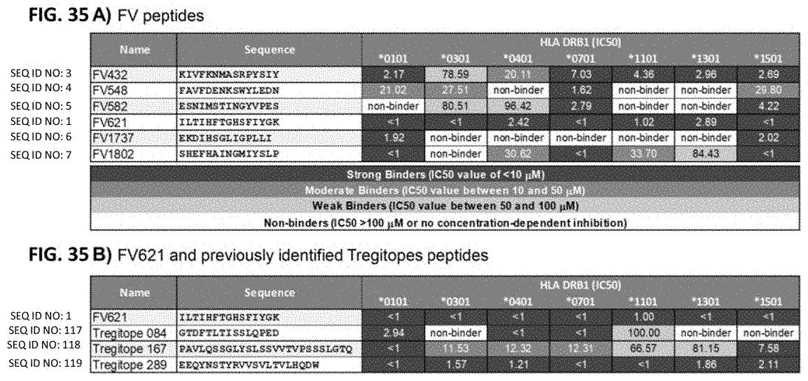

The major histocompatibility complex proteins (MHC, also known as HLA in humans) play a critical role in the development of an effective immune response or in activating regulatory T cells to prevent or diminish immune responses as they bind to and present processed peptide antigens on the surface of antigen presenting cells to diverse T cell populations. In investigating potential tolerogenic peptides in FV, a high abundance and highly conserved human protein, we used EpiMatrix to identify peptides likely to bind multiple human class II HLA DRB1 molecules and evaluated them in an in vitro binding assay. We found that six FV peptides which had been selected for promiscuous binding affinity to HLA DR showed moderate to strong binding to the panel of HLA alleles evaluated in this assay, although FV548, FV582 and FV1737 were somewhat more restricted in the breadth of binding to the full range of HLA DR alleles (FIG. 35A). Both FV621 and FV432 demonstrated binding to nearly all of the HLA DRB1 alleles tested (HLA DRB1*0101, *0301, *0401, *0701, *1101, *1301 and *1501) with strong affinity as reflected by assay results showing very low half-maximal inhibitory concentration (IC₅₀) values, calculated from the dose-response curve (FIG. 35A).

The in vitro HLA binding assay was repeated so as to compare, in the same assay, the binding affinity of FV621 to IgG-derived Tregitopes 289, 167 and 84. As shown in FIG. 35B, human Tregitope 289 binds with strong affinity across multiple HLA alleles, while Tregitope 167 binds with moderate affinity to HLA DRB1*0301, *0401, *0701 and with strong affinity to HLA DRB1*0101 and *1501. Tregitope 84 (a Tregitope found in the Fab domain of IgG) binds with strong affinity to HLA DRB1*0101, *0401, *0701 and DRB1*1501, and with moderate affinity to HLA-DRB1*1101. Thus, as predicted, the six FV peptides including FV621 showed moderate to high binding affinity that is very similar to IgG Tregitopes, binding to multiple HLA-DRB1 alleles. High-binding Tregitope 289 was identified as the most appropriate “positive control” comparator for FV621 for subsequent in vitro evaluations using human PBMC. FV621 peptides featuring single amino acid mutations at the HLA-binding P1 and TCR-facing P5 were also synthesized and tested in binding assays for correlation with in vitro results in the TTBSA.

### Immunomodulatory Effect of Factor V-Derived Peptides on the CD4+ Tetanus Toxoid Recall Response

Stimulating PBMCs from healthy donors with TT generates a robust memory CD4+ T cell recall response to TT, as demonstrated by the upregulation of several activation markers on the cell surface by flow cytometry. TT consistently stimulated CD4+ T cells by 5-60% in different donors (data not shown). The variation of TT response in healthy donors is likely due to the TT vaccination history of the donors as well as to the distribution of TT-specific precursor memory CD4+ T cells in the PBMC sample thawed for each of the TTBSA assays.

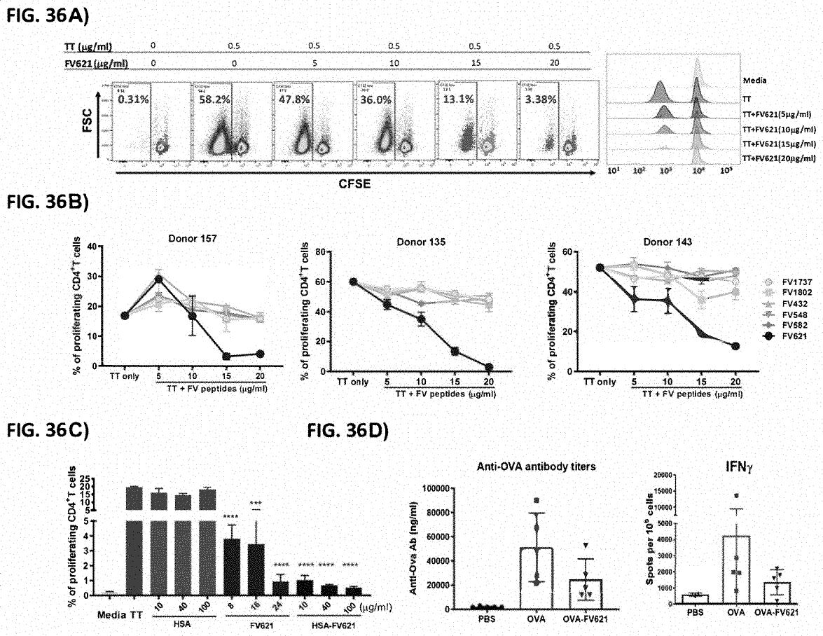

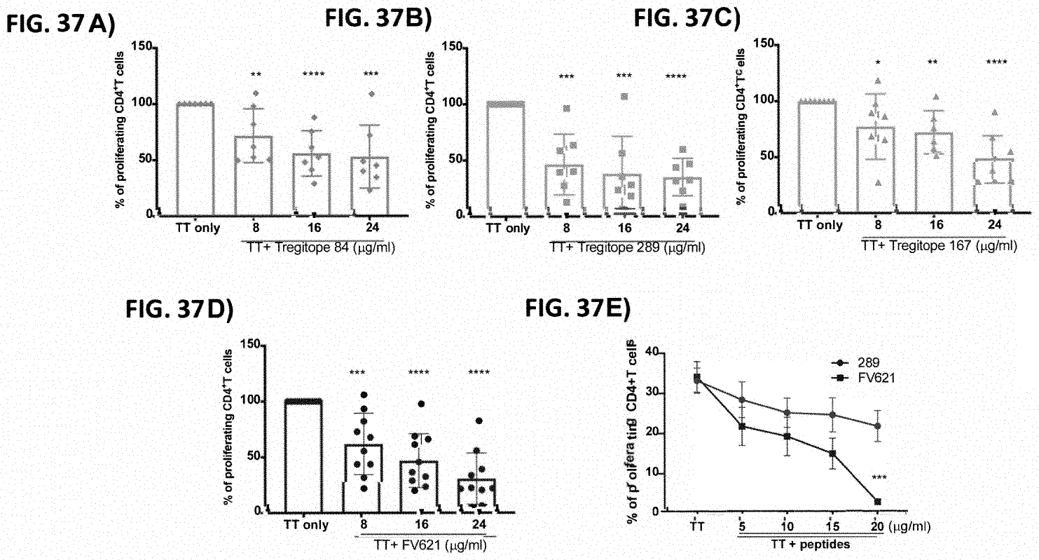

Using the Tetanus Toxoid Bystander assay (TTBSA), we evaluated the six selected FV peptides for their capacity to inhibit the T effector memory response to TT in PBMCs derived from a panel of donors (FIGS. 36A-D). FV peptides which demonstrated moderate to strong binding affinity to two or more HLA DRB1 alleles were selected for additional testing in vitro and compared with three previously identified Tregitope peptides (Tregitope 289, 167 and 84) in the TTBSA (FIGS. 37A-E).

As shown for one representative donor in FIG. 36A (left), Tetanus Toxoid stimulation increased CD4+ T cell proliferation by roughly 60-fold over control in a CFSE dilution assay. The histogram in FIG. 3A, right side, shows the degree of inhibition of CD4 T cell proliferation by FV621. The addition of FV621 significantly suppressed proliferation of CD4+ T cells to TT in a dose-dependent manner by as much as 80% at the highest dose (20.0□g/ml) of FV621. This observation was repeated for multiple donors (FIG. 3).

For comparison, five other FV peptides (FV432, FV548, FV582, FV1737 and FV1802) were also tested for their ability to inhibit TT-specific immune-response in the TT-bystander suppression assay using PBMC from three HLA-diverse donors. Despite binding nearly as well as FV621, as shown in FIG. 36B, the five other FV peptides did not inhibit the CD4+T memory response in the TTBSA, for the same donors. In particular, despite having very similar HLA DR binding affinity across multiple alleles to FV621 (FIGS. 35A-B), FV432 failed to significantly inhibit CD4+ T cell proliferation in the TTBSA to the same extent as FV621. We also examined the effect of FV621 and other FV peptides (FV 432, FV548, FV582, FV1737 and FV1802) on CD4+ T cell proliferation or the CD4+ T cell activation markers CD69 and HLA DR on CD4+ T cells. Of the six FV peptides, only FV621 inhibited T effector proliferation and modified CD69 and HLA DR expression (data not shown).

### Effect of FV621 Delivery in HSA-Conjugate in TTBSA

Translation of Tregitope peptides such as FV621 to potential clinical use will necessitate establishing optimal pharmacokinetics, prolonging the half-life of the peptide and enhancing its stability under physiological conditions. Albumin prolongs the half-life of conjugated payloads by binding to the neonatal Fc receptor (FcRn) and recirculating. Albumin is also avidly taken up by antigen-presenting cells, potentially decreasing the need for more frequent or repeat dosing of Tregitopes in human therapy. We recently demonstrated that a human serum albumin (HSA)-Tregitope (IgG Tregitopes 084+167) fusion protein administered with Pre-pro-insulin (PPI) peptides promoted antigen-specific tolerance in Non Obese Diabetes (NOD) mice. The HSA-Tregitope fusion, when administered with the target PPI peptide, significantly delayed onset of diabetes, as compared to HSA-Tregitope fusion without PPI, demonstrating improved glucose control over 49 days after treatment a 20 week period. However, Tregitope peptides fused to albumin were subsequently shown to be cleaved in yeast expression systems, making large-scale production of this fusion product unlikely.

We therefore evaluated the maleimide linkage method of attaching the FV621 Tregitope to albumin, and evaluated whether FV621-linked HSA would perform well in the TTBSA, as compared to free FV621 peptide. FV621 peptide alone (no conjugation) inhibited CD4+ T cell proliferation in a dose-dependent manner, while FV621 conjugated with HSA also significantly inhibited CD4+ T cell proliferation but at tenfold lower doses of total Tregitope. The amount of FV621 delivered by the FV621-HSA-conjugate was 0.25, 1.0, 2.5 μg/ml (for 10, 40 and 100 μg/ml of HSA-conjugate, respectively), which is 10-30 fold lower than that represented in the free peptide stimulation (8, 16, 24 μg/ml). In comparison, HSA alone (not conjugated to FV621) did not have any impact on the TT mediated CD4+ T cell proliferation (FIG. 36C).

The greater efficacy of the conjugated FV621 product on suppression of T effector memory cells in vitro shown in FIG. 36C may be related to the increased half-life of the conjugates or to increased efficiency of Tregitope presentation. These data further suggest that HSA-conjugation using the maleimide linkage is worth exploring as a flexible delivery vehicle for FV621 alone, or in combination with target antigens, for inducing tolerance in vivo.

### Effect of FV621 on Immune Response to OVA In Vivo

We have previously reported that Tregitopes derived from IgG were effective in a wide range of in vivo autoimmune disease models. Prior to performing more intensive autoimmune model studies, we decided to evaluate the effect of FV621 treatment in a simple in vivo model involving OVA immunization of C57B1/6 mice. C57B1/6 mice were immunized with OVA emulsified in Complete and Incomplete Freund's Adjuvant (CFA/IFA) on days zero and 14. In the treatment arm, FV621 was added to the OVA in CFA at day zero. In the control arm, saline was added to the OVA/CFA. Titers of anti-OVA antibodies were measured in serum of treated mice and compared to untreated mice. OVA-induced IFNγ production by ELISpot was also measured in T cells obtained from mouse spleens at day 17. As compared to mice given a sham (saline) treatment with OVA, co-administration of FV621 peptide administered with OVA antigen inhibited anti-OVA antibody titers in serum and also suppressed IFNg production in spleen cells (FIG. 36D).

### FV621 Peptide Inhibits CD4+ T Cell Proliferation in High and Lower Level TT Responsive Donors Compared to IgG Tregitope

We then asked whether there was a dose response effect of FV621 in suppression in the TTBSA and whether FV621 could inhibit very high level responders to TT. We therefore performed a dose-response assessment of FV621 and IgG Tregitopes and compared their inhibition of TT induced memory CD4+ T effector responses (FIGS. 37A-E). Tregitope 289 (FIG. 37A), Tregitope 84 (FIG. 37B) and Tregitope 167 (FIG. 37C) significantly inhibited the TT induced memory response to CD4 T cell proliferation. Comparing the effect of Tregitopes 289, 167, 84 with FV621 (FIG. 37D) in donors that respond to TT at the 5-20% level of proliferation, we found that the inhibition by FV621 Tregitope is similar to the inhibition by Tregitopes 289, 167 and 84. For this comparison across donors, data representing the maximum (%) of CD4+ T cell proliferation in response to TT was normalized to 100% of the total; all other conditions resulted in a reduction of the TT response by as much as 80%.

To better elucidate the inhibitory capacity of FV621 as compared to Tregitope 289 we evaluated their suppressive effect in robust TT responsive donors, (30-40% CD4+ T cell proliferation in response to TT), in the TTBSA. We found that the FV621 Tregitope is better able to inhibit CD4+ T cell proliferation compared to Tregitopes 289 in high TT responsive donors with significant reduction in proliferation at the highest dose tested (20.0 μg/ml) compared to Tregitope 289 (FIG. 37E). Thus, while exhibiting similar HLA binding affinity, FV621 more potently suppresses robust TT-induced CD4+ T memory cell proliferation as compared to Tregitope 289. Variation in Tregitope-specific Treg precursor frequencies and/or alternative modes of action by which FV621 Tregitopes suppress T effectors may explain the observed differences in inhibitory capacity by the recruited Tregs.

The strong suppressive effect of FV621 was confirmed in TTBSA performed using PBMCs from 20 healthy donors at in vitro Tregitope dosing levels of 15.0 μg/ml and 20.0 μg/ml (data not shown). FV621 also inhibited TT induced CD4+ T memory responses across a broad spectrum of donor HLA types (data not shown) suggesting that FV621 has the potential to suppress effector T cell responses across population groups.

Next, we evaluated whether a modification of the TCR facing residue within the predicted HLA-binding domain of FV621 would impact the suppressive effect of FV621. A point mutation at position G628H (glycine changed to histidine) significantly diminished suppressive capacity of FV621 on TT-induced memory CD4 T cell proliferation in TTBSA (data not shown). The HLA binding capacity of this peptide (G628H) was identical to FV621, suggesting that the impact of the modification is limited to TCR recognition by CD4 T cells. In contrast, a peptide containing a single modification to the HLA-facing amino acids (1624A) bound with slightly lower affinity (data not shown), but did not diminish the suppressive effect of FV621.

### FV621 Peptide Inhibits T Effector Cell Activation and Induces T Regulatory Cell Proliferation

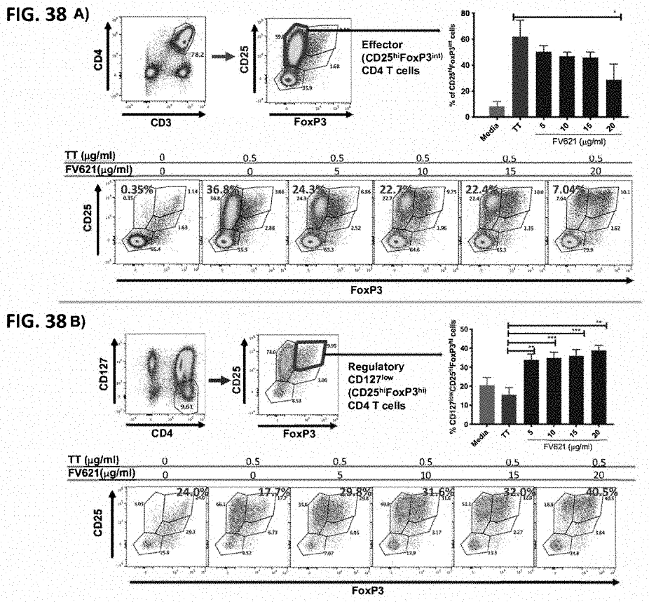

To further characterize the inhibitory capacity of FV621 on the CD4+ T effector cell populations and investigate its impact on T regulatory cells, we evaluated the effect of FV621 in culture with PBMC from donors with diverse HLA DR haplotypes. CFSE labeled PBMCs from healthy donors were stimulated with TT in the presence or absence of FV621 for 6 days and the proliferation of T effector and regulatory T cells was assessed. T regulatory cells were identified by the expression of CD127^{low}, CD25^{hi} and FoxP3^{hi} (FoxP3 is a transcription factor and major regulator of Treg development but is also transiently expressed in activated T effector cells), while CD4+ T effector cells were identified as CD25^{hi}FoxP3^{int} in the CD4+ gated population. As shown for a single representative donor FIG. 38A (lower panel), proliferation of CD4+CD25^{hi}FoxP3^{int} T effector cells was detected in the presence of TT alone, while co-treatment of the cultures with increasing concentrations of FV621 significantly inhibited the percentage of activated CD4+ T effector cells. Reciprocally, we observed a dose-dependent increase in percentage of Tregs in cultures treated with FV621 plus TT (FIG. 38B).

The observed increase in the absolute number of Tregs with increasing dose levels of FV621 may be due to (1) conversion of TT-specific T effectors to adaptive or iTregs (likely); (2) elimination of TT-specific T effectors from the mixed population of cells (possible); or (3) proliferation of natural Tregs (unlikely). We hypothesize that FV621 treatment may be increasing the proportion of antigen-specific T regulatory cells by decreasing the TT-specific T effector population as indicated in the same TTBSA assay (FIGS. 38A-B); this is supported by our granzyme B studies (see below). Alternatively, the shift in Treg to T effector ratios may be due to conversion of T effectors to adaptive or iTregs. This hypothesis is supported by previous studies that showed that Tregitope treatment of human PBMCs in vitro converted tetramer-stained Birch Pollen specific T effector cells to adaptive or iTregs. Conversion of antigen-specific T effector cells to antigen-specific T regs has also been documented in D011.10 mice treated with Tregitopes 167 and 289 [8].

Regardless of the mechanism, we note that the ratio of activated T regulatory cells to T effector cells has been described as a determining factor in the maintenance of tolerance and in the potential for treatment of allergic and autoimmune diseases. FV621 treatment in the presence of TT shifts the balance of T effector cells and T regulatory cells by increasing the overall ratio of Treg to Teff cells (data not shown).

### Inhibition of CD8 T Cell Response by FV621

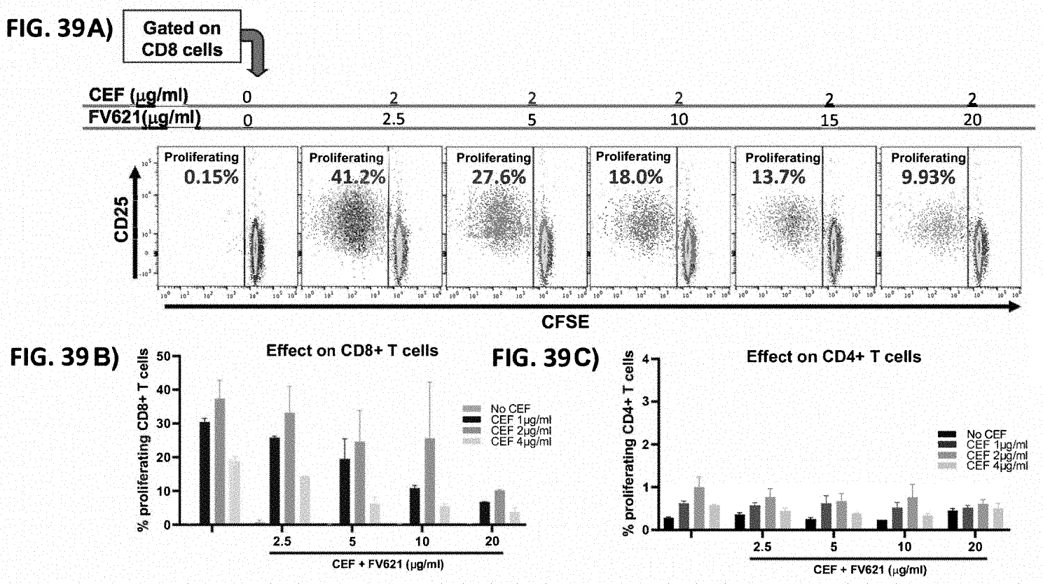

Another potential mechanism by which FV621 activated Tregs could mediate an inhibitory effect on T eff cells is by secretion of suppressor cytokines and/or modulation of cell surface inhibitory receptors on dendritic cells, and which could explain the effect on both activated CD4+ and CD8+ T cells via inhibition arising from APCs. CD8+ T effector memory cells can be induced to proliferate in response to engagement with peptide-loaded MHC class I molecules wherein the peptides are derived from antigens to which most of the population have been previously exposed, either through vaccination or by natural infection. As shown in FIG. 39A, where FV621 was incubated with class I restricted T cell epitopes, FV621 strongly inhibited memory CD8+ T cell activation (CD25^{hi}) and proliferation (CFSE^{low}) in response to CEF stimulation of PBMCs in a dose-dependent manner (FIG. 39A and FIG. 39B) but CD4+ T cells were not significantly not stimulated by CEF peptides (FIG. 39C). This suggests that the inhibitory effect of FV621 on T cells is not due to competition for HLA binding on the APC (binding of Tregitope FV621 to MHC class II cannot interfere with CEF peptide binding to MHC Class I, and that Tregitopes can modulate both CD4+ and CD8+ effector T cell responses. The independent effect of Tregitopes on Class I-restricted T cell responses was also previously observed using individual MHC Class I-restricted, virus-derived class I epitopes. This effect appeared to be due to direct contact between Tregs and CD8+ T cells, as suggested by the observation that IgG Tregitope-stimulated PBMCs were not suppressive of CD8 T cell responses when separated from the CD8+ T cells in transwell plates.

### FV621 Modulation of T Cell Response Following Non-Specific Activation by CD3/CD28

To further evaluate whether FV621 is broadly suppressive and corroborate the previous observation, we also performed an in vitro polyclonal T cell activation assay using healthy donor PBMCs. Cells were labelled with CFSE, cultured in complete media in a 96 well round bottom plate overnight and stimulated with anti-human CD3/CD28 dynabeads at a cell to bead ratio of 5:1 for 6 days alone or in combination with FV621. After six days of incubation, cells were stained with surface and intracellular markers and analyzed by flow cytometry. FV621 inhibited CD4+ T cell proliferation similar to its effects in the TTBSA (data not shown) following polyclonal CD4+ T cell stimulation, supporting our previous observation, that the effect of FV621 is not due to binding competition with antigen at the MHC-T cell interface.

We have also tested the relative contribution of T regulatory peptide (FV621) in the presence of multiple immune stimulatory CD4 epitopes (CEFT: pool of 23 peptides for MHC Class II-restricted effect) using an in vitro bystander suppression assay. We demonstrated that CEFT stimulates CD4 T cells and co-incubation with FV621 significantly inhibit proliferation of CD4 T cells in a dose dependent manner (data not shown), confirming that FV621 is immunomodulatory, whether added to peptide or to whole antigen, in vitro.

### FV621 Stimulation Downregulates Antigen Presenting Cells

We then investigated whether FV621 and other Tregitopes may modulate T cell responses (both CD8 and CD4) by modulating antigen presenting cells (APCs) from an immunostimulatory to a tolerogenic phenotype (also known as TolDC). The expression of HLA-DR, CD80 and CD86 by APCs critically contributes to the activation of antigen specific Treg and T effector cells. The dendritic cell surface markers CD11c, HLA DR, CD80 and CD86 have been shown to be down-modulated by known immune suppressive agents including IVIG and other Tregitope-like peptides.

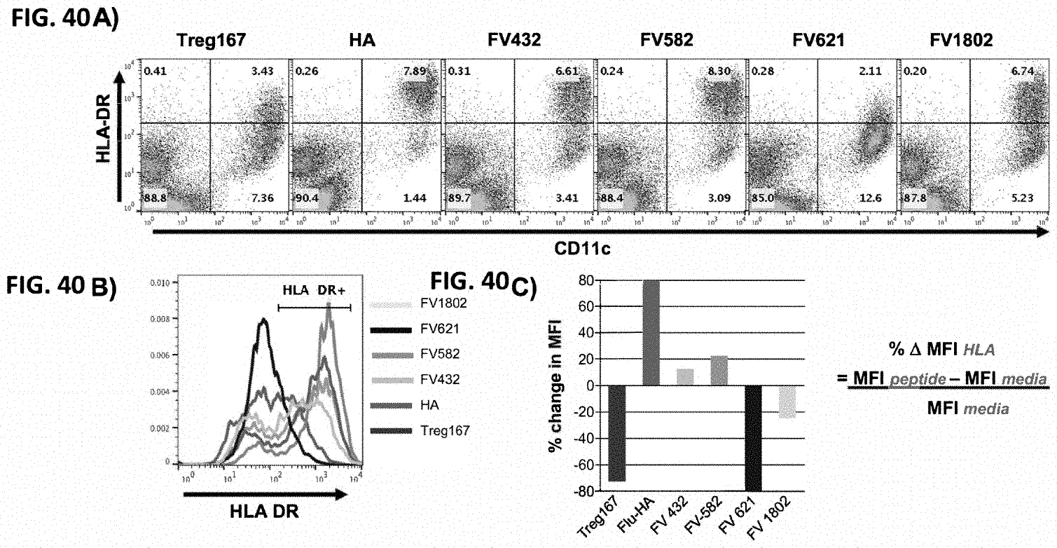

We therefore compared the effect of incubating FV621 and Tregitope 167 with PBMCs on the expression of CD11c and HLA-DR (FIGS. 40A-C) in vitro. Healthy donor PBMCs were incubated with the immunostimulatory Influenza hemagglutinin HA306-318 (negative control), Tregitope 167 (positive control) and FV-derived peptides, and cells were stained for the surface expression of HLA-DR on day 4. Compared to stimulation with HA306-318 alone, both Treg167 and FV621 stimulation induced significant downregulation of HLA-DR (FIG. 40A). We also compared the mean fluorescence intensity (MFI) of HLA-DR in CD11c⁺ cells (FIG. 40B) and percent changes in MFI over media alone stimulation (FIG. 40C). Comparing both MFI and percent changes to the expression of HLA DR demonstrated that FV621 was more efficient than Tregitope 167 and the other FV peptides at reducing HLA DR expression on antigen presenting cells. FV621 stimulation also downmodulated CD86 expression in CD11c+ cells compared to HA stimulation (data not shown). A recent observation that Tregs “steal” HLA DR from dendritic cells [39] suggests that this may be one mechanism by which CD11c dendritic cells lose their HLA DR when co-cultured with FV621-activated Treg cells.

### Effect of FV621 Peptide on Granzyme B Expression in Regulatory T Cells Upon Activation

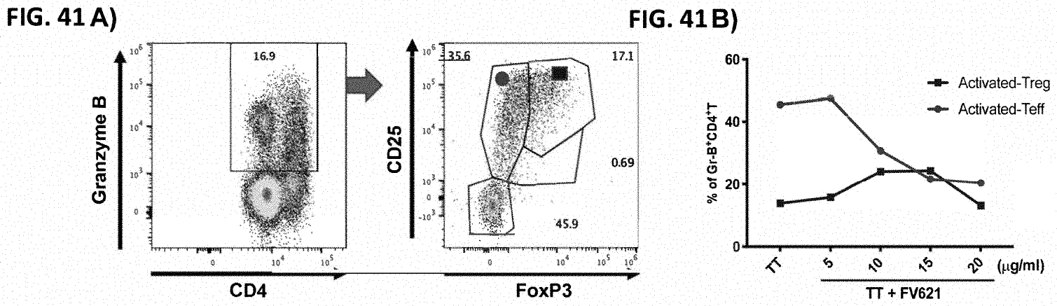

T regulatory cells have been shown to upregulate the expression of granzyme-B and perforin, which can lead to cytolysis of T effector cells and induce immune suppression by this means. We thus evaluated the expression of granzyme-B in T regulatory cells after stimulating with TT in the presence or absence of FV621. We gated on highly activated Tregs (CD25^{hi}FoxP3^{hi}) in the granzyme-B expressing cell population (FIG. 41A). FV621 treatment increased the (%) frequency of granzyme-B⁺ activated T regulatory cells and decreased activated T effector cells in a dose-dependent manner (FIG. 41B). Thus, granzyme B release may be one mechanism by which FV621-responding Tregs regulate CD4+ T effector function.

### FV621 Stimulation Shows Differential Gene Expression Profile as Compared to Conventional T Cell Stimulation

To understand the mechanism of action of FV621 treatment in suppressing T effector memory T cell response, we analyzed PBMC gene expression analysis by Affymetrix Clariom S assays. The heatmap was generated (data not shown) that shows that the effect of FV621 on cultured PBMC has a differential effect on expressed genes, as compared to conventional stimulation with CD3/CD28. Comparing with selected gene clusters between FV621 and CD3/CD28 stimulation we found that FV621 stimulation differentially upregulates cluster 1 genes and down-regulates cluster 2 genes.

### Effect of FV621 Toxicity on Cell Viability

To rule out the possibility that the effect of FV621 was related to toxic effects of the peptide, which might simulate a suppressive effect in vitro, we evaluated the viability of cells in culture with FV621 using two different methods. Cells were cultured in the presence of FV621 in a 96 well plate for 2, 3, 5 and 7 days after which cells were collected and stained with live-dead viability dye and analyzed by flow cytometry (data not shown) and by trypan blue exclusion and counting with a cellometer (Nexcelom). The absolute number of live cells that was counted at days 2, 3, 5 and 7 after stimulation with different doses of FV621 Tregitope was similar by flow cytometry. Although we observed a slight (10-15%) decreased viability at the highest dose of FV621 peptides by trypan blue exclusion, this may have been due to assay variability in the measurement of live cells or active killing of bystanders by activated Tregs (data not shown).

### Equivalents

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1-88. (canceled)

89. A polypeptide comprising one or more T-cell epitope polypeptides linked to a heterologous polypeptide, wherein the T-cell epitope polypeptide consists of an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

90. The polypeptide composition of claim 89, wherein the T-cell epitope polypeptide is linked to the N-terminus of the heterologous polypeptide.

91. The polypeptide of claim 89, wherein the T-cell epitope polypeptide is linked to the C-terminus of the heterologous polypeptide.

92. The polypeptide of claim 89, wherein the T cell epitope is fused to or inserted internally within the heterologous polypeptide.

93. The polypeptide of claim 89, wherein the heterologous polypeptide comprises a biologically active molecule and wherein the biologically active molecule is selected from the group consisting of an immunogenic molecule, a T-cell epitope, a viral protein, and a bacterial protein.

94. The polypeptide of claim 89, wherein the heterologous polypeptide is a Factor VIII molecule or Factor VIII replacement protein or supplement.

95. The polypeptide of claim 89, wherein the heterologous polypeptide is a Factor V molecule or Factor V replacement protein or supplement.

96. The chimeric polypeptide composition of claim 89, wherein the heterologous polypeptide is operatively linked to the T-cell epitope polypeptide.

97. A method of inducing regulatory T-cells to suppress immune response in a subject comprising administrating to the subject a therapeutically effective amount of a polypeptide, wherein the polypeptide composition comprises one or more T-cell epitope polypeptides linked to a heterologous polypeptide, wherein the T-cell epitope polypeptide comprises an amino acid sequence selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

98. The method of claim 97, wherein the T-cell epitope polypeptide is fused to the N-terminus of the heterologous polypeptide.

99. The method of claim 97, wherein the T-cell epitope polypeptide is fused to the C-terminus of the heterologous polypeptide.

100. The method of claim 97, wherein the T cell epitope is fused to or inserted internally within the heterologous polypeptide.

101. The method of claim 97, wherein the heterologous polypeptide comprises a biologically active molecule and wherein the biologically active molecule is selected from the group consisting of an immunogenic molecule, a T-cell epitope, a viral protein, and a bacterial protein.

102. The method of claim 97, wherein the heterologous polypeptide is a Factor VIII molecule or Factor VIII replacement protein or supplement.

103. The method of claim 97, wherein the heterologous polypeptide is a Factor V molecule or Factor V replacement protein or supplement.

104. The method of claim 97, wherein the polypeptide composition further comprises an effective amount of one or more antigens and/or allergens.

105. The method of claim 97, wherein the T-cell epitope composition suppresses an effector T-cell response.

106. The method of claim 97, wherein the T-cell epitope composition suppresses a helper T-cell response.

107. The method of claim 97, wherein the T-cell epitope composition suppresses a B-cell response.
